(19) United States
(12) Patent Application Publication
(10) Pub. No.: US 2024/0191281 A1
(43) Pub. Date: Jun. 13, 2024

(54) PROGRAMMABLE NUCLEASES AND METHODS OF USE

(71) Applicant: Mammoth Biosciences, Inc., Brisbane, CA (US)

(72) Inventors: Benjamin Julius RAUCH, San Francisco, CA (US); Aaron DELOUGHERY, San Francisco, CA (US); Matan DRORY RETWITZER, Foster City, CA (US); David PAEZ-ESPINO, Concord, CA (US); Caleb TRECAZZI, San Francisco, CA (US); Lucas Benjamin HARRINGTON, San Francisco, CA (US); Janice Sha CHEN, San Francisco, CA (US); James Paul BROUGHTON, South San Francisco, CA (US); Clarissa Oriel RHINES, San Mateo, CA (US); William Douglass WRIGHT, Fairfield, CA (US); Matthew VEROSLOFF, South San Francisco, CA (US); Carley Gelenter HENDRIKS, Burlingame, CA (US)

(21) Appl. No.: 18/364,359

(22) Filed: Aug. 2, 2023

Related U.S. Application Data
(63) Continuation of application No. PCT/US22/15709, filed on Feb. 8, 2022.
(60) Provisional application No. 63/147,683, filed on Feb. 9, 2021.
(60) Provisional application No. 63/147,684, filed on Feb. 9, 2021.
(60) Provisional application No. 63/147,685, filed on Feb. 9, 2021.
(60) Provisional application No. 63/147,686, filed on Feb. 9, 2021.
(60) Provisional application No. 63/209,900, filed on Jun. 11, 2021.

(51) Int. Cl. C12Q 1/6823; C12N 9/22; C12N 15/11

(52) U.S. Cl. CPC C12Q 1/6823; C12N 9/22; C12N 15/11; C12N 2310/20; C12N 2310/3517

(57) ABSTRACT

The present disclosure provides compositions and methods of use for a Type VI CRISPR/Cas nuclease. Type VI CRISPR/Cas nucleases are able to bind to a target nucleic acid, thereby activating trans-collateral nuclease activity on nucleic acid reports. Furthermore, the present disclosure provides methods to modify ribonucleic acid sequences using the programmable nucleases disclosed herein.

### CROSS-REFERENCE

This application is a continuation of International Patent Application No. PCT/US2022/015709, filed Feb. 8, 2022, which claims the benefit of priority to U.S. Provisional Patent Application No. 63/147,683, filed Feb. 9, 2021, U.S. Provisional Patent Application No. 63/209,900, filed Jun. 11, 2021, U.S. Provisional Patent Application No. 63/147,685, filed Feb. 9, 2021, U.S. Provisional Patent Application No. 63/147,686, filed Feb. 9, 2021, and U.S. Provisional Patent Application No. 63/147,684, filed Feb. 9, 2021, the entire contents of each of which are incorporated herein by reference.

### SEQUENCE LISTING

The instant application contains a Sequence Listing which has been submitted via Patent Center and is hereby incorporated by reference in its entirety. Said Sequence Listing, created on Aug. 2, 2023, is named 203477-739301.xml and is 119,850 bytes in size.

### BACKGROUND OF THE INVENTION

Certain programmable nucleases can be used for genome editing of nucleic acid molecules and/or detection of nucleic acid molecules. There is a need for high efficiency, programmable nucleases that are capable of working under various sample conditions and can be used for genome editing and/or diagnostics.

### SUMMARY OF THE INVENTION

Disclosed herein is a non-naturally occurring composition that comprises in an aspect, a programmable nuclease and an engineered guide nucleic acid, wherein the programmable nuclease comprises an amino acid sequence that is at least 75% identical to any one of SEQ ID NOs: 1-27. In an embodiment, the programmable nuclease comprises an amino acid sequence that is at least 80% identical to any one of SEQ ID NOs: 1-27. In an embodiment, the programmable nuclease comprises an amino acid sequence that is at least 85% identical to any one of SEQ ID NOs: 1-27. In an embodiment, the programmable nuclease comprises an amino acid sequence that is at least 90% identical to any one of SEQ ID NOs: 1-27. In an embodiment, the programmable nuclease comprises an amino acid sequence that is at least 95% identical to any one of SEQ ID NOs: 1-27. In an embodiment, the programmable nuclease comprises an amino acid sequence that is at least 98% identical to any one of SEQ ID NOs: 1-27. In an embodiment, the programmable nuclease comprises an amino acid sequence that is at least 99% identical to any one of SEQ ID NOs: 1-27. In an embodiment, the programmable nuclease comprises an amino acid sequence of any one of SEQ ID NOs: 1-27. In an embodiment, the amino acid sequence of the programmable nuclease is at least 75% identical to any one of SEQ ID NOs: 1-27. In an embodiment, the amino acid sequence of the programmable nuclease is at least 80% identical to any one of SEQ ID NOs: 1-27. In an embodiment, the amino acid sequence of the programmable nuclease is at least 85% identical to any one of SEQ ID NOs: 1-27. In an embodiment, the amino acid sequence of the programmable nuclease is at least 90% identical to any one of SEQ ID NOs: 1-27. In an embodiment, the amino acid sequence of the programmable nuclease is at least 95% identical to any one of SEQ ID NOs: 1-27. In an embodiment, the amino acid sequence of the programmable nuclease is at least 98% identical to any one of SEQ ID NOs: 1-27. In an embodiment, the amino acid sequence of the programmable nuclease is at least 99% identical to any one of SEQ ID NOs: 1-27. In an embodiment, the amino acid sequence of the programmable nuclease is any one of SEQ ID NOs: 1-27. In an embodiment, the programmable nuclease comprises an amino acid sequence that is at least 75% identical to SEQ ID NO: 1, and the engineered guide nucleic acid comprises a sequence that is at least 75% identical to SEQ ID NO: 28; the programmable nuclease comprises an amino acid sequence that is at least 75% identical to SEQ ID NO: 2, and the engineered guide nucleic acid comprises a sequence that is at least 75% identical to SEQ ID NO: 29; the programmable nuclease comprises an amino acid sequence that is at least 75% identical to SEQ ID NO: 3, and the engineered guide nucleic acid comprises a sequence that is at least 75% identical to SEQ ID NO: 30; the programmable nuclease comprises an amino acid sequence that is at least 75% identical to SEQ ID NO: 4, and the engineered guide nucleic acid comprises a sequence that is at least 75% identical to SEQ ID NO: 31; the programmable nuclease comprises an amino acid sequence that is at least 75% identical to SEQ ID NO: 5, and the engineered guide nucleic acid comprises a sequence that is at least 75% identical to SEQ ID NO: 32; the programmable nuclease comprises an amino acid sequence that is at least 75% identical to SEQ ID NO: 6, and the engineered guide nucleic acid comprises a sequence that is at least 75% identical to any one of SEQ ID NOs; 28-32; the programmable nuclease comprises an amino acid sequence that is at least 75% identical to SEQ ID NO: 7, and the engineered guide nucleic acid comprises a sequence that is at least 75% identical to any one of SEQ ID NOs; 28-32; the programmable nuclease comprises an amino acid sequence that is at least 75% identical to SEQ ID NO: 8, and the engineered guide nucleic acid comprises a sequence that is at least 75% identical to any one of SEQ ID NOs; 28-32; the programmable nuclease comprises an amino acid sequence that is at least 75% identical to SEQ ID NO: 9, and the engineered guide nucleic acid comprises a sequence that is at least 75% identical to any one of SEQ ID NOs; 28-32; the programmable nuclease comprises an amino acid sequence that is at least 75% identical to SEQ ID NO: 10, and the engineered guide nucleic acid comprises a sequence that is at least 75% identical to any one of SEQ ID NOs; 28-32; the programmable nuclease comprises an amino acid sequence that is at least 75% identical to SEQ ID NO: 11, and the engineered guide nucleic acid comprises a sequence that is at least 75% identical to any one of SEQ ID NOs; 28-32; the programmable nuclease comprises an amino acid sequence that is at least 75% identical to SEQ ID NO: 12, and the engineered guide nucleic acid comprises a sequence that is at least 75% identical to any one of SEQ ID NOs; 28-32; the programmable nuclease comprises an amino acid sequence that is at least 75% identical to SEQ ID NO: 13, and the engineered guide nucleic acid comprises a sequence that is at least 75% identical to any one of SEQ ID NOs; 28-32; the programmable nuclease comprises an amino acid sequence that is at least 75% identical to SEQ ID NO: 14, and the engineered guide nucleic acid comprises a sequence that is at least 75% identical to any one of SEQ ID NOs; 28-32; the programmable nuclease comprises an amino acid sequence that is at least 75% identical to SEQ ID NO: 15, and the engineered guide nucleic acid comprises a sequence that is at least 75% identical to SEQ ID NO: 60; the programmable nuclease comprises an amino acid sequence that is at least 75% identical to SEQ ID NO: 16, and the engineered guide nucleic acid comprises a sequence that is at least 75% identical to SEQ ID NO: 61; the programmable nuclease comprises an amino acid sequence that is at least 75% identical to SEQ ID NO: 17, and the engineered guide nucleic acid comprises a sequence that is at least 75% identical to SEQ ID NO: 62; the programmable nuclease comprises an amino acid sequence that is at least 75% identical to SEQ ID NO: 18, and the engineered guide nucleic acid comprises a sequence that is at least 75% identical to SEQ ID NO: 63; the programmable nuclease comprises an amino acid sequence that is at least 75% identical to SEQ ID NO: 19, and the engineered guide nucleic acid comprises a sequence that is at least 75% identical to SEQ ID NO: 60; the programmable nuclease comprises an amino acid sequence that is at least 75% identical to SEQ ID NO: 20, and the engineered guide nucleic acid comprises a sequence that is at least 75% identical to SEQ ID NO: 64; the programmable nuclease comprises an amino acid sequence that is at least 75% identical to SEQ ID NO: 21, and the engineered guide nucleic acid comprises a sequence that is at least 75% identical to SEQ ID NO: 61; the programmable nuclease comprises an amino acid sequence that is at least 75% identical to SEQ ID NO: 22, and the engineered guide nucleic acid comprises a sequence that is at least 75% identical to SEQ ID NO: 65; the programmable nuclease comprises an amino acid sequence that is at least 75% identical to SEQ ID NO: 23, and the engineered guide nucleic acid comprises a sequence that is at least 75% identical to SEQ ID NO: 60; the programmable nuclease comprises an amino acid sequence that is at least 75% identical to SEQ ID NO: 24, and the engineered guide nucleic acid comprises a sequence that is at least 75% identical to SEQ ID NO: 65; the programmable nuclease comprises an amino acid sequence that is at least 75% identical to SEQ ID NO: 25, and the engineered guide nucleic acid comprises a sequence that is at least 75% identical to SEQ ID NO: 66; the programmable nuclease comprises an amino acid sequence that is at least 75% identical to SEQ ID NO: 26, and the engineered guide nucleic acid comprises a sequence that is at least 75% identical to SEQ ID NO: 67; or the programmable nuclease comprises an amino acid sequence that is at least 75% identical to SEQ ID NO: 27, and the engineered guide nucleic acid comprises a sequence that is at least 75% identical to SEQ ID NO: 68. In some embodiments, the programmable nuclease comprises an amino acid sequence that is at least 95% identical to SEQ ID NO: 1, and the engineered guide nucleic acid comprises a sequence that is at least 95% identical to SEQ ID NO: 28; the programmable nuclease comprises an amino acid sequence that is at least 95% identical to SEQ ID NO: 2, and the engineered guide nucleic acid comprises a sequence that is at least 95% identical to SEQ ID NO: 29; the programmable nuclease comprises an amino acid sequence that is at least 95% identical to SEQ ID NO: 3, and the engineered guide nucleic acid comprises a sequence that is at least 95% identical to SEQ ID NO: 30; the programmable nuclease comprises an amino acid sequence that is at least 95% identical to SEQ ID NO: 4, and the engineered guide nucleic acid comprises a sequence that is at least 95% identical to SEQ ID NO: 31; the programmable nuclease comprises an amino acid sequence that is at least 95% identical to SEQ ID NO: 5, and the engineered guide nucleic acid comprises a sequence that is at least 95% identical to SEQ ID NO: 32; the programmable nuclease comprises an amino acid sequence that is at least 95% identical to SEQ ID NO: 6, and the engineered guide nucleic acid comprises a sequence that is at least 95% identical to any one of SEQ ID NOs; 28-32; the programmable nuclease comprises an amino acid sequence that is at least 95% identical to SEQ ID NO: 7, and the engineered guide nucleic acid comprises a sequence that is at least 95% identical to any one of SEQ ID NOs; 28-32; the programmable nuclease comprises an amino acid sequence that is at least 95% identical to SEQ ID NO: 8, and the engineered guide nucleic acid comprises a sequence that is at least 95% identical to any one of SEQ ID NOs; 28-32; the programmable nuclease comprises an amino acid sequence that is at least 95% identical to SEQ ID NO: 9, and the engineered guide nucleic acid comprises a sequence that is at least 95% identical to any one of SEQ ID NOs; 28-32; the programmable nuclease comprises an amino acid sequence that is at least 95% identical to SEQ ID NO: 10, and the engineered guide nucleic acid comprises a sequence that is at least 95% identical to any one of SEQ ID NOs; 28-32; the programmable nuclease comprises an amino acid sequence that is at least 95% identical to SEQ ID NO: 11, and the engineered guide nucleic acid comprises a sequence that is at least 95% identical to any one of SEQ ID NOs; 28-32; the programmable nuclease comprises an amino acid sequence that is at least 95% identical to SEQ ID NO: 12, and the engineered guide nucleic acid comprises a sequence that is at least 95% identical to any one of SEQ ID NOs; 28-32; the programmable nuclease comprises an amino acid sequence that is at least 95% identical to SEQ ID NO: 13, and the engineered guide nucleic acid comprises a sequence that is at least 95% identical to any one of SEQ ID NOs; 28-32; the programmable nuclease comprises an amino acid sequence that is at least 95% identical to SEQ ID NO: 14, and the engineered guide nucleic acid comprises a sequence that is at least 95% identical to any one of SEQ ID NOs; 28-32; the programmable nuclease comprises an amino acid sequence that is at least 95% identical to SEQ ID NO: 15, and the engineered guide nucleic acid comprises a sequence that is at least 95% identical to SEQ ID NO: 60; the programmable nuclease comprises an amino acid sequence that is at least 95% identical to SEQ ID NO: 16, and the engineered guide nucleic acid comprises a sequence that is at least 95% identical to SEQ ID NO: 61; the programmable nuclease comprises an amino acid sequence that is at least 95% identical to SEQ ID NO: 17, and the engineered guide nucleic acid comprises a sequence that is at least 95% identical to SEQ ID NO: 62; the programmable nuclease comprises an amino acid sequence that is at least 95% identical to SEQ ID NO: 18, and the engineered guide nucleic acid comprises a sequence that is at least 95% identical to SEQ ID NO: 63; the programmable nuclease comprises an amino acid sequence that is at least 95% identical to SEQ ID NO: 19, and the engineered guide nucleic acid comprises a sequence that is at least 95% identical to SEQ ID NO: 60; the programmable nuclease comprises an amino acid sequence that is at least 95% identical to SEQ ID NO: 20, and the engineered guide nucleic acid comprises a sequence that is at least 95% identical to SEQ ID NO: 64; the programmable nuclease comprises an amino acid sequence that is at least 95% identical to SEQ ID NO: 21, and the engineered guide nucleic acid comprises a sequence that is at least 95% identical to SEQ ID NO: 61; the programmable nuclease comprises an amino acid sequence that is at least 95% identical to SEQ ID NO: 22, and the engineered guide nucleic acid comprises a sequence that is at least 95% identical to SEQ ID NO: 65; the programmable nuclease comprises an amino acid sequence that is at least 95% identical to SEQ ID NO: 23, and the engineered guide nucleic acid comprises a sequence that is at least 95% identical to SEQ ID NO: 60; the programmable nuclease comprises an amino acid sequence that is at least 95% identical to SEQ ID NO: 24, and the engineered guide nucleic acid comprises a sequence that is at least 95% identical to SEQ ID NO: 65; the programmable nuclease comprises an amino acid sequence that is at least 95% identical to SEQ ID NO: 25, and the engineered guide nucleic acid comprises a sequence that is at least 95% identical to SEQ ID NO: 66; the programmable nuclease comprises an amino acid sequence that is at least 95% identical to SEQ ID NO: 26, and the engineered guide nucleic acid comprises a sequence that is at least 95% identical to SEQ ID NO: 67; or the programmable nuclease comprises an amino acid sequence that is at least 95% identical to SEQ ID NO: 27, and the engineered guide nucleic acid comprises a sequence that is at least 95% identical to SEQ ID NO: 68. In some embodiments, the programmable nuclease comprises an amino acid sequence of SEQ ID NO: 1, and the engineered guide nucleic acid comprises a sequence of SEQ ID NO: 28; the programmable nuclease comprises an amino acid sequence of SEQ ID NO: 2, and the engineered guide nucleic acid comprises a sequence of SEQ ID NO: 29; the programmable nuclease comprises an amino acid sequence of SEQ ID NO: 3, and the engineered guide nucleic acid comprises a sequence of SEQ ID NO: 30; the programmable nuclease comprises an amino acid sequence of SEQ ID NO: 4, and the engineered guide nucleic acid comprises a sequence of SEQ ID NO: 31; the programmable nuclease comprises an amino acid sequence of SEQ ID NO: 5, and the engineered guide nucleic acid comprises a sequence of SEQ ID NO: 32; the programmable nuclease comprises an amino acid sequence of SEQ ID NO: 6, and the engineered guide nucleic acid comprises a sequence of any one of SEQ ID NOs; 28-32; the programmable nuclease comprises an amino acid sequence of SEQ ID NO: 7, and the engineered guide nucleic acid comprises a sequence of any one of SEQ ID NOs; 28-32; the programmable nuclease comprises an amino acid sequence of SEQ ID NO: 8, and the engineered guide nucleic acid comprises a sequence of any one of SEQ ID NOs; 28-32; the programmable nuclease comprises an amino acid sequence of SEQ ID NO: 9, and the engineered guide nucleic acid comprises a sequence of any one of SEQ ID NOs; 28-32; the programmable nuclease comprises an amino acid sequence of SEQ ID NO: 10, and the engineered guide nucleic acid comprises a sequence of any one of SEQ ID NOs; 28-32; the programmable nuclease comprises an amino acid sequence of SEQ ID NO: 11, and the engineered guide nucleic acid comprises a sequence of any one of SEQ ID NOs; 28-32; the programmable nuclease comprises an amino acid sequence of SEQ ID NO: 12, and the engineered guide nucleic acid comprises a sequence of any one of SEQ ID NOs; 28-32; the programmable nuclease comprises an amino acid sequence of SEQ ID NO: 13, and the engineered guide nucleic acid comprises a sequence of any one of SEQ ID NOs; 28-32; the programmable nuclease comprises an amino acid sequence of SEQ ID NO: 14, and the engineered guide nucleic acid comprises a sequence of any one of SEQ ID NOs; 28-32; the programmable nuclease comprises an amino acid sequence of SEQ ID NO: 15, and the engineered guide nucleic acid comprises a sequence of SEQ ID NO: 60; the programmable nuclease comprises an amino acid sequence of SEQ ID NO: 16, and the engineered guide nucleic acid comprises a sequence of SEQ ID NO: 61; the programmable nuclease comprises an amino acid sequence of SEQ ID NO: 17, and the engineered guide nucleic acid comprises a sequence of SEQ ID NO: 62; the programmable nuclease comprises an amino acid sequence of SEQ ID NO: 18, and the engineered guide nucleic acid comprises a sequence of SEQ ID NO: 63; the programmable nuclease comprises an amino acid sequence of SEQ ID NO: 19, and the engineered guide nucleic acid comprises a sequence of SEQ ID NO: 60; the programmable nuclease comprises an amino acid sequence of SEQ ID NO: 20, and the engineered guide nucleic acid comprises a sequence of SEQ ID NO: 64; the programmable nuclease comprises an amino acid sequence of SEQ ID NO: 21, and the engineered guide nucleic acid comprises a sequence of SEQ ID NO: 61; the programmable nuclease comprises an amino acid sequence of SEQ ID NO: 22, and the engineered guide nucleic acid comprises a sequence of SEQ ID NO: 65; the programmable nuclease comprises an amino acid sequence of SEQ ID NO: 23, and the engineered guide nucleic acid comprises a sequence of SEQ ID NO: 60; the programmable nuclease comprises an amino acid sequence of SEQ ID NO: 24, and the engineered guide nucleic acid comprises a sequence of SEQ ID NO: 65; the programmable nuclease comprises an amino acid sequence of SEQ ID NO: 25, and the engineered guide nucleic acid comprises a sequence of SEQ ID NO: 66; the programmable nuclease comprises an amino acid sequence of SEQ ID NO: 26, and the engineered guide nucleic acid comprises a sequence of SEQ ID NO: 67; or the programmable nuclease comprises an amino acid sequence of SEQ ID NO: 27, and the engineered guide nucleic acid comprises a sequence of SEQ ID NO: 68. In an embodiment, the engineered guide nucleic acid comprises a crRNA, a tracrRNA, or a combination thereof. In some embodiments, the engineered guide nucleic acid is a single guide nucleic acid. In some embodiments, the composition comprises i) a programmable nuclease comprising at least one HEPN or HEPN-like domain; and ii) an engineered guide nucleic acid. In some embodiments, the programmable nuclease comprises at least 75% sequence identity to a sequence selected from a group consisting of: SEQ ID NO: 1-SEQ ID NO: 27. In some embodiments, the engineered guide nucleic comprises at least 75% sequence identity to a sequence selected from a group consisting of: SEQ ID NO: 28-SEQ ID NO: 32. In some embodiments, the engineered guide nucleic acid comprises a sequence selected from a group consisting of: SEQ ID NO: 28-SEQ ID NO: 32. In some embodiments, the engineered guide nucleic acid comprises a first region (FR1) complementary to a target sequence and the second region (FR2) that is not complementary to the target sequence. In some embodiments, the first region and second region are oriented: FR1-FR2. In some embodiments, the first region and second region are oriented FR2-FR1. In some embodiments, FR1 comprises at least 75% sequence identity to a sequence selected from a group consisting of: SEQ ID NO: 28-SEQ ID NO: 32. In some embodiments, FR2 is a sequence comprising at least 75% sequence identity to SEQ ID NO: 41.

In an aspect, this disclosure describes a non-naturally occurring composition comprising a programmable nuclease and an engineered guide nucleic acid capable of catalyzing cRNA-directed, RNA-targeted trans-cleavage activity at a temperature of at least about 55° C. to at least about 85° C., wherein the programmable nuclease comprises at least one HEPN or HEPN-like domain.

In an aspect, this disclosure describes a non-naturally occurring composition comprising a programmable nuclease and engineered guide nucleic acid capable of catalyzing cRNA-directed, RNA-targeted trans-cleavage activity, wherein the programmable nuclease comprises at least one HEPN or HEPN-like domain, and wherein the programmable nuclease exhibits increased trans-cleavage activity when the spacer region is about 20 to about 30 nucleotides in length, compared to the cleavage produced by a composition comprising the same programmable nuclease and a guide nucleic acid comprising a spacer region less than 20 nucleobases in length, or greater than 30 nucleobases in length.

In an aspect, this disclosure describes a non-naturally occurring composition comprising a programmable nuclease comprising at least one HEPN or HEPN-like domain and an engineered guide nucleic acid capable of catalyzing at least a 1.5 fold change in cRNA-directed, RNA-targeted trans-cleavage activity. In an embodiment, fold change is determined by quantifying cleavage of a labeled detector RNA present in an in vitro sample in a reaction, performed at a temperature of about 37° C. and comprising: at least 160 nM of the RNA-guided endonuclease, at least 160 nM of the guide RNA, at least 5 nM of a target RNA, and 200 nM of the labeled detector RNA. In an embodiment, the programmable nuclease and engineered guide nucleic acid are capable of catalyzing at least a 25 fold change in cRNA-directed, RNA-targeted trans-cleavage activity. In an embodiment, the programmable nuclease and engineered guide nucleic acid are capable of catalyzing at least a 60 fold change in cRNA-directed, RNA-targeted trans-cleavage activity. In an embodiment, the programmable nuclease and engineered guide nucleic acid are capable of catalyzing at least a 80 fold change in cRNA-directed, RNA-targeted trans-cleavage activity. In an embodiment, the amino acid sequence of the programmable nuclease is about 780 to about 850 amino acids in length. In an embodiment, the amino acid sequence of the programmable nuclease is about 700 to about 900 amino acids in length. In an embodiment, the programmable nuclease exhibits increased trans-cleavage activity when the guide RNA comprises a spacer region of about 25 nucleotides in length, as compared to the cleavage produced by a composition comprising the same programmable nuclease and a guide nucleic acid comprising a spacer region less than 20 nucleotides in length, or greater than 30 nucleotides in length. In an embodiment, the cleavage exhibited by the programmable nuclease when the guide nucleic acid comprises a spacer region of about 20 to about 30 nucleotides in length is at least 2-fold greater than the cleavage produced by a composition comprising the same programmable nuclease and a guide nucleic acid comprising a spacer region less than 20 nucleotides in length, or greater than 30 nucleotides in length. In an embodiment, the cleavage exhibited by the programmable nuclease when the guide nucleic acid comprises a spacer region of about 20 to about 30 nucleotides in length is at least 5-fold greater than the cleavage produced by a composition comprising the same programmable nuclease and a guide nucleic acid comprising a spacer region less than 20 nucleotides in length, or greater than 30 nucleotides in length. In an embodiment, the cleavage exhibited by the programmable nuclease when the guide nucleic acid comprises a spacer region of about 20 to about 30 nucleotides in length is at least 10-fold greater than the cleavage produced by a composition comprising the same programmable nuclease and a guide nucleic acid comprising a spacer region less than 20 nucleotides in length, or greater than 30 nucleotides in length. In an embodiment, the amino acid sequence of the programmable nuclease is at least about 50% identical to a Cas13 protein. In an embodiment, the amino acid sequence of the programmable nuclease is at least about 60% identical to a Cas13 protein. In an embodiment, the amino acid sequence of the programmable nuclease is at least about 70% identical to a Cas13 protein. In an embodiment, the amino acid sequence of the programmable nuclease is at least about 80% identical to a Cas13 protein. In an embodiment, the amino acid sequence of the programmable nuclease is at least about 90% identical to a Cas13 protein. In an embodiment, the programmable nuclease comprises an amino acid sequence that is at least 75% identical to any one of SEQ ID NOS: 15-27. In an embodiment, the programmable nuclease comprises an amino acid sequence that is at least 85% identical to any one of SEQ ID NOS: 15-27. In an embodiment, the programmable nuclease comprises an amino acid sequence that is at least 95% identical to any one of SEQ ID NOS: 15-27. In an embodiment, the programmable nuclease comprises an amino acid sequence of any one of SEQ ID NOS: 15-27. In an embodiment, the engineered guide nucleic acid comprises a nucleotide sequence of any one of SEQ ID NOS: 60-68. In an embodiment, the programmable nuclease and engineered guide nucleic acid are capable of catalyzing cRNA-directed, RNA-targeted trans-cleavage activity at a temperature of about 20° C. to about 70° C., or about 50° C. to about 70° C. In an embodiment, the programmable nuclease and engineered guide nucleic acid are capable of catalyzing cRNA-directed, RNA-targeted trans-cleavage activity at a temperature of about 20° C. In an embodiment, the programmable nuclease and engineered guide nucleic acid are capable of catalyzing cRNA-directed, RNA-targeted trans-cleavage activity at a temperature of about 30° C. In an embodiment, the programmable nuclease and engineered guide nucleic acid are capable of catalyzing cRNA-directed, RNA-targeted trans-cleavage activity at a temperature of about 40° C. In an embodiment, the programmable nuclease and engineered guide nucleic acid are capable of catalyzing cRNA-directed, RNA-targeted trans-cleavage activity at a temperature of about 50° C. In an embodiment, the programmable nuclease and engineered guide nucleic acid are capable of catalyzing cRNA-directed, RNA-targeted trans-cleavage activity at a temperature of about 55° C. In an embodiment, the programmable nuclease and engineered guide nucleic acid are capable of catalyzing cRNA-directed, RNA-targeted trans-cleavage activity at a temperature of about 60° C. In an embodiment, the programmable nuclease and engineered guide nucleic acid are capable of catalyzing cRNA-directed, RNA-targeted trans-cleavage activity at a temperature of about 65° C. In an embodiment, the programmable nuclease and engineered guide nucleic acid are capable of catalyzing cRNA-directed, RNA-targeted trans-cleavage activity at a temperature of about 70° C. In some embodiments, the programmable nuclease and engineered guide nucleic acid are capable of catalyzing cRNA-directed, RNA-targeted trans-cleavage activity at a temperature of about 20° C. In some embodiments, the programmable nuclease and engineered guide nucleic acid are capable of catalyzing cRNA-directed, RNA-targeted trans-cleavage activity at a temperature of not greater than 20° C. In some embodiments, the programmable nuclease and engineered guide nucleic acid are capable of catalyzing cRNA-directed, RNA-targeted trans-cleavage activity at a temperature of at least 20° C. In an embodiment, the programmable nuclease comprises two HEPN or HEPN-like domains. In an embodiment, the programmable nuclease is a Cas13c nuclease. In an embodiment, the programmable nuclease is identified in a wild-type bacterial genome by association with a locus comprising a CRISPR array and lacking a cas1 gene or a cas2 gene. In an embodiment, a system for detecting a target nucleic acid comprises the composition and at least one of a buffering agent, a salt, a crowding agent, a detergent, a reducing agent, a competitor, and a reporter nucleic acid. In some embodiments, the system comprises a solution comprising the at least one of a buffering agent, salt, crowding agent, detergent, reducing agent, competitor, and detection agent. In some embodiments, the pH of the solution is at least about 6.0. In some embodiments, the pH of the solution is at least about 6.5. In some embodiments, the pH of the solution is at least about 7.0. In some embodiments, the pH of the solution is at least about 7.5. In some embodiments, the pH of the solution is at least about 8.0. In some embodiments, the pH of the solution is at least about 8.5. In some embodiments, the pH of the solution is at least about 9.0. In some embodiments, the salt is selected from a magnesium salt, a potassium salt, a sodium salt and a calcium salt. In some embodiments, the concentration of the salt in the solution is at least about 1 mM. In some embodiments, the concentration of the salt in the solution is at least about 1 mM. In some embodiments, the concentration of the salt in the solution is at least about 3 mM. In some embodiments, the concentration of the salt in the solution is at least about 5 mM. In some embodiments, the concentration of the salt in the solution is at least about 7 mM. In some embodiments, the concentration of the salt in the solution is at least about 9 mM. In some embodiments, the concentration of the salt in the solution is at least about 11 mM. In some embodiments, the concentration of the salt in the solution is at least about 13 mM. In some embodiments, the concentration of the salt in the solution is at least about 15 mM. In some embodiments, the reporter nucleic acid comprises a sequence selected from SEQ ID NOS: 33-40. In some embodiments, the detection reagent is the reporter nucleic acid. In some embodiments, the reporter nucleic acid comprises a detection moiety, a quencher, or a combination thereof. In some embodiments, the detection moiety and the quencher are selected from Table 3. In some embodiments, the detection moiety comprises a fluorophore. In some embodiments, the reporter nucleic acid comprises the quencher. In some embodiments, the reporter nucleic acid comprises at least one of a fluorophore and a quencher. In some embodiments, the reporter nucleic acid is in the form of a single-stranded RNA. In some embodiments, the system comprises at least one amplification reagent for amplifying a sample. In some embodiments, the at least one amplification reagent is selected from the group consisting of a primer, an activator, a deoxynucleoside triphosphate (dNTP), a ribonucleoside triphosphate (rNTP), and combinations thereof. In some embodiments, amplifying comprises isothermal amplification or polymerase chain reaction (PCR). In some embodiments, the system does not include at least one amplification reagent for amplifying a sample. In some embodimets, the system does not include isothermal amplification or PCR. In some embodiments, a pharmaceutical composition comprises a therapeutically effective amount of the composition described herein, and a pharmaceutically acceptable diluent or excipient. In some embodiments, the pharmaceutically acceptable diluent is selected from phosphate buffered saline and water.

In an aspect, this disclosure describes a method of altering the sequence of a nucleic acid comprises contacting a target nucleic acid molecule with a composition described herein or a system described herein. In an aspect, this disclosure describes a method of introducing a break in a target nucleic acid comprises contacting a target nucleic acid molecule with a composition described herein or a system described herein. In some embodiments, the target nucleic acid is single stranded. In some embodiments, the target nucleic acid is double stranded. In some embodiments, the target nucleic acid comprises RNA. In some embodiments, the target nucleic acid comprises DNA. In some embodiments, the programmable nuclease further comprises an editing domain. In some embodiments, the editing domain comprises ADAR1/2 or a functional variant thereof. In some embodiments, the contacting occurs in vitro. In some embodiments, the contacting occurs ex vivo. In some embodiments, the contacting occurs in vivo. In some embodiments, the contacting occurs in a sample, wherein the sample is selected from an environmental sample and a biological sample. In some embodiments, the biological sample is selected from blood, plasma, saliva, a buccal swab, a nasal swab, and urine.

In an aspect, this disclosure describes a method of detecting a target nucleic acid in a sample comprises contacting a target nucleic acid with a composition described herein or a system described herein. In some embodiments, the method comprises contacting the sample with a reporter nucleic acid. In some embodiments, the method comprises measuring a detectable signal produced by cleavage of the reporter nucleic acid. In some embodiments, the method comprises contacting at a temperature of at least about 40° C. In some embodiments, the method comprises contacting at a temperature of at least about 50° C. In some embodiments, the method comprises contacting at a temperature of at least about 55° C. In some embodiments, the method comprises contacting at a temperature of at least about 60° C. In some embodiments, the method comprises contacting at a temperature of at least about 65° C. In some embodiments, the method comprises contacting at a temperature of at least about 70° C. In some embodiments, contacting occurs at a temperature not greater than 45° C. In some embodiments, contacting occurs at a temperature of about 45° C. In some embodiments, contacting occurs at a temperature of about 50° C. In some embodiments, contacting occurs at a temperature of about 55° C. In some embodiments, contacting occurs at a temperature of about 60° C. In some embodiments, contacting occurs at a temperature of about 65° C. In some embodiments, contacting occurs at a temperature of about 70° C. In some embodiments, the method comprises amplifying the target nucleic acid. In some embodiments, the amplifying is performed before contacting. In some embodiments, the amplifying is performed during contacting. In some embodiments, the amplifying occurs at a temperature of at least about 50° C. In some embodiments, the amplifying occurs at a temperature of at least about 55° C. In some embodiments, the amplifying occurs at a temperature of at least about 60° C. In some embodiments, the amplifying occurs at a temperature of at least about 65° C. In some embodiments, the amplifying occurs at a temperature not greater than 70° C. In some embodiments, the amplifying occurs at a temperature of about 20° C. In some embodiments, the amplifying occurs at a temperature of about 30° C. In some embodiments, the amplifying occurs at a temperature of about 40° C. In some embodiments, the amplifying occurs at a temperature of about 50° C. In some embodiments, the amplifying occurs at a temperature of about 55° C. In some embodiments, the amplifying occurs at a temperature of about 60° C. In some embodiments, the amplifying occurs at a temperature of about 65° C. In some embodiments, the amplifying occurs at a temperature of about 70° C. In some embodiments, the amplifying comprises isothermal amplification or polymerase chain reaction (PCR). In some embodiments, the method comprises transcribing DNA in the sample to produce the target nucleic acid. In some embodiments, the contacting and the transcribing are carried out at the same temperature. In some embodiments, the contacting, detecting, amplifying, transcribing, or any combination thereof, are carried out at the same temperature. In some embodiments, the contacting, detecting, amplifying, transcribing, or any combination thereof, are carried out in a single reaction chamber. In some embodiments, the method comprises not amplifying the target nucleic acid. In some embodiments, the method does not include isothermal amplification or PCR. In some embodiments, the sample, or portion thereof, is from a pathogen. In some embodiments, the pathogen is a virus or a bacterium. In some embodiments, the virus is a coronavirus. In some embodiments, the coronavirus is SARS-CoV-2 virus. In some embodiments, the virus is an influenza virus. In some embodiments, the influenza virus is influenza A virus or influenza B virus. In some embodiments, the virus is a human papillomavirus or a herpes simplex virus. In some embodiments, the virus is a respiratory syncytial virus. In some embodiments, the pathogen is a bacterium. In some embodiments, the bacterium is a Chlamydia trachomatis. In some embodiments, the sample, or portion thereof, comprises a target nucleic acid from a coronavirus MERS-CoV, SARS-CoV-2, a human metapneumovirus, a rhinovirus, an enterovirus, influenza A, influenza B, parainfluenza 1, 2, 3, 4, or 4a, a respiratory syncytial virus A (RSV-A), a respiratory syncytial virus B, a gammacoronavirus, a deltacoronavirus, a betacoronavirus, an alphacoronavirus, a sarbecovirus subgenus, a SARS-related virus, Bordetella pertussis, Bordetella parapertussis, Bordetella bronchoseptica, Bordetella holmesii, Chlamydophila pneumoniae, Legionella pneumophila, Mycoplasma pneumoniae, a human bocavirus, or a human adenovirus, or a combination thereof. In some embodiments, the programmable nuclease provides cis-cleavage activity on the target nucleic acid. In some embodiments, the programmable nuclease provides transcollateral cleavage activity on the target nucleic acid a DNA/RNA Endonuclease Targeted CRISPR TransReporter (DETECTR) assay.

In an aspect, this disclosure describes a system or device for use to detect a target nucleic acid in a sample, wherein the system or device uses a method described herein.

In an aspect, this disclosure describes a programmable nuclease comprising a sequence with at least 75% sequence identity to SEQ ID NO: 1-SEQ ID NO: 27 which binds to an engineered guide nucleic acid, and wherein the engineered guide nucleic acid comprises a sequence with at least 75% sequence identity to SEQ ID NO: 28-SEQ ID NO: 32. In some embodiments, the programmable nuclease comprises at least one HEPN or HEPN-like domain.

In an aspect, this disclosure describes a composition comprising a programmable nuclease comprising at least one HEPN or HEPN-like domain and an engineered guide nucleic acid. In some embodiments, the programmable nuclease comprises at least 75% sequence identity to a sequence selected from a group consisting: SEQ ID NO: 1-SEQ ID NO: 27. In some embodiments, the engineered guide nucleic comprises at least 75% sequence identity to a sequence selected from a group consisting of: SEQ ID NO: 28-SEQ ID NO: 32. In some embodiments, the engineered guide nucleic acid comprises a sequence selected from a group consisting of: SEQ ID NO: 28-SEQ ID NO: 32. In some embodiments, the engineered guide nucleic acid comprises a first region (FR1) complementary to a target sequence and the second region (FR2) that is not complementary to the target sequence. In some embodiments, the first region and second region are oriented FR1-FR2. In some embodiments, the first region and second region are oriented FR2-FR1. In some embodiments, FR1 comprises at least 75% sequence identity to a sequence selected from a group consisting of: SEQ ID NO: 28-SEQ ID NO: 32. In some embodiments, FR2 comprises at least 75% sequence identity to SEQ ID NO: 41.

In an aspect, this disclosure describes a method of detecting a nucleic acid in a sample, comprising the steps of: i) contacting a sample with: a) a programmable nuclease; b) a reporter; and c) an engineered guide nucleic acid; ii) measuring a detectable signal produced by cleavage of the reporter, wherein the measuring provides detection of the target nucleic acid in the sample. In some embodiments, at least one programmable nuclease comprises at least 75% sequence identity to a sequence selected from a group consisting of: SEQ ID NO: 1-SEQ ID NO: 27. In some embodiments, the nucleic acid comprises influenza A virus or influenza B virus. In some embodiments, at least one programmable nuclease comprises SEQ ID NO: 24, and wherein at least one engineered guide nucleic acid comprises any one of SEQ ID NOs: 70-72. In some embodiments, at least one programmable nuclease comprises SEQ ID NO: 26, and wherein contacting occurs at a temperature not greater than 45° C. In some embodiments, at least one programmable nuclease comprises SEQ ID NO: 26, and wherein contacting occurs at a temperature of about 45° C. In some embodiments, at least one programmable nuclease comprises SEQ ID NO: 27, and wherein contacting occurs at a temperature not greater than 50° C. In some embodiments, at least one programmable nuclease comprises SEQ ID NO: 27, and wherein contacting occurs at a temperature of about 50° C. In some embodiments, at least one programmable nuclease comprises SEQ ID NO: 22, and wherein contacting occurs at a temperature not greater than 55° C. In some embodiments, at least one programmable nuclease comprises SEQ ID NO: 22, and wherein contacting occurs at a temperature of about 55° C. In some embodiments, at least one programmable nuclease comprises SEQ ID NO: 23, and wherein contacting occurs at a temperature not greater than 45° C. In some embodiments, at least one programmable nuclease comprises SEQ ID NO: 23, and wherein contacting occurs at a temperature of about 45° C. In some embodiments, at least one programmable nuclease comprises SEQ ID NO: 25, and wherein contacting occurs at a temperature not greater than 60° C. In some embodiments, at least one programmable nuclease comprises SEQ ID NO: 25, and wherein contacting occurs at a temperature of about 60° C. In some embodiments, at least one programmable nuclease comprises SEQ ID NO: 24, and wherein contacting occurs at a temperature not greater than 60° C. In some embodiments, at least one programmable nuclease comprises SEQ ID NO: 24, and wherein contacting occurs at a temperature of about 60° C. In some embodiments, at least one programmable nuclease comprises SEQ ID NO: 20, and wherein contacting occurs at a temperature not greater than 50° C. In some embodiments, at least one programmable nuclease comprises SEQ ID NO: 20, and wherein contacting occurs at a temperature of about 50° C. In some embodiments, the reporter comprises a detection moiety and a quencher. In some embodiments, the detection moiety and the quencher are selected from Table 3. In some embodiments, the reporter comprises a nucleic acid sequence. In some embodiments, the nucleic acid sequence is selected from a group consisting of: SEQ ID NO: 33-SEQ ID NO: 40. In some embodiments, the engineered guide nucleic acid comprises a first region (FR1) complementary to a target sequence and the second region (FR2) that is not complementary to the target sequence. In some embodiments, the first region and second region are oriented FR1-FR2. In some embodiments, the first region and second region are oriented FR2-FR1. In some embodiments, FR1 comprises at least 75% sequence identity to a sequence selected from a group consisting pf: SEQ ID NO: 28-SEQ ID NO: 32. In some embodiments, FR2 comprises at least 75% sequence identity to SEQ ID NO: 41. In some embodiments, at least one programmable nuclease comprising SEQ ID NO: 22, and contacting occurs at a temperature of less than 30° C.; b) at least one programmable nuclease comprising SEQ ID NO: 23, and contacting occurs at a temperature of less than 30° C.; c) at least one programmable nuclease comprising SEQ ID NO: 24, and contacting occurs at a temperature of less than 30° C.; or d) at least one programmable nuclease comprising SEQ ID NO: 25, and contacting occurs at a temperature of less than 30° C. In some embodiments, at least one programmable nuclease comprising SEQ ID NO: 22, and contacting occurs at a temperature of about 20° C.; b) at least one programmable nuclease comprising SEQ ID NO: 23, and contacting occurs at a temperature of about 20° C.; c) at least one programmable nuclease comprising SEQ ID NO: 24, and contacting occurs at a temperature of about 20° C.; or d) at least one programmable nuclease comprising SEQ ID NO: 25, and contacting occurs at a temperature of about 20° C. In some embodiments, the target nucleic acid is single-stranded RNA (ssRNA) and wherein the break in the target nucleic acid is trans cleavage. In some embodiments, the programmable nuclease is a Cas13 protein. In some embodiments, the amino acid sequence of the programmable nuclease is at least about 50% identical to a Cas13 protein. In some embodiments, the amino acid sequence of the programmable nuclease is at least about 60% identical to a Cas13 protein. In some embodiments, the amino acid sequence of the programmable nuclease is at least about 70% identical to a Cas13 protein. In some embodiments, the amino acid sequence of the programmable nuclease is at least about 80% identical to a Cas13 protein. In some embodiments, the amino acid sequence of the programmable nuclease is at least about 90% identical to a Cas13 protein. In some embodiments, the amino acid sequence of the programmable nuclease is a Cas13 protein. In some embodiments, the amino acid sequence of the programmable nuclease is at least about 50% identical to a Cas13c protein. In some embodiments, the amino acid sequence of the programmable nuclease is at least about 60% identical to a Cas13c protein. In some embodiments, the amino acid sequence of the programmable nuclease is at least about 70% identical to a Cas13c protein. In some embodiments, the amino acid sequence of the programmable nuclease is at least about 80% identical to a Cas13c protein. In some embodiments, the amino acid sequence of the programmable nuclease is at least about 90% identical to a Cas13c protein. In some embodiments, the amino acid sequence of the programmable nuclease is a Cas13c protein. In some embodiments, the programmable nuclease comprises any one of SEQ ID NO: 22-25. In some embodiments, the target nucleic acid comprises a plant gene or expression product thereof. In some embodiments, use of the method described herein comprises performing the method in a plant cell or plant cell lysate.

In an aspect, this disclosure describes a method of altering the sequence of a nucleic acid, the method comprising: i) contacting a nucleic acid molecule with: a) a programmable nuclease; and b) an engineered guide nucleic acid. In some embodiments, the nucleic acid is a single stranded ribonucleic acid. In some embodiments, the programmable nuclease comprises at least 75% sequence identity to a sequence selected from a group consisting of: SEQ ID NO: 1-SEQ ID NO: 27. In some embodiments, the programmable nuclease further comprises an editing domain. In some embodiments, the editing domain comprises ADAR1/2 or a functional variant thereof. In some embodiments, the engineered guide nucleic acid comprises a first region (FR1) complementary to a target sequence and the second region (FR2) that is not complementary to the target sequence. In some embodiments, the first region and second region are oriented FR1-FR2. In some embodiments, the first region and second region are oriented FR2-FR1. In some embodiments, FR1 comprises at least 75% sequence identity to a sequence selected from a group consisting of: SEQ ID NO: 28-SEQ ID NO: 32. In some embodiments, FR2 comprises at least 75% sequence identity to SEQ ID NO: 41.

In an aspect, this disclosure describes a method of introducing a break in a target nucleic acid, the method comprising: i) contacting the target nucleic acid with: a) an engineered guide nucleic acid; and b) a programmable nuclease. In some embodiments, the nucleic acid is a single stranded ribonucleic acid. In some embodiments, the programmable nuclease is selected from SEQ ID NO: 1-SEQ ID NO: 27. In some embodiments, the guide nucleic acid comprises a first region (FR1) complementary to a target sequence and the second region (FR2) that is not complementary to the target sequence. In some embodiments, the first region and second region are oriented: FR1-FR2. In some embodiments, the first region and second region are oriented FR2-FR1. In some embodiments, FR1 comprises at least 75% sequence identity to a sequence selected from a group consisting of: SEQ ID NO: 28-SEQ ID NO: 32. In some embodiments, FR2 comprises at least 75% sequence identity to SEQ ID NO: 41.

In an aspect, this disclosure describes a recombinant nucleic acid encoding a programmable nuclease comprising an amino acid sequence that at least 75% identical to any one of SEQ ID NOs: 1-27. In some embodiments, the nucleic acid comprises a nucleotide sequence encoding the programmable nuclease operatively linked to a promoter. In some embodiments, a vector comprises a recombinant nucleic acid as described herein. In some embodiments, a non-naturally occurring host cell comprises a recombinant nucleic acid as described herein. In some embodiments, the non-naturally occurring host cell is a microbial organism.

In an aspect, this disclosure describes a method for producing a programmable nuclease comprising culturing a non-naturally occurring host cell as described herein under a condition suitable for production of the programmable nuclease.

In an aspect, this disclosure describes a method for producing a programmable nuclease using a host cell, wherein the method comprises introducing into the host cell a recombinant nucleic acid as described herein or a vector as described herein and culturing the host cell under a condition suitable for production of the programmable nuclease. In some embodiments, the method comprises isolating the programmable nuclease. In some embodiments, the introduction of the recombinant nucleic acid into the host cell comprises electroporation, nucleofection, chemical methods, transfection, transduction, transformation, or microinjection. In some embodiments, the host cell is a prokaryotic cell or a eukaryotic cell. In some embodiments, the host cell is in vivo. In some embodiments, the host cell is ex vivo. In some embodiments, the host cell is in vitro. In some embodiments, the host cell is a bacterial cell, a yeast cell, a plant cell, or a mammalian cell. In some embodiments, the host cell is a human cell. In some embodiments, the host cell is a non-human mammalian cell. In some embodiments, the host cell is an insect cell. In some embodiments, the host cell is an arthropod cell. In some embodiments, the host cell is a fungal cell. In some embodiments, the host cell is an algal cell.

Provided herein is a programmable nuclease comprising a sequence with at least 75% sequence identity to SEQ ID NO: 1-SEQ ID NO: 27 which binds to an engineered guide nucleic acid, and wherein the engineered guide nucleic acid comprises a sequence with at least 75% sequence identity to SEQ ID NO: 28-SEQ ID NO: 32. In some embodiments, the programmable nuclease comprises at least one HEPN or HEPN-like domain.

Provided herein is a system for modifying a target nucleic acid comprising: i) a programmable nuclease comprising at least one HEPN or HEPN-like domain, ii) an engineered guide nucleic acid, wherein the engineered guide nucleic acid comprises a nucleotide sequence that can bind to the target nucleic acid. In some embodiments, the programmable nuclease comprises at least 97%, at least 98%, or at least 99% sequence identity to a sequence selected from a group consisting of: SEQ ID NO: 1-SEQ ID NO: 27. In some embodiments, the engineered guide nucleic comprises at least 75% sequence identity to a sequence selected from a group consisting of: SEQ ID NO: 28-SEQ ID NO: 32. In some embodiments, the engineered guide nucleic acid comprises a sequence selected from a group consisting of: SEQ ID NO: 28-SEQ ID NO: 32. In some embodiments, the engineered guide nucleic acid comprises a first region (FR1) complementary to a target sequence and the second region (FR2) that is not complementary to the target sequence. In some embodiments, the first region and second region are oriented FR1-FR2. In some embodiments, the first region and second region are oriented FR2-FR1. In some embodiments, FR1 comprises at least 75% sequence identity to a sequence selected from a group consisting of: SEQ ID NO: 28-SEQ ID NO: 32. In some embodiments, FR2 comprises at least 75% sequence identity to SEQ ID NO: 41.

Provided herein is a method of detecting a nucleic acid in a sample, comprising the steps of i) contacting a sample with: a) a programmable nuclease; b) a reporter; and c) an engineered guide nucleic acid; and ii) measuring a detectable signal produced by cleavage of the reporter, wherein the measuring provides detection of the target nucleic acid in the sample. In some embodiments, at least one programmable nuclease comprises at least 75% sequence identity to a sequence selected from a group consisting of: SEQ ID NO: 1-SEQ ID NO: 27. In some embodiments, the nucleic acid comprises influenza A virus or influenza B virus. In some embodiments, at least one programmable nuclease comprises SEQ ID NO: 24, and at least one engineered guide nucleic acid comprises any one of SEQ ID NOs: 70-72. In some embodiments, at least one programmable nuclease comprises SEQ ID NO: 26, and contacting occurs at a temperature not greater than 45° C. In some embodiments, at least one programmable nuclease comprises SEQ ID NO: 26, and contacting occurs at a temperature of about 45° C. In some embodiments, at least one programmable nuclease comprises SEQ ID NO: 27, and contacting occurs at a temperature not greater than 50° C. In some embodiments, at least one programmable nuclease comprises SEQ ID NO: 27, and contacting occurs at a temperature of about 50° C. In some embodiments, at least one programmable nuclease comprises SEQ ID NO: 22, and contacting occurs at a temperature not greater than 55° C. In some embodiments, at least one programmable nuclease comprises SEQ ID NO: 22, and contacting occurs at a temperature of about 55° C. In some embodiments, at least one programmable nuclease comprises SEQ ID NO: 23, and contacting occurs at a temperature not greater than 45° C. In some embodiments, at least one programmable nuclease comprises SEQ ID NO: 23, and contacting occurs at a temperature of about 45° C. In some embodiments, at least one programmable nuclease comprises SEQ ID NO: 25, and contacting occurs at a temperature not greater than 60° C. In some embodiments, at least one programmable nuclease comprises SEQ ID NO: 25, and contacting occurs at a temperature of about 60° C. In some embodiments, at least one programmable nuclease comprises SEQ ID NO: 24, and contacting occurs at a temperature not greater than 60° C. In some embodiments, at least one programmable nuclease comprises SEQ ID NO: 24, and contacting occurs at a temperature of about 60° C. In some embodiments, at least one programmable nuclease comprises SEQ ID NO: 20, and contacting occurs at a temperature not greater than 50° C. In some embodiments, at least one programmable nuclease comprises SEQ ID NO: 20, and contacting occurs at a temperature of about 50° C. In some embodiments, the reporter comprises a detection moiety and a quencher. In some embodiments, the detection moiety and the quencher are selected from Table 3. In some embodiments, the reporter comprises a nucleic acid sequence. In some embodiments, the nucleic acid sequence is selected from a group consisting of: SEQ ID NO: 33-SEQ ID NO: 40. In some embodiments, the engineered guide nucleic acid comprises a first region (FR1) complementary to a target sequence and the second region (FR2) that is not complementary to the target sequence. In some embodiments, the first region and second region are oriented FR1-FR2. In some embodiments, the first region and second region are oriented FR2-FR1. In some embodiments, FR1 comprises at least 75% sequence identity to a sequence selected from a group consisting of: SEQ ID NO: 28-SEQ ID NO: 32. In some embodiments, FR2 comprises at least 75% sequence identity to SEQ ID NO: 41. In some embodiments, the method comprises a) at least one programmable nuclease comprising SEQ ID NO: 22, and contacting occurs at a temperature of less than 30° C.; b) at least one programmable nuclease comprising SEQ ID NO: 23, and contacting occurs at a temperature of less than 30° C.; c) at least one programmable nuclease comprising SEQ ID NO: 24, and contacting occurs at a temperature of less than 30° C.; or d) at least one programmable nuclease comprising SEQ ID NO: 25, and contacting occurs at a temperature of less than 30° C. In some embodiments, the method comprises a) at least one programmable nuclease comprising SEQ ID NO: 22, and contacting occurs at a temperature of about 20° C.; b) at least one programmable nuclease comprising SEQ ID NO: 23, and contacting occurs at a temperature of about 20° C.; c) at least one programmable nuclease comprising SEQ ID NO: 24, and contacting occurs at a temperature of about 20° C.; or d) at least one programmable nuclease comprising SEQ ID NO: 25, and contacting occurs at a temperature of about 20° C. In some embodiments, the target nucleic acid is single-stranded RNA (ssRNA) and the break in the target nucleic acid is introduced by trans cleavage. In some embodiments, the programmable nuclease is a Cas13 protein. In some embodiments, the amino acid sequence of the programmable nuclease is at least about 50% identical to a Cas13 protein. In some embodiments, the amino acid sequence of the programmable nuclease is at least about 60% identical to a Cas13 protein. In some embodiments, the amino acid sequence of the programmable nuclease is at least about 70% identical to a Cas13 protein. In some embodiments, the amino acid sequence of the programmable nuclease is at least about 80% identical to a Cas13 protein. In some embodiments, the amino acid sequence of the programmable nuclease is at least about 90% identical to a Cas13 protein. In some embodiments, the programmable nuclease is a Cas13c protein. In some embodiments, the amino acid sequence of the programmable nuclease is at least about 50% identical to a Cas13c protein. In some embodiments, the amino acid sequence of the programmable nuclease is at least about 60% identical to a Cas13c protein. In some embodiments, the amino acid sequence of the programmable nuclease is at least about 70% identical to a Cas13c protein. In some embodiments, the amino acid sequence of the programmable nuclease is at least about 80% identical to a Cas13c protein. In some embodiments, the amino acid sequence of the programmable nuclease is at least about 90% identical to a Cas13c protein. In some embodiments, the programmable nuclease comprises any one of SEQ ID NO: 22-25. In some embodiments, the target nucleic acid comprises a plant gene or expression product thereof. In some embodiments, the use comprises performing the method in a plant cell or plant cell lysate.

Provided herein is a method of altering the sequence of a nucleic acid, comprising the steps of i) contacting a nucleic acid molecule with a) a programmable nuclease; and b) an engineered guide nucleic acid. In some embodiments, the nucleic acid is a single stranded ribonucleic acid. In some embodiments, the programmable nuclease comprises at least 75% sequence identity to a sequence selected from a group consisting of: SEQ ID NO: 1-SEQ ID NO: 27. In some embodiments, the programmable nuclease further comprises an editing domain. In some embodiments, the editing domain comprises ADAR1/2 or a functional variant thereof. In some embodiments, the engineered guide nucleic acid comprises a first region (FR1) complementary to a target sequence and the second region (FR2) that is not complementary to the target sequence. In some embodiments, the first region and second region are oriented FR1-FR2. In some embodiments, the first region and second region are oriented FR2-FR1. In some embodiments, FR1 comprises at least 75% sequence identity to a sequence selected from a group consisting of: SEQ ID NO: 28-SEQ ID NO: 32. In some embodiments, FR2 comprises at least 75% sequence identity to SEQ ID NO: 41.

Provided herein is a method of introducing a break in a target nucleic acid, the method comprising: i) contacting the target nucleic acid with a) an engineered guide nucleic acid; and b) a programmable nuclease. In some embodiments, the target nucleic acid is a single stranded ribonucleic acid. In some embodiments, the programmable nuclease comprises a sequence selected from a group consisting of: SEQ ID NO: 1-SEQ ID NO: 27. In some embodiments, the engineered guide nucleic acid comprises a first region (FR1) complementary to a target sequence and the second region (FR2) that is not complementary to the target sequence. In some embodiments, the first region and second region are oriented FR1-FR2. In some embodiments, the first region and second region are oriented FR2-FR1. In some embodiments, FR1 comprises at least 75% sequence identity to a sequence selected from a group consisting of: SEQ ID NO: 28-SEQ ID NO: 32. In some embodiments, FR2 comprises at least 75% sequence identity to SEQ ID NO: 41.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The features and advantages of the present disclosure is obtained by reference to the following detailed description that sets forth illustrative embodiments, in which the principles of the invention are utilized, and the accompanying drawings of which:

FIG. 1 shows use of a Type VI nuclease (SEQ ID NOs: 1-5 and 15-27) for detection of a nucleic acid in a sample using a DNA/RNA Endonuclease Targeted CRISPR Trans Reporter (DETECTR) system.

FIG. 2 shows that Type VI CRISPR/Cas proteins (SEQ ID NOs: 1-5 and 15-27) of the disclosure can provide trans cleavage at 60° C.

FIG. 3 provides a phylogenetic tree of Type VI CRISPR/Cas proteins (SEQ ID NOs: 1-5 and 15-27).

FIGS. 4A-4B show use of a Type VI nuclease (SEQ ID NO: 6-11) for detection of a nucleic acid in a sample using a DNA/RNA Endonuclease Targeted CRISPR Trans Reporter (DETECTR) system.

FIG. 5 show use of a Type VI nuclease (SEQ ID NO: 12) for detection of a nucleic acid in a sample using a DNA/RNA Endonuclease Targeted CRISPR Trans Reporter (DETECTR) system.

FIG. 6 show use of a Type VI nuclease (SEQ ID NO: 13-14) for detection of a nucleic acid in a sample using a DNA/RNA Endonuclease Targeted CRISPR Trans Reporter (DETECTR) system.

FIGS. 7A-7B depict screens of each effector protein with each guide sequence, showing trans-cleavage reporter preferences of various enzymes described herein.

FIG. 8 depicts the ability of CasM.26-SEQ ID NO: 69 and CasM.1740-SEQ ID NO: 27 to exhibit trans cleavage activity above room temperature.

FIG. 9 depicts the ability of CasM.1422-SEQ ID NO: 26 to exhibit trans cleavage activity above room temperature.

FIGS. 10A-10C depicts the ability of CasM.1862921-SEQ ID NO: 24 (FIG. 10A), CasM.1862895-SEQ ID NO: 20 and CasM.1862909-SEQ ID NO: 22 (FIG. 10B), and CasM.1862917-SEQ ID NO: 23 (FIG. 10C) to exhibit trans cleavage activity above room temperature.

FIG. 11 depicts the trans cleavage activity of CasM.1862909-SEQ ID NO: 22 and CasM.1862921-SEQ ID NO: 24 with CasM.26-SEQ ID NO: 69 as a control.

FIGS. 12A-12D depict the trans cleavage activity of CasM.1862909-SEQ ID NO: 22 (FIG. 12B) and CasM.1862921-SEQ ID NO: 24 (FIG. 12C) on an HRP-based reporter immobilized to a solid support with CasM.26-SEQ ID NO: 69 (FIG. 12A) as a control.

FIG. 13 depicts the ability of CasM1862921-SEQ ID NO: 24 to detect two strains of Influenza A RNA with various guide RNA (SEQ ID NOs: 70-72).

FIGS. 14A-14F depict the ability of SEQ ID NOs: 20, 21 and 69 to detect a target nucleic acid at temperatures between 4-37° C.

FIGS. 15A-15F depict the ability of SEQ ID NOs: 22, 23, and 69 to detect a target nucleic acid at temperatures between 4-37° C.

FIGS. 16A-16F depict the ability of SEQ ID NOs: 24, 25, and 69 to detect a target nucleic acid at temperatures between 4-37° C.

### DETAILED DESCRIPTION

Programmable nucleases can be proteins that cleave a target nucleic acid at a specific sequence in a programmable manner. For example, a Type VI CRISPR/Cas protein is a programmable nuclease, which when bound to an engineered guide nucleic acid, binds to a target nucleic acid molecule. In some embodiments, a Type VI CRISPR/Cas protein is a protein that can cleave a target nucleic acid molecule at a specific sequence in a programmable manner. Type VI CRISPR/Cas proteins can also have trans-cleavage activity in which the protein, when activated by its target nucleic acid molecule, non-specifically cleaves other non-target nucleic acid molecules. This “collateral activity” in the presence of a reporter molecule can be used to detect specific target nucleic acid molecules making Type VI CRISPR/Cas proteins a useful tool for molecular diagnostics. Exemplary Type VI CRISPR/Cas proteins are CRISPR/Cas proteins comprising a HEPN domain, such as Cas13.

The present disclosure provides methods, compositions, systems, and kits comprising programmable nucleases, such as Type VI CRISPR/Cas proteins which are phylogenetically distinct from Group 1, Group 2, and Group 3 Cas13 (e.g, Cas13a, Cas13b, and Cas13c, respectively) proteins. An illustrative programmable Type VI CRISPR/Cas protein comprises a Type VI CRISPR/Cas protein or a nucleic acid encoding the Type VI Cas protein, wherein Type VI CRISPR/Cas protein comprises at least 50%, 55%, 60%, 65%, 70%, 75%, 80%, 85%, 90%, 95%, 96%, 97%, 98%, 99%, 100% sequence identity to a sequence selected from the group consisting of SEQ ID NO: 1-27. In some embodiments, the Type VI Cas protein is phylogenetically distinct from Group 1, Group 2, and Group 3 Cas13 (e.g. Cas13a, Cas13b, or Cas13c) proteins. In some embodiments, the composition further comprises an engineered guide nucleic acid or a nucleic acid encoding the engineered guide nucleic acid, wherein the engineered guide nucleic acid comprises a region comprising a nucleotide sequence that is complementary to a target nucleic acid sequence and an additional region, wherein the region and the additional region are heterologous to each other. The Type VI CRISPR/Cas protein and the guide nucleic acid may be complexed together in a ribonucleoprotein complex. Alternatively, compositions consistent with the present disclosure include nucleic acids encoding for the Type VI CRISPR/Cas protein and the engineered guide nucleic acid. In some embodiments, the engineered guide nucleic acid comprises a repeat sequence with at least about 50%, 55%, 60%, 65%, 70%, 75%, 80%, 85%, 90%, 95%, 96%, 97%, 98%, 99%, 100% sequence identity to a sequence selected from the group consisting of SEQ ID NO: 28-SEQ ID NO: 32.

Also disclosed herein are compositions, methods, and systems for modifying a target nucleic acid sequence. An illustrative method for modifying a target nucleic acid sequence comprises contacting a target nucleic acid sequence with a Type VI CRISPR/Cas protein comprising at least 50%, 55%, 60%, 65%, 70%, 75%, 80%, 85%, 90%, 95%, 96%, 97%, 98%, 99%, 100% sequence identity to a sequence selected from the group consisting of SEQ ID NO: 1-27 and a guide nucleic acid, wherein the Type VI CRISPR/Cas protein cleaves the target nucleic acid sequence, thereby modifying the target nucleic acid sequence. In some embodiments, the Type VI CRISPR/Cas protein introduces a single-stranded break.

Also disclosed herein are compositions, methods, and systems for modifying a target nucleic acid sequence comprising use of two or more Type VI CRISPR/Cas proteins. An illustrative method for introducing a break in a target nucleic acid comprises contacting the target nucleic acid with: (a) a first engineered guide nucleic acid comprising a region that binds to a first programmable nuclease comprising at least 50%, 55%, 60%, 65%, 70%, 75%, 80%, 85%, 90%, 95%, 96%, 97%, 98%, 99%, 100% sequence identity to a sequence selected from the group consisting of SEQ ID NO: 1-27; and (b) a second engineered guide nucleic acid comprising a region that binds to a second programmable nuclease comprising at least 50%, 55%, 60%, 65%, 70%, 75%, 80%, 85%, 90%, 95%, 96%, 97%, 98%, 99%, 100% sequence identity to a sequence selected from the group consisting of SEQ ID NO: 1-27, wherein the first engineered guide nucleic acid comprises an additional region that binds to the target nucleic acid and wherein the second engineered guide nucleic acid comprises an additional region that binds to the target nucleic acid.

Also disclosed herein are compositions, methods, and systems for detecting a target nucleic acid molecule in a sample. An illustrative method for detecting a target nucleic acid molecule in a sample comprises contacting the sample comprising the target nucleic acid molecule with (a) a Type VI CRISPR/Cas protein comprising at least 50%, 55%, 60%, 65%, 70%, 75%, 80%, 85%, 90%, 95%, 96%, 97%, 98%, 99%, 100% sequence identity to a sequence selected from the group consisting of SEQ ID NO: 1-27; and (b) an engineered guide RNA comprising a region that binds to the Type VI CRISPR/Cas protein and an additional region that binds to the target nucleic acid; and (c) a labeled, single stranded RNA reporter; cleaving the labeled single stranded RNA reporter by the Type VI CRISPR/Cas protein to release a detectable label; and detecting the target nucleic acid by measuring a signal from the detectable label.

### DEFINITIONS AND GENERAL DESCRIPTION

Unless otherwise indicated, all technical terms used herein have the same meaning as commonly understood by one of ordinary skill in the art to which this invention belongs. Unless otherwise indicated or obvious from context, the following terms have the following meanings:

As used in this specification and the appended claims, the singular forms “a,” “an,” and “the” include plural references unless the context clearly dictates otherwise.

Any reference to “or” herein is intended to encompass “and/or” unless otherwise stated. As used herein, the term “and/or” includes any and all combinations of one or more of the associated listed items.

Use of the term “including” as well as other forms, such as “includes” and “included,” is not limiting.

As used herein, the term “comprising” and its grammatical equivalents specifies the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

As used herein, the term “about” in reference to a number or range of numbers is understood to mean the stated number and numbers+/−10% thereof, or 10% below the lower listed limit and 10% above the higher listed limit for the values listed for a range.

As used herein the terms “individual,” “subject,” and “patient” are used interchangeably and include any member of the animal kingdom, including humans.

As used herein, the terms, “percent identity (% identity) and “percent identical,” refer to the extent to which two sequences (nucleotide or amino acid) have the same residue at the same positions in an alignment. For example, “an amino acid sequence is X % identical to SEQ ID NO: Y” can refer to % identity of the amino acid sequence to SEQ ID NO: Y and is elaborated as X % of residues in the amino acid sequence are identical to the residues of sequence disclosed in SEQ ID NO: Y. Generally, computer programs can be employed for such calculations. Illustrative programs that compare and align pairs of sequences, include ALIGN (Myers and Miller, Comput Appl Biosci. 1988 March; 4(1):11-7), FASTA (Pearson and Lipman, Proc Natl Acad Sci USA. 1988 April; 85(8):2444-8; Pearson, Methods Enzymol. 1990; 183:63-98) and gapped BLAST (Altschul et al., Nucleic Acids Res. 1997 Sep. 1; 25(17):3389-40), BLASTP, BLASTN, or GCG (Devereux et al., Nucleic Acids Res. 1984 Jan. 11; 12(1 Pt 1):387-95).

As used herein, the term “heterologous” may be used to describe/indicate that a first sequence is different from a second sequence and do not naturally occur together. As used herein, the term “heterologous” may be used to describe that a first moiety (e.g., a first sequence) is different from a second moiety (e.g., a second sequence) and, as such, the two moieties do not naturally occur together and are engineered to be a part of one entity. For example, a guide nucleic acid sequence comprising a region and an additional region that are heterologous to each other may indicate that the guide nucleic acid sequence is engineered to include the region and the additional region.

In some embodiments, a heterologous nucleotide or polypeptide sequence is a nucleotide or polypeptide sequence that is not found in a native nucleic acid or protein, respectively. In some embodiments, fusion proteins comprise a programmable nuclease and a fusion partner protein, wherein the fusion partner protein is heterologous to a programmable nuclease. These fusion proteins may be referred to as a “heterologous protein.” A protein that is heterologous to the programmable nuclease is a protein that is not covalently linked via an amide bond to the programmable nuclease in nature. In some embodiments, a heterologous protein is not encoded by a species that encodes the programmable nuclease. In some instances, the heterologous protein exhibits an activity (e.g., enzymatic activity) when it is fused to the programmable nuclease. In some instances, the heterologous protein exhibits increased or reduced activity (e.g., enzymatic activity) when it is fused to the programmable nuclease, relative to when it is not fused to the programmable nuclease. In some instances, the heterologous protein exhibits an activity (e.g., enzymatic activity) that it does not exhibit when it is fused to the programmable nuclease. A guide nucleic acid may comprise a first sequence and a second sequence, wherein the first sequence and the second sequence are not found covalently linked via a phosphodiester bond in nature. Thus, the first sequence is considered to be heterologous with the second sequence, and the guide nucleic acid may be referred to as a heterologous guide nucleic acid.

### Programmable Nucleases

The present disclosure provides methods and compositions comprising programmable nucleases. The programmable nucleases can be complexed with an engineered guide nucleic acid of the disclosure for targeting a target nucleic acid for detection, editing, modification, or regulation of the target nucleic acid.

In some embodiments, a programmable nuclease is a protein, polypeptide, or peptide that non-covalently binds to a guide nucleic acid to form a complex that contacts a target nucleic acid, wherein at least a portion of the guide nucleic acid hybridizes to a target sequence of the target nucleic acid.

A complex between a programmable nuclease and a guide nucleic acid can include multiple programmable nucleases or a single programmable nuclease. In some instances, the programmable nuclease modifies the target nucleic acid when the complex contacts the target nucleic acid.

A non-limiting example of a programmable nuclease modifying a target nucleic acid is cleaving of a phosphodiester bond of the target nucleic acid. Additional examples of modifications a programmable nuclease can make to target nucleic acids are described herein and throughout. A programmable nuclease may be brought into proximity of a target nucleic acid in the presence of a guide nucleic acid when the guide nucleic acid includes a nucleotide sequence that is complementary with a target sequence in the target nucleic acid. In some embodiments, complementary or complementarity, with reference to a nucleic acid molecule or nucleotide sequence, is the characteristic of a polynucleotide having nucleotides that base pair with their Watson-Crick counterparts (C with G; or A with T) in a reference nucleic acid. For example, when every nucleotide in a polynucleotide forms a base pair with a reference nucleic acid, that polynucleotide is said to be 100% complementary to the reference nucleic acid. In a double stranded DNA or RNA sequence, the upper (sense) strand sequence is in general, understood as going in the direction from its 5′- to 3′-end, and the complementary sequence is thus understood as the sequence of the lower (antisense) strand in the same direction as the upper strand. Following the same logic, the reverse sequence is understood as the sequence of the upper strand in the direction from its 3′- to its 5′-end, while the ‘reverse complement’ sequence or the ‘reverse complementary’ sequence is understood as the sequence of the lower strand in the direction of its 5′- to its 3′-end. Each nucleotide in a double stranded DNA or RNA molecule that is paired with its Watson-Crick counterpart called its complementary nucleotide.

The ability of a programmable nuclease to modify a target nucleic acid may be dependent upon the programmable nuclease being bound to a guide nucleic acid and the guide nucleic acid being hybridized to a target nucleic acid. A programmable nuclease may also recognize a protospacer adjacent motif (PAM) sequence present in the target nucleic acid, which may direct the modification activity of the programmable nuclease. In some embodiments, a protospacer adjacent motif (PAM) is a nucleotide sequence found in a target nucleic acid that directs a programmable nuclease to modify the target nucleic acid at a specific location. A PAM sequence may be required for a complex having a programmable nuclease and a guide nucleic acid to hybridize to and modify the target nucleic acid. However, a given programmable nuclease may not require a PAM sequence being present in a target nucleic acid for the programmable nuclease to modify the target nucleic acid.

A programmable nuclease may modify a nucleic acid by cis cleavage or trans cleavage. The modification of the target nucleic acid generated by a programmable nuclease may, as a non-limiting example, result in modulation of the expression of the nucleic acid (e.g., increasing or decreasing expression of the nucleic acid) or modulation of the activity of a translation product of the target nucleic acid (e.g., inactivation of a protein binding to an RNA molecule or hybridization). A programmable nuclease may be a CRISPR-associated (“Cas”) protein. A programmable nuclease may function as a single protein, including a single protein that is capable of binding to a guide nucleic acid and modifying a target nucleic acid. Alternatively, a programmable nuclease may function as part of a multiprotein complex, including, for example, a complex having two or more programmable nucleases, including two or more of the same programmable nucleases (e.g., dimer or multimer). A programmable nuclease, when functioning in a multiprotein complex, may have only one functional activity (e.g., binding to a guide nucleic acid), while other programmable nucleases present in the multiprotein complex are capable of the other functional activity (e.g., modifying a target nucleic acid). A programmable nuclease may be a modified programmable nuclease having reduced modification activity (e.g., a catalytically defective programmable nuclease) or no modification activity (e.g., a catalytically inactive programmable nuclease). Accordingly, a programmable nuclease as used herein encompasses a modified or programmable nuclease that does not have nuclease activity.

The programmable nuclease can be used for detecting a target nucleic acid. For example, in certain embodiments, when the programmable nuclease is complexed with the engineered guide nucleic acid and the target nucleic acid hybridizes to the guide nucleic acid, trans-collateral cleavage of RNA or DNA, such as an RNA reporter or a single stranded DNA reporter, by the programmable nuclease is activated. Detection of trans-collateral cleavage of an RNA or a single stranded DNA can be used to determine a target nucleic acid in a sample. In some embodiments, a sample is something comprising a target nucleic acid. In some instances, the sample is a biological sample, such as a biological fluid or tissue sample. In some instances, the sample is an environmental sample. The sample may be a biological sample or environmental sample that is modified or manipulated. By way of non-limiting example, samples may be modified or manipulated with purification techniques, heat, nucleic acid amplification, salts and buffers.

The programmable nuclease can be used for editing or modifying a target nucleic acid, for example, by site-specific cleavage of a target sequence, donor nucleic acid insertion, or a combination thereof.

The programmable nucleases of the present disclosure can show enhanced activity, as measured by enhanced cleavage of a reporter (e.g., an RNA-FQ reporter), under certain conditions in the presence of the target nucleic acid. For example, the programmable nucleases of the present disclosure can have variable levels of activity based on a buffer formulation, a pH level, temperature, or salt. Buffers consistent with the present disclosure include phosphate buffers, Tris buffers, and HEPES buffers. Programmable nucleases of the present disclosure can show optimal activity in phosphate buffers, Tris buffers, and HEPES buffers. In some embodiments, the target nucleic acid is DNA or RNA.

Programmable nucleases can also exhibit varying levels or single-stranded cleavage activity at different pH levels. For example, enhanced cleavage can be observed between pH 7 and pH 9. In some embodiments, programmable nuclease of the present disclosure exhibit enhanced cleavage at about pH 7, about pH 7.1, about pH 7.2, about pH 7.3, about pH 7.4, about pH 7.5, about pH 7.6, about pH 7.7, about pH 7.8, about pH 7.9, about pH 8, about pH 8.1, about pH 8.2, about pH 8.3, about pH 8.4, about pH 8.5, about pH 8.6, about pH 8.7, about pH 8.8, about pH 8.9, about pH 9, from pH 7 to 7.5, from pH 7.5 to 8, from pH 8 to 8.5, from pH 8.5 to 9, or from pH 7 to 8.5.

In some embodiments, the programmable nucleases of the present disclosure exhibits enhanced cleavage of reporters (e.g., ssDNA-FQ or ssRNA-FQ reporters) at a temperature of 25° C. to 50° C. in the presence of target DNA. For example, the programmable nucleases of the present disclosure can exhibit enhanced cleavage of a reporter (e.g., an ssRNA-FQ or ssDNA-FQ reporter) at about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., from 30° C. to 40° C., from 35° C. to 45° C., or from 35° C. to 40° C.

The programmable nucleases of the present disclosure may not be sensitive to salt concentrations in a sample in the presence of the target nucleic acid. Advantageously, said programmable nucleases can be active and capable of cleaving a reporter (e.g., an ssRNA-FQ or ssDNA-FQ reporter) sequences under varying salt concentrations from 25 nM salt to 200 mM salt. Various salts are consistent with this property of the programmable nucleases disclosed herein, including NaCl or KCl. The programmable nucleases of the present disclosure can be active at salt concentrations of from 25 nM to 500 nM salt, from 500 nM to 1000 nM salt, from 1000 nM to 2000 nM salt, from 2000 nM to 3000 nM salt, from 3000 nM to 4000 nM salt, from 4000 nM to 5000 nM salt, from 5000 nM to 6000 nM salt, from 6000 nM to 7000 nM salt, from 7000 nM to 8000 nM salt, from 8000 nM to 9000 nM salt, from 9000 nM to 0.01 mM salt, from 0.01 mM to 0.05 mM salt, from 0.05 mM to 0.1 mM salt, from 0.1 mM to 10 mM salt, from 10 mM to 100 mM salt, or from 100 mM to 500 mM salt. Thus, the programmable nucleases of the present disclosure can exhibit cleavage activity independent of the salt concentration in a sample.

Programmable nucleases of the present disclosure can be capable of cleaving any a reporter (e.g., an ssRNA-FQ or ssDNA-FQ reporter), regardless of its sequence. The programmable nucleases provided herein can, thus, be capable of cleaving a universal a reporter (e.g., an ssRNA-FQ or ssDNA-FQ reporter). In some embodiments, the programmable nucleases provided herein cleave homopolymer a reporter (e.g., an ssRNA-FQ or ssDNA-FQ reporter) comprising 5 to 20 adenines, 5 to 20 thymines, 5 to 20 cytosines, or 5 to 20 guanines. Programmable nucleases of the present disclosure, thus, are capable of cleaving ssRNA-FQ reporters also cleaved by programmable nucleases, as disclosed elsewhere herein, allowing for facile multiplexing of multiple programmable nucleases and programmable nucleases in a single assay having a single ssRNA-FQ reporter.

Programmable nucleases of the present disclosure can bind a wild type protospacer adjacent motif protospacer flanking site (PFS) or mutated PFS.

In some embodiments, the programmable nuclease is a programmable nuclease comprising site-specific nucleic acid cleavage activity. In some embodiments, a cleavage, with reference to a nucleic acid molecule or nuclease activity of a programmable nuclease, is the hydrolysis of a phosphodiester bond of a nucleic acid molecule that results in breakage of that bond. The result of this breakage can be a nick (hydrolysis of a single phosphodiester bond on one side of a double-stranded molecule), single strand break (hydrolysis of a single phosphodiester bond on a single-stranded molecule) or double strand break (hydrolysis of two phosphodiester bonds on both sides of a double-stranded molecule) depending upon whether the nucleic acid molecule is single-stranded (e.g., ssDNA or ssRNA) or double-stranded (e.g., dsDNA) and the type of nuclease activity being catalyzed by the programmable nuclease.

In some embodiments, the programmable nuclease is a programmable nuclease comprising RNA cleavage activity. In some embodiments, the programmable nuclease is a programmable nuclease comprising a catalytically inactive nuclease domain. In some embodiments, the programmable nuclease comprising a catalytically inactive nuclease domain can include at least 1, at least 2, at least 3, at least 4, or at least 5 mutations relative to a wild type nuclease domain. Said mutations may be present within the cleaving or active site of the nuclease. In some embodiments, the programmable nuclease comprises two nuclease domains.

In some embodiments, the programmable nuclease is a programmable RNA nuclease. In some embodiments, the programmable nuclease is a Type VI CRISPR/Cas protein. A Type VI CRISPR/Cas protein can function as an endonuclease that catalyzes cleavage at a specific sequence in a target nucleic acid. A Type VI CRISPR/Cas protein of the present disclosure can have a single active site in a HEPN domain that can cleave nucleic acids. A Type VI CRISPR/Cas protein of the present disclosure can preferably have two active sites in two HEPN domains that can cleave nucleic acids. The HEPN catalytic site can render the programmable Type VI CRISPR/Cas protein nuclease especially advantageous for genome engineering and new functionalities for genome manipulation. In some embodiments, the Type VI CRISPR/Cas protein is a Cas13 protein or a Cas13-like protein.

A programmable nuclease of the present disclosure can comprise at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity with any one of SEQ ID NO: 1 to SEQ ID NO: 27.

Provided herein, in some embodiments, are compositions that comprise one or more Type VI CRISPR/Cas proteins. TABLE 1 provides illustrative amino acid sequences of Type VI CRISPR/Cas proteins (e.g., any one of SEQ ID NO: 1-27, or fragments or variants thereof). In some embodiments, the amino acid sequence of the Type VI CRISPR/Cas is at least 65%, at least 70%, at least 75%, at least 80%, at least 85%, at least 90%, at least 95%, at least 97%, or at least 98%, at least 99%, or 100% identical to any one of SEQ ID NOs: 1-27.

TABLE 1
Amino acid sequences of Type VI CRISPR/Cas proteins

| SEQ | |
|---|---|
| ID | |
| NO: | Sequence |
| 1 | MEDDKKTKESTNMLDNKHFWAAFLNLARHNVYITVNHINKILEEG |
| | EINRDGYETTLKNSWDEIKDINKKDRLSKLIIKHFPFLEAATYRQNP |
| | TDTTKQKEEKQAEAQSLESLKKSFFVFIYKLRDLRNHYSHYKHSKS |
| | LERPKFEEDLQEKMYNIFDVSICLVKKDYQYNKDINPDEDFKHLDR |
| | KRKGKFKYSFADNEGNITESGLLFFVSLFLEKKDAIWVQKKLEGFK |
| | CSNESYQKMTNEVFCRSRMLLPKLRLESTQTQDWILLDMLNELIRC |
| | PKSLYERLRGDDREKFRVPLDPIDEDYDAEQEPFKNTLVRHQDRFP |
| | YFALRYFDYNEIFTNLRFQIDLGTYHFSIYKKLIGGQKEDRHLTHKL |
| | YGFERIQEFDKQNRPDEWKAIVKDSDTFKKKEEKEEEKPYISETTPH |
| | YHLENKKIGIAFKNHNIWPSTQTELTNNKRKKYNLGTSIKAEAFLS |
| | VHELLPMMFYYLLLKTENTKNDNKVEGKKETKKQEKHKIESIIESK |
| | IKDMYALYDAFANGEINSEKELEEYLKGKDIEIVHLPKQMIAILKNE |
| | HKDMAEKAEAKQEKMKLATENRLKTLDKQLKGKIQNGKRYNSAP |
| | KSGEIASWLVNDMMRFQPVQRDENGEPLNNSKANSTEYQLLQRTL |
| | AFFGSEHERLAPYFKQTKLIESSNPHPFLNDTEWEKCSNILSFYRSY |
| | LKAKKNFLESLKPEDWEKNQYFLMLKEPKTNRKTLVQGWKNGFN |
| | LPRGIFTEPIRKWFMEHWKNIKVDDSKRVGLVAKVIPLFFSEKYKD |
| | SVQPFYNYPFNVGDVNKPKEEDFLHREERIELWDKKKDKFKGYKA |
| | KKKFKEMTDKEKEEHRSYLEFQSWNKFERELRLVRNQDIVTWLLC |
| | TELINKLKIDELNIKELKKLRLKDINTDTAKEEKNNILNRVMPMELP |
| | VTVYEVNKGGYIIKNKPLHTIYIKEAKTKLLKQGNFKALVKDRRLN |
| | GLFSFVKTPSEAESESNPISKSRVESELGKYQNARLNIIEDMLALEKT |
| | LIDKYNSLDTDNFHNMLTGWLKLKGKANKARFQNEVDLLVAVRN |
| | AFSHNQYPMYNENLFGNIERFSLSSSNIIESKGLDIAVKLKEEVSKA |
| | AKKIQNEEDNKKEKET |
| 2 | MENDKKLEESACYTLNDKHFWAAFLNLARHNVYITVNHINKTLGE |
| | GEINRDGYETTLENTWNEIKDINKKARLRELIIKHFPFLEAATYQQR |
| | STDSTKQKEEKQAEAQSLESLKHCLFPFLKKLQKSRDHYSHYKHSK |
| | SLERPKFEEDLQKKMYNIFDVSIRLVKEDYKHNTDINLKEDFKHLN |
| | RTGKFKYSFADNKGNITESGLLFFISLFLEKKDAIWMQKKLKGFKD |
| | SREKYQKMTNEVFCRSRILLPKLRLESTQTQDWILLDMLNELIRCP |
| | KSLYERLREKERKEFKVPIEIADEDYDAEQEPFRNTLVRHQDRFPYF |
| | ALRYFDYNEIFTNLRFQIDLGTYHFSIYKKLIGGNKEDRHLTHKLYG |
| | FERIQEFAKQNRPDEWKALVKDLDTFNKEEEKLYISETTPHYHLEN |
| | EKIGIVFKNHNIWPSTQTELTNNNRKKYNLGVSIKAEAFLSVHELLP |
| | MMFYYLLLKTKNTHNGNEVEAKKKGTKNKKQEKHKIEAIIESKIK |
| | DIYNLYDAFANGEINSIEELEEHCKGKDIEIGHLPKQMIAILKDEHK |
| | DMAKKAETKQEKMILATENRLKTLDKKLKGKIRNGKRCNSALKS |
| | GEIASWLVNDMMRFQPVQRDEEGKPLNNSKANSTEYQLLQRTLAF |
| | FGSEHERLDPYFKQMNLIGDNNPHPFLNDTEWEKCNNILSFYRSYL |
| | KARKNFLESLKPEDWEKNQYFLMLKEPKTNRKTLVQGWKNGFNL |
| | PRGIFTEPIRKWFMEHWKSIKVDDLKRVGLVAKVIPLFFSEKYKDS |
| | VQPFYNYPFNIGNINKPEEKDFLDYEKRKKLWDKKQAKFKGKTDK |
| | EKREDPSYPDFKSWKKFERELRLVRNQDIVTWMLCMELFEEQKVD |
| | GLNTGQIHLKDIVTNVDEEGNNNILNQIKPMQLPITVYDKENSPKR |
| | KPLHTIYIEETGTKLLKQGNFKALVKDRRLNGLFSFVKTSSEAESKS |
| | KPISKLRVEYELGAYQKARIDIIKDMLALEKTLIDNDENLPTNKFSD |
| | MLKSWLKGKGEANKARLQNDVDLLVAVRNAFSHNQYPMYNSEV |
| | FKGMKLLSLSSDIPEKEGLGIAKQLKDKIKETIERIIEIEKEIRN |
| 3 | MKEEEKGKTPVVSTYNKDDKHFWAAFLNLARHNVYITVNHINKIL |
| | GEAEINIDGYDISLKNIWSGIRDINKKARLEELMIKHFPFLEAMTHQ |
| | QRLTGNTKQKEEKQAKIQSFEILKDNLFLLLKMLQDLRNHYSHYK |
| | HSKSLERPKFDEDIRNKLYNIFDTSIRLVKEDYKHNPDIKPKEHFKH |
| | LDRKRNEKFRYSFADNEGNITISGLLFFVSLFLEKKDAIWVQKKLE |
| | GFRCSNESYQKMTNEVFCRNRILMPKLRLESTQTQDWILLDMLNE |
| | LIRCPKSLYERLKGEDRKKFEVPFDSTDEDYDAEQDPFKNTLIRYQ |
| | DRFPYFVLRYFDYNEIFENLRFQIDLGTYHFSIYKKLIGGQKEDRHL |
| | THKLYGFERLQEFAKQNRPDEWKAIIKDLDTFKKQEDEKVNEKPYI |
| | SETTPHYHLENEKIGIAFKNHNIWPTTNTEFTNNKRKKYNLGTSIKA |
| | EAFLSVHELLPMMFYYMLLKIKNEGPRKVEAIIESKIKDIFTLYDAF |
| | ANDEIDNTDELEKYCEDKDIKIGHLPKQMIAILYNKHKDMVEKAK |
| | VKQEKMKLATENRLKTLDKLIEGKMKYAKRGISPLKSGEIASWLV |
| | NDMMRFQPVQRDENGEPLNNSKANSTEYQLLQRTLALFGSEHKRL |
| | TPYFKQMNLIGDNNSHPFLKQTEWEECNNILSFYRSYLEARKNFLK |
| | NLEPKDWKKNQYFLKIKEPKSNRQTLVQGWKNGFNLPRGIFTEPIK |
| | KWFIEHRKKMKVDELKSVGLVAKVIPLYFNEKYKDSVQPFYNYPF |
| | NVGDINKPKEKDFLDNGKRRKLWDKKQLEFKGKTEEEKRKNPSYL |
| | DFKSWKKFERELRLIRNQDIVTWLMCIELYNNQKVENLNTGHIYLK |
| | DIITNVKEERNNNILNQIKSMQLPVTVYENESSPKKKYLRTIYIEENG |
| | TKLLKQGNFKALVKDRRLNGLFSFVKTSSEAESKRNPISKLRVEHE |
| | LREYQKARIDIIKDMLALEKTLIDNYDSLDTDNFHNMLTGWLELKG |
| | EAIKTKFNNDVKLLTAVRNAFSHNQYPMYDENLFINIERFSLSSSRI |
| | LESKGLGIAVKLKEEVSNAIEKIKNEEDAKKET |
| 4 | MKNQNSLPSNPTDILEDKPFWAAFFNLARHNVYLTVNHINKLIDLE |
| | KLYDEDKHEEIFEREDILNISDDVMNDVNSNGKKRKLDIKKIWDDL |
| | DTDLTRKYLLRELILKHFPFIQPAIIGAQTKERTTIDKDKRNTSTSND |
| | SLKQTGEGDINDPLSLSNVKSMFFSLLQMLEQLRNYYSHVKHSKSA |
| | TMPNFDEDLLNWMRYIFIDSVNKVKEDYSSNSVIDPNTCFSHLIYK |
| | DKQGKIKPCRYPFTSKDGSINAFGLLFFVSLFLEKQDSIWMQKKIPG |
| | FKKASENYMKMTNEVFCRNHILLPKIRLETVYDKDWMLLDMLNE |
| | VVRCPLSLYKRLTPADQNKFKVPEKSSDNANRQEDDNPFSRILVRH |
| | QNRFPYFVLRFFDLNEVFTTLRFQINLGCYHFAICKKQIGDKKEVH |
| | HLTRTLYGFSRLQNFTQNTRPEEWNTLVKTTEPSSGNDGKTVQGV |
| | PLPYISYTIPHYQIENEKIGIKIFDGDTAVDTDIWPSVSTEKQLNKPD |
| | KYTLTPGFKADVFLSVHELLPMMFYYQLLLCEGMLKTDAGNAVE |
| | KVLIDTRNAIFNLYDAFVQEKINTITDLENYLQDKPILIGHLPKQMID |
| | LLKGHQRDMLKAAEQKKAMLIKDTERRLERLNKQPEQKPNVAAK |
| | NIGALLRNGQIADWLVKDMMRFQPVKRDKEGNPINCSKANSTEYQ |
| | MLQRAFAFYATDSCRLPRYFEQLHLINCDNSHLFLSRFEYDKQPNL |
| | IAFYAAYLKAKLDFLNELQPQNWVSDNYFLLLRAPKNNRQKLAEG |
| | WKNGFNLPRGLFTEKIKTWFNEHKTIVDISDCDIFKNRVGQVARLIP |
| | VFFDKKFKDHSQPFYRYNFNVGNVSKPTEAKYLSKEKREELFKSY |
| | QNKFKNNIPAEKTKEYREYKNFSLWKKFERELRLIKNQDILTWLM |
| | CKNLFDEKIEQGIDIPYIKLDSLQTNTSTKGSLNALAQVLPMVLAIYI |
| | GNSESNNGTGANEEENKGPMVYIKEEGTKLLKWGNFKTLLADRRI |
| | KGLFSYIEHDDIDLKQHPLTKRRVDLELDLYQTCRIDIFQQTLGLEA |
| | QLLNKYSDLNTDNFYQMLIGWRKKEGIPRDIKEDTDFLKDVRNAF |
| | SHNQYPDSKKIAFSRIRKENPKKTILNEKKGLGIAKQMYEEVEKVV |
| | NRIKGIELFD |
| 5 | MKNQNTLPSNPTDILKDKPFWAAFFNLARHNVYLTVNHINKLLDL |
| | EKLYNKDKHKEIFEHEDIFNISDDVMNDVNSNGKKRKLDIKKIWA |
| | NLDTDLTRKYQLRELILKHFPFIQPAIIGAQTKERTTIDKDKRSTSTS |
| | NDSLKPTGEGDINDPLSLSNVKSIFFRLLQMLEQLRNYYSHVKHSK |
| | SATMPNFDEGLLKSMYNIFIDSVNKVKEDYSSNSVIDPNTSFSHLIS |
| | KDEQGEIKPCRYSFTSKDGSINASGLLFFVSLFLEKQDSIWMQKKIP |
| | GFKKTSENYMKMTNEVFCRNHILLPKMRLETVYDKDWMLLDML |
| | NEVVRCPLSLYKRLAPADQNKFKVPEKSSDNANRQEDDNPFSRILV |
| | RHQNRFPYFALRFFDLNEVFTTLRFQINLGCYHFAICKKQIGDKKEV |
| | HHLTRTLYGFSRLQNFTQNTRPEEWNTLVKTTEPSSGNDGKTVQG |
| | VPLPYISYTIPHYQIENEKIGIKIFDGDTAVDTDIWPSVSTEKQLNKP |
| | DKYTLTPGFKADVFLSVHELLPMMFYYQLLLCEGMLKTDAGNAV |
| | EKVLIDTRNAIFNLYDAFVQEKINTITDLENYLQDKPILIGHLPKQMI |
| | DLLKGHQRDMLKAVEQKKAMLIKDTERRLERLNKQPEQKPNVAA |
| | KNTGTLLRNGQIADWLVKDMMRFQPVKRDKEGNPINCSKANSTE |
| | YQMLQRAFAFYTTDSYRLPRYFEQLHLINCDNSHLFLSRFEYDKQP |
| | NLIAFYAAYLEAKLEFLNELQPQNWASDNYFLLLRAPKNDRQKLA |
| | EGWKNGFNLPRGLFTEKIKTWFNEHKTIVDISDCDIFKNRVGQVAR |
| | LIPVFFDKKFKDHSQPFYTYNFNVGNVSKITEANYLSKEKRENLFK |
| | SYQNKFKNNIPAEKTKEYREYKNFSSWKKFERELRLIKNQDILTWL |
| | MCKNLFDEKIKPKKDILEPRIAVSYIKLDSLQTNTSTAGSLNALAKV |
| | VPMTLAIHIDSPKPKGKAGNNEKENKEFTVYIKEEGTKLLKWGNFK |
| | TLLADRRIKGLFSYIEHDDINLEKYPLTKYQVDSELDLYQKYRIDIF |
| | KQTLDLEAQLLDKYSDLNTDNFNQMLSGWSEKEGIPRNIKQDVAF |
| | LIGVRNGFSHNQYPDSKRIAFSRIKKFNPKTSSLQESEGLNIAKQMY |
| | EEAQQVVNKIKNIESFD |
| 6 | MKITKIDGISHKKYIKEGKLVKSTSEENKTDERLSELLTIRLDTYIKN |
| | PDNASEEENRIRRENLKEFFSNKVLYLKDGILYLKDRREKNQLQNK |
| | NYSEEDISEYDLKNKNSFLVLKKILLNEDINSEELEIFRKDVEAKLN |
| | KINSLKYSFEENKANYQKINENNVEKVGGKSKRNIIYDYYRESAKR |
| | NDYINNVQEAFDKLYKKEDIEKLFFLIENSKKHEKYKIRECYHKIIG |
| | RKNDKENFAKIIYEEIQNVNNIKELIEKVPDMSELKKSQVFYKYYL |
| | DKEELNDKNIKYAFCHFVEIEMSQLLKNYVYKRLSNISNDKIKRIFE |
| | YQNLKKLIENKLLNKLDTYVRNCGKYNYYLQDGEIATSDFIAGNR |
| | QNEAFLRNIIGVSSVAYFSLRNILETENKDDITGKMRGKTRIDSKTG |
| | EEKYIPGEVDQIYYENKQNEVKNKLKMFYGYDFDMDNKKEIEDFF |
| | ANIDEAISSIRHGIVHFNLDLDGKDIFAFKNIVPSEISKKMFQNEINEK |
| | KLKLKIFRQLNSANVFRYLEKYKILNYLKRTRFEFVNKNIPFVPSFT |
| | KLYSRIDDLKNSLGIYWKTPKTNDDNKTKEIIDAQIYLLKNIYYGEF |
| | LNYFMSNNGNFFEISREIIELNKNDKRNLKTGFYKLQKFEDIQEKTP |
| | KKYLANIQSLYMINAGNQDEEEKDTYIDFIQKIFLKGFMTYLANNG |
| | RLSLMYIGNDEQINTSLAGKKQEFDKFLKKYEQNNNIEIPHEINEFL |
| | REIKLGKILKYTESLNMFYLILKLLNHKELTNLKGSLEKYQSANKEE |
| | TFSDELELINLLNLDNNRVTEDFELEANEIGKFLDFNGNKIKDRKEL |
| | KKFDTKKIYFDGENIIKHRAFYNIKKYGMLNLLEKIADKAKYKISL |
| | KELKEYSNKKNEIEKNYTMQQNLHRKYARPKKDEKFNDEDYKEY |
| | EKAIGNIQKYTHLKNKVEFNELNLLQGLLLKILHRLVGYTSIWERD |
| | LRFRLKGEFPENQYIEEIFNFDNSKNVKYKSGQIVEKYINFYKELYK |
| | DNVEKRSIYSDKKVKKLKQEKKDLYIRNYIAHFNYIPHAEISLLEVL |
| | ENLRKLLSYDRKLKNAIMKSVVDILKEYGFVATFKIGADKKIGIQT |
| | LESEKIVHLKNLKKKKLMTDRNSEELCKLVKVMFEYKMEEKNLKT |
| | KKCKVI |
| 7 | MKITKIDGILHKKYIKEGKLVKSTSEENKTDERLSELLTIRLDTYIKN |
| | PDNASEEENRIRRETLKEFFSNKVLYLKDSILYLKDRREKNQLQNK |
| | NYSEEDISEYDLKNKNSFLVLKKILLNEDINSEELEIFRNDFEKKLDK |
| | INSLKYSLEENKANYQKINENNIKKVEGKSKRNIFYNYYKDSAKRN |
| | DYINNIQEAFDKLYKKEDIENLFFLIENSKKHEKYKIRECYHKIIGRK |
| | NDKENFATIIYEEIQNVNNMKELIEKVPNVSELKKSQVFYKYYLNK |
| | EKLNDENIKYVFCHFVEIEMSKLLKNYVYKKPSNISNDKVKRIFEY |
| | QSLKKLIENKLLNKLDTYVRNCGKYSFYLQDGEIATSDFIVGNRQN |
| | EAFLRNIIGVSSTAYFSLRNILETENENDITGRIKGKTVKNKKGEEK |
| | YISGEIDKLYDNNKQNEVKKNLKMFYSYDFNMNRKKEIEDFFSNID |
| | EAISSIRHGIVHFNLELEGKDIFTFKNIVPSQISKKMFQNEINEKKLKL |
| | KIFRQLNSANVFRYLEKYKILNYLNRTRFEFVNKNIPFVPSFTKLYS |
| | RIDDLKNSLCIYWKIPKANDNNKTKEITDAQIYLLKNIYYGEFLNYF |
| | MSNNGNFFEIIKEIIELNKNDKRNLKTGFYKLQKFENLQEKTPKEYL |
| | ANIQSFYMIDAGNKDEEEKDAYIDFIQKIFLKGFMTYLANNGRLSL |
| | MYIGNDEQINTSLAGKKQEFDKFLKKYEQNNNIEIPHEINEFVREIK |
| | LGKILKYTESLNMFYLILKLLNHKELTNLKGSLEKYQSANKEEAFS |
| | DQLELINLLNLDNNRVTEDFELEADEIGKFLDFNGNKVKDNKELKK |
| | FDTNKIYFDGENIIKHRAFYNIKKYGILNLLEKISDEAKYKISIEELK |
| | NYSNKKIEIEKNHTTQENLHRKYARPRKDEKFNDEDYKKYEKTIRN |
| | IQQYTHLKNKVEFNELNLLQSLLLRILHRLVGYTSIWERDLRFRLK |
| | GEFPENQYIEEIFNFDNSKNVKYKNGQIVEKYISFYKELYKDDMEKI |
| | SIYSDKKVKELKKEKKDLYIRNYIAHFNYIPNAEVSLLEVLENLRKL |
| | LSYDRKLKNAIMKSIVDILKEYGFVVTFKIEKDKKIRIESLKSEEVV |
| | HLKKLKLKDNDKKKEPIKTYRNSKELCKLVKVMFEYKMKEKKSE |
| | N |
| 8 | MKVTKIDGISHKKFEDEGKLVKFTGHFNIKNEMKERLEKLKELKLS |
| | NYIKNPENVKNKDKNKEKETKSRRENLKKYFSEIILRKKEEKYLLK |
| | KTRKFKNITEEINYDDIKKRENQQKIFDVLKELLEQRINENDKEEIL |
| | NFDSVKLKEAFEEDFIKKELKIKAIEESLEKNRADYRKDYVELENE |
| | KYEDVKGQNKRSLVFEYYKNPENREKFKENIKYAFENLYTEENIK |
| | NLYSEIKEIFEKVHLKSKVRYFYQNEIIGESEFSEKDEEGISILYKQII |
| | NSVEKKEKFIEFLQKVKIKDLTRSQIFYKYFLENEELNDENIKYVFS |
| | YFVEIEVNKLLKENVYKTKKFNEGNKYRVKNIFNYDKLKNLVVYK |
| | LENKLNNYVRNCGKYNYHMENGDIATSDINMKNRQTEAFLRSILG |
| | VSSFGYFSLRNILGVNDDDFYKIEKDERKNENFILKKAKEDFTSKNI |
| | FEKVVDKSFEKKGIYQIKENLKMFYGNSFDKVDKDELKKFFVNML |
| | EAITSVRHRIVHYNINTNSENIFDFSNIEVSKLLKNIFEKEIDTRELKL |
| | KIFRQLNSAGVFDYWESWVIKKYLENVKFEFVNKNVPFVPSFKKL |
| | YDRIDNLKGWNALKLGNNINIPKRKEAKDSQIYLLKNIYYGEFVEK |
| | FVNDNKNFEKIVKEIIEINRGAGTNKKTGFYKLEKFETLKANTPTKY |
| | LEKLQSLHKISYDKEKIEEDKDVYVDFVQKIFLKGFVNYLKKLDSL |
| | KSLNLLNLRKDETITDKKSVHDEKLKLWENSGSNLSKMPEEIYEYV |
| | KKIKISNINYNDRMSIFYLLLKLIDYRELTNLRGNLEKYESMNKNKI |
| | YSEELTIINLVNLDNNKVRTNFSLEAEDIGKFLKSSITIKNIAQLNNFS |
| | KIFADGENVIKHRSFYNIKKYGILDLLEKIVAKADLKITKEEIKKYE |
| | NLQNELKRNDFYKIQEQIHRNYNQKPFSIKKIENKKDFEKYKKVIE |
| | KIQDYTQLKNKIEFNDLNLLQSLIFRILHRLAGYTSLWERDLQFKLK |
| | GEFPEDKYIDEIFNSDGNNNQKYKHGGIADKYANFLIEKKEEKSGEI |
| | LNKKQRKKKIKEDLEIRNYIAHFNYLPNAEKSILEILEELRELLKHD |
| | RKLKNAVMKSIKDIFREYGFIVEFTISHTKNGKKIKVCSVKSEKIKH |
| | LKNNELITTRNSEDLCELVKIMLEHKELQK |
| 9 | MCMKITKIDGISHKKYKEKGKLIKNNDTAKDIIEERFNDIEKKTKEL |
| | FQKTLDFYVKNYEKCKEQNKERREKAKNYFSKVKILVDNKKITIC |
| | NENTEKMEIEDFNEYDVRSGKYFNVLNKILNGENYTEEDLEVFEND |
| | LQKRTGRIKSIKNSLEENKAHFKKESINNNIIYDRVKGNNKKSLFYE |
| | YYRISSKHQEYVNNIFEAFDKLYSNSHEAMNNLFSEITKDSKDRNIR |
| | KIREAYHEILNKNKTEFGEELYKKIQDNRNNFDKLLEIEPEIKELTKS |
| | QIFYKYYIDKVNLDETSIKHCFCHLVEIEVNQLLKNYVYSKRNINKE |
| | KLENIFEYCKLKNLIKNKLVNKLNNYIRNCGKYNAYISNNDVVVNS |
| | EKISEIRTKEAFLRSIIGVSSSAYFSLRNILNTDNTQDITNKVDKEVD |
| | KLYQENKKIELEERLKLFFGNYFDINNQQEIKVFLMNIDKIISSIRHEI |
| | IHFKMETNAQNIFDENNVNLGNTAKNIFSNEINEEKIKFKIFKQLNS |
| | ANVFDYLSNKDITEYMDKVVFSFTNRNVSFVPSFTKIYNRVQDLAN |
| | SLEIKKWKIPDKSEGKDAQIYLLKNIYYGKFLDEFLNEENGIFISIKD |
| | KIIELNRNQNKRTGFYKLEKFEKIEETNPKKYLEIIQSLYMINIEEIDS |
| | EGKNIFLDFIQKIFLKGFFEFIKNNYNYLLELKKIQDKKNIFDSEMSE |
| | YIAGEKTLEDIGEINEIIQDIKITEIDKILNQTDKINCFYLLLKLLNYKE |
| | ITELKGNLEKYQILSKTNVYEKELMLLNIVNLDNNKVKIENFKILAE |
| | EIGKFIEKINIEEINKNKKIKTFEELRNFEKGENTGEYYNIYSDDKNIK |
| | NIRNLYNIKKYGMLDLLEKISEKTNYCIKKKDLEEYSELRKQLEDE |
| | KTNFYKIQEYLHSKYQQKPKKILLKNNKNDYEKYKKSIENIEKYVH |
| | LKNKIEFNELNLLQSLLLKILHRLVGFTSIWERDLRFRLIGEFPDELD |
| | VEDIFDHRKRYKGTGKGICKKYDRFINTHTEYKNNNKMENVKFAD |
| | NNPVRNYIAHFNYLPNPKYSILKMMEKLRKLLDYDRKLKNAVMK |
| | SIKDILEEYGFKAEFIINSDKEIILNLVKSVEIIHLGKEDLKSRRNSED |
| | LCKLVKAMLEYSK* |
| 10 | MKVTKIDGLSHKKFEDEGKLVKFKNNKNINEIKERLKKLKELKLD |
| | NYIKNPENVKNKDKDAEKETKIRRTNLKKYFSEIILRKEDEKYILKK |
| | TKKFKNINQEIDYYDVKSKKNQQEIFDVLKEIKIKETEKEEIITFDSE |
| | KLKKVFGEDFVKKEAKIKAIEKSLKINKANYKKDSIKIGDDKYSNV |
| | KGENKRSCIYEYYKKSENLKKFEENIREAFEKLYTEENIKELYSKIE |
| | EVLKKTHLKSIVREFYQNEIIGESEFSKKNGDGISILYNQIKDSIKKEE |
| | NFIEFIENIGNLELKDLTKSQIFYKYFLENEELNDENIKFAFCYFVEIE |
| | VNNLLKENVYKIKRFNEGNKKRIKNIFEYDKLKKLIVYKLENKLNN |
| | YVRNCGKYNYHMENGDIATSDINMRNRQTEAFLRSIIGVSSFGYFS |
| | LRNILGVNDDDFYEIEEGLTEEERKNESNVLKKAKEDITSKSIFEKV |
| | VDKSFEKKGIRSIKENLKMFYGDSFDKANGDELKQFFVNMLNAITS |
| | IRHSVVHYDMNTNSENIFNFSDIEVSRLLKSIFEKETDKRELKLKIFR |
| | QLNSAGVFDYWENWKIKKYLENIKFEFVNKNIPFVPSFTKLYNRID |
| | NLKGSNALNLGYINIPKRKEARDSQIYLLKNIYYGKFVENFVNNKE |
| | NFEKVFKKIIEINKNAGRNKQTNFYKLEKFETLKANTPTEYLEKLQS |
| | LHKINYDKEKVEEDKDTYVDFVQKIFLKGFINYLQKSNSLKPLNLL |
| | NLKKDEVINSEKSSYDEKLKQWENNGSKLSEMPKEIYEYIKKIQIN |
| | KINYSDRMSIFYLLLKLIDHKELTNLRGNLEKYESMNKNKIYSEELN |
| | IVNLVSLDNNKVRTNFSVEAEDIGKFLKTETSIKNNKQLNNFSEIFA |
| | DGQNVIKHRSFYNIKKYGILDLLEKIVAKADLKITKEEIKKYKNLQK |
| | ELEKNDFYKIQEKIHRKYNQKPNLISRTENKKDFNDYKKAIENIQN |
| | YTQLKNKIEFNDLNLLQDLLFRILHRLAGYTSLWERDLQFKLKGEF |
| | PEDKYIDEIFNSDRNNNQKYKSGGIAYKYVDFLIEKEEGKRAGKNK |
| | VKKRSEKEGSFIIRNYIAHFNYIPDAEKSILEILEELRELLKYDRKLK |
| | NAVMKSIKDIFKEYGFIIEFGISHESNSKKIKVVNVESEKIKHLKNNG |
| | LVTTRNSEDLCNLVKVMLEYKKS |
| 11 | MKITKRKWEDVPPLFFHRDKDSGRLIRQDDREHDYTDELFRKTTV |
| | DSIFIRSLQNRLLRSQDKEGNKIGRNEITELVENLCKGAEIQKSIDEIK |
| | DSKSNVKALCDKTLTDYGKSFAAEEGRTTLYEAILNLAKGNAIQK |
| | AMAEAELKQIHEALKKEKEDRINRVQNSIKNNKIPLKVNEDGTISA |
| | NNDRASWLLELLKPVASEHDKGDRYPVLTEMEGIVSFDKLCEEVR |
| | NKIHGRKGRTRSIAMSVDEAVKEYLKELWKKHADRHRDMRYYFQ |
| | AVKEYFKENFPIRTKQKGAGIRQSLLKEDTVVARLLDPKHMAKAV |
| | RRKLINQSIQMHILYGKLYEYCCREDRNLPANSETLQLIQVLEAFKK |
| | QLMTAVLWSVSRLCYFYEFYSGDILLAEEYRRDFLNQKNDHKDIQ |
| | ACKEKLQDFFPLAELQEKIRECADKDINNIKDKITNDVLACNNLLEE |
| | CVFCISKLRNNIFHYKNLSFTQALKHIANENKEEDIPIILRLYKQDCE |
| | NVNKAFANRISSMNLPLYYSTEFLLRIFKKQGAELSLYSTKNQMTP |
| | SFKRVYERGKNLRLEYEREPMRSNIANEYDKDPQSDYWMRWFSQ |
| | FATGTVDTNAGIKGEMEDYPHASVNLGIEIEAGTNIDAQRAMRNLL |
| | QLIYKHHFLPEVQKDETVVTDVIPKVLERNRKLSEVRNRSDKTYGY |
| | KEIEELCKKNKGKKDSLSELMKDLQRKVSETEKEKRELAREKTDY |
| | AQRFILDIFAEAFCNFMDERYEDEYQEIMSPIKNEEEAKKWTEMFT |
| | DKADKDNKDKLLVLKTSFDKDNVEKYLLVLYPLMRLLDDKELSE |
| | LQQQMLRFRASVSGWQGGTDFSEDIAMAEKIEELAELVKLTEPESQ |
| | YAEEAWKNRAIGVFEQFVEGKMAEYGTFYIQNDSLTQVPRGNMR |
| | KLLRSGVIGVYNKVLASCKQATKADYGAYSGDHWSIEDPDGNPIK |
| | SAEQAQALLQRLHKKYTESPSQFSDDDHKLYDKTLRQLDAYNRA |
| | MKNLSFGSLYEICRIHMETLSRWVGFVQDWERDMYFLLLAWTKQ |
| | GKLLNVTETDVIEIFEKGSVIYKMNKKLKNGDRYAFTSIYRNNNDE |
| | KLEFLQIRNFIAHLHLLRKSEWKGDKKAGYSVIEDYINKLRFLLSYD |
| | QKRMNAVTKAMQQIFEKHKVIVKFMVEKGGMLKVKDVKPGIIEH |
| | LKQTKRIEIPCHGERFTDSLLVLMKYQ |
| 12 | MEQKQLESRFNQIFNNKGHTGPIAEKAVKNFETIRQHKVSPRERLH |
| | YFAVGHALRNIDKDLKESIFEYNLDEEQKKQKPTQFTTLQSDFFRFE |
| | NALLTLLKDIRNCNGHYVHTFDKLQLDEILKLQEKNKEHGILNKDA |
| | GCQIIEFLKEAFEFSILIQFLKEKPKEYEKFKKRKNENKNQSLRNLIG |
| | GYEKKLVKYLCDKFFPNEEKQKEIRDKFIEHNLEEAIEDLLFIPVDE |
| | DIEWKLGEEHVVFVIKKGKYLSFYAQLFLLSMFLYKQEANQLISKI |
| | RGFKRSEDEFQYKRNIFTFFSKKVSSQDIHSEEKHLIYFRDIIQYLNR |
| | FPTAWNEYLSPERKNLPMTKLLEKYILEEEIFRTFSTYKNDCNRELF |
| | LKYTIKRLFCKKAELFDAEKISIDDNLRKKFNYEIDTSPELKNIHEKL |
| | KGKLKPKDYYKNIKRKEELEKEENPEKLKLTKKVTEEKLFTAYGR |
| | NRDRFMDFAVRYLAEQNYFGKDAEFKMYMFETTNEQENYLKEQK |
| | NTADKKVIDQNKYHQGRLTCFKTYQKHKDDYQNWDDPFVFQNN |
| | AFQIILTFSNGERKKFSIQRKLLIYLLEDALFNHSDSIEDKGKQLLED |
| | YFFNTLMPDFEDAKESYKTSDDVNWKHRKLLPKRLIYTVHPPRRT |
| | DSEEQIHPFEKILRETQEQERRYRLLLGKAKNMKLKEEFIKRNKGK |
| | HFKLRFIRKAWHLMYFREIYERRAKEHSHHKSFHITRDEINDFSRW |
| | MYAFDEVPPYKVYLRNMLQRKKFMENEEFAELFEKGKSLDDFYRI |
| | TKKEFSKRIKNNLFQLKVDSERQYAEILSKKLVYINLSHFIKYLNAK |
| | GKLTVENGIIQYKASTNKKYLIDEYYYTEVLPREEYKVHKHLFNKL |
| | RATKLEDALLYELAMKYLREDNDIVEKAKSKVSDIQSSEISFDIKDY |
| | YGNHLYTLIVPFNKLETLSILIRYKTKQEKNSKLQRTSFLGNIYHLL |
| | AFLDKNYKQLSKYKDDGGFKKIVKNFKNKKRLSFVELNTINGYIIS |
| | GAVKFSKVHMELERYFINKHKIKANHIYIDLEDIKDDSNKQVFGNY |
| | YDSKLRIRNKAFHFGVPTNFFFNIEIEKIEKKFILEEVKLQNVSSFDK |
| | LNQNAKSVCRVFMEVLHSDLYRRDKNKSKEELRREFEEKYFNEIIT |
| | TV |
| 13 | MMTKKPANRHALPKVIISEVDNTNILEFKIKYEKLARLDRVEVKAM |
| | HYEDGRIIFDEVVVNGGLIEVEYQDDHKTLFVQVGEKSYSISGQKV |
| | GGKQRLLEDRVSKTKVQLELSDGSSERVSRTERELIVADNIKLYSQI |
| | VGHEVKTTKEIYLAKRFLGYRSDLLFYYGFVDNFFRESKNLKYGK |
| | QPVELWEDKFQVNDKLTAYTKFMFNDDLQNSESYLKEYVKDNHK |
| | IKNDLESARDIFATFRHNLMHFNYSFFTRLFNGEDVKIKNLQTKKFE |
| | SLSDVLRNVEFLNKVIQSIDKLNIDTRKEFIDKEKITLFNEELDLQQL |
| | YGFFAYTAINRVAFNKLINSFIIKDGIENEQLKEYFNQRVDGTAYEI |
| | DIHQNREYKELYKKHKNLVSKVSTLSDGKEIARGNTEISVLKEQMN |
| | KITKANSLKRLEHKLRLAFGFIYTEYGSYKAFVSRFNEDTKRKKIK |
| | NVEFEKIGVEKQKEYYESTFTSNNKDKLGELIQEYEKLSLNDLIEND |
| | TFLKVILLLFIFMPKEVKGDFLGFIKKYYHDTKHIEEDTKEKDEGFT |
| | NTLPIGLKLKIVERNIAKLSVLKHSLSLKVKYNRGQYEEDNTYRKV |
| | FKKLNISHNQEEFHKSMFSPLLRYYASLYKLINDFEIYTLSHYITDK |
| | YSTLNKVIASEQFHYRYGWNREEKKGELVKTDNYTFSTLLSKKYG |
| | HKNSQEISEMRNKISHFDEKILFKFPLEEVSSVPKGKGKYKKDEPIK |
| | SLKEKREEIVSLMEKQTDMQKVLGYDAINDFRMKTVQFQTKLKVY |
| | SNKEETIKKMIVEAKTPNDYYNIYKVKGVEGINEHLLNVIGETEAE |
| | KSIQEQIAEGNKVNV |
| 14 | MEKIKKPSNRNSIPSIIISDYDANKIKEIKVKYLKLARLDKITIQDMEI |
| | VDNIVEFKKILLNGVEHTIIDNQKIEFDNYEITGCIKPSNKRRDGRIS |
| | QAKYVVTITDKYLRENEKEKRFKSTERELPNNTLLSRYKQISGFDT |
| | LTSKDIYKIKRYIDFKNEMLFYFQFIEEFFNPLLPKGKNFYDLNIEQN |
| | KDKVAKFIVYRLNDDFKNKSLNSYITDTCMIINDFKKIQKILSDFRH |
| | ALAHFDFDFIQKFFDDQLDKNKFDINTISLIETLLDQKEEKNYQEKN |
| | NYIDDNDILTIFDEKGSKFSKLHNFYTKISQKKPAFNKLINSFLSQDG |
| | VPNEEFKSYLVTKKLDFFEDIHSNKEYKKIYIQHKNLVIKKQKEESQ |
| | EKPDGQKLKNYNDELQKLKDEMNTITKQNSLNRLEVKLRLAFGFI |
| | ANEYNYNFKNFNDEFTNDVKNEQKIKAFKNSSNEKLKEYFESTFIE |
| | KRFFHFSVNFFNKKTKKEETKQKNIFNSIENETLEELVKESPLLQIIT |
| | LLYLFIPRELQGEFVGFILKIYHHTKNITSDTKEDEISIEDAQNSFSLK |
| | FKILAKNLRGLQLFHYSLSHNTLYNNKQCFFYEKGNRWQSVYKSF |
| | QISHNQDEFDIHLVIPVIKYYINLNKLMGDFEIYALLKYADKNSITV |
| | KLSDITSRDDLKYNGHYNFATLLFKTFGIDTNYKQNKVSIQNIKKT |
| | RNNLAHQNIENMLKAFENSEIFAQREEIVNYLQTEHRMQEVLHYNP |
| | INDFTMKTVQYLKSLSVHSQKEGKIADIHKKESLVPNDYYLIYKLK |
| | AIELLKQKVIEVIGESEDEKKIKNAIAKEEQIKKGNN |
| 15 | MIKNPSNRHSLPKVIISEVDHEKILEFKIKYEKLARLDRFEVKAMHY |
| (CasM.1584) | EGKEIVFDEVLVNGGLIEVEYQDDNKTLFVKVGEKSYSIRGKKVGG |
| | KQRLLEDRVSKTKVQLELSDGVVDNKGNLRKSRTERELIVADNIKL |
| | YSQIVGREVTTTKEIYLVKRFLAYRSDLLFYYSFVDNFFKVAGNEK |
| | ELWKINFDDATSAQFMGYIPFMVNDNLKNDNAYLKDYVRNDVQI |
| | KDDLKKVQTIFSALRHTLLHFNYEFFEKLFNGEDVGFDFDIGFLNLL |
| | IENIDKLNIDAKKEFIDNEKIRLFGENLSLAKVYRLYSDICVNRVGF |
| | NKFINSMLIKDGVENQVLKAEFNRKFGGNAYTIDIHSNQEYKRIYN |
| | EHKKLVIKVSTLKDGQAIRRGNKKISELKEQMKSMTKKNSLARLE |
| | CKMRLAFGFLYGEYNNYKAFKNNFDTNIKNSQFDVNDVEKSKAY |
| | FLSTYERRKPRTREKLEKVAKDIESLELKTVIANDTLLKFILLMFVF |
| | MPQELKGDFLGFVKKYYHDVHSIDDDTKEQEEDVVEAMSTSLKLK |
| | ILGRNIRSLTLFKYALSSQVNYNSTDNIFYVEGNRYGKIYKKLGISH |
| | NQEEFDKTLVVPLLRYYSSLFKLMNDFEIYSLAKANPTAVSLQELV |
| | DDETSPYKQGNYFNFNKMLRDIYGLTSDEIKSGQVVFMRNKIAHF |
| | DTEVLLSKPLLGQTKMNLQRKDIVSFIEARGDIKELLGYDAINDFR |
| | MKVIHLRTKMRVYSDKLQTMMDLLRNAKTPNDFYNVYKVKGVE |
| | SINKHLLEVLAQTAEERTVEKQIRDGNEKYDL |
| 16 | MLKHKRKNKNSLARVVLSNYDSNNIYEIKIKYEKLAKLDKINIIEM |
| (CasM.1730) | DYDADNNVMFKKVLFNNKEIDLSHKDKTKINIELDNKKYNISAKK |
| | QIGKTHLVVRDKQTSKISRIKKIQDTYYRGKDVFILDNNIEILDKKQ |
| | TKDKFIVTLNDITNDKTTSTEAELIDDTKDIFKKISAKKDLKSSDIYK |
| | IKRFISIRSNFSFYYTFVDNYFKIFHAKKDKNKEELYKIKFKDEINIKP |
| | YLENILDNMKNKNGILYDYADDREKVLNDLKNIQYVFTEFRHKLA |
| | HFDYNFLDNFFSNSVTDQYKQKVNEIKLLDILLDNIDSLNVVPKQN |
| | YIEDETISVFDAKDIKLKRLYTYYIKLTINYPGFKKLINSFFIQDGIEN |
| | QELKEYINNKEKDTQVLKELDNKAYYMDISQYRKYKNIYNKHKEL |
| | VSEKELSSDGQKINSLNQKINKLKIEMKNITKPNALNRLIYRLRVAF |
| | GFIYKEYATINNFNKSFLQDTKIKRFENISQQDIKNYLDISYQDKGK |
| | FFVKSKKTFKNKTTIKYTFEDLDLTLNEIITQDDIFVKVIFLFSIFMPK |
| | ELNGDFFGFINMYYHKMKNISYDTKDIDMLDTISQNMKLKILEQNI |
| | KKTYVFKYYLDLDSSIYSKLVQNIKITEDIDSKKYLYAKIFKYYQHL |
| | YKLISDVEIYLLYKYNSKENLSITIDKDELKHRGYYNFQSLLIKNNIN |
| | KDDAYWSIVNMRNNLSHQNIDELVGHFCKGCLRKSTTDIAELWLR |
| | KDILTITNEIINKIESFKDIKITLGYDCVNDFTQKVKQYKQKLKASNE |
| | RLAKKIEEKQNQVVDEKNKEELEKKILNMKNIQKINRYILDIL |
| 17 | MNQYIHANKKENKKRPNKSSIIRIMVSDFDDEYIQEIKVLYIKQGGV |
| (CasM.1770) | DTFKINKMSYDSASKKIIFEEVATQNMLSVEDSNLNFKRPMVECKN |
| | GDIYVVKPSEKVNEKGQAIEPLRSYKIHGKYLDLTEEIGKDNAEQS |
| | GKKQIYLHVEDLLGMHTTADSIDRRRLESETQRTLLSKEVMENYA |
| | LIMGHEIKLDESDETYKLASSKEIYKANRFLDYRSRLLYYYSFINHF |
| | LVGLSKGATYEYAGKSLVAKIPDGEVWQLCELEVTAYHGIYINKK |
| | RNELVNENSLKIDAIYAQMAKNMKETINEFVDNYNAMIEKQNELK |
| | DNKSYKINKKAQNTTKFENFTEEKINQDLHNIVYILSDLRHKLMHF |
| | EYHYFELLMTGKKMGEKSEVIVCVPDRNSELPASKNEPSKDKFRK |
| | EKKLSELLDLNILKELDTFVKVKESYQTTYLETNDKIEILGKLKTAK |
| | SIYQIYHQICQRKNGFNKFINSFFTVDGEENTEVKDCINEVFRKEIHY |
| | FEMVIAKSNEDTLNDKNKNKSKKTRDRMKNQIQECKRYQDDESNI |
| | DTWVAYHKDIHYSKRYKKLYCEHITLVDNLNSAVSNGLNGQVIKE |
| | INDDIAIKKREMNEITKANSKSRLRYKMQMAYGFLFVEYGLKIPKF |
| | LNDFDLSHVRTASKIKGYKSPVKVTQYLTNDEGNKDNFNLETLME |
| | DIDKKSKINFEFLKSNEDNNLIKLYILIYQLLPRELKGDFLGFVKNN |
| | YYDLKHVDFQTRETEAKDQFFHNMRLFEKNVKAFDLIQYSIGDEM |
| | SQLGNETFNFSQALSKIVSDDTILNSATEIPNINRLVYGSLLKYYENA |
| | FRLSSEIEIRALIKIARGKQIDNHSIDEAYKDALIFQKSGQTVKFSSIL |
| | KYFNIDDLNKKNDSKIYNKASNLRNKIAHCDYTVLFITNIICEAENI |
| | NVKAKYLIDVSNKIGLNTVDLGNDMVNDYLMSYDKQMTYLAKTS |
| | EELLKESSLDKSKEKKERRDNLKNETRSHSEIYMEYEWISDYLVKL |
| | KDNHEILSKKNKTEDLKLNRAYELIKNYNVALNKKSKIKIRALTTD |
| | HINNNWVNIWGALTHELQEIKGLYLYKVTPKIKKGIYFVLTDKKN |
| | YCLNINLYTLKKVPSKDDSEMTEERYLRDISEKYYFTVGEDSIKAL |
| | KERLQTINDKCIITYVNEIDCKNENIKEKPEFKIAEDYNSWDKKYLSI |
| | SNEELHHWSVETRRDKKYKENLILNLMEKSSFKLNLHQL |
| 18 | MSQLKNPSNKNSLPRIIISDFNEIKINEIKIKYHKLDRLDKIIVKEMEII |
| (CasM.1816) | NNKIFFKKILFNNQIKDINSENIELENYILAGEVKPSNTKIILNRDGKE |
| | KSFIVYDGFTFKYKPNDKRISETKTNAKYILTIKDKTRHRESSTQRDI |
| | LKSSIIETYKQISGFENITSKDIYTIKRYIDFKNEMMFYYTFIDDFFFPI |
| | TGKNKQDKKNNFYNYKIKENAKKFISLINYRINDDFKNKNGILYDY |
| | LSNKEEIIINDFIHIQTILKDVRHAIAHFNFDFIQKLFDNEQAFNSKFD |
| | GIEILNILFNQKQEKYFEAQTNYIEEETIKILDEKELSFKKLHSFYSQI |
| | CQKKPAFNKLINSFIIQDGIENKELKDYISQKYNSKFDYYLDIHTCKI |
| | YKDIYNQHKKFVADKQFLENQKTDGQKIKKLNDQINQLKTKMNN |
| | LTKKNSLKRLEIKFRLAFGFIFTEYQTFKNFNERFIEDIKANKYSTKI |
| | ELLDYGKIKEYISITHEEKRFFNYKTFNKKTNKNINKTIFQSLEKETF |
| | ENLVKNDNLIKMMFLFQLLLPRELKGEFLGFILKIYHDLKNIDNDT |
| | KPDEKSLSELNISTALKLKILVKNIRQINLFNYTISNNTKYEEKEKRF |
| | YEEGNQWKDIYKKLYISHDFDIFDIHLIIPIIKYNINLYKLIGDFEVYL |
| | LLKYLERNTNYKTLDKLIEAEELKYKGYYNFTTLLSKAINIALNDK |
| | EYHNITHLRNNTSHQDIQNIISSFKNNKLLEQRENIIELISKESLKKKL |
| | HFDPINDFTMKTLQLLKSLEVHSDKSEKIENLLKKEPLLPNDVYLLY |
| | KLKGIEFIKKELISNIGITKYEEKIQEKIAKGVEK |
| 19 | ELCKIDFTDARSSSLIEYFKFAINDNLKNDRGYLKAYVNDVEQIRA |
| (CasM.1862939) | DLKKVGGKQRNLEDRVSRTKVQLTLTNHIEDREGKQRVSRTERELI |
| | VPQNIKLYSQIVGREVKTTKEIYLIKRFLEYRSDLLFYYGFVDNFFK |
| | VEGNKKELCKIDFTDARSSSLIEYFKFAINDNLKNDRGYLKAYVND |
| | VEQIRTDLQKVKTIFSKLRHALMHFDYDFFEKLFNGEEVGFDFDIKF |
| | LNIMIDKVEKLNIETKKEFIEDEVITLFGERLSLKKLYGLFSHIAINRV |
| | AFNKFINSFLIKDGIENRALKDFFNDEKGSQAYEIDIHSNAEYKALY |
| | VQHKKLVMATSAMSDGNEIAKKNQEISELKEKMNAITKANSLARL |
| | EYKLRLAFGFIYTEYGDYTAFKNSFDRDVKSAKYKELSVERLKAY |
| | YLATFKASKPQSHEKLEEVAKKIDRLSLKQLIENETLLKFVLLLFTF |
| | MPQELKGEFLGFIKKYYHDKKHIEQDTKEKEEEREGLSTGLKLKVL |
| | EKNIRSLSILKHALSFQVKYNKKDKNFYEEGNLHGKFYKKLAISHN |
| | QEEFNKSVYAPLFRYYVALYKLINDFEIYSLAQHIVNNETLADQVG |
| | KAQFRQRGYFNFRKLVNCTYATAQNSSYNVLIFMRNDISHLSYEPL |
| | FNCPLEEKASYKQKIRGREKIISVKPLSESRAEIVRFIASQTDMKKLL |
| | GYDAVNDFNMKMVQLRRRLSVYANKQETIEKMINKAKTPNDFYN |
| | LYKLKGIECINQHLLKVIGVTEAEKRIEKQIEEGNEKY |
| 20 | MLKKPSNRYALPKVILSTVDHEKILEFKVKYEKLARLDRLVVERM |
| (CasM.1862895) | HFDGESVVFDEVIANSGDLEIAYQDDHRKLLIQAAGKSYTITGKKV |
| | GGKKRKLEERISRAKIQLTLTDGQEDQHRRIRATVTEKALLEPKED |
| | RDIYSKISDRKIKTSKEIYLVKRFLSYRSDLLFYYFFVDNFFKVGNN |
| | KQELWKIKFQNQPELIEYFRFIINDRFKNAKNDKFDNYLKNDKAIQ |
| | EDLEKIQKVFEKLRHALMHYDYGFFEKLFGGEDQGFDLDIAFLDNF |
| | VKKIDKLNIDTKKEFVDDEKIKIFGEDLNLADLYKLYASISINRVGF |
| | NRVVNEMIIKDGIEKSELKRAFEKKLDKTYALDIHSDPSYKKLYNE |
| | HKRLVTEVSTYTDGNKIKEGNQKIAKLKYEMKEITKKNALVRLEC |
| | KMRLAFGLIYGRYDTHEAFKNGFDTDLKRGEFAQIGSEEAIGYFNT |
| | TFEKSKPKSKEEIKKIARQIDNLSLSTLIEDDPLMKFIVLMFLFVPRE |
| | LKGEFLGFWRKYYHDIHSIDSDAKSDEMPDEVSLSLKLKILTRNIRR |
| | LNLFEYSLSEKIKYSPKNTQFYTDKSPYQKVYKRLKISHNKEEFDKT |
| | LLVPLFRYYSILFKLINDFEIYSLAKANPDASSLSELTKTKHGFRGHY |
| | NFTTLMMDAHKVSQGDSKKHFGIRGEIAHINTKDLIYDPLFRKSKM |
| | AQQRNDVIDFVLKYEKEIKAVLGYDAINDFRMKVVQLRTKLKVYS |
| | DKTQTIEKLLNEVEAPDDFYVLYKVKGVEAINKYLLEIVSVTQAEE |
| | EIERKIITGNKRYNT |
| 21 | MLKHKRKNKNSLARVVLSNYDSNNIYEIKIKYEKLAKLDKINIIEM |
| (CasM.1862903) | DYDADNNVMFKKVLFNNKEIDLSHKDKTKINIELDNKKYNISAKK |
| | QIGKTHLVVRNKQTSKISRIKKIQDTYYRGKDVFILDNNIEILDKKQ |
| | TKDKFIVTLNDITNNKTTSTEAELIDDTKDIFKKISAKKDLKSSDIYK |
| | IKRFISIRSNFSFYYTFVDNYFKIFHAKKDKNKEELYKIKFKDEINIKP |
| | YLENILDNMKNKNGILYNYANDRKKVLNDLRNIQYVFKEFRHKLA |
| | HFDYNFLDNFFSNSVEEKYKQKVNEIKLLDILLDNIDSLNVVPKQN |
| | YIEDETISVFDAKDIKLKRLYTYYIKLTINYPGFKKLINSFFIQDGIEN |
| | QELKEYINNKEKDTQVLKELDNKAYYMDISQYRKYKNIYNKHKEL |
| | VSEKELSSDGKKINSLNQKINKLKIDMKNITKPNALNRLIYRLRVAF |
| | GFIYKEYATINNFNKSFLQDTKTKRFENISQQDIKSYLDISYQDKGK |
| | FFVKSKKTFKNKTTVKYTFEDLDLTLNEIITQDDIFVKVIFLFSIFMP |
| | KELNGDFFGFINMYYHKMKNISYDTKDIDMLDTISQNMKLKILEQN |
| | IKKTYVFKYYLDLDSSIYSKLVQNIKITEDIDSKKYLYAKIFKYYQH |
| | LYKLISDVEIYLLYKYNSKENLSITIDKDELKHRGYYNFQSLLIKNNI |
| | NKDDAYWSIVNMRNNLSHQNIDELVGHFCKGCLRKSTTDIAELWL |
| | RKDILTITNEIINKIESFKDIKITLGYDCVNDFTQKVKQYKQKLKASN |
| | ERLAKKIEEKQNQVVDEKNKEELEKNILNMKNIQKINRYILDIL |
| 22 | MIKKPSNRHALPKVIISKVDNQNILEFKIKYKKLSRLDRVEIKTMHY |
| (CasM.1862909) | DDRAIVFDEVIINGGLIDVEYRDNHKTIFVKVGDKSYSISGQKVGGK |
| | ERLLENRISQTKVQLELKDEATNRVSKTERELIVDDNIKLYSQIVGR |
| | DVKTTKDIYLIKRFLGYRSDLLFYYGFVNNFFHVANNRPEFWKIDF |
| | NDNRNSKLIEYFIFTINDHLKNDENYLKDYISDRGQIVDDLENIKHIF |
| | SALRHGLMHFDYDFFEALFNGEDIDIKMDNQGNTQPLSSLNIKFLDI |
| | MIDKLDKLNIDTKKEFIDAEKITIFGEELSLAKLYRFYAHTAINRVAF |
| | NKLINSFIIENGVENQSLKEYFNQQAGGIAYEIDIHQNREYKNLYNE |
| | HKKLVSRVLSISDGQEIATLNQKIVELKEQMKQITKINSIKRLEYKL |
| | RLAFGFIYTEYKNYEEFKNSFDTDIKNGRFTPKDEDGNKRAFDSRE |
| | LEHLKGYYKATLQTQKPQTDEKMEEVSKRVDRLSLKSLIGDDTLL |
| | KFILLMFTFMPQELKGEFLGFIKKYYHDTKHIDQDTISDSDDTIEEG |
| | LSIGLKLKILDKNIRSLSILKHSLSFQTKYNKKDRSYYEDGNIHGKFF |
| | KKLGISHNQEEFNKSVYAPLFRYYSALYKLINDFEIYTLSLHIVGNE |
| | TLSDQVNKPQFLSGRYFNFRKLLTQSYNISNNSTHSVIFNAVINMRN |
| | DISHLSYEPLLDCPLNGKKSYKRKIRNQFRTINIKPLVESRKMIIDFIT |
| | LQTDMQKVLGCDAVNDFTMKIVQLRTRLKAYANKEQTIEKMITEA |
| | KTPNDFYNIYKVKGVEAINKYLLEVIGETQVEKEIREEIERGNIANS |
| 23 | MVKNPANRHALPKVIISEVDNNNILEFKIKYEKLARLDKVEVKSMH |
| (CasM.1862917) | FDNNKQVVFDEVVINGGLIEPTYEDKHKKLVVTAGEKSYSIVGQK |
| | VGGKPRLLEDRVSKTKVQLELTNYVEDKEGKKRVSKTERELIVAD |
| | NIELYSQIVGREVKTTKEIYLIKRFLEYRSDLLFYYGFVDNFFKVAG |
| | NGKELWKIDFTNSDSLHLIEYFKFSINDNLKNDENYLKNYVSDNTK |
| | IENDLVKCQNNFNSLRHALMHFDYDFFEKLFNGEDVGFDFDIEFLN |
| | IMIDKVDKLNIDTKKEFIDDEEVTLFGEALSLKKLYGLFSHIAINRV |
| | AFNKLINSFIIEDGIENKELKDFFNNKKESQAYEIDIHSNAEYKALYV |
| | QHKKLVMATSAMTDGDEIAKKNQEISDLKEKMKVITKENSLARLE |
| | HKLRLAFGFIYTEYKDYKTFKKHFDQDIKGAKYKGLNVEKLKEYY |
| | ETTLKNSKPKTDEKLEDVAKKIDKLSLKELIDDDTLLKFVLLLFIFM |
| | PQELKGDFLGFIKKYYHDKKHIDQDTKDKDTEIEELSTGLKLKVLD |
| | KNIRSLSILKHSFSFQVKYNRKDKNFYEDGNLHGKFYKKLSISHNQ |
| | EEFNKSVYAPLFRYYSALYKLINDFEIYALAQHVENHETLADQVNK |
| | SQFIQKSYFNFRKLLDNTDSISQSSSYNTLIVMRNDISHLSYEPLFNY |
| | PLDERKSYKKKTQKGVKTFHVELLYISRAKIIELISLQTDMKKLLGY |
| | DAVNDFNMKVVHLRKRLSVYANKEESIRKMQADAKTPNDFYNIY |
| | KVKGVESINQHLLKVIGVTEAEKSIEKQINEGNKKHNT |
| 24 | MIKNPSNRYALPKVIISKIDNQNILEFKIKYKKLSKLDIVKVKSMHY |
| (CasM.1862921) | DDRAIIFDEVIVNDGLIDVEYRDNHKTIFVKVGNKSYSISGQKVGGK |
| | ERLLENRVSKTKVQLELKDKATNRVSKTERELIVDDNIKIYSQIVGR |
| | DVKTTKDIYLIKRFLAYRSDLLFYYGFVNNFFHVANNRSEFWKIDF |
| | NDSNNSKLIEYFKFTINDHLKNDENYLKDYISDNEKLKNDLIKVKN |
| | SFEKIRHALMHFDYDFFVKLFNGEDVGLELDIEFLDIMIDKLDKLNI |
| | DTKKEFIDDEKITIFGEELSLAKLYRFYAHTAINRVAFNKLINSFIIEN |
| | GVENQSLKEYFNQQAGGIAYEIDIHQNREYKNLYNEHKKLVSRVL |
| | SISDGQEIAILNQKIAKLKDQMKQITKANSIKRLEYKLRLALGFIYTE |
| | YENYEEFKNNFDTDIKNGRFTPKDNDGNKRAFDSRELEQLKGYYE |
| | ATIQTQKPKTDEKIEEVSKKIDRLSLKSLIADDILLKFILLMFTFMPQ |
| | ELKGEFLGFIKKYYHDTKHIDQDTISDSDDTIETLSIGLKLKILDKNI |
| | RSLSILKHSLSFQTKYNKKDRNYYEDGNIHGKFFKKLGISHNQEEF |
| | NKSVYAPLFRYYSALYKLINDFEIYTLSLHIVGSETLTDQVNKSQFL |
| | SGRYFNFRKLLTQSYHINNNSTHSTIFNAVINMRNDISHLSYEPLFD |
| | CPLNGKKSYKRKIRNQFKTINIKPLVESRKIIIDFITLQTDMQKVLGY |
| | DAVNDFTMKIVQLRTRLKAYANKEQTIQKMITEAKTPNDFYNIYK |
| | VQGVEEINKYLLEVIGETQAEKEIREKIERGNIANF |
| 25 | MTKKPSNRNSLPKVIINKVDESSILEFKIKYEKLARLDRFEVRSMRY |
| (CasM.1862947) | DGDGRIIFDEVVANAGLLDVDYEDDNRTIVVKIENKAYNIYGKKV |
| | GGEKRLNGKISKAKVQLILTDSIRKNANDTHRHSLTERELINKNEV |
| | DLYSKIAEREISTTKDIYLVKRFLAYRSDLLLYYAFINHYVRVNGNK |
| | KEFWKTEIDDKIIDYFIYTINDTLKNKEGYLEKYIVDRDQIKKDLEKI |
| | KQIFSHLRHKLMHYDFRFFTDLFDGKDVDIKVDNSIQKISELLDIEF |
| | LNIVIDKLEKLNIDAKKEFIDDEKITLFGQEIELKKLYSLYAHTSINR |
| | VAFNKLINSFLIKDGVENKELKEYFNAHNQGKESYYIDIHQNQEYK |
| | KLYIEHKNLVAKLSATTDGKEIAKINRELADKKEQMKQITKANSLK |
| | RLEYKLRLAFGFIYTEYKDYERFKNSFDTDTKKKKFDAIDNAKIIEY |
| | FEATNKAKKIEKLEEILKGIDKLSLKTLIQDDILLKFLLLFFTFLPQEI |
| | KGEFLGFIKKYYHDITSLDEDTKDKDDEITELPRSLKLKIFSKNIRKL |
| | SILKHSLSYQIKYNKKESSYYEAGNVFNKMFKKQAISHNLEEFGKSI |
| | YLPMLKYYSALYKLINDFEIYALYKDMDTSETLSQQVDKQEYKRN |
| | EYFNFETLLRKKFGNDIEKVLVTYRNKIAHLDFNFLYDKPINKFISL |
| | YKSREKIVNYIKNHDIQAVLKYDAVNDFVMKVIQLRTKLKVYADK |
| | EQTIESMIQNTQNPNGFYNIYKVKAVENINRHLLKVIGYTESEKAVE |
| | EKIRAGNTSKS |
| 26 | MEKIKKPSNRNSIPSIIISDYDANKIKEIKVKYLKLARLDKITIQDMEI |
| (CasM.1422) | VDNIVEFKKILLNGVEHTIIDNQKIEFDNYEITGCIKPSNKRRDGRIS |
| | QAKYVVTITDKYLRENEKEKRFKSTERELPNNTLLSRYKQISGFDT |
| | LTSKDIYKIKRYIDFKNEMLFYFQFIEEFFNPLLPKGKNFYDLNIEQN |
| | KDKVAKFIVYRLNDDFKNKSLNSYITDTCMIINDFKKIQKILSDFRH |
| | ALAHFDFDFIQKFFDDQLDKNKFDINTISLIETLLDQKEEKNYQEKN |
| | NYIDDNDILTIFDEKGSKFSKLHNFYTKISQKKPAFNKLINSFLSQDG |
| | VPNEEFKSYLVTKKLDFFEDIHSNKEYKKIYIQHKNLVIKKQKEESQ |
| | EKPDGQKLKNYNDELQKLKDEMNTITKQNSLNRLEVKLRLAFGFI |
| | ANEYNYNFKNFNDEFTNDVKNEQKIKAFKNSSNEKLKEYFESTFIE |
| | KRFFHFSVNFFNKKTKKEETKQKNIFNSIENETLEELVKESPLLQIIT |
| | LLYLFIPRELQGEFVGFILKIYHHTKNITSDTKEDEISIEDAQNSFSLK |
| | FKILAKNLRGLQLFHYSLSHNTLYNNKQCFFYEKGNRWQSVYKSF |
| | QISHNQDEFDIHLVIPVIKYYINLNKLMGDFEIYALLKYADKNSITV |
| | KLSDITSRDDLKYNGHYNFATLLFKTFGIDTNYKQNKVSIQNIKKT |
| | RNNLAHQNIENMLKAFENSEIFAQREEIVNYLQTEHRMQEVLHYNP |
| | INDFTMKTVQYLKSLSVHSQKEGKIADIHKKESLVPNDYYLIYKLK |
| | AIELLKQKVIEVIGESEDEKKIKNAIAKEEQIKKGNN |
| 27 | MMTKKPANRHALPKVIISEVDNTNILEFKIKYEKLARLDRVEVKAM |
| (CasM.1740) | HYEDGRIIFDEVVVNGGLIEVEYQDDHKTLFVQVGEKSYSISGQKV |
| | GGKQRLLEDRVSKTKVQLELSDGSSERVSRTERELIVADNIKLYSQI |
| | VGHEVKTTKEIYLAKRFLGYRSDLLFYYGFVDNFFRESKNLKYGK |
| | QPVELWEDKFQVNDKLTAYTKFMFNDDLQNSESYLKEYVKDNHK |
| | IKNDLESARDIFATFRHNLMHFNYSFFTRLFNGEDVKIKNLQTKKFE |
| | SLSDVLRNVEFLNKVIQSIDKLNIDTRKEFIDKEKITLFNEELDLQQL |
| | YGFFAYTAINRVAFNKLINSFIIKDGIENEQLKEYFNQRVDGTAYEI |
| | DIHQNREYKELYKKHKNLVSKVSTLSDGKEIARGNTEISVLKEQMN |
| | KITKANSLKRLEHKLRLAFGFIYTEYGSYKAFVSRFNEDTKRKKIK |
| | NVEFEKIGVEKQKEYYESTFTSNNKDKLGELIQEYEKLSLNDLIEND |
| | TFLKVILLLFIFMPKEVKGDFLGFIKKYYHDTKHIEEDTKEKDEGFT |
| | NTLPIGLKLKIVERNIAKLSVLKHSLSLKVKYNRGQYEEDNTYRKV |
| | FKKLNISHNQEEFHKSMFSPLLRYYASLYKLINDFEIYTLSHYITDK |
| | YSTLNKVIASEQFHYRYGWNREEKKGELVKTDNYTFSTLLSKKYG |
| | HKNSQEISEMRNKISHFDEKILFKFPLEEVSSVPKGKGKYKKDEPIK |
| | SLKEKREEIVSLMEKQTDMQKVLGYDAINDFRMKTVQFQTKLKVY |
| | SNKEETIKKMIVEAKTPNDYYNIYKVKGVEGINEHLLNVIGETEAE |
| | KSIQEQIAEGNKVNV |

Provided herein is a non-naturally occurring composition comprising a programmable nuclease and an engineered guide nucleic acid, wherein the programmable nuclease comprises an amino acid sequence that is at least 75% identical to any one of SEQ ID NOs: 1-5 and 15-27. In some embodiments, the programmable nuclease comprises an amino acid sequence that is at least 80% identical to any one of SEQ ID NOs: 1-5 and 15-27. In some embodiments, the programmable nuclease comprises an amino acid sequence that is at least 85% identical to any one of SEQ ID NOs: 1-5 and 15-27. In some embodiments, the programmable nuclease comprises an amino acid sequence that is at least 90% identical to any one of SEQ ID NOs: 1-5 and 15-27. In some embodiments, the programmable nuclease comprises an amino acid sequence that is at least 95% identical to any one of SEQ ID NOs: 1-5 and 15-27. In some embodiments, the programmable nuclease comprises an amino acid sequence that is at least 98% identical to any one of SEQ ID NOs: 1-5 and 15-27. In some embodiments, the programmable nuclease comprises an amino acid sequence that is at least 99% identical to any one of SEQ ID NOs: 1-5 and 15-27. In some embodiments, the programmable nuclease comprises an amino acid sequence of any one of SEQ ID NOs: 1-5 and 15-27. In some embodiments, the amino acid sequence of the programmable nuclease is at least 75% identical to any one of SEQ ID NOs: 1-5 and 15-27. In some embodiments, the amino acid sequence of the programmable nuclease is at least 80% identical to any one of SEQ ID NOs: 1-5 and 15-27. In some embodiments, the amino acid sequence of the programmable nuclease is at least 85% identical to any one of SEQ ID NOs: 1-5 and 15-27. In some embodiments, the amino acid sequence of the programmable nuclease is at least 90% identical to any one of SEQ ID NOs: 1-5 and 15-27. In some embodiments, the amino acid sequence of the programmable nuclease is at least 95% identical to any one of SEQ ID NOs: 1-5 and 15-27. In some embodiments, the amino acid sequence of the programmable nuclease is at least 98% identical to any one of SEQ ID NOs: 1-5 and 15-27. In some embodiments, the amino acid sequence of the programmable nuclease is at least 99% identical to any one of SEQ ID NOs: 1-5 and 15-27. In some embodiments, the amino acid sequence of the programmable nuclease is any one of SEQ ID NOs: 1-5 and 15-27. In some embodiments, the programmable nuclease comprises an amino acid sequence that is at least 75% identical to SEQ ID NO: 1, and the engineered guide nucleic acid comprises a sequence that is at least 75% identical to SEQ ID NO: 28; the programmable nuclease comprises an amino acid sequence that is at least 75% identical to SEQ ID NO: 2, and the engineered guide nucleic acid comprises a sequence that is at least 75% identical to SEQ ID NO: 29; the programmable nuclease comprises an amino acid sequence that is at least 75% identical to SEQ ID NO: 3, and the engineered guide nucleic acid comprises a sequence that is at least 75% identical to SEQ ID NO: 30; the programmable nuclease comprises an amino acid sequence that is at least 75% identical to SEQ ID NO: 4, and the engineered guide nucleic acid comprises a sequence that is at least 75% identical to SEQ ID NO: 31; the programmable nuclease comprises an amino acid sequence that is at least 75% identical to SEQ ID NO: 5, and the engineered guide nucleic acid comprises a sequence that is at least 75% identical to SEQ ID NO: 32; the programmable nuclease comprises an amino acid sequence that is at least 75% identical to SEQ ID NO: 6, and the engineered guide nucleic acid comprises a sequence that is at least 75% identical to any one of SEQ ID NOs; 28-32; the programmable nuclease comprises an amino acid sequence that is at least 75% identical to SEQ ID NO: 7, and the engineered guide nucleic acid comprises a sequence that is at least 75% identical to any one of SEQ ID NOs; 28-32; the programmable nuclease comprises an amino acid sequence that is at least 75% identical to SEQ ID NO: 8, and the engineered guide nucleic acid comprises a sequence that is at least 75% identical to any one of SEQ ID NOs; 28-32; the programmable nuclease comprises an amino acid sequence that is at least 75% identical to SEQ ID NO: 9, and the engineered guide nucleic acid comprises a sequence that is at least 75% identical to any one of SEQ ID NOs; 28-32; the programmable nuclease comprises an amino acid sequence that is at least 75% identical to SEQ ID NO: 10, and the engineered guide nucleic acid comprises a sequence that is at least 75% identical to any one of SEQ ID NOs; 28-32; the programmable nuclease comprises an amino acid sequence that is at least 75% identical to SEQ ID NO: 11, and the engineered guide nucleic acid comprises a sequence that is at least 75% identical to any one of SEQ ID NOs; 28-32; the programmable nuclease comprises an amino acid sequence that is at least 75% identical to SEQ ID NO: 12, and the engineered guide nucleic acid comprises a sequence that is at least 75% identical to any one of SEQ ID NOs; 28-32; the programmable nuclease comprises an amino acid sequence that is at least 75% identical to SEQ ID NO: 13, and the engineered guide nucleic acid comprises a sequence that is at least 75% identical to any one of SEQ ID NOs; 28-32; the programmable nuclease comprises an amino acid sequence that is at least 75% identical to SEQ ID NO: 14, and the engineered guide nucleic acid comprises a sequence that is at least 75% identical to any one of SEQ ID NOs; 28-32; the programmable nuclease comprises an amino acid sequence that is at least 75% identical to SEQ ID NO: 15, and the engineered guide nucleic acid comprises a sequence that is at least 75% identical to SEQ ID NO: 60; the programmable nuclease comprises an amino acid sequence that is at least 75% identical to SEQ ID NO: 16, and the engineered guide nucleic acid comprises a sequence that is at least 75% identical to SEQ ID NO: 61; the programmable nuclease comprises an amino acid sequence that is at least 75% identical to SEQ ID NO: 17, and the engineered guide nucleic acid comprises a sequence that is at least 75% identical to SEQ ID NO: 62; the programmable nuclease comprises an amino acid sequence that is at least 75% identical to SEQ ID NO: 18, and the engineered guide nucleic acid comprises a sequence that is at least 75% identical to SEQ ID NO: 63; the programmable nuclease comprises an amino acid sequence that is at least 75% identical to SEQ ID NO: 19, and the engineered guide nucleic acid comprises a sequence that is at least 75% identical to SEQ ID NO: 60; the programmable nuclease comprises an amino acid sequence that is at least 75% identical to SEQ ID NO: 20, and the engineered guide nucleic acid comprises a sequence that is at least 75% identical to SEQ ID NO: 64; the programmable nuclease comprises an amino acid sequence that is at least 75% identical to SEQ ID NO: 21, and the engineered guide nucleic acid comprises a sequence that is at least 75% identical to SEQ ID NO: 61; the programmable nuclease comprises an amino acid sequence that is at least 75% identical to SEQ ID NO: 22, and the engineered guide nucleic acid comprises a sequence that is at least 75% identical to SEQ ID NO: 65; the programmable nuclease comprises an amino acid sequence that is at least 75% identical to SEQ ID NO: 23, and the engineered guide nucleic acid comprises a sequence that is at least 75% identical to SEQ ID NO: 60; the programmable nuclease comprises an amino acid sequence that is at least 75% identical to SEQ ID NO: 24, and the engineered guide nucleic acid comprises a sequence that is at least 75% identical to SEQ ID NO: 65; the programmable nuclease comprises an amino acid sequence that is at least 75% identical to SEQ ID NO: 25, and the engineered guide nucleic acid comprises a sequence that is at least 75% identical to SEQ ID NO: 66; the programmable nuclease comprises an amino acid sequence that is at least 75% identical to SEQ ID NO: 26, and the engineered guide nucleic acid comprises a sequence that is at least 75% identical to SEQ ID NO: 67; or the programmable nuclease comprises an amino acid sequence that is at least 75% identical to SEQ ID NO: 27, and the engineered guide nucleic acid comprises a sequence that is at least 75% identical to SEQ ID NO: 68. In some embodiments, the programmable nuclease comprises an amino acid sequence that is at least 85% identical to SEQ ID NO: 1, and the engineered guide nucleic acid comprises a sequence that is at least 85% identical to SEQ ID NO: 28; the programmable nuclease comprises an amino acid sequence that is at least 85% identical to SEQ ID NO: 2, and the engineered guide nucleic acid comprises a sequence that is at least 85% identical to SEQ ID NO: 29; the programmable nuclease comprises an amino acid sequence that is at least 85% identical to SEQ ID NO: 3, and the engineered guide nucleic acid comprises a sequence that is at least 85% identical to SEQ ID NO: 30; the programmable nuclease comprises an amino acid sequence that is at least 85% identical to SEQ ID NO: 4, and the engineered guide nucleic acid comprises a sequence that is at least 85% identical to SEQ ID NO: 31; the programmable nuclease comprises an amino acid sequence that is at least 85% identical to SEQ ID NO: 5, and the engineered guide nucleic acid comprises a sequence that is at least 85% identical to SEQ ID NO: 32; the programmable nuclease comprises an amino acid sequence that is at least 85% identical to SEQ ID NO: 6, and the engineered guide nucleic acid comprises a sequence that is at least 85% identical to any one of SEQ ID NOs; 28-32; the programmable nuclease comprises an amino acid sequence that is at least 85% identical to SEQ ID NO: 7, and the engineered guide nucleic acid comprises a sequence that is at least 85% identical to any one of SEQ ID NOs; 28-32; the programmable nuclease comprises an amino acid sequence that is at least 85% identical to SEQ ID NO: 8, and the engineered guide nucleic acid comprises a sequence that is at least 85% identical to any one of SEQ ID NOs; 28-32; the programmable nuclease comprises an amino acid sequence that is at least 85% identical to SEQ ID NO: 9, and the engineered guide nucleic acid comprises a sequence that is at least 85% identical to any one of SEQ ID NOs; 28-32; the programmable nuclease comprises an amino acid sequence that is at least 85% identical to SEQ ID NO: 10, and the engineered guide nucleic acid comprises a sequence that is at least 85% identical to any one of SEQ ID NOs; 28-32; the programmable nuclease comprises an amino acid sequence that is at least 85% identical to SEQ ID NO: 11, and the engineered guide nucleic acid comprises a sequence that is at least 85% identical to any one of SEQ ID NOs; 28-32; the programmable nuclease comprises an amino acid sequence that is at least 85% identical to SEQ ID NO: 12, and the engineered guide nucleic acid comprises a sequence that is at least 85% identical to any one of SEQ ID NOs; 28-32; the programmable nuclease comprises an amino acid sequence that is at least 85% identical to SEQ ID NO: 13, and the engineered guide nucleic acid comprises a sequence that is at least 85% identical to any one of SEQ ID NOs; 28-32; the programmable nuclease comprises an amino acid sequence that is at least 85% identical to SEQ ID NO: 14, and the engineered guide nucleic acid comprises a sequence that is at least 85% identical to any one of SEQ ID NOs; 28-32; the programmable nuclease comprises an amino acid sequence that is at least 85% identical to SEQ ID NO: 15, and the engineered guide nucleic acid comprises a sequence that is at least 85% identical to SEQ ID NO: 60; the programmable nuclease comprises an amino acid sequence that is at least 85% identical to SEQ ID NO: 16, and the engineered guide nucleic acid comprises a sequence that is at least 85% identical to SEQ ID NO: 61; the programmable nuclease comprises an amino acid sequence that is at least 85% identical to SEQ ID NO: 17, and the engineered guide nucleic acid comprises a sequence that is at least 85% identical to SEQ ID NO: 62; the programmable nuclease comprises an amino acid sequence that is at least 85% identical to SEQ ID NO: 18, and the engineered guide nucleic acid comprises a sequence that is at least 85% identical to SEQ ID NO: 63; the programmable nuclease comprises an amino acid sequence that is at least 85% identical to SEQ ID NO: 19, and the engineered guide nucleic acid comprises a sequence that is at least 85% identical to SEQ ID NO: 60; the programmable nuclease comprises an amino acid sequence that is at least 85% identical to SEQ ID NO: 20, and the engineered guide nucleic acid comprises a sequence that is at least 85% identical to SEQ ID NO: 64; the programmable nuclease comprises an amino acid sequence that is at least 85% identical to SEQ ID NO: 21, and the engineered guide nucleic acid comprises a sequence that is at least 85% identical to SEQ ID NO: 61; the programmable nuclease comprises an amino acid sequence that is at least 85% identical to SEQ ID NO: 22, and the engineered guide nucleic acid comprises a sequence that is at least 85% identical to SEQ ID NO: 65; the programmable nuclease comprises an amino acid sequence that is at least 85% identical to SEQ ID NO: 23, and the engineered guide nucleic acid comprises a sequence that is at least 85% identical to SEQ ID NO: 60; the programmable nuclease comprises an amino acid sequence that is at least 85% identical to SEQ ID NO: 24, and the engineered guide nucleic acid comprises a sequence that is at least 85% identical to SEQ ID NO: 65; the programmable nuclease comprises an amino acid sequence that is at least 85% identical to SEQ ID NO: 25, and the engineered guide nucleic acid comprises a sequence that is at least 85% identical to SEQ ID NO: 66; the programmable nuclease comprises an amino acid sequence that is at least 85% identical to SEQ ID NO: 26, and the engineered guide nucleic acid comprises a sequence that is at least 85% identical to SEQ ID NO: 67; the programmable nuclease comprises an amino acid sequence that is at least 85% identical to SEQ ID NO: 27, and the engineered guide nucleic acid comprises a sequence that is at least 85% identical to SEQ ID NO: 68. In some embodiments, the programmable nuclease comprises an amino acid sequence that is at least 95% identical to SEQ ID NO: 1, and the engineered guide nucleic acid comprises a sequence that is at least 95% identical to SEQ ID NO: 28; the programmable nuclease comprises an amino acid sequence that is at least 95% identical to SEQ ID NO: 2, and the engineered guide nucleic acid comprises a sequence that is at least 95% identical to SEQ ID NO: 29; the programmable nuclease comprises an amino acid sequence that is at least 95% identical to SEQ ID NO: 3, and the engineered guide nucleic acid comprises a sequence that is at least 95% identical to SEQ ID NO: 30; the programmable nuclease comprises an amino acid sequence that is at least 95% identical to SEQ ID NO: 4, and the engineered guide nucleic acid comprises a sequence that is at least 95% identical to SEQ ID NO: 31; the programmable nuclease comprises an amino acid sequence that is at least 95% identical to SEQ ID NO: 5, and the engineered guide nucleic acid comprises a sequence that is at least 95% identical to SEQ ID NO: 32; the programmable nuclease comprises an amino acid sequence that is at least 95% identical to SEQ ID NO: 15, and the engineered guide nucleic acid comprises a sequence that is at least 95% identical to SEQ ID NO: 60; the programmable nuclease comprises an amino acid sequence that is at least 95% identical to SEQ ID NO: 16, and the engineered guide nucleic acid comprises a sequence that is at least 95% identical to SEQ ID NO: 61; the programmable nuclease comprises an amino acid sequence that is at least 95% identical to SEQ ID NO: 17, and the engineered guide nucleic acid comprises a sequence that is at least 95% identical to SEQ ID NO: 62; the programmable nuclease comprises an amino acid sequence that is at least 95% identical to SEQ ID NO: 18, and the engineered guide nucleic acid comprises a sequence that is at least 95% identical to SEQ ID NO: 63; the programmable nuclease comprises an amino acid sequence that is at least 95% identical to SEQ ID NO: 19, and the engineered guide nucleic acid comprises a sequence that is at least 95% identical to SEQ ID NO: 60; the programmable nuclease comprises an amino acid sequence that is at least 95% identical to SEQ ID NO: 20, and the engineered guide nucleic acid comprises a sequence that is at least 95% identical to SEQ ID NO: 64; the programmable nuclease comprises an amino acid sequence that is at least 95% identical to SEQ ID NO: 21, and the engineered guide nucleic acid comprises a sequence that is at least 95% identical to SEQ ID NO: 61; the programmable nuclease comprises an amino acid sequence that is at least 95% identical to SEQ ID NO: 22, and the engineered guide nucleic acid comprises a sequence that is at least 95% identical to SEQ ID NO: 65; the programmable nuclease comprises an amino acid sequence that is at least 95% identical to SEQ ID NO: 23, and the engineered guide nucleic acid comprises a sequence that is at least 95% identical to SEQ ID NO: 60; the programmable nuclease comprises an amino acid sequence that is at least 95% identical to SEQ ID NO: 24, and the engineered guide nucleic acid comprises a sequence that is at least 95% identical to SEQ ID NO: 65; the programmable nuclease comprises an amino acid sequence that is at least 95% identical to SEQ ID NO: 25, and the engineered guide nucleic acid comprises a sequence that is at least 95% identical to SEQ ID NO: 66; the programmable nuclease comprises an amino acid sequence that is at least 95% identical to SEQ ID NO: 26, and the engineered guide nucleic acid comprises a sequence that is at least 95% identical to SEQ ID NO: 67; or the programmable nuclease comprises an amino acid sequence that is at least 95% identical to SEQ ID NO: 27, and the engineered guide nucleic acid comprises a sequence that is at least 95% identical to SEQ ID NO: 68. In some embodiments, the programmable nuclease comprises an amino acid sequence of SEQ ID NO: 1, and the engineered guide nucleic acid comprises a sequence of SEQ ID NO: 28; the programmable nuclease comprises an amino acid sequence of SEQ ID NO: 2, and the engineered guide nucleic acid comprises a sequence of SEQ ID NO: 29; the programmable nuclease comprises an amino acid sequence of SEQ ID NO: 3, and the engineered guide nucleic acid comprises a sequence of SEQ ID NO: 30; the programmable nuclease comprises an amino acid sequence of SEQ ID NO: 4, and the engineered guide nucleic acid comprises a sequence of SEQ ID NO: 31; the programmable nuclease comprises an amino acid sequence of SEQ ID NO: 5, and the engineered guide nucleic acid comprises a sequence of SEQ ID NO: 32; the programmable nuclease comprises an amino acid sequence of SEQ ID NO: 6, and the engineered guide nucleic acid comprises a sequence of any one of SEQ ID NOs; 28-32; the programmable nuclease comprises an amino acid sequence of SEQ ID NO: 7, and the engineered guide nucleic acid comprises a sequence of any one of SEQ ID NOs; 28-32; the programmable nuclease comprises an amino acid sequence of SEQ ID NO: 8, and the engineered guide nucleic acid comprises a sequence of any one of SEQ ID NOs; 28-32; the programmable nuclease comprises an amino acid sequence of SEQ ID NO: 9, and the engineered guide nucleic acid comprises a sequence of any one of SEQ ID NOs; 28-32; the programmable nuclease comprises an amino acid sequence of SEQ ID NO: 10, and the engineered guide nucleic acid comprises a sequence of any one of SEQ ID NOs; 28-32; the programmable nuclease comprises an amino acid sequence of SEQ ID NO: 11, and the engineered guide nucleic acid comprises a sequence of any one of SEQ ID NOs; 28-32; the programmable nuclease comprises an amino acid sequence of SEQ ID NO: 12, and the engineered guide nucleic acid comprises a sequence of any one of SEQ ID NOs; 28-32; the programmable nuclease comprises an amino acid sequence of SEQ ID NO: 13, and the engineered guide nucleic acid comprises a sequence of any one of SEQ ID NOs; 28-32; the programmable nuclease comprises an amino acid sequence of SEQ ID NO: 14, and the engineered guide nucleic acid comprises a sequence of any one of SEQ ID NOs; 28-32; the programmable nuclease comprises an amino acid sequence of SEQ ID NO: 15, and the engineered guide nucleic acid comprises a sequence of SEQ ID NO: 60; the programmable nuclease comprises an amino acid sequence of SEQ ID NO: 16, and the engineered guide nucleic acid comprises a sequence of SEQ ID NO: 61; the programmable nuclease comprises an amino acid sequence of SEQ ID NO: 17, and the engineered guide nucleic acid comprises a sequence of SEQ ID NO: 62; the programmable nuclease comprises an amino acid sequence of SEQ ID NO: 18, and the engineered guide nucleic acid comprises a sequence of SEQ ID NO: 63; the programmable nuclease comprises an amino acid sequence of SEQ ID NO: 19, and the engineered guide nucleic acid comprises a sequence of SEQ ID NO: 60; the programmable nuclease comprises an amino acid sequence of SEQ ID NO: 20, and the engineered guide nucleic acid comprises a sequence of SEQ ID NO: 64; the programmable nuclease comprises an amino acid sequence of SEQ ID NO: 21, and the engineered guide nucleic acid comprises a sequence of SEQ ID NO: 61; the programmable nuclease comprises an amino acid sequence of SEQ ID NO: 22, and the engineered guide nucleic acid comprises a sequence of SEQ ID NO: 65; the programmable nuclease comprises an amino acid sequence of SEQ ID NO: 23, and the engineered guide nucleic acid comprises a sequence of SEQ ID NO: 60; the programmable nuclease comprises an amino acid sequence of SEQ ID NO: 24, and the engineered guide nucleic acid comprises a sequence of SEQ ID NO: 65; the programmable nuclease comprises an amino acid sequence of SEQ ID NO: 25, and the engineered guide nucleic acid comprises a sequence of SEQ ID NO: 66; the programmable nuclease comprises an amino acid sequence of SEQ ID NO: 26, and the engineered guide nucleic acid comprises a sequence of SEQ ID NO: 67; or the programmable nuclease comprises an amino acid sequence of SEQ ID NO: 27, and the engineered guide nucleic acid comprises a sequence of SEQ ID NO: 68. In some embodiments, the engineered guide nucleic acid comprises a crRNA, a tracrRNA, or a combination thereof. In some embodiments, CRISPR RNA (crRNA) is a type of guide nucleic acid, wherein the nucleic acid is RNA comprising a first sequence, often referred to herein as a spacer sequence, that hybridizes to a target sequence of a target nucleic acid, and a second sequence that either a) hybridizes to a portion of a tracrRNA or b) is capable of being non-covalently bound by a programmable nuclease. In some embodiments, the crRNA is covalently linked to an additional nucleic acid (e.g., a tracrRNA) that interacts with the programmable nuclease.

In some embodiments, guide nucleic acids and portions thereof may be found in or identified from a CRISPR array present in the genome of a host organism. A crRNA may be the product of processing of a longer precursor CRISPR RNA (pre-crRNA) transcribed from the CRISPR array by cleavage of the pre-crRNA within each direct repeat sequence to afford shorter, mature crRNAs. A crRNA may be generated by a variety of mechanisms, including the use of dedicated endonucleases (e.g., Cas6 or Cas5d in Type I and III systems), coupling of a host endonuclease (e.g., RNase III) with tracrRNA (Type II systems), or a ribonuclease activity endogenous to the programmable nuclease itself (e.g., Cpfl, from Type V systems). A crRNA may also be specifically generated outside of processing of a pre-crRNA and individually contacted to a programmable nuclease in vivo or in vitro.

In some embodiments, the engineered guide nucleic acid is a single guide nucleic acid. In some embodiments, the amino acid sequence of the programmable nuclease is about 500 to about 850 amino acids in length. In some embodiments, the amino acid sequence of the programmable nuclease is about 780 to about 850 amino acids in length. In some embodiments, the amino acid sequence of the programmable nuclease is about 500 to about 600 amino acids in length. In some embodiments, the programmable nuclease exhibits increased trans-cleavage activity when the guide RNA comprises a spacer region about 25 nucleotides in length, as compared to the cleavage produced by a composition comprising the same programmable nuclease and a guide nucleic acid comprising a spacer region less than 20 nucleotides in length, or greater than 30 nucleotides in length. In some embodiments, the cleavage exhibited by the programmable nuclease when the guide nucleic acid comprises a spacer region of about 20 to about 30 nucleotides in length is at least 2-fold greater than the cleavage produced by a composition comprising the same programmable nuclease and a guide nucleic acid comprising a spacer region less than 20 nucleotides in length, or greater than 30 nucleotides in length. In some embodiments, the cleavage exhibited by the programmable nuclease when the guide nucleic acid comprises a spacer region of about 20 to about 30 nucleotides in length is at least 5-fold greater than the cleavage produced by a composition comprising the same programmable nuclease and a guide nucleic acid comprising a spacer region less than 20 nucleotides in length, or greater than 30 nucleotides in length. In some embodiments, the cleavage exhibited by the programmable nuclease when the guide nucleic acid comprises a spacer region of about 20 to about 30 nucleotides in length is at least 10-fold greater than the cleavage produced by a composition comprising the same programmable nuclease and a guide nucleic acid comprising a spacer region less than 20 nucleotides in length, or greater than 30 nucleotides in length. In some embodiments, the programmable nuclease and engineered guide nucleic acid are capable of catalyzing cRNA-directed, RNA-targeted trans-cleavage activity at a temperature of about 0° C., about 10° C., about 20° C., about 30° C., about 40° C., about 50° C., about 55° C., about 60° C., about 65° C., or about 70° C. In some embodiments, the programmable nuclease and engineered guide nucleic acid are capable of catalyzing cRNA-directed, RNA-targeted trans-cleavage activity at a temperature of about 55° C. In some embodiments, the programmable nuclease and engineered guide nucleic acid are capable of catalyzing cRNA-directed, RNA-targeted trans-cleavage activity at a temperature of about 60° C. In some embodiments, the programmable nuclease and engineered guide nucleic acid are capable of catalyzing cRNA-directed, RNA-targeted trans-cleavage activity at a temperature of about 65° C. In some embodiments, the programmable nuclease and engineered guide nucleic acid are capable of catalyzing cRNA-directed, RNA-targeted trans-cleavage activity at a temperature of about 70° C. In some embodiments, the programmable nuclease and engineered guide nucleic acid are capable of catalyzing cRNA-directed, RNA-targeted trans-cleavage activity at a temperature of about 20° C. In some embodiments, the programmable nuclease and engineered guide nucleic acid are capable of catalyzing cRNA-directed, RNA-targeted trans-cleavage activity at a temperature of not greater than 20° C. In some embodiments, the programmable nuclease and engineered guide nucleic acid are capable of catalyzing cRNA-directed, RNA-targeted trans-cleavage activity at a temperature of at least 20° C. In some embodiments, the programmable nuclease and engineered guide nucleic acid are capable of catalyzing cRNA-directed, RNA-targeted trans-cleavage activity at room temperature. In some embodiments, the programmable nuclease and engineered guide nucleic acid are capable of catalyzing cRNA-directed, RNA-targeted cleavage activity at a temperature of around 20° C.-70° C. In some embodiments, the programmable nuclease and engineered guide nucleic acid are capable of catalyzing cRNA-directed, RNA-targeted cleavage activity at a temperature of around 0° C.-10° C., 0° C.-20° C., 10° C.-20° C., 20° C.-40° C., 25° C.-40° C., 30° C.-40° C., 35° C.-40° C., 30° C.-50° C., 35° C.-50° C., 40° C.-50° C., 45° C.-50° C., 45° C.-60° C., 50° C.-60° C., 55° C.-60° C., 50° C.-70° C., 55° C.-70° C., or 60° C.-70° C. In some embodiments, the programmable nuclease is from a mesophilic organism. In some embodiments, the programmable nuclease is active between 20° C.-70° C. In some embodiments, the programmable nuclease is active between 0° C.-10° C., 0° C.-20° C., 10° C.-20° C., 20° C.-40° C., 25° C.-40° C., 30° C.-40° C., 35° C.-40° C., 30° C.-50° C., 35° C.-50° C., 40° C.-50° C., 45° C.-50° C., 45° C.-60° C., 50° C.-60° C., 55° C.-60° C., 50° C.-70° C., 55° C.-70° C., or 60° C.-70° C. In some embodiments, the programmable nuclease is active at room temperature. In some embodiments, the programmable nuclease comprises two HEPN or HEPN-like domains. In some embodiments, the programmable nuclease is a Cas13c nuclease. In some embodiments, the programmable nuclease is identified in a wild-type bacterial genome by association with a locus comprising a CRISPR array and lacking a cas1 gene or a cas2 gene. In some embodiments, clustered regularly interspaced short palindromic repeats (CRISPR) is a segment of DNA found in the genomes of certain prokaryotic organisms, including some bacteria and archaea, that includes repeated short sequences of nucleotides interspersed at regular intervals between unique sequences of nucleotides derived from the DNA of a pathogen (e.g., virus) that had previously infected the organism and that functions to protect the organism against future infections by the same pathogen.

Provided herein, in some embodiments, is a non-naturally occurring composition comprising a programmable nuclease and an engineered guide nucleic acid capable of catalyzing cRNA-directed, RNA-targeted trans-cleavage activity at a temperature of at least about 55° C. to at least about 85° C., wherein the programmable nuclease comprises at least one HEPN or HEPN-like domain. In some embodiments, the amino acid sequence of the programmable nuclease is about 500 to about 850 amino acids in length. In some embodiments, the amino acid sequence of the programmable nuclease is about 780 to about 850 amino acids in length. In some embodiments, the amino acid sequence of the programmable nuclease is about 500 to about 600 amino acids in length. In some embodiments, the programmable nuclease exhibits increased trans-cleavage activity when the guide RNA comprises a spacer region about 25 nucleotides in length, as compared to the cleavage produced by a composition comprising the same programmable nuclease and a guide nucleic acid comprising a spacer region less than 20 nucleotides in length, or greater than 30 nucleotides in length. In some embodiments, the cleavage exhibited by the programmable nuclease when the guide nucleic acid comprises a spacer region of about 20 to about 30 nucleotides in length is at least 2-fold greater than the cleavage produced by a composition comprising the same programmable nuclease and a guide nucleic acid comprising a spacer region less than 20 nucleotides in length, or greater than 30 nucleotides in length. In some embodiments, the cleavage exhibited by the programmable nuclease when the guide nucleic acid comprises a spacer region of about 20 to about 30 nucleotides in length is at least 5-fold greater than the cleavage produced by a composition comprising the same programmable nuclease and a guide nucleic acid comprising a spacer region less than 20 nucleotides in length, or greater than 30 nucleotides in length. In some embodiments, the cleavage exhibited by the programmable nuclease when the guide nucleic acid comprises a spacer region of about 20 to about 30 nucleotides in length is at least 10-fold greater than the cleavage produced by a composition comprising the same programmable nuclease and a guide nucleic acid comprising a spacer region less than 20 nucleotides in length, or greater than 30 nucleotides in length. In some embodiments, the programmable nuclease and engineered guide nucleic acid are capable of catalyzing cRNA-directed, RNA-targeted trans-cleavage activity at a temperature of about 0° C., about 10° C., about 20° C., about 30° C., about 40° C., about 50° C., about 55° C., about 60° C., about 65° C., or about 70° C. In some embodiments, the programmable nuclease and engineered guide nucleic acid are capable of catalyzing cRNA-directed, RNA-targeted trans-cleavage activity at a temperature of about 30° C. In some embodiments, the programmable nuclease and engineered guide nucleic acid are capable of catalyzing cRNA-directed, RNA-targeted trans-cleavage activity at a temperature of about 40° C. In some embodiments, the programmable nuclease and engineered guide nucleic acid are capable of catalyzing cRNA-directed, RNA-targeted trans-cleavage activity at a temperature of about 50° C. In some embodiments, the programmable nuclease and engineered guide nucleic acid are capable of catalyzing cRNA-directed, RNA-targeted trans-cleavage activity at a temperature of about 55° C. In some embodiments, the programmable nuclease and engineered guide nucleic acid are capable of catalyzing cRNA-directed, RNA-targeted trans-cleavage activity at a temperature of about 60° C. In some embodiments, the programmable nuclease and engineered guide nucleic acid are capable of catalyzing cRNA-directed, RNA-targeted trans-cleavage activity at a temperature of about 65° C. In some embodiments, the programmable nuclease and engineered guide nucleic acid are capable of catalyzing cRNA-directed, RNA-targeted trans-cleavage activity at a temperature of about 70° C. In some embodiments, the programmable nuclease and engineered guide nucleic acid are capable of catalyzing cRNA-directed, RNA-targeted trans-cleavage activity at a temperature of about 20° C. In some embodiments, the programmable nuclease and engineered guide nucleic acid are capable of catalyzing cRNA-directed, RNA-targeted trans-cleavage activity at a temperature of not greater than 20° C. In some embodiments, the programmable nuclease and engineered guide nucleic acid are capable of catalyzing cRNA-directed, RNA-targeted trans-cleavage activity at a temperature of at least 20° C. In some embodiments, the programmable nuclease and engineered guide nucleic acid are capable of catalyzing cRNA-directed, RNA-targeted trans-cleavage activity at room temperature. In some embodiments, the programmable nuclease comprises two HEPN or HEPN-like domains. In some embodiments, the programmable nuclease is a Cas13c nuclease. In some embodiments, the programmable nuclease is identified in a wild-type bacterial genome by association with a locus comprising a CRISPR array and lacking a cas1 gene or a cas2 gene. In some embodiments, the amino acid sequence of the programmable nuclease is at least about 50% identical to a Cas13 protein. In some embodiments, the amino acid sequence of the programmable nuclease is at least about 60% identical to a Cas13 protein. In some embodiments, the amino acid sequence of the programmable nuclease is at least about 70% identical to a Cas13 protein. In some embodiments, the amino acid sequence of the programmable nuclease is at least about 80% identical to a Cas13 protein. In some embodiments, the amino acid sequence of the programmable nuclease is at least about 90% identical to a Cas13 protein. In some embodiments, the programmable nuclease comprises an amino acid sequence that is at least 75%, at least 80%, at least 85%, at least 90%, at least 95% or 100% identical to any one of SEQ ID NOS: 38-520. In some embodiments, the programmable nuclease comprises an amino acid sequence that is at least 85% identical to any one of SEQ ID NOS: 38-52. In some embodiments, the programmable nuclease comprises an amino acid sequence that is at least 95% identical to any one of SEQ ID NOS: 38-52. In some embodiments, the programmable nuclease comprises an amino acid sequence of any one of SEQ ID NOS: 38-52. In some embodiments, the engineered guide nucleic acid comprises a nucleotide sequence of any one of SEQ ID NOS: 53-61.

Also provided herein is a non-naturally occurring composition comprising: i) a programmable nuclease comprising at least one HEPN or HEPN-like domain; and ii) an engineered guide nucleic acid. In some embodiments, the programmable nuclease comprises at least 75% sequence identity to a sequence selected from a group consisting of: SEQ ID NO: 1-SEQ ID NO: 5. In some embodiments, the engineered guide nucleic comprises at least 75% sequence identity to a sequence selected from a group consisting of: SEQ ID NO: 28-SEQ ID NO: 32. In some embodiments, the engineered guide nucleic acid comprises a sequence selected from a group consisting of: SEQ ID NO: 28-SEQ ID NO: 32. In some embodiments, the engineered guide nucleic acid comprises a first region (FR1) complementary to a target sequence and the second region (FR2) that is not complementary to the target sequence. In some embodiments, the first region and second region are oriented: FR1-FR2. In some embodiments, the first region and second region are oriented FR2-FR1. In some embodiments, FR1 comprises at least 75% sequence identity to a sequence selected from a group consisting of: SEQ ID NO: 28-SEQ ID NO: 32. In some embodiments, FR2 is a sequence comprising at least 75% sequence identity to SEQ ID NO: 41.

In some embodiments, the HEPN domain is a HEPN-like domain. Various HEPN-like domains are known in the art and are easily identified using online tools such as InterPro.

Provided herein is a programmable nuclease comprising a sequence with at least 75% sequence identity to SEQ ID NO: 6-SEQ ID NO: 11 which binds to an engineered guide nucleic acid, and wherein the engineered guide nucleic acid comprises a sequence with at least 75% sequence identity to SEQ ID NO: 28-SEQ ID NO: 32. In some embodiments, the programmable nuclease comprises at least one HEPN or HEPN-like domain.

Provided herein is a composition comprising i) a programmable nuclease comprising at least one HEPN or HEPN-like domain, and ii) an engineered guide nucleic acid. In some embodiments, the programmable nuclease comprises at least 75% sequence identity to a sequence selected from a group consisting of: SEQ ID NO: 6-SEQ ID NO: 11. In some embodiments, the engineered guide nucleic comprises at least 75% sequence identity to a sequence selected from a group consisting of: SEQ ID NO: 28-SEQ ID NO: 32. In some embodiments, the engineered guide nucleic acid comprises a sequence selected from a group consisting of: SEQ ID NO: 28-SEQ ID NO: 32. In some embodiments, the engineered guide nucleic acid comprises a first region (FR1) complementary to a target sequence and the second region (FR2) that is not complementary to the target sequence. In some embodiments, the first region and second region are oriented FR1-FR2. In some embodiments, the first region and second region are oriented FR2-FR1. In some embodiments, FR1 comprises at least 75% sequence identity to a sequence selected from a group consisting of: SEQ ID NO: 28-SEQ ID NO: 32. In some embodiments, FR2 comprises at least 75% sequence identity to SEQ ID NO: 41.

Provided herein is a programmable nuclease comprising a sequence with at least 75% sequence identity to SEQ ID NO: 12 which binds to an engineered guide nucleic acid, and wherein the engineered guide nucleic acid comprises a sequence with at least 75% sequence identity to SEQ ID NO: 28-SEQ ID NO: 32. In some embodiments, the programmable nuclease comprises at least one HEPN or HEPN-like domain.

Provided here is a composition comprising i) a programmable nuclease comprising at least one HEPN or HEPN-like domain; and ii) an engineered guide nucleic acid. In some embodiments, the programmable nuclease comprises at least 75% sequence identity to SEQ ID NO: 12. In some embodiments, the engineered guide nucleic comprises at least 75% sequence identity to a sequence selected from a group consisting of: SEQ ID NO: 28-SEQ ID NO: 32. In some embodiments, the engineered guide nucleic acid comprises a sequence selected from a group consisting of: SEQ ID NO: 28-SEQ ID NO: 32. In some embodiments, the engineered guide nucleic acid comprises a first region (FR1) complementary to a target sequence and the second region (FR2) that is not complementary to the target sequence. In some embodiments, the first region and second region are oriented FR1-FR2. In some embodiments, the first region and second region are oriented FR2-FR1. In some embodiments, FR1 comprises at least 75% sequence identity to a sequence selected from a group consisting of: SEQ ID NO: 28-SEQ ID NO: 32. In some embodiments, FR2 comprises at least 75% sequence identity to SEQ ID NO: 41.

Provided herein is a programmable nuclease comprising a sequence with at least 75% sequence identity to SEQ ID NO: 13 or SEQ ID NO: 14 which binds to an engineered guide nucleic acid, and wherein the engineered guide nucleic acid comprises a sequence with at least 75% sequence identity to SEQ ID NO: 28-SEQ ID NO: 32. In some embodiments, the programmable nuclease comprises at least one HEPN or HEPN-like domain.

Provided herein is a composition comprising i) a programmable nuclease comprising at least one HEPN or HEPN-like domain; and ii) an engineered guide nucleic acid. In some embodiments, the programmable nuclease comprises at least 75% sequence identity to a sequence selected from a group consisting of: SEQ ID NO: 13 and SEQ ID NO: 14. In some embodiments, the engineered guide nucleic comprises at least 75% sequence identity to a sequence selected from a group consisting of: SEQ ID NO: 28-SEQ ID NO: 32. In some embodiments, the engineered guide nucleic acid comprises a sequence selected from a group consisting of: SEQ ID NO: 28-SEQ ID NO: 32. In some embodiments, the engineered guide nucleic acid comprises a first region (FR1) complementary to a target sequence and the second region (FR2) that is not complementary to the target sequence. In some embodiments, the first region and second region are oriented: FR1-FR2. In some embodiments, wherein the first region and second region are oriented FR2-FR1. In some embodiments, FR1 comprises at least 75% sequence identity to a sequence selected from a group consisting of: SEQ ID NO: 28-SEQ ID NO: 32. In some embodiments, FR2 comprises at least 75% sequence identity to SEQ ID NO: 41.

### Methods of Detecting a Nucleic Acid in a Sample

Provided herein is a method of detecting a nucleic acid in a sample, comprising the steps of i) contacting a sample with: a) a programmable nuclease; b) a reporter; and c) an engineered guide nucleic acid; and ii) measuring a detectable signal produced by or indicative of cleavage of the reporter, wherein the measuring provides detection of the target nucleic acid in the sample. In some embodiments, at least one programmable nuclease comprises at least 75% sequence identity to a sequence selected from a group consisting of: SEQ ID NO: 6-SEQ ID NO: 11. In some embodiments, the reporter comprises a detection moiety and optionally a quencher. In some embodiments, the detection moiety and the quencher are selected from Table 3. In some embodiments, the detection moiety comprises an enzyme (e.g., horseradish peroxidase, HRP) which, when applied to an enzyme substrate, produces a detectable signal indicative of cleavage of the reporter. In some embodiments, the reporter comprises a nucleic acid sequence. In some embodiments, the nucleic acid sequence is selected from a group consisting of SEQ ID NO: 33-SEQ ID NO: 40. In some embodiments, the engineered guide nucleic acid comprises a first region (FR1) complementary to a target sequence and the second region (FR2) that is not complementary to the target sequence. In some embodiments, the first region and second region are oriented FR1-FR2. In some embodiments, the first region and second region are oriented FR2-FR1. In some embodiments, FR1 comprises at least 75% sequence identity to a sequence selected from a group consisting of: SEQ ID NO: 28-SEQ ID NO: 32. In some embodiments, FR2 comprises at least 75% sequence identity to SEQ ID NO: 41.

Provided herein is a method of detecting a nucleic acid in a sample, comprising the steps of i) contacting a sample with: a) a programmable nuclease; b) a reporter; and c) an engineered guide nucleic acid; and ii) measuring a detectable signal produced by or indicative of cleavage of the reporter, wherein the measuring provides detection of the target nucleic acid in the sample. In some embodiments, at least one programmable nuclease comprises at least 75% sequence identity to SEQ ID NO: 12. In some embodiments, the reporter comprises a detection moiety and optionally a quencher. In some embodiments, the detection moiety and the quencher are selected from Table 3. In some embodiments, the detection moiety comprises an enzyme (e.g., horseradish peroxidase, HRP) which, when applied to an enzyme substrate, produces a detectable signal indicative of cleavage of the reporter. In some embodiments, the reporter comprises a nucleic acid sequence. In some embodiments, the nucleic acid sequence is selected from a group consisting of: SEQ ID NO: 33-SEQ ID NO: 40. In some embodiments, the engineered guide nucleic acid comprises a first region (FR1) complementary to a target sequence and the second region (FR2) that is not complementary to the target sequence. In some embodiments, the first region and second region are oriented FR1-FR2. In some embodiments, the first region and second region are oriented FR2-FR1. In some embodiments, FR1 comprises at least 75% sequence identity to a sequence selected from a group consisting of: SEQ ID NO: 28-SEQ ID NO: 32. In some embodiments, FR2 comprises at least 75% sequence identity to SEQ ID NO: 41.

Provided herein is a method of detecting a nucleic acid in a sample, comprising the steps of i) contacting a sample with: a) a programmable nuclease; b) a reporter; and c) an engineered guide nucleic acid; and ii) measuring a detectable signal produced by or indicative of cleavage of the reporter, wherein the measuring provide detection of the target nucleic acid in the sample. In some embodiments, at least one programmable nuclease comprises at least 75% sequence identity to a sequence selected from a group consisting of: SEQ ID NO: 13 and SEQ ID NO: 14. In some embodiments, the reporter comprises a detection moiety and optionally a quencher. In some embodiments, the detection moiety and the quencher are selected from Table 3. In some embodiments, the detection moiety comprises an enzyme (e.g., horseradish peroxidase, HRP) which, when applied to an enzyme substrate, produces a detectable signal indicative of cleavage of the reporter. In some embodiments, the reporter comprises a nucleic acid sequence. In some embodiments, the nucleic acid sequence is selected from a group consisting: SEQ ID NO: 33-SEQ ID NO: 40. In some embodiments, the engineered guide nucleic acid comprises a first region (FR1) complementary to a target sequence and the second region (FR2) that is not complementary to the target sequence. In some embodiments, the first region and second region are oriented FR1-FR2. In some embodiments, the first region and second region are oriented FR2-FR1. In some embodiments, FR1 comprises at least 75% sequence identity to a sequence selected from a group consisting of: SEQ ID NO: 28-SEQ ID NO: 32. In some embodiments, FR2 comprises at least 75% sequence identity to SEQ ID NO: 41.

### Methods of Altering the Sequence of a Nucleic Acid

Provided herein is a method of altering the sequence of a nucleic acid, the method comprising: i) contacting a nucleic acid molecule with a) a programmable nuclease; and b) an engineered guide nucleic acid. In some embodiments, the nucleic acid is a single stranded ribonucleic acid. In some embodiments, the programmable nuclease comprises at least 75% sequence identity to a sequence selected from a group consisting of: SEQ ID NO: 6-SEQ ID NO: 11. In some embodiments, the programmable nuclease further comprises an editing domain. In some embodiments, the editing domain comprises ADAR1/2 or a functional variant thereof. In some embodiments, the engineered guide nucleic acid comprises a first region (FR1) complementary to a target sequence and the second region (FR2) that is not complementary to the target sequence. In some embodiments, the first region and second region are oriented FR1-FR2. In some embodiments, the first region and second region are oriented FR2-FR1. In some embodiments, FR1 comprises at least 75% sequence identity to a sequence selected from a group consisting of: SEQ ID NO: 28-SEQ ID NO: 32. In some embodiments, FR2 comprises at least 75% sequence identity to SEQ ID NO: 41.

Provided herein is a method of altering the sequence of a nucleic acid, comprising the steps of i) contacting a nucleic acid molecule with a) a programmable nuclease; and b) an engineered guide nucleic acid. In some embodiments, the nucleic acid is a single stranded ribonucleic acid. In some embodiments, the programmable nuclease comprises at least 75% sequence identity to a sequence selected from a group consisting of: SEQ ID NO: 12. In some embodiments, the programmable nuclease further comprises an editing domain. In some embodiments, the editing domain comprises ADAR1/2 or a functional variant thereof. In some embodiments, the engineered guide nucleic acid comprises a first region (FR1) complementary to a target sequence and the second region (FR2) that is not complementary to the target sequence. In some embodiments, the first region and second region are oriented FR1-FR2. In some embodiments, the first region and second region are oriented FR2-FR1. In some embodiments, FR1 comprises at least 75% sequence identity to a sequence selected from a group consisting of: SEQ ID NO: 28-SEQ ID NO: 32. In some embodiments, FR2 comprises at least 75% sequence identity to SEQ ID NO: 41.

Provided herein is a method of altering the sequence of a nucleic acid, comprising the steps of i) contacting a nucleic acid molecule with a) a programmable nuclease; and b) an engineered guide nucleic acid. In some embodiments, the nucleic acid is a single stranded ribonucleic acid. In some embodiments, the programmable nuclease comprises at least 75% sequence identity to a sequence selected from a group consisting of: SEQ ID NO: 13 and SEQ ID NO: 14. In some embodiments, the programmable nuclease further comprises an editing domain. In some embodiments, the editing domain comprises ADAR1/2 or a functional variant thereof. In some embodiments, the engineered guide nucleic acid comprises a first region (FR1) complementary to a target sequence and the second region (FR2) that is not complementary to the target sequence. In some embodiments, the first region and second region are oriented FR1-FR2. In some embodiments, the first region and second region are oriented FR2-FR1. In some embodiments, FR1 comprises at least 75% sequence identity to a sequence selected from a group consisting of: SEQ ID NO: 28-SEQ ID NO: 32. In some embodiments, FR2 comprises at least 75% sequence identity to SEQ ID NO: 41.

### Methods of Introducing a Break in a Target Nucleic Acid

Provided herein is a method of introducing a break in a target nucleic acid, the method comprising: i) contacting the target nucleic acid with a) an engineered guide nucleic acid; and b) a programmable nuclease. In some embodiments, the nucleic acid is a single stranded ribonucleic acid. In some embodiments, the programmable nuclease is selected from SEQ ID NO: 6-SEQ ID NO: 11. In some embodiments, the guide nucleic acid comprises a first region (FR1) complementary to a target sequence and the second region (FR2) that is not complementary to the target sequence. In some embodiments, the first region and second region are oriented FR1-FR2. In some embodiments, the first region and second region are oriented FR2-FR1. In some embodiments, FR1 comprises at least 75% sequence identity to a sequence selected from a group consisting: SEQ ID NO: 28-SEQ ID NO: 32. In some embodiments, FR2 comprises at least 75% sequence identity to SEQ ID NO: 41.

Provided herein is a method of introducing a break in a target nucleic acid, the method comprising: i) contacting the target nucleic acid with a) an engineered guide nucleic acid; and b) a programmable nuclease. In some embodiments, the target nucleic acid is a single stranded ribonucleic acid. In some embodiments, the programmable nuclease comprises SEQ ID NO: 12. In some embodiments, the engineered guide nucleic acid comprises a first region (FR1) complementary to a target sequence and the second region (FR2) that is not complementary to the target sequence. In some embodiments, the first region and second region are oriented FR1-FR2. In some embodiments, the first region and second region are oriented FR2-FR1. In some embodiments, FR1 comprises at least 75% sequence identity to a sequence selected from a group consisting of: SEQ ID NO: 28-SEQ ID NO: 32. In some embodiments, FR2 comprises at least 75% sequence identity to SEQ ID NO: 41.

Provided herein is a method of introducing a break in a target nucleic acid, the method comprising i) contacting the target nucleic acid with a) an engineered guide nucleic acid; and b) a programmable nuclease. In some embodiments, the target nucleic acid is a single stranded ribonucleic acid. In some embodiments, the programmable nuclease comprises SEQ ID NO: 13 or SEQ ID NO: 14. In some embodiments, the engineered guide nucleic acid comprises a first region (FR1) complementary to a target sequence and the second region (FR2) that is not complementary to the target sequence. In some embodiments, the first region and second region are oriented FR1-FR2. In some embodiments, the first region and second region are oriented FR2-FR1. In some embodiments, FR1 comprises at least 75% sequence identity to a sequence selected from a group consisting of: SEQ ID NO: 28-SEQ ID NO: 32. In some embodiments, FR2 comprises at least 75% sequence identity to SEQ ID NO: 41.

### Nuclear Localization Signal

In some embodiments, any of Type VI CRISPR/Cas proteins of the present disclosure (e.g., any one of SEQ ID NO: 1-27, or fragments or variants thereof) may include a nuclear localization signal (NLS). In some embodiments, a nuclear localization signal is an entity (e.g., peptide) that facilitates localization of a nucleic acid, protein, or small molecule to the nucleus, when present in a cell that contains a nuclear compartment. In some cases, said NLS may have a sequence of KRPAATKKAGQAKKKKEF (SEQ ID NO: 43). The NLS can be selected to match the cell type of interest, for example several NLSs are known to be functional in different types of eukaryotic cell e.g. in mammalian cells. Suitable NLSs include the SV40 large T antigen NLS (PKKKRKV, SEQ ID NO: 44) and the c Myc NLS (PAAKRVKLD SEQ ID NO: 45). In some embodiments, an NLS may be the SV40 large T antigen NLS or the c Myc NLS. NLSs that are functional in plant cells are described in Chang et al., (Plant Signal Behav. 2013 October; 8(10):e25976). In some embodiments, an NLS sequence can be selected from the following consensus sequences: KR(K/R)R, K(K/R)RK; (P/R)XXKR(\hat{ }DE)(K/R); KRX(W/F/Y)XXAF(SEQ ID NO: 73); (R/P)XXKR(K/R)(\hat{ }DE); LGKR(K/R)(W/F/Y)(SEQ ID NO: 74); KRX10-12K(KR)(KR) or KRX10-12K(KR)X(K/R).

Other exemplary NLSs can be, but are not limited to, RQRRNELKRSP (SEQ ID NO: 47); the hRNPA1 M9 NLS having the sequence NQSSNFGPMKGGNFGGRSSGPYGGGGQYFAKPRNQGGY (SEQ ID NO: 48); the sequence RMRIZFKNKGKDTAELRRRRVEVSVELRKAKKDEQILKRRNV (SEQ ID NO: 49) of the IBB domain from importin-alpha; the sequences VSRKRPRP (SEQ ID NO: 50) and PPKKARED (SEQ ID NO: 51) of the myoma T protein; the sequence PQPKKKPL (SEQ ID NO: 52) of human p53; the sequence SALIKKKKKMAP (SEQ ID NO: 53) of mouse c-abl IV; the sequences DRLRR (SEQ ID NO: 54) and PKQKKRK (SEQ ID NO: 55) of the influenza virus NS1; the sequence RKLKKKIKKL (SEQ ID NO: 56) of the Hepatitis virus delta antigen; the sequence REKKKFLKRR (SEQ ID NO: 57) of the mouse Mx1 protein; the sequence KRKGDEVDGVDEVAKKKSKK (SEQ ID NO: 58) of the human poly(ADP-ribose) polymerase; and the sequence RKCLQAGMNLEARKTKK (SEQ ID NO: 59) of the steroid hormone receptors (human) glucocorticoid.

In some embodiments, one or more NLS are fused or linked to the N-terminus of the Type VI CRISPR/Cas protein. In some embodiments, one or more NLS are fused or linked to the C-terminus of the Type VI CRISPR/Cas protein. In some embodiments, one or more NLS are fused or linked to the N-terminus and/or the C-terminus of the programmable Type VI CRISPR/Cas nuclease. In some embodiments, the link between the NLS and the Type VI CRISPR/Cas protein comprises a tag.

### Compositions and Methods Comprising Type VI CRISPR/Cas Proteins and Uses Thereof

In some embodiments, the Type VI CRISPR/Cas protein comprises more than 200 amino acids, more than 300 amino acids, more than 400 amino acids, more than 500 amino acids, more than 600 amino acids, more than 700 amino acids, or more than 800 amino acids. In some embodiments, the Type VI CRISPR/Cas protein comprises less than 1200 amino acids, less than 1100 amino acids, less than 1000 amino acids, or less than 900 amino acids. In some embodiments, the Type VI CRISPR/Cas protein comprises from 600 and 1500 amino acids, from 700 and 1500 amino acids, from 800 and 1200 amino acids, or from 800 to 1200 amino acids, or any amino acid number therebetween. In preferred embodiments, the Type VI CRISPR/Cas protein comprises between 800 and 1300 amino acids.

A Type VI CRISPR/Cas protein or a variant thereof can comprise at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity with any one of SEQ ID NO: 1 to SEQ ID NO: 27.

Compositions and methods of the disclosure can comprise a Type VI CRISPR/Cas polypeptide comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 1.

Compositions and methods of the disclosure can comprise a Type VI CRISPR/Cas polypeptide comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 2.

Compositions and methods of the disclosure can comprise a Type VI CRISPR/Cas polypeptide comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 3.

Compositions and methods of the disclosure can comprise a Type VI CRISPR/Cas polypeptide comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 4.

Compositions and methods of the disclosure can comprise a Type VI CRISPR/Cas polypeptide comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 5.

Compositions and methods of the disclosure can comprise a Type VI CRISPR/Cas polypeptide comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 6.

Compositions and methods of the disclosure can comprise a Type VI CRISPR/Cas polypeptide comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 7.

Compositions and methods of the disclosure can comprise a Type VI CRISPR/Cas polypeptide comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 8.

Compositions and methods of the disclosure can comprise a Type VI CRISPR/Cas polypeptide comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 9.

Compositions and methods of the disclosure can comprise a Type VI CRISPR/Cas polypeptide comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 10.

Compositions and methods of the disclosure can comprise a Type VI CRISPR/Cas polypeptide comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 11.

Compositions and methods of the disclosure can comprise a Type VI CRISPR/Cas polypeptide comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 12.

Compositions and methods of the disclosure can comprise a Type VI CRISPR/Cas polypeptide comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 13.

Compositions and methods of the disclosure can comprise a Type VI CRISPR/Cas polypeptide comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 14.

Compositions and methods of the disclosure can comprise a Type VI CRISPR/Cas polypeptide comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 15.

Compositions and methods of the disclosure can comprise a Type VI CRISPR/Cas polypeptide comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 16.

Compositions and methods of the disclosure can comprise a Type VI CRISPR/Cas polypeptide comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 17.

Compositions and methods of the disclosure can comprise a Type VI CRISPR/Cas polypeptide comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 18.

Compositions and methods of the disclosure can comprise a Type VI CRISPR/Cas polypeptide comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 19.

Compositions and methods of the disclosure can comprise a Type VI CRISPR/Cas polypeptide comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 20.

Compositions and methods of the disclosure can comprise a Type VI CRISPR/Cas polypeptide comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 21.

Compositions and methods of the disclosure can comprise a Type VI CRISPR/Cas polypeptide comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 22.

Compositions and methods of the disclosure can comprise a Type VI CRISPR/Cas polypeptide comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 23.

Compositions and methods of the disclosure can comprise a Type VI CRISPR/Cas polypeptide comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 24.

Compositions and methods of the disclosure can comprise a Type VI CRISPR/Cas polypeptide comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 25.

Compositions and methods of the disclosure can comprise a Type VI CRISPR/Cas polypeptide comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 26.

Compositions and methods of the disclosure can comprise a Type VI CRISPR/Cas polypeptide comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 27.

The Type VI CRISPR/Cas protein disclosed herein can be codon optimized for expression in a specific cell, for example, a bacterial cell, a plant cell, a eukaryotic cell, an animal cell, a mammalian cell, or a human cell. In some embodiments, the Type VI CRISPR/Cas protein is codon optimized for a human cell.

The Type VI CRISPR/Cas proteins presented in TABLE 1 or variants thereof comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity with any one of SEQ ID NO: 1-SEQ ID NO: 27 can comprise single-stranded RNA cleavage activity. Compositions and methods of the disclosure can comprise a Type VI CRISPR/Cas protein capable of introducing a single-stranded break in a target RNA sequence and comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to any one of SEQ ID NO: 1-SEQ ID NO: 27. Compositions and methods of the disclosure can comprise a Type VI CRISPR/Cas protein capable of introducing a single-stranded break in a target RNA sequence and comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 1. Compositions and methods of the disclosure can comprise a Type VI CRISPR/Cas protein capable of introducing a single-stranded break in a target RNA sequence and comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 2. Compositions and methods of the disclosure can comprise a Type VI CRISPR/Cas protein capable of introducing a single-stranded break in a target RNA sequence and comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 3. Compositions and methods of the disclosure can comprise a Type VI CRISPR/Cas protein capable of introducing a single-stranded break in a target RNA sequence and comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 4. Compositions and methods of the disclosure can comprise a Type VI CRISPR/Cas protein capable of introducing a single-stranded break in a target RNA sequence and comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 5. Compositions and methods of the disclosure can comprise a Type VI CRISPR/Cas protein capable of introducing a single-stranded break in a target RNA sequence and comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 6. Compositions and methods of the disclosure can comprise a Type VI CRISPR/Cas protein capable of introducing a single-stranded break in a target RNA sequence and comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 7. Compositions and methods of the disclosure can comprise a Type VI CRISPR/Cas protein capable of introducing a single-stranded break in a target RNA sequence and comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 8. Compositions and methods of the disclosure can comprise a Type VI CRISPR/Cas protein capable of introducing a single-stranded break in a target RNA sequence and comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 9. Compositions and methods of the disclosure can comprise a Type VI CRISPR/Cas protein capable of introducing a single-stranded break in a target RNA sequence and comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 10. Compositions and methods of the disclosure can comprise a Type VI CRISPR/Cas protein capable of introducing a single-stranded break in a target RNA sequence and comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 11. Compositions and methods of the disclosure can comprise a Type VI CRISPR/Cas protein capable of introducing a single-stranded break in a target RNA sequence and comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 12. Compositions and methods of the disclosure can comprise a Type VI CRISPR/Cas protein capable of introducing a single-stranded break in a target RNA sequence and comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 13. Compositions and methods of the disclosure can comprise a Type VI CRISPR/Cas protein capable of introducing a single-stranded break in a target RNA sequence and comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 14. Compositions and methods of the disclosure can comprise a Type VI CRISPR/Cas protein capable of introducing a single-stranded break in a target RNA sequence and comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 15. Compositions and methods of the disclosure can comprise a Type VI CRISPR/Cas protein capable of introducing a single-stranded break in a target RNA sequence and comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 16. Compositions and methods of the disclosure can comprise a Type VI CRISPR/Cas protein capable of introducing a single-stranded break in a target RNA sequence and comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 17. Compositions and methods of the disclosure can comprise a Type VI CRISPR/Cas protein capable of introducing a single-stranded break in a target RNA sequence and comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 18. Compositions and methods of the disclosure can comprise a Type VI CRISPR/Cas protein capable of introducing a single-stranded break in a target RNA sequence and comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 19. Compositions and methods of the disclosure can comprise a Type VI CRISPR/Cas protein capable of introducing a single-stranded break in a target RNA sequence and comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 20. Compositions and methods of the disclosure can comprise a Type VI CRISPR/Cas protein capable of introducing a single-stranded break in a target RNA sequence and comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 21. Compositions and methods of the disclosure can comprise a Type VI CRISPR/Cas protein capable of introducing a single-stranded break in a target RNA sequence and comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 22. Compositions and methods of the disclosure can comprise a Type VI CRISPR/Cas protein capable of introducing a single-stranded break in a target RNA sequence and comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 23. Compositions and methods of the disclosure can comprise a Type VI CRISPR/Cas protein capable of introducing a single-stranded break in a target RNA sequence and comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 24. Compositions and methods of the disclosure can comprise a Type VI CRISPR/Cas protein capable of introducing a single-stranded break in a target RNA sequence and comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 25. Compositions and methods of the disclosure can comprise a Type VI CRISPR/Cas protein capable of introducing a single-stranded break in a target RNA sequence and comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 26. Compositions and methods of the disclosure can comprise a Type VI CRISPR/Cas protein capable of introducing a single-stranded break in a target RNA sequence and comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 27.

In some embodiments, the Type VI CRISPR/Cas polypeptide comprises a sequence with at least 50% identity to SEQ ID NO: 1. In some embodiments, the Type VI CRISPR/Cas polypeptide comprises a sequence with at least 55% identity to SEQ ID NO: 1. In some embodiments, the Type VI CRISPR/Cas polypeptide comprises a sequence with at least 60% identity to SEQ ID NO: 1. In some embodiments, the Type VI CRISPR/Cas polypeptide comprises a sequence with at least 65% identity to SEQ ID NO: 1. In some embodiments, the Type VI CRISPR/Cas polypeptide comprises a sequence with at least 70% identity to SEQ ID NO: 1. In some embodiments, the Type VI CRISPR/Cas polypeptide comprises a sequence with at least 75% identity to SEQ ID NO: 1. In some embodiments, the Type VI CRISPR/Cas polypeptide comprises a sequence with at least 80% identity to SEQ ID NO: 1. In some embodiments, the Type VI CRISPR/Cas polypeptide comprises a sequence with at least 85% identity to SEQ ID NO: 1. In some embodiments, the Type VI CRISPR/Cas polypeptide comprises a sequence with at least 90% identity to SEQ ID NO: 1. In some embodiments, the Type VI CRISPR/Cas polypeptide comprises a sequence with at least 92% identity to SEQ ID NO: 1. In some embodiments, the Type VI CRISPR/Cas polypeptide comprises a sequence with at least 95% identity to SEQ ID NO: 1. In some embodiments, the Type VI CRISPR/Cas polypeptide comprises a sequence with at least 97% identity to SEQ ID NO: 1. In some embodiments, the Type VI CRISPR/Cas polypeptide comprises a sequence with at least 98% identity to SEQ ID NO: 1. In some embodiments, the Type VI CRISPR/Cas polypeptide comprises a sequence with at least 99% identity to SEQ ID NO: 1. In some embodiments, the Type VI CRISPR/Cas polypeptide comprises a sequence of SEQ ID NO: 1.

In some embodiments, the Type VI CRISPR/Cas polypeptide comprises a sequence with at least 50% identity to SEQ ID NO: 2. In some embodiments, the Type VI CRISPR/Cas polypeptide comprises a sequence with at least 55% identity to SEQ ID NO: 2. In some embodiments, the Type VI CRISPR/Cas polypeptide comprises a sequence with at least 60% identity to SEQ ID NO: 2. In some embodiments, the Type VI CRISPR/Cas polypeptide comprises a sequence with at least 65% identity to SEQ ID NO: 2. In some embodiments, the Type VI CRISPR/Cas polypeptide comprises a sequence with at least 70% identity to SEQ ID NO: 2. In some embodiments, the Type VI CRISPR/Cas polypeptide comprises a sequence with at least 75% identity to SEQ ID NO: 2. In some embodiments, the Type VI CRISPR/Cas polypeptide comprises a sequence with at least 80% identity to SEQ ID NO: 2. In some embodiments, the Type VI CRISPR/Cas polypeptide comprises a sequence with at least 85% identity to SEQ ID NO: 2. In some embodiments, the Type VI CRISPR/Cas polypeptide comprises a sequence with at least 90% identity to SEQ ID NO: 2. In some embodiments, the Type VI CRISPR/Cas polypeptide comprises a sequence with at least 92% identity to SEQ ID NO: 2. In some embodiments, the Type VI CRISPR/Cas polypeptide comprises a sequence with at least 95% identity to SEQ ID NO: 2. In some embodiments, the Type VI CRISPR/Cas polypeptide comprises a sequence with at least 97% identity to SEQ ID NO: 2. In some embodiments, the Type VI CRISPR/Cas polypeptide comprises a sequence with at least 98% identity to SEQ ID NO: 2. In some embodiments, the Type VI CRISPR/Cas polypeptide comprises a sequence with at least 99% identity to SEQ ID NO: 2. In some embodiments, the Type VI CRISPR/Cas polypeptide comprises a sequence of SEQ ID NO: 2.

In some embodiments, the Type VI CRISPR/Cas polypeptide comprises a sequence with at least 50% identity to SEQ ID NO: 3. In some embodiments, the Type VI CRISPR/Cas polypeptide comprises a sequence with at least 55% identity to SEQ ID NO: 3. In some embodiments, the Type VI CRISPR/Cas polypeptide comprises a sequence with at least 60% identity to SEQ ID NO: 3. In some embodiments, the Type VI CRISPR/Cas polypeptide comprises a sequence with at least 65% identity to SEQ ID NO: 3. In some embodiments, the Type VI CRISPR/Cas polypeptide comprises a sequence with at least 70% identity to SEQ ID NO: 3. In some embodiments, the Type VI CRISPR/Cas polypeptide comprises a sequence with at least 75% identity to SEQ ID NO: 3. In some embodiments, the Type VI CRISPR/Cas polypeptide comprises a sequence with at least 80% identity to SEQ ID NO: 3. In some embodiments, the Type VI CRISPR/Cas polypeptide comprises a sequence with at least 85% identity to SEQ ID NO: 3. In some embodiments, the Type VI CRISPR/Cas polypeptide comprises a sequence with at least 90% identity to SEQ ID NO: 3. In some embodiments, the Type VI CRISPR/Cas polypeptide comprises a sequence with at least 92% identity to SEQ ID NO: 3. In some embodiments, the Type VI CRISPR/Cas polypeptide comprises a sequence with at least 95% identity to SEQ ID NO: 3. In some embodiments, the Type VI CRISPR/Cas polypeptide comprises a sequence with at least 97% identity to SEQ ID NO: 3. In some embodiments, the Type VI CRISPR/Cas polypeptide comprises a sequence with at least 98% identity to SEQ ID NO: 3. In some embodiments, the Type VI CRISPR/Cas polypeptide comprises a sequence with at least 99% identity to SEQ ID NO: 3. In some embodiments, the Type VI CRISPR/Cas polypeptide comprises a sequence of SEQ ID NO: 3.

In some embodiments, the Type VI CRISPR/Cas polypeptide comprises a sequence with at least 50% identity to SEQ ID NO: 4. In some embodiments, the Type VI CRISPR/Cas polypeptide comprises a sequence with at least 55% identity to SEQ ID NO: 4. In some embodiments, the Type VI CRISPR/Cas polypeptide comprises a sequence with at least 60% identity to SEQ ID NO: 4. In some embodiments, the Type VI CRISPR/Cas polypeptide comprises a sequence with at least 65% identity to SEQ ID NO: 4. In some embodiments, the Type VI CRISPR/Cas polypeptide comprises a sequence with at least 70% identity to SEQ ID NO: 4. In some embodiments, the Type VI CRISPR/Cas polypeptide comprises a sequence with at least 75% identity to SEQ ID NO: 4. In some embodiments, the Type VI CRISPR/Cas polypeptide comprises a sequence with at least 80% identity to SEQ ID NO: 4. In some embodiments, the Type VI CRISPR/Cas polypeptide comprises a sequence with at least 85% identity to SEQ ID NO: 4. In some embodiments, the Type VI CRISPR/Cas polypeptide comprises a sequence with at least 90% identity to SEQ ID NO: 4. In some embodiments, the Type VI CRISPR/Cas polypeptide comprises a sequence with at least 92% identity to SEQ ID NO: 4. In some embodiments, the Type VI CRISPR/Cas polypeptide comprises a sequence with at least 95% identity to SEQ ID NO: 4. In some embodiments, the Type VI CRISPR/Cas polypeptide comprises a sequence with at least 97% identity to SEQ ID NO: 4. In some embodiments, the Type VI CRISPR/Cas polypeptide comprises a sequence with at least 98% identity to SEQ ID NO: 4. In some embodiments, the Type VI CRISPR/Cas polypeptide comprises a sequence with at least 99% identity to SEQ ID NO: 4. In some embodiments, the Type VI CRISPR/Cas polypeptide comprises a sequence of SEQ ID NO: 4.

In some embodiments, the Type VI CRISPR/Cas polypeptide comprises a sequence with at least 50% identity to SEQ ID NO: 5. In some embodiments, the Type VI CRISPR/Cas polypeptide comprises a sequence with at least 55% identity to SEQ ID NO: 5. In some embodiments, the Type VI CRISPR/Cas polypeptide comprises a sequence with at least 60% identity to SEQ ID NO: 5. In some embodiments, the Type VI CRISPR/Cas polypeptide comprises a sequence with at least 65% identity to SEQ ID NO: 5. In some embodiments, the Type VI CRISPR/Cas polypeptide comprises a sequence with at least 70% identity to SEQ ID NO: 5. In some embodiments, the Type VI CRISPR/Cas polypeptide comprises a sequence with at least 75% identity to SEQ ID NO: 5. In some embodiments, the Type VI CRISPR/Cas polypeptide comprises a sequence with at least 80% identity to SEQ ID NO: 5. In some embodiments, the Type VI CRISPR/Cas polypeptide comprises a sequence with at least 85% identity to SEQ ID NO: 5. In some embodiments, the Type VI CRISPR/Cas polypeptide comprises a sequence with at least 90% identity to SEQ ID NO: 5. In some embodiments, the Type VI CRISPR/Cas polypeptide comprises a sequence with at least 92% identity to SEQ ID NO: 5. In some embodiments, the Type VI CRISPR/Cas polypeptide comprises a sequence with at least 95% identity to SEQ ID NO: 5. In some embodiments, the Type VI CRISPR/Cas polypeptide comprises a sequence with at least 97% identity to SEQ ID NO: 5. In some embodiments, the Type VI CRISPR/Cas polypeptide comprises a sequence with at least 98% identity to SEQ ID NO: 5. In some embodiments, the Type VI CRISPR/Cas polypeptide comprises a sequence with at least 99% identity to SEQ ID NO: 5. In some embodiments, the Type VI CRISPR/Cas polypeptide comprises a sequence of SEQ ID NO: 5.

In some embodiments, the Type VI CRISPR/Cas polypeptide comprises a sequence with at least 50% identity to SEQ ID NO: 6. In some embodiments, the Type VI CRISPR/Cas polypeptide comprises a sequence with at least 55% identity to SEQ ID NO: 6. In some embodiments, the Type VI CRISPR/Cas polypeptide comprises a sequence with at least 60% identity to SEQ ID NO: 6. In some embodiments, the Type VI CRISPR/Cas polypeptide comprises a sequence with at least 65% identity to SEQ ID NO: 6. In some embodiments, the Type VI CRISPR/Cas polypeptide comprises a sequence with at least 70% identity to SEQ ID NO: 6. In some embodiments, the Type VI CRISPR/Cas polypeptide comprises a sequence with at least 75% identity to SEQ ID NO: 6. In some embodiments, the Type VI CRISPR/Cas polypeptide comprises a sequence with at least 80% identity to SEQ ID NO: 6. In some embodiments, the Type VI CRISPR/Cas polypeptide comprises a sequence with at least 85% identity to SEQ ID NO: 6. In some embodiments, the Type VI CRISPR/Cas polypeptide comprises a sequence with at least 90% identity to SEQ ID NO: 6. In some embodiments, the Type VI CRISPR/Cas polypeptide comprises a sequence with at least 92% identity to SEQ ID NO: 6. In some embodiments, the Type VI CRISPR/Cas polypeptide comprises a sequence with at least 95% identity to SEQ ID NO: 6. In some embodiments, the Type VI CRISPR/Cas polypeptide comprises a sequence with at least 97% identity to SEQ ID NO: 6. In some embodiments, the Type VI CRISPR/Cas polypeptide comprises a sequence with at least 98% identity to SEQ ID NO: 6. In some embodiments, the Type VI CRISPR/Cas polypeptide comprises a sequence with at least 99% identity to SEQ ID NO: 6. In some embodiments, the Type VI CRISPR/Cas polypeptide comprises a sequence of SEQ ID NO: 6.

In some embodiments, the Type VI CRISPR/Cas polypeptide comprises a sequence with at least 50% identity to SEQ ID NO: 7. In some embodiments, the Type VI CRISPR/Cas polypeptide comprises a sequence with at least 55% identity to SEQ ID NO: 7. In some embodiments, the Type VI CRISPR/Cas polypeptide comprises a sequence with at least 60% identity to SEQ ID NO: 7. In some embodiments, the Type VI CRISPR/Cas polypeptide comprises a sequence with at least 65% identity to SEQ ID NO: 7. In some embodiments, the Type VI CRISPR/Cas polypeptide comprises a sequence with at least 70% identity to SEQ ID NO: 7. In some embodiments, the Type VI CRISPR/Cas polypeptide comprises a sequence with at least 75% identity to SEQ ID NO: 7. In some embodiments, the Type VI CRISPR/Cas polypeptide comprises a sequence with at least 80% identity to SEQ ID NO: 7. In some embodiments, the Type VI CRISPR/Cas polypeptide comprises a sequence with at least 85% identity to SEQ ID NO: 7. In some embodiments, the Type VI CRISPR/Cas polypeptide comprises a sequence with at least 90% identity to SEQ ID NO: 7. In some embodiments, the Type VI CRISPR/Cas polypeptide comprises a sequence with at least 92% identity to SEQ ID NO: 7. In some embodiments, the Type VI CRISPR/Cas polypeptide comprises a sequence with at least 95% identity to SEQ ID NO: 7. In some embodiments, the Type VI CRISPR/Cas polypeptide comprises a sequence with at least 97% identity to SEQ ID NO: 7. In some embodiments, the Type VI CRISPR/Cas polypeptide comprises a sequence with at least 98% identity to SEQ ID NO: 7. In some embodiments, the Type VI CRISPR/Cas polypeptide comprises a sequence with at least 99% identity to SEQ ID NO: 7. In some embodiments, the Type VI CRISPR/Cas polypeptide comprises a sequence of SEQ ID NO: 7.

In some embodiments, the Type VI CRISPR/Cas polypeptide comprises a sequence with at least 50% identity to SEQ ID NO: 8. In some embodiments, the Type VI CRISPR/Cas polypeptide comprises a sequence with at least 55% identity to SEQ ID NO: 8. In some embodiments, the Type VI CRISPR/Cas polypeptide comprises a sequence with at least 60% identity to SEQ ID NO: 8. In some embodiments, the Type VI CRISPR/Cas polypeptide comprises a sequence with at least 65% identity to SEQ ID NO: 8. In some embodiments, the Type VI CRISPR/Cas polypeptide comprises a sequence with at least 70% identity to SEQ ID NO: 8. In some embodiments, the Type VI CRISPR/Cas polypeptide comprises a sequence with at least 75% identity to SEQ ID NO: 8. In some embodiments, the Type VI CRISPR/Cas polypeptide comprises a sequence with at least 80% identity to SEQ ID NO: 8. In some embodiments, the Type VI CRISPR/Cas polypeptide comprises a sequence with at least 85% identity to SEQ ID NO: 8. In some embodiments, the Type VI CRISPR/Cas polypeptide comprises a sequence with at least 90% identity to SEQ ID NO: 8. In some embodiments, the Type VI CRISPR/Cas polypeptide comprises a sequence with at least 92% identity to SEQ ID NO: 8. In some embodiments, the Type VI CRISPR/Cas polypeptide comprises a sequence with at least 95% identity to SEQ ID NO: 8. In some embodiments, the Type VI CRISPR/Cas polypeptide comprises a sequence with at least 97% identity to SEQ ID NO: 8. In some embodiments, the Type VI CRISPR/Cas polypeptide comprises a sequence with at least 98% identity to SEQ ID NO: 8. In some embodiments, the Type VI CRISPR/Cas polypeptide comprises a sequence with at least 99% identity to SEQ ID NO: 8. In some embodiments, the Type VI CRISPR/Cas polypeptide comprises a sequence of SEQ ID NO: 8.

In some embodiments, the Type VI CRISPR/Cas polypeptide comprises a sequence with at least 50% identity to SEQ ID NO: 9. In some embodiments, the Type VI CRISPR/Cas polypeptide comprises a sequence with at least 55% identity to SEQ ID NO: 9. In some embodiments, the Type VI CRISPR/Cas polypeptide comprises a sequence with at least 60% identity to SEQ ID NO: 9. In some embodiments, the Type VI CRISPR/Cas polypeptide comprises a sequence with at least 65% identity to SEQ ID NO: 9. In some embodiments, the Type VI CRISPR/Cas polypeptide comprises a sequence with at least 70% identity to SEQ ID NO: 9. In some embodiments, the Type VI CRISPR/Cas polypeptide comprises a sequence with at least 75% identity to SEQ ID NO: 9. In some embodiments, the Type VI CRISPR/Cas polypeptide comprises a sequence with at least 80% identity to SEQ ID NO: 9. In some embodiments, the Type VI CRISPR/Cas polypeptide comprises a sequence with at least 85% identity to SEQ ID NO: 9. In some embodiments, the Type VI CRISPR/Cas polypeptide comprises a sequence with at least 90% identity to SEQ ID NO: 9. In some embodiments, the Type VI CRISPR/Cas polypeptide comprises a sequence with at least 92% identity to SEQ ID NO: 9. In some embodiments, the Type VI CRISPR/Cas polypeptide comprises a sequence with at least 95% identity to SEQ ID NO: 9. In some embodiments, the Type VI CRISPR/Cas polypeptide comprises a sequence with at least 97% identity to SEQ ID NO: 9. In some embodiments, the Type VI CRISPR/Cas polypeptide comprises a sequence with at least 98% identity to SEQ ID NO: 9. In some embodiments, the Type VI CRISPR/Cas polypeptide comprises a sequence with at least 99% identity to SEQ ID NO: 9. In some embodiments, the Type VI CRISPR/Cas polypeptide comprises a sequence of SEQ ID NO: 9.

In some embodiments, the Type VI CRISPR/Cas polypeptide comprises a sequence with at least 50% identity to SEQ ID NO: 10. In some embodiments, the Type VI CRISPR/Cas polypeptide comprises a sequence with at least 55% identity to SEQ ID NO: 10. In some embodiments, the Type VI CRISPR/Cas polypeptide comprises a sequence with at least 60% identity to SEQ ID NO: 10. In some embodiments, the Type VI CRISPR/Cas polypeptide comprises a sequence with at least 65% identity to SEQ ID NO: 10. In some embodiments, the Type VI CRISPR/Cas polypeptide comprises a sequence with at least 70% identity to SEQ ID NO: 10. In some embodiments, the Type VI CRISPR/Cas polypeptide comprises a sequence with at least 75% identity to SEQ ID NO: 10. In some embodiments, the Type VI CRISPR/Cas polypeptide comprises a sequence with at least 80% identity to SEQ ID NO: 10. In some embodiments, the Type VI CRISPR/Cas polypeptide comprises a sequence with at least 85% identity to SEQ ID NO: 10. In some embodiments, the Type VI CRISPR/Cas polypeptide comprises a sequence with at least 90% identity to SEQ ID NO: 10. In some embodiments, the Type VI CRISPR/Cas polypeptide comprises a sequence with at least 92% identity to SEQ ID NO: 10. In some embodiments, the Type VI CRISPR/Cas polypeptide comprises a sequence with at least 95% identity to SEQ ID NO: 10. In some embodiments, the Type VI CRISPR/Cas polypeptide comprises a sequence with at least 97% identity to SEQ ID NO: 10. In some embodiments, the Type VI CRISPR/Cas polypeptide comprises a sequence with at least 98% identity to SEQ ID NO: 10. In some embodiments, the Type VI CRISPR/Cas polypeptide comprises a sequence with at least 99% identity to SEQ ID NO: 10. In some embodiments, the Type VI CRISPR/Cas polypeptide comprises a sequence of SEQ ID NO: 10.

In some embodiments, the Type VI CRISPR/Cas polypeptide comprises a sequence with at least 50% identity to SEQ ID NO: 11. In some embodiments, the Type VI CRISPR/Cas polypeptide comprises a sequence with at least 55% identity to SEQ ID NO: 11. In some embodiments, the Type VI CRISPR/Cas polypeptide comprises a sequence with at least 60% identity to SEQ ID NO: 11. In some embodiments, the Type VI CRISPR/Cas polypeptide comprises a sequence with at least 65% identity to SEQ ID NO: 11. In some embodiments, the Type VI CRISPR/Cas polypeptide comprises a sequence with at least 70% identity to SEQ ID NO: 11. In some embodiments, the Type VI CRISPR/Cas polypeptide comprises a sequence with at least 75% identity to SEQ ID NO: 11. In some embodiments, the Type VI CRISPR/Cas polypeptide comprises a sequence with at least 80% identity to SEQ ID NO: 11. In some embodiments, the Type VI CRISPR/Cas polypeptide comprises a sequence with at least 85% identity to SEQ ID NO: 11. In some embodiments, the Type VI CRISPR/Cas polypeptide comprises a sequence with at least 90% identity to SEQ ID NO: 11. In some embodiments, the Type VI CRISPR/Cas polypeptide comprises a sequence with at least 92% identity to SEQ ID NO: 11. In some embodiments, the Type VI CRISPR/Cas polypeptide comprises a sequence with at least 95% identity to SEQ ID NO: 11. In some embodiments, the Type VI CRISPR/Cas polypeptide comprises a sequence with at least 97% identity to SEQ ID NO: 11. In some embodiments, the Type VI CRISPR/Cas polypeptide comprises a sequence with at least 98% identity to SEQ ID NO: 11. In some embodiments, the Type VI CRISPR/Cas polypeptide comprises a sequence with at least 99% identity to SEQ ID NO: 11. In some embodiments, the Type VI CRISPR/Cas polypeptide comprises a sequence of SEQ ID NO: 11.

In some embodiments, the Type VI CRISPR/Cas polypeptide comprises a sequence with at least 50% identity to SEQ ID NO: 12. In some embodiments, the Type VI CRISPR/Cas polypeptide comprises a sequence with at least 55% identity to SEQ ID NO: 12. In some embodiments, the Type VI CRISPR/Cas polypeptide comprises a sequence with at least 60% identity to SEQ ID NO: 12. In some embodiments, the Type VI CRISPR/Cas polypeptide comprises a sequence with at least 65% identity to SEQ ID NO: 12. In some embodiments, the Type VI CRISPR/Cas polypeptide comprises a sequence with at least 70% identity to SEQ ID NO: 12. In some embodiments, the Type VI CRISPR/Cas polypeptide comprises a sequence with at least 75% identity to SEQ ID NO: 12. In some embodiments, the Type VI CRISPR/Cas polypeptide comprises a sequence with at least 80% identity to SEQ ID NO: 12. In some embodiments, the Type VI CRISPR/Cas polypeptide comprises a sequence with at least 85% identity to SEQ ID NO: 12. In some embodiments, the Type VI CRISPR/Cas polypeptide comprises a sequence with at least 90% identity to SEQ ID NO: 12. In some embodiments, the Type VI CRISPR/Cas polypeptide comprises a sequence with at least 92% identity to SEQ ID NO: 12. In some embodiments, the Type VI CRISPR/Cas polypeptide comprises a sequence with at least 95% identity to SEQ ID NO: 12. In some embodiments, the Type VI CRISPR/Cas polypeptide comprises a sequence with at least 97% identity to SEQ ID NO: 12. In some embodiments, the Type VI CRISPR/Cas polypeptide comprises a sequence with at least 98% identity to SEQ ID NO: 12. In some embodiments, the Type VI CRISPR/Cas polypeptide comprises a sequence with at least 99% identity to SEQ ID NO: 12. In some embodiments, the Type VI CRISPR/Cas polypeptide comprises a sequence of SEQ ID NO: 12.

In some embodiments, the Type VI CRISPR/Cas polypeptide comprises a sequence with at least 50% identity to SEQ ID NO: 13. In some embodiments, the Type VI CRISPR/Cas polypeptide comprises a sequence with at least 55% identity to SEQ ID NO: 13. In some embodiments, the Type VI CRISPR/Cas polypeptide comprises a sequence with at least 60% identity to SEQ ID NO: 13. In some embodiments, the Type VI CRISPR/Cas polypeptide comprises a sequence with at least 65% identity to SEQ ID NO: 13. In some embodiments, the Type VI CRISPR/Cas polypeptide comprises a sequence with at least 70% identity to SEQ ID NO: 13. In some embodiments, the Type VI CRISPR/Cas polypeptide comprises a sequence with at least 75% identity to SEQ ID NO: 13. In some embodiments, the Type VI CRISPR/Cas polypeptide comprises a sequence with at least 80% identity to SEQ ID NO: 13. In some embodiments, the Type VI CRISPR/Cas polypeptide comprises a sequence with at least 85% identity to SEQ ID NO: 13. In some embodiments, the Type VI CRISPR/Cas polypeptide comprises a sequence with at least 90% identity to SEQ ID NO: 13. In some embodiments, the Type VI CRISPR/Cas polypeptide comprises a sequence with at least 92% identity to SEQ ID NO: 13. In some embodiments, the Type VI CRISPR/Cas polypeptide comprises a sequence with at least 95% identity to SEQ ID NO: 13. In some embodiments, the Type VI CRISPR/Cas polypeptide comprises a sequence with at least 97% identity to SEQ ID NO: 13. In some embodiments, the Type VI CRISPR/Cas polypeptide comprises a sequence with at least 98% identity to SEQ ID NO: 13. In some embodiments, the Type VI CRISPR/Cas polypeptide comprises a sequence with at least 99% identity to SEQ ID NO: 13. In some embodiments, the Type VI CRISPR/Cas polypeptide comprises a sequence of SEQ ID NO: 13.

In some embodiments, the Type VI CRISPR/Cas polypeptide comprises a sequence with at least 50% identity to SEQ ID NO: 14. In some embodiments, the Type VI CRISPR/Cas polypeptide comprises a sequence with at least 55% identity to SEQ ID NO: 14. In some embodiments, the Type VI CRISPR/Cas polypeptide comprises a sequence with at least 60% identity to SEQ ID NO: 14. In some embodiments, the Type VI CRISPR/Cas polypeptide comprises a sequence with at least 65% identity to SEQ ID NO: 14. In some embodiments, the Type VI CRISPR/Cas polypeptide comprises a sequence with at least 70% identity to SEQ ID NO: 14. In some embodiments, the Type VI CRISPR/Cas polypeptide comprises a sequence with at least 75% identity to SEQ ID NO: 14. In some embodiments, the Type VI CRISPR/Cas polypeptide comprises a sequence with at least 80% identity to SEQ ID NO: 14. In some embodiments, the Type VI CRISPR/Cas polypeptide comprises a sequence with at least 85% identity to SEQ ID NO: 14. In some embodiments, the Type VI CRISPR/Cas polypeptide comprises a sequence with at least 90% identity to SEQ ID NO: 14. In some embodiments, the Type VI CRISPR/Cas polypeptide comprises a sequence with at least 92% identity to SEQ ID NO: 14. In some embodiments, the Type VI CRISPR/Cas polypeptide comprises a sequence with at least 95% identity to SEQ ID NO: 14. In some embodiments, the Type VI CRISPR/Cas polypeptide comprises a sequence with at least 97% identity to SEQ ID NO: 14. In some embodiments, the Type VI CRISPR/Cas polypeptide comprises a sequence with at least 98% identity to SEQ ID NO: 14. In some embodiments, the Type VI CRISPR/Cas polypeptide comprises a sequence with at least 99% identity to SEQ ID NO: 14. In some embodiments, the Type VI CRISPR/Cas polypeptide comprises a sequence of SEQ ID NO: 14.

In some embodiments, the Type VI CRISPR/Cas polypeptide comprises a sequence with at least 50% identity to SEQ ID NO: 15. In some embodiments, the Type VI CRISPR/Cas polypeptide comprises a sequence with at least 55% identity to SEQ ID NO: 15. In some embodiments, the Type VI CRISPR/Cas polypeptide comprises a sequence with at least 60% identity to SEQ ID NO: 15. In some embodiments, the Type VI CRISPR/Cas polypeptide comprises a sequence with at least 65% identity to SEQ ID NO: 15. In some embodiments, the Type VI CRISPR/Cas polypeptide comprises a sequence with at least 70% identity to SEQ ID NO: 15. In some embodiments, the Type VI CRISPR/Cas polypeptide comprises a sequence with at least 75% identity to SEQ ID NO: 15. In some embodiments, the Type VI CRISPR/Cas polypeptide comprises a sequence with at least 80% identity to SEQ ID NO: 15. In some embodiments, the Type VI CRISPR/Cas polypeptide comprises a sequence with at least 85% identity to SEQ ID NO: 15. In some embodiments, the Type VI CRISPR/Cas polypeptide comprises a sequence with at least 90% identity to SEQ ID NO: 15. In some embodiments, the Type VI CRISPR/Cas polypeptide comprises a sequence with at least 92% identity to SEQ ID NO: 15. In some embodiments, the Type VI CRISPR/Cas polypeptide comprises a sequence with at least 95% identity to SEQ ID NO: 15. In some embodiments, the Type VI CRISPR/Cas polypeptide comprises a sequence with at least 97% identity to SEQ ID NO: 15. In some embodiments, the Type VI CRISPR/Cas polypeptide comprises a sequence with at least 98% identity to SEQ ID NO: 15. In some embodiments, the Type VI CRISPR/Cas polypeptide comprises a sequence with at least 99% identity to SEQ ID NO: 15. In some embodiments, the Type VI CRISPR/Cas polypeptide comprises a sequence of SEQ ID NO: 15.

In some embodiments, the Type VI CRISPR/Cas polypeptide comprises a sequence with at least 50% identity to SEQ ID NO: 16. In some embodiments, the Type VI CRISPR/Cas polypeptide comprises a sequence with at least 55% identity to SEQ ID NO: 16. In some embodiments, the Type VI CRISPR/Cas polypeptide comprises a sequence with at least 60% identity to SEQ ID NO: 16. In some embodiments, the Type VI CRISPR/Cas polypeptide comprises a sequence with at least 65% identity to SEQ ID NO: 16. In some embodiments, the Type VI CRISPR/Cas polypeptide comprises a sequence with at least 70% identity to SEQ ID NO: 16. In some embodiments, the Type VI CRISPR/Cas polypeptide comprises a sequence with at least 75% identity to SEQ ID NO: 16. In some embodiments, the Type VI CRISPR/Cas polypeptide comprises a sequence with at least 80% identity to SEQ ID NO: 16. In some embodiments, the Type VI CRISPR/Cas polypeptide comprises a sequence with at least 85% identity to SEQ ID NO: 16. In some embodiments, the Type VI CRISPR/Cas polypeptide comprises a sequence with at least 90% identity to SEQ ID NO: 16. In some embodiments, the Type VI CRISPR/Cas polypeptide comprises a sequence with at least 92% identity to SEQ ID NO: 16. In some embodiments, the Type VI CRISPR/Cas polypeptide comprises a sequence with at least 95% identity to SEQ ID NO: 16. In some embodiments, the Type VI CRISPR/Cas polypeptide comprises a sequence with at least 97% identity to SEQ ID NO: 16. In some embodiments, the Type VI CRISPR/Cas polypeptide comprises a sequence with at least 98% identity to SEQ ID NO: 16. In some embodiments, the Type VI CRISPR/Cas polypeptide comprises a sequence with at least 99% identity to SEQ ID NO: 16. In some embodiments, the Type VI CRISPR/Cas polypeptide comprises a sequence of SEQ ID NO: 16.

In some embodiments, the Type VI CRISPR/Cas polypeptide comprises a sequence with at least 50% identity to SEQ ID NO: 17. In some embodiments, the Type VI CRISPR/Cas polypeptide comprises a sequence with at least 55% identity to SEQ ID NO: 17. In some embodiments, the Type VI CRISPR/Cas polypeptide comprises a sequence with at least 60% identity to SEQ ID NO: 17. In some embodiments, the Type VI CRISPR/Cas polypeptide comprises a sequence with at least 65% identity to SEQ ID NO: 17. In some embodiments, the Type VI CRISPR/Cas polypeptide comprises a sequence with at least 70% identity to SEQ ID NO: 17. In some embodiments, the Type VI CRISPR/Cas polypeptide comprises a sequence with at least 75% identity to SEQ ID NO: 17. In some embodiments, the Type VI CRISPR/Cas polypeptide comprises a sequence with at least 80% identity to SEQ ID NO: 17. In some embodiments, the Type VI CRISPR/Cas polypeptide comprises a sequence with at least 85% identity to SEQ ID NO: 17. In some embodiments, the Type VI CRISPR/Cas polypeptide comprises a sequence with at least 90% identity to SEQ ID NO: 17. In some embodiments, the Type VI CRISPR/Cas polypeptide comprises a sequence with at least 92% identity to SEQ ID NO: 17. In some embodiments, the Type VI CRISPR/Cas polypeptide comprises a sequence with at least 95% identity to SEQ ID NO: 17. In some embodiments, the Type VI CRISPR/Cas polypeptide comprises a sequence with at least 97% identity to SEQ ID NO: 17. In some embodiments, the Type VI CRISPR/Cas polypeptide comprises a sequence with at least 98% identity to SEQ ID NO: 17. In some embodiments, the Type VI CRISPR/Cas polypeptide comprises a sequence with at least 99% identity to SEQ ID NO: 17. In some embodiments, the Type VI CRISPR/Cas polypeptide comprises a sequence of SEQ ID NO: 17.

In some embodiments, the Type VI CRISPR/Cas polypeptide comprises a sequence with at least 50% identity to SEQ ID NO: 18. In some embodiments, the Type VI CRISPR/Cas polypeptide comprises a sequence with at least 55% identity to SEQ ID NO: 18. In some embodiments, the Type VI CRISPR/Cas polypeptide comprises a sequence with at least 60% identity to SEQ ID NO: 18. In some embodiments, the Type VI CRISPR/Cas polypeptide comprises a sequence with at least 65% identity to SEQ ID NO: 18. In some embodiments, the Type VI CRISPR/Cas polypeptide comprises a sequence with at least 70% identity to SEQ ID NO: 18. In some embodiments, the Type VI CRISPR/Cas polypeptide comprises a sequence with at least 75% identity to SEQ ID NO: 18. In some embodiments, the Type VI CRISPR/Cas polypeptide comprises a sequence with at least 80% identity to SEQ ID NO: 18. In some embodiments, the Type VI CRISPR/Cas polypeptide comprises a sequence with at least 85% identity to SEQ ID NO: 18. In some embodiments, the Type VI CRISPR/Cas polypeptide comprises a sequence with at least 90% identity to SEQ ID NO: 18. In some embodiments, the Type VI CRISPR/Cas polypeptide comprises a sequence with at least 92% identity to SEQ ID NO: 18. In some embodiments, the Type VI CRISPR/Cas polypeptide comprises a sequence with at least 95% identity to SEQ ID NO: 18. In some embodiments, the Type VI CRISPR/Cas polypeptide comprises a sequence with at least 97% identity to SEQ ID NO: 18. In some embodiments, the Type VI CRISPR/Cas polypeptide comprises a sequence with at least 98% identity to SEQ ID NO: 18. In some embodiments, the Type VI CRISPR/Cas polypeptide comprises a sequence with at least 99% identity to SEQ ID NO: 18. In some embodiments, the Type VI CRISPR/Cas polypeptide comprises a sequence of SEQ ID NO: 18.

In some embodiments, the Type VI CRISPR/Cas polypeptide comprises a sequence with at least 50% identity to SEQ ID NO: 19. In some embodiments, the Type VI CRISPR/Cas polypeptide comprises a sequence with at least 55% identity to SEQ ID NO: 19. In some embodiments, the Type VI CRISPR/Cas polypeptide comprises a sequence with at least 60% identity to SEQ ID NO: 19. In some embodiments, the Type VI CRISPR/Cas polypeptide comprises a sequence with at least 65% identity to SEQ ID NO: 19. In some embodiments, the Type VI CRISPR/Cas polypeptide comprises a sequence with at least 70% identity to SEQ ID NO: 19. In some embodiments, the Type VI CRISPR/Cas polypeptide comprises a sequence with at least 75% identity to SEQ ID NO: 19. In some embodiments, the Type VI CRISPR/Cas polypeptide comprises a sequence with at least 80% identity to SEQ ID NO: 19. In some embodiments, the Type VI CRISPR/Cas polypeptide comprises a sequence with at least 85% identity to SEQ ID NO: 19. In some embodiments, the Type VI CRISPR/Cas polypeptide comprises a sequence with at least 90% identity to SEQ ID NO: 19. In some embodiments, the Type VI CRISPR/Cas polypeptide comprises a sequence with at least 92% identity to SEQ ID NO: 19. In some embodiments, the Type VI CRISPR/Cas polypeptide comprises a sequence with at least 95% identity to SEQ ID NO: 19. In some embodiments, the Type VI CRISPR/Cas polypeptide comprises a sequence with at least 97% identity to SEQ ID NO: 19. In some embodiments, the Type VI CRISPR/Cas polypeptide comprises a sequence with at least 98% identity to SEQ ID NO: 19. In some embodiments, the Type VI CRISPR/Cas polypeptide comprises a sequence with at least 99% identity to SEQ ID NO: 19. In some embodiments, the Type VI CRISPR/Cas polypeptide comprises a sequence of SEQ ID NO: 19.

In some embodiments, the Type VI CRISPR/Cas polypeptide comprises a sequence with at least 50% identity to SEQ ID NO: 20. In some embodiments, the Type VI CRISPR/Cas polypeptide comprises a sequence with at least 55% identity to SEQ ID NO: 20. In some embodiments, the Type VI CRISPR/Cas polypeptide comprises a sequence with at least 60% identity to SEQ ID NO: 20. In some embodiments, the Type VI CRISPR/Cas polypeptide comprises a sequence with at least 65% identity to SEQ ID NO: 20. In some embodiments, the Type VI CRISPR/Cas polypeptide comprises a sequence with at least 70% identity to SEQ ID NO: 20. In some embodiments, the Type VI CRISPR/Cas polypeptide comprises a sequence with at least 75% identity to SEQ ID NO: 20. In some embodiments, the Type VI CRISPR/Cas polypeptide comprises a sequence with at least 80% identity to SEQ ID NO: 20. In some embodiments, the Type VI CRISPR/Cas polypeptide comprises a sequence with at least 85% identity to SEQ ID NO: 20. In some embodiments, the Type VI CRISPR/Cas polypeptide comprises a sequence with at least 90% identity to SEQ ID NO: 20. In some embodiments, the Type VI CRISPR/Cas polypeptide comprises a sequence with at least 92% identity to SEQ ID NO: 20. In some embodiments, the Type VI CRISPR/Cas polypeptide comprises a sequence with at least 95% identity to SEQ ID NO: 20. In some embodiments, the Type VI CRISPR/Cas polypeptide comprises a sequence with at least 97% identity to SEQ ID NO: 20. In some embodiments, the Type VI CRISPR/Cas polypeptide comprises a sequence with at least 98% identity to SEQ ID NO: 20. In some embodiments, the Type VI CRISPR/Cas polypeptide comprises a sequence with at least 99% identity to SEQ ID NO: 20. In some embodiments, the Type VI CRISPR/Cas polypeptide comprises a sequence of SEQ ID NO: 20.

In some embodiments, the Type VI CRISPR/Cas polypeptide comprises a sequence with at least 50% identity to SEQ ID NO: 21. In some embodiments, the Type VI CRISPR/Cas polypeptide comprises a sequence with at least 55% identity to SEQ ID NO: 21. In some embodiments, the Type VI CRISPR/Cas polypeptide comprises a sequence with at least 60% identity to SEQ ID NO: 21. In some embodiments, the Type VI CRISPR/Cas polypeptide comprises a sequence with at least 65% identity to SEQ ID NO: 21. In some embodiments, the Type VI CRISPR/Cas polypeptide comprises a sequence with at least 70% identity to SEQ ID NO: 21. In some embodiments, the Type VI CRISPR/Cas polypeptide comprises a sequence with at least 75% identity to SEQ ID NO: 21. In some embodiments, the Type VI CRISPR/Cas polypeptide comprises a sequence with at least 80% identity to SEQ ID NO: 21. In some embodiments, the Type VI CRISPR/Cas polypeptide comprises a sequence with at least 85% identity to SEQ ID NO: 21. In some embodiments, the Type VI CRISPR/Cas polypeptide comprises a sequence with at least 90% identity to SEQ ID NO: 21. In some embodiments, the Type VI CRISPR/Cas polypeptide comprises a sequence with at least 92% identity to SEQ ID NO: 21. In some embodiments, the Type VI CRISPR/Cas polypeptide comprises a sequence with at least 95% identity to SEQ ID NO: 21. In some embodiments, the Type VI CRISPR/Cas polypeptide comprises a sequence with at least 97% identity to SEQ ID NO: 21. In some embodiments, the Type VI CRISPR/Cas polypeptide comprises a sequence with at least 98% identity to SEQ ID NO: 21. In some embodiments, the Type VI CRISPR/Cas polypeptide comprises a sequence with at least 99% identity to SEQ ID NO: 21. In some embodiments, the Type VI CRISPR/Cas polypeptide comprises a sequence of SEQ ID NO: 21.

In some embodiments, the Type VI CRISPR/Cas polypeptide comprises a sequence with at least 50% identity to SEQ ID NO: 22. In some embodiments, the Type VI CRISPR/Cas polypeptide comprises a sequence with at least 55% identity to SEQ ID NO: 22. In some embodiments, the Type VI CRISPR/Cas polypeptide comprises a sequence with at least 60% identity to SEQ ID NO: 22. In some embodiments, the Type VI CRISPR/Cas polypeptide comprises a sequence with at least 65% identity to SEQ ID NO: 22. In some embodiments, the Type VI CRISPR/Cas polypeptide comprises a sequence with at least 70% identity to SEQ ID NO: 22. In some embodiments, the Type VI CRISPR/Cas polypeptide comprises a sequence with at least 75% identity to SEQ ID NO: 22. In some embodiments, the Type VI CRISPR/Cas polypeptide comprises a sequence with at least 80% identity to SEQ ID NO: 22. In some embodiments, the Type VI CRISPR/Cas polypeptide comprises a sequence with at least 85% identity to SEQ ID NO: 22. In some embodiments, the Type VI CRISPR/Cas polypeptide comprises a sequence with at least 90% identity to SEQ ID NO: 22. In some embodiments, the Type VI CRISPR/Cas polypeptide comprises a sequence with at least 92% identity to SEQ ID NO: 22. In some embodiments, the Type VI CRISPR/Cas polypeptide comprises a sequence with at least 95% identity to SEQ ID NO: 22. In some embodiments, the Type VI CRISPR/Cas polypeptide comprises a sequence with at least 97% identity to SEQ ID NO: 22. In some embodiments, the Type VI CRISPR/Cas polypeptide comprises a sequence with at least 98% identity to SEQ ID NO: 22. In some embodiments, the Type VI CRISPR/Cas polypeptide comprises a sequence with at least 99% identity to SEQ ID NO: 22. In some embodiments, the Type VI CRISPR/Cas polypeptide comprises a sequence of SEQ ID NO: 22.

In some embodiments, the Type VI CRISPR/Cas polypeptide comprises a sequence with at least 50% identity to SEQ ID NO: 23. In some embodiments, the Type VI CRISPR/Cas polypeptide comprises a sequence with at least 55% identity to SEQ ID NO: 23. In some embodiments, the Type VI CRISPR/Cas polypeptide comprises a sequence with at least 60% identity to SEQ ID NO: 23. In some embodiments, the Type VI CRISPR/Cas polypeptide comprises a sequence with at least 65% identity to SEQ ID NO: 23. In some embodiments, the Type VI CRISPR/Cas polypeptide comprises a sequence with at least 70% identity to SEQ ID NO: 23. In some embodiments, the Type VI CRISPR/Cas polypeptide comprises a sequence with at least 75% identity to SEQ ID NO: 23. In some embodiments, the Type VI CRISPR/Cas polypeptide comprises a sequence with at least 80% identity to SEQ ID NO: 23. In some embodiments, the Type VI CRISPR/Cas polypeptide comprises a sequence with at least 85% identity to SEQ ID NO: 23. In some embodiments, the Type VI CRISPR/Cas polypeptide comprises a sequence with at least 90% identity to SEQ ID NO: 23. In some embodiments, the Type VI CRISPR/Cas polypeptide comprises a sequence with at least 92% identity to SEQ ID NO: 23. In some embodiments, the Type VI CRISPR/Cas polypeptide comprises a sequence with at least 95% identity to SEQ ID NO: 23. In some embodiments, the Type VI CRISPR/Cas polypeptide comprises a sequence with at least 97% identity to SEQ ID NO: 23. In some embodiments, the Type VI CRISPR/Cas polypeptide comprises a sequence with at least 98% identity to SEQ ID NO: 23. In some embodiments, the Type VI CRISPR/Cas polypeptide comprises a sequence with at least 99% identity to SEQ ID NO: 23. In some embodiments, the Type VI CRISPR/Cas polypeptide comprises a sequence of SEQ ID NO: 23.

In some embodiments, the Type VI CRISPR/Cas polypeptide comprises a sequence with at least 50% identity to SEQ ID NO: 24. In some embodiments, the Type VI CRISPR/Cas polypeptide comprises a sequence with at least 55% identity to SEQ ID NO: 24. In some embodiments, the Type VI CRISPR/Cas polypeptide comprises a sequence with at least 60% identity to SEQ ID NO: 24. In some embodiments, the Type VI CRISPR/Cas polypeptide comprises a sequence with at least 65% identity to SEQ ID NO: 24. In some embodiments, the Type VI CRISPR/Cas polypeptide comprises a sequence with at least 70% identity to SEQ ID NO: 24. In some embodiments, the Type VI CRISPR/Cas polypeptide comprises a sequence with at least 75% identity to SEQ ID NO: 24. In some embodiments, the Type VI CRISPR/Cas polypeptide comprises a sequence with at least 80% identity to SEQ ID NO: 24. In some embodiments, the Type VI CRISPR/Cas polypeptide comprises a sequence with at least 85% identity to SEQ ID NO: 24. In some embodiments, the Type VI CRISPR/Cas polypeptide comprises a sequence with at least 90% identity to SEQ ID NO: 24. In some embodiments, the Type VI CRISPR/Cas polypeptide comprises a sequence with at least 92% identity to SEQ ID NO: 24. In some embodiments, the Type VI CRISPR/Cas polypeptide comprises a sequence with at least 95% identity to SEQ ID NO: 24. In some embodiments, the Type VI CRISPR/Cas polypeptide comprises a sequence with at least 97% identity to SEQ ID NO: 24. In some embodiments, the Type VI CRISPR/Cas polypeptide comprises a sequence with at least 98% identity to SEQ ID NO: 24. In some embodiments, the Type VI CRISPR/Cas polypeptide comprises a sequence with at least 99% identity to SEQ ID NO: 24. In some embodiments, the Type VI CRISPR/Cas polypeptide comprises a sequence of SEQ ID NO: 24.

In some embodiments, the Type VI CRISPR/Cas polypeptide comprises a sequence with at least 50% identity to SEQ ID NO: 25. In some embodiments, the Type VI CRISPR/Cas polypeptide comprises a sequence with at least 55% identity to SEQ ID NO: 25. In some embodiments, the Type VI CRISPR/Cas polypeptide comprises a sequence with at least 60% identity to SEQ ID NO: 25. In some embodiments, the Type VI CRISPR/Cas polypeptide comprises a sequence with at least 65% identity to SEQ ID NO: 25. In some embodiments, the Type VI CRISPR/Cas polypeptide comprises a sequence with at least 70% identity to SEQ ID NO: 25. In some embodiments, the Type VI CRISPR/Cas polypeptide comprises a sequence with at least 75% identity to SEQ ID NO: 25. In some embodiments, the Type VI CRISPR/Cas polypeptide comprises a sequence with at least 80% identity to SEQ ID NO: 25. In some embodiments, the Type VI CRISPR/Cas polypeptide comprises a sequence with at least 85% identity to SEQ ID NO: 25. In some embodiments, the Type VI CRISPR/Cas polypeptide comprises a sequence with at least 90% identity to SEQ ID NO: 25. In some embodiments, the Type VI CRISPR/Cas polypeptide comprises a sequence with at least 92% identity to SEQ ID NO: 25. In some embodiments, the Type VI CRISPR/Cas polypeptide comprises a sequence with at least 95% identity to SEQ ID NO: 25. In some embodiments, the Type VI CRISPR/Cas polypeptide comprises a sequence with at least 97% identity to SEQ ID NO: 25. In some embodiments, the Type VI CRISPR/Cas polypeptide comprises a sequence with at least 98% identity to SEQ ID NO: 25. In some embodiments, the Type VI CRISPR/Cas polypeptide comprises a sequence with at least 99% identity to SEQ ID NO: 25. In some embodiments, the Type VI CRISPR/Cas polypeptide comprises a sequence of SEQ ID NO: 25.

In some embodiments, the Type VI CRISPR/Cas polypeptide comprises a sequence with at least 50% identity to SEQ ID NO: 26. In some embodiments, the Type VI CRISPR/Cas polypeptide comprises a sequence with at least 55% identity to SEQ ID NO: 26. In some embodiments, the Type VI CRISPR/Cas polypeptide comprises a sequence with at least 60% identity to SEQ ID NO: 26. In some embodiments, the Type VI CRISPR/Cas polypeptide comprises a sequence with at least 65% identity to SEQ ID NO: 26. In some embodiments, the Type VI CRISPR/Cas polypeptide comprises a sequence with at least 70% identity to SEQ ID NO: 26. In some embodiments, the Type VI CRISPR/Cas polypeptide comprises a sequence with at least 75% identity to SEQ ID NO: 26. In some embodiments, the Type VI CRISPR/Cas polypeptide comprises a sequence with at least 80% identity to SEQ ID NO: 26. In some embodiments, the Type VI CRISPR/Cas polypeptide comprises a sequence with at least 85% identity to SEQ ID NO: 26. In some embodiments, the Type VI CRISPR/Cas polypeptide comprises a sequence with at least 90% identity to SEQ ID NO: 26. In some embodiments, the Type VI CRISPR/Cas polypeptide comprises a sequence with at least 92% identity to SEQ ID NO: 26. In some embodiments, the Type VI CRISPR/Cas polypeptide comprises a sequence with at least 95% identity to SEQ ID NO: 26. In some embodiments, the Type VI CRISPR/Cas polypeptide comprises a sequence with at least 97% identity to SEQ ID NO: 26. In some embodiments, the Type VI CRISPR/Cas polypeptide comprises a sequence with at least 98% identity to SEQ ID NO: 26. In some embodiments, the Type VI CRISPR/Cas polypeptide comprises a sequence with at least 99% identity to SEQ ID NO: 26. In some embodiments, the Type VI CRISPR/Cas polypeptide comprises a sequence of SEQ ID NO: 26.

In some embodiments, the Type VI CRISPR/Cas polypeptide comprises a sequence with at least 50% identity to SEQ ID NO: 27. In some embodiments, the Type VI CRISPR/Cas polypeptide comprises a sequence with at least 55% identity to SEQ ID NO: 27. In some embodiments, the Type VI CRISPR/Cas polypeptide comprises a sequence with at least 60% identity to SEQ ID NO: 27. In some embodiments, the Type VI CRISPR/Cas polypeptide comprises a sequence with at least 65% identity to SEQ ID NO: 27. In some embodiments, the Type VI CRISPR/Cas polypeptide comprises a sequence with at least 70% identity to SEQ ID NO: 27. In some embodiments, the Type VI CRISPR/Cas polypeptide comprises a sequence with at least 75% identity to SEQ ID NO: 27. In some embodiments, the Type VI CRISPR/Cas polypeptide comprises a sequence with at least 80% identity to SEQ ID NO: 27. In some embodiments, the Type VI CRISPR/Cas polypeptide comprises a sequence with at least 85% identity to SEQ ID NO: 27. In some embodiments, the Type VI CRISPR/Cas polypeptide comprises a sequence with at least 90% identity to SEQ ID NO: 27. In some embodiments, the Type VI CRISPR/Cas polypeptide comprises a sequence with at least 92% identity to SEQ ID NO: 27. In some embodiments, the Type VI CRISPR/Cas polypeptide comprises a sequence with at least 95% identity to SEQ ID NO: 27. In some embodiments, the Type VI CRISPR/Cas polypeptide comprises a sequence with at least 97% identity to SEQ ID NO: 27. In some embodiments, the Type VI CRISPR/Cas polypeptide comprises a sequence with at least 98% identity to SEQ ID NO: 27. In some embodiments, the Type VI CRISPR/Cas polypeptide comprises a sequence with at least 99% identity to SEQ ID NO: 27. In some embodiments, the Type VI CRISPR/Cas polypeptide comprises a sequence of SEQ ID NO: 27.

The Type VI CRISPR/Cas proteins presented in TABLE 1 or variants thereof comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity with any one of SEQ ID NO: 1-SEQ ID NO: 27 can comprise reduced or substantially no nucleic acid cleavage activity.

### DETECTR Assays

In some embodiments, the Type VI CRISPR/Cas protein disclosed herein can be used in DNA/RNA Endonuclease Targeted CRISPR TransReporter (DETECTR) assays. A DETECTR assay can utilize the trans-cleavage abilities of some programmable nucleases to achieve fast and high-fidelity detection of a target nucleic acid in a sample. The target nucleic acid can be DNA or RNA. For example, following target RNA extraction from a biological sample, crRNA comprising a portion that is complementary to the target RNA of interest can bind to the target RNA sequence, initiating indiscriminate ssRNase or ssDNAse activity by the programmable nuclease. Upon hybridization with the target RNA, the trans-cleavage activity of the programmable nuclease is activated, which can then cleave an ssDNA or ssRNA reporter (e.g., fluorescence-quenching (FQ) reporter or HRP reporter) molecule. Cleavage of the reporter molecule can provide a fluorescentdetectable readout (e.g., fluorescence, colorimetric, amperometric, etc.) indicating the presence of the target RNA in the sample. In some embodiments, the programmable nucleases disclosed herein can be combined, or multiplexed, with other programmable nucleases in a DETECTR assay. The principles of the DETECTR assay are described in Chen et al. (Science 2018 Apr. 27; 360(6387):436-439) and can be modified to facilitate the use of the programmable nucleases described herein. In some embodiments, the programmable nucleases disclosed herein can be used in a specific high-sensitivity enzymatic reporter unlocking (SHERLOCK) assay. The principles of the SHERLOCK assay are described in Kellner et al. (Nat Protoc. 2019 October; 14(10):2986-3012) and can be modified to facilitate the use of the programmable nucleases described herein.

Herein, detection of reporter cleavage to determine the presence of a target nucleic acid sequence may be referred to as ‘DETECTR’. In some embodiments described herein is a method of assaying for a target nucleic acid in a sample comprising contacting the target nucleic acid with a programmable nuclease, anon-naturally occurring guide nucleic acid that hybridizes to a segment of the target nucleic acid, and a reporter nucleic acid, and assaying for a change in a signal, wherein the change in the signal is produced by cleavage of the reporter nucleic acid. In some embodiments, the target nucleic acid may be an amplified target nucleic acid.

### Buffers

The Type VI CRISPR/Cas protein and other reagents (e.g., a guide nucleic acid) can be formulated in a buffer disclosed herein. A wide variety of buffered solutions are compatible with the methods, compositions, reagents, enzymes, and kits disclosed herein. Buffers are compatible with different programmable nucleases described herein. Any of the methods, compositions, reagents, enzymes, or kits disclosed herein may comprise a buffer. These buffers may be compatible with the other reagents, samples, and support mediums as described herein for detection of an ailment, such as a disease, cancer, or genetic disorder, or genetic information, such as for phenotyping, genotyping, or determining ancestry. A buffer, as described herein, can enhance the cis- or trans-cleavage rates of any of the programmable nucleases described herein. The buffer can increase the discrimination of the programmable nucleases for the target nucleic acid. The methods as described herein can be performed in the buffer.

In some embodiments, a buffer may comprise one or more of a buffering agent, a salt, a crowding agent, or a detergent, or any combination thereof. A buffer may comprise a reducing agent. A buffer may comprise a competitor. Exemplary buffering agents include HEPES, TRIS, MES, ADA, PIPES, ACES, MOPSO, BIS-TRIS propane, BES, MOPS, TES, DISO, Trizma, TRICINE, GLY-GLY, HEPPS, BICINE, TAPS, A MPD, A MPSO, CHES, CAPSO, AMP, CAPS, phosphate, citrate, acetate, imidazole, or any combination thereof. A buffering agent may be compatible with a programmable nuclease. A buffer compatible with a programmable nuclease may comprise a buffering agent at a concentration of from 1 mM to 200 mM. A buffer compatible with a programmable nuclease may comprise a buffering agent at a concentration of from 10 mM to 30 mM. A buffer compatible with a programmable nuclease may comprise a buffering agent at a concentration of about 20 mM. A composition (e.g., a composition comprising a programmable nucleases) may have a pH of from 2.5 to 3.5. A composition (e.g., a composition comprising a programmable nucleases) may have a pH of from 3 to 4. A composition (e.g., a composition comprising a programmable nucleases) may have a pH of from 3.5 to 4.5. A composition (e.g., a composition comprising a programmable nucleases) may have a pH of from 4 to 5. A composition (e.g., a composition comprising a programmable nucleases) may have a pH of from 4.5 to 5.5. A composition (e.g., a composition comprising a programmable nucleases) may have a pH of from 5 to 6. A composition (e.g., a composition comprising a programmable nucleases) may have a pH of from 5.5 to 6.5. A composition (e.g., a composition comprising a programmable nucleases) may have a pH of from 6 to 7. A composition (e.g., a composition comprising a programmable nucleases) may have a pH of from 6.5 to 7.5. A composition (e.g., a composition comprising a programmable nucleases) may have a pH of from 7 to 8. A composition (e.g., a composition comprising a programmable nucleases) may have a pH of from 7.5 to 8.5. A composition (e.g., a composition comprising a programmable nucleases) may have a pH of from 8 to 9. A composition (e.g., a composition comprising a programmable nucleases) may have a pH of from 8.5 to 9.5. A composition (e.g., a composition comprising a programmable nucleases) may have a pH of from 9 to 10. A composition (e.g., a composition comprising a programmable nucleases) may have a pH of from 9.5 to 10.5.

A buffer may comprise a salt. Exemplary salts include NaCl, KCl, magnesium acetate, potassium acetate, CaCl2 and MgCl2. A buffer may comprise potassium acetate, magnesium acetate, sodium chloride, magnesium chloride, or any combination thereof. A buffer compatible with a programmable nuclease may comprise a salt at a concentration of from 5 mM to 100 mM. A buffer compatible with a programmable nuclease may comprise a salt at a concentration of from 5 mM to 10 mM. In some embodiments, a buffer compatible with a programmable nuclease comprises a salt from 1 mM to 60 mM. In some embodiments, a buffer compatible with a programmable nuclease comprises a salt from 1 mM to 10 mM. In some embodiments, a buffer compatible with a programmable nuclease comprises a salt at about 105 mM. In some embodiments, a buffer compatible with a programmable nuclease comprises a salt at about 55 mM. In some embodiments, a buffer compatible with a programmable nuclease comprises a salt at about 7 mM. In some embodiments, a buffer compatible with a programmable nuclease comprises a salt, wherein the salt comprises potassium acetate and magnesium acetate. In some embodiments, a buffer compatible with a programmable nuclease comprises a salt, wherein the salt comprises sodium chloride and magnesium chloride. In some embodiments, a buffer compatible with a programmable nuclease comprises a salt, wherein the salt comprises potassium chloride and magnesium chloride.

A buffer may comprise a crowding agent. Exemplary crowding agents include glycerol and bovine serum albumin. A buffer may comprise glycerol. A crowding agent may reduce the volume of solvent available for other molecules in the solution, thereby increasing the effective concentrations of said molecules. A buffer compatible with a programmable nuclease may comprise a crowding agent at a concentration of from 0.01% (v/v) to 10% (v/v). A buffer compatible with a programmable nuclease may comprise a crowding agent at a concentration of from 0.5% (v/v) to 10% (v/v).

A buffer may comprise a detergent. Exemplary detergents include Tween, Triton-X, and IGEPAL. A buffer may comprise Tween, Triton-X, or any combination thereof. A buffer compatible with a programmable nuclease may comprise Triton-X. A buffer compatible with a programmable nuclease may comprise IGEPAL CA-630. In some embodiments, a buffer compatible with a programmable nuclease comprises a detergent at a concentration of 2% (v/v) or less. A buffer compatible with a programmable nuclease may comprise a detergent at a concentration of 2% (v/v) or less. A buffer compatible with a programmable nuclease may comprise a detergent at a concentration of from 0.00001% (v/v) to 0.01% (v/v). A buffer compatible with a programmable nuclease may comprise a detergent at a concentration of about 0.01% (v/v).

A buffer may comprise a reducing agent. Exemplary reducing agents comprise dithiothreitol (DTT), β-mercaptoethanol (BME), or tris(2-carboxyethyl)phosphine (TCEP). A buffer compatible with a programmable nuclease may comprise DTT. A buffer compatible with a programmable nuclease may comprise a reducing agent at a concentration of from 0.01 mM to 100 mM. A buffer compatible with a programmable nuclease may comprise a reducing agent at a concentration of from 0.1 mM to 10 mM. A buffer compatible with a programmable nuclease may comprise a reducing agent at a concentration of from 0.5 mM to 2 mM. A buffer compatible with a programmable nuclease may comprise a reducing agent at a concentration of from 0.01 mM to 100 mM. A buffer compatible with a programmable nuclease may comprise a reducing agent at a concentration of from 0.1 mM to 10 mM. A buffer compatible with a programmable nuclease may comprise a reducing agent at a concentration of about 1 mM.

A buffer compatible with a programmable nuclease may comprise a competitor. Exemplary competitors compete with the target nucleic acid or the reporter nucleic acid for cleavage by the programmable nuclease. Exemplary competitors include heparin, and imidazole, and salmon sperm DNA. A buffer compatible with a programmable nuclease may comprise a competitor at a concentration of from 1 μg/mL to 100 μg/mL. A buffer compatible with a programmable nuclease may comprise a competitor at a concentration of from 40 μg/mL to 60 μg/mL.

### Cleavage by Type VI CRISPR/Cas Nuclease

In some embodiments, a programmable Type VI CRISPR/Cas nuclease rapidly cleaves a strand of a single-stranded target nucleic acid. The cleavage of target nucleic acid strands can be assessed in an in vitro cis-cleavage assay. In some embodiments, a cleavage assay is an assay designed to visualize, quantitate or identify cleavage of a nucleic acid. In some cases, the cleavage activity is cis-cleavage activity. In some cases, the cleavage activity is trans-cleavage activity. To perform such as assay, the programmable Type VI CRISPR/Cas nuclease is complexed to its native crRNA, e.g. Cas13.2 nuclease with the Cas13.2 repeat, in buffer comprising 50 mM potassium acetate, 20 mM Tris-acetate, 10 mM magnesium acetate, 100 ug/ml BSA, and which is pH 7.9 at 25° C. The complexing is carried out for 20 minutes at room temperature, e.g. 20-22° C. The RNP is at a concentration of 200 nM. At time “0” 30 equal volumes of target plasmid, at 20 nM, and complexed RNP are mixed, so that the concentration of target plasmid is 10 nM and the concentration of complexed RNP is 100 nM. The incubation temperature is 37° C. The reaction is quenched at desired time points, e.g. 1, 3, 6, 15, 30 and 60 minutes, with reaction quench comprising 1 mg/ml proteinase K, 0.08% SDS and 15 mM EDTA. The sample incubates for 30 minutes at 37° C. to deproteinize. The cleavage is quantified by agarose gel analysis.

In some embodiments, a programmable Type VI CRISPR/Cas nuclease creates at least 50%, at least 55%, at least 60%, at least 65%, at least 70%, at least 75%, at least 80%, at least 85%, at least 90 or at least 95% of the maximum amount of product within 1 minute, where the maximum amount of product is the maximum amount detected within a 60 minute period from when the target plasmid is mixed with the programmable Type VI CRISPR/Cas nuclease. In preferred embodiments, at least 80% of the maximum amount of product is created within 1 minute. In more preferred embodiments, at least 90% of the maximum amount of product is created within 1 minute.

In some embodiments, a programmable Type VI CRISPR/Cas nuclease creates at least 50%, at least 55%, at least 60%, at least 65%, at least 70%, at least 75%, at least 80%, at least 85%, at least 90 or at least 95% of the maximum amount of linearized product is created within 1 minute, where the maximum amount of linearized product is the maximum amount detected within a 60 minute period from when the target plasmid is mixed with the programmable Type VI CRISPR/Cas nuclease. In preferred embodiments, at least 80% of the maximum amount of linearized product is created within 1 minute. In more preferred embodiments, at least 90% of the maximum amount of linearized product is created within 1 minute.

In some embodiments, a programmable Type VI CRISPR/Cas nuclease uses a co-factor. In some embodiments, the co-factor allows the programmable Type VI CRISPR/Cas nuclease to perform a function. In some embodiments, the function is pre-crRNA processing and/or target nucleic acid cleavage. As discussed in Jiang F. and Doudna J. A. (Annu. Rev. Biophys. 2017. 46:505-29), Cas9 uses divalent metal ions as co-factors. The suitability of a divalent metal ion as a cofactor can easily be assessed, such as by methods based on those described by Sundaresan et al. (Cell Rep. 2017 Dec. 26; 21(13): 3728-3739). In some embodiments, the co-factor is a divalent metal ion. In some embodiments, the divalent metal ion is selected from Mg2+, Mn2+, Zn2+, Ca2+, Cu2+. In a preferred embodiment, the divalent metal ion is Mg2+. In some embodiments, a programmable Type VI CRISPR/Cas nuclease forms a complex with a divalent metal ion. In preferred embodiments, a programmable Type VI CRISPR/Cas nuclease forms a complex with Mg2+.

### Compositions Including Cells

In some aspects, the disclosure provides a composition comprising a programmable Type VI CRISPR/Cas nuclease disclosed herein and a cell, preferably wherein the cell is a eukaryotic cell. In some embodiments, a programmable Type VI CRISPR/Cas nuclease disclosed herein is in a cell, preferably wherein the cell is a eukaryotic cell.

In some aspects, the disclosure provides a composition comprising a nucleic acid encoding a programmable Type VI CRISPR/Cas nuclease disclosed herein and a cell, preferably wherein the cell is a eukaryotic cell. In some embodiments, a nucleic acid encoding a programmable Type VI CRISPR/Cas nuclease disclosed herein is in a cell, preferably wherein the cell is a eukaryotic cell.

### Systems

Provided herein is a system for detecting a target nucleic acid comprising any one of the compositions provided herein and at least one of a buffering agent, a salt, a crowding agent, a detergent, a reducing agent, a competitor, and a reporter nucleic acid. In some embodiments, a reporter and a reporter nucleic acid are non-target nucleic acid molecules that can provide a detectable signal upon cleavage by a programmable nuclease. Examples of detectable signals and detectable moieties that generate detectable signals are provided herein.

In some embodiments, the system comprises a solution comprising the at least one of a buffering agent, salt, crowding agent, detergent, reducing agent, competitor, and detection agent. In some embodiments, the pH of the solution is at least about 6.0. In some embodiments, the pH of the solution is at least about 6.5. In some embodiments, the pH of the solution is at least about 7.0. In some embodiments, the pH of the solution is at least about 7.5. In some embodiments, the pH of the solution is at least about 8.0. In some embodiments, the pH of the solution is at least about 8.5. In some embodiments, the pH of the solution is at least about 9.0. In some embodiments, the salt is selected from a magnesium salt, a potassium salt, a sodium salt and a calcium salt. In some embodiments, the concentration of the salt in the solution is at least about 1 mM. In some embodiments, the concentration of the salt in the solution is at least about 3 mM. In some embodiments, the concentration of the salt in the solution is at least about 7 mM. In some embodiments, the concentration of the salt in the solution is at least about 9 mM. In some embodiments, the concentration of the salt in the solution is at least about 11 mM. In some embodiments, the concentration of the salt in the solution is at least about 13 mM. In some embodiments, the concentration of the salt in the solution is at least about 15 mM.

In some embodiments, the reporter nucleic acid comprises a sequence selected from SEQ ID NOS: 33-40. In some embodiments, the detection reagent is the reporter nucleic acid. In some embodiments, the reporter nucleic acid comprises a detection moiety, a quencher, or a combination thereof, and optionally, wherein the detection moiety and the quencher are selected from Table 3. In some embodiments, the detection moiety comprises a fluorophore. In some embodiments, the reporter nucleic acid comprises the quencher. In some embodiments, the reporter nucleic acid comprises at least one of a fluorophore and a quencher. In some embodiments, the reporter nucleic acid is in the form of a single-stranded RNA. In some embodiments, the system comprises at least one amplification reagent for amplifying a sample. In some embodiments, the at least one amplification reagent is selected from the group consisting of a primer, an activator, a deoxynucleoside triphosphate (dNTP), a ribonucleoside triphosphate (rNTP), and combinations thereof. In some embodiments, amplification is isothermal amplification or polymerase chain reaction (PCR).

### Pharmaceutical Compositions

Provided herein is a pharmaceutical composition comprising a therapeutically effective amount of any one of the compositions described herein, and a pharmaceutically acceptable diluent or excipient. In some embodiments, a pharmaceutically acceptable excipient, carrier or diluent is any substance formulated alongside the active ingredient of a pharmaceutical composition that allows the active ingredient to retain biological activity and is non-reactive with the subject's immune system. Such a substance can be included for the purpose of long-term stabilization, bulking up solid formulations that contain potent active ingredients in small amounts, or to confer a therapeutic enhancement on the active ingredient in the final dosage form, such as facilitating absorption, reducing viscosity, or enhancing solubility. The selection of appropriate substance can depend upon the route of administration and the dosage form, as well as the active ingredient and other factors. Compositions having such substances can be formulated by well-known conventional methods (see, e.g., Remington's Pharmaceutical Sciences, 18th edition, A. Gennaro, ed., Mack Publishing Co., Easton, Pa., 1990; and Remington, The Science and Practice of Pharmacy 21st Ed. Mack Publishing, 2005). In some embodiments, the pharmaceutically acceptable diluent is selected from phosphate buffered saline and water.

### Guide Nucleic Acids and Target Nucleic Acids

The methods and compositions of the disclosure may comprise an engineered guide nucleic acid. The engineered guide nucleic acid can bind to a target nucleic acid (e.g., a single strand of a target nucleic acid) or portion thereof. For example, the guide nucleic acid can bind to a target nucleic acid such as nucleic acid from a virus or a bacterium or other agents responsible for a disease, or an amplicon thereof, as described herein. In some embodiments, a guide nucleic acid is a nucleic acid comprising: a first nucleotide sequence that hybridizes to a target nucleic acid; and a second nucleotide sequence that is capable of being non-covalently bound by a programmable nuclease. In some embodiments, a target sequence such as a target nucleic acid can be a sequence of nucleotides found within a target nucleic acid. Such a sequence of nucleotides can, for example, hybridize to an equal length portion of a guide nucleic acid. Hybridization of the guide nucleic acid to the target sequence may bring a programmable nuclease into contact with the target nucleic acid. The first sequence can be a spacer sequence. The second sequence can be a repeat sequence. In some instances, the first sequence is located 5′ of the second nucleotide sequence. In some instances, the first sequence is located 3′ of the second nucleotide sequence. Guide nucleic acids, when complexed with a programmable nuclease, may bring the programmable nuclease into proximity of a target nucleic acid. Sufficient conditions for hybridization of a guide nucleic acid to a target nucleic acid and/or for binding of a guide nucleic acid to a programmable nuclease include in vivo physiological conditions of a desired cell type or in vitro conditions sufficient for assaying catalytic activity of a protein, polypeptide or peptide described herein, such as the nuclease activity of a programmable nuclease. In some embodiments, a nuclease activity is the enzymatic activity of an enzyme which allows the enzyme to cleave the phosphodiester bonds between the nucleotide subunits of nucleic acids; endonuclease activity is the enzymatic activity of an enzyme which allows the enzyme to cleave the phosphodiester bond within a polynucleotide chain. An enzyme with nuclease activity may be referred to as a “nuclease.” Guide nucleic acids may comprise DNA, RNA, or a combination thereof (e.g., RNA with a thymine base). Guide nucleic acids may include a chemically modified nucleobase or phosphate backbone. Guide nucleic acids can be a guide RNA (gRNA). However, a guide RNA is not limited to ribonucleotides, but may comprise deoxyribonucleotides and other chemically modified nucleotides. A guide nucleic acid may comprise a CRISPR RNA (crRNA), a short-complementarity untranslated RNA (scoutRNA), an associated trans-activating RNA (tracrRNA) or a combination thereof. The combination of a crRNA with a tracrRNA may be referred to herein as a single guide RNA (sgRNA), wherein the crRNA and the tracrRNA are covalently linked. In some embodiments, the crRNA and tracrRNA are linked by a phosphodiester bond. In some instances, the crRNA and tracrRNA are linked by one or more linked nucleotides. A guide nucleic acid may comprise a naturally occurring guide nucleic acid. A guide nucleic acid may comprise a non-naturally occurring guide nucleic acid, including a guide nucleic acid that is designed to contain a chemical or biochemical modification. In some embodiments, non-naturally occurring and engineered may be used interchangeably and indicate the involvement of the hand of man. Non-naturally occurring and engineered, when referring to a nucleic acid, nucleotide, protein, polypeptide, peptide or amino acid, refer to a nucleic acid, nucleotide, protein, polypeptide, peptide or amino acid that is at least substantially free from at least one other feature with which it is naturally associated in nature and as found in nature, and/or contains a modification (e.g., chemical modification, nucleotide sequence, or amino acid sequence) that is not present in the naturally occurring nucleic acid, nucleotide, protein, polypeptide, peptide, or amino acid. Non-naturally occurring and engineered, when referring to a composition or system described herein, refer to a composition or system having at least one component that is not naturally associated with the other components of the composition or system. By way of a non-limiting example, a composition may include a programmable nuclease and a guide nucleic acid that do not naturally occur together. Conversely, and as a non-limiting further clarifying example, a programmable nuclease or guide nucleic acid that is “natural,” “naturally-occurring,” or “found in nature” includes a programmable nuclease and a guide nucleic acid from a cell or organism that have not been genetically modified by the hand of man. In some embodiments, a trans-activating RNA (tracrRNA) is a nucleic acid that comprises a first sequence that is capable of being non-covalently bound by a programmable nuclease. TracrRNAs may comprise a second sequence that hybridizes to a portion of a crRNA, which may be referred to as a repeat hybridization sequence. In some embodiments, tracrRNAs are covalently linked to a crRNA. A tracrRNA may include deoxyribonucleosides, ribonucleosides, chemically modified nucleosides, or any combination thereof. A tracrRNA may be separate from, but form a complex with, a guide nucleic acid and a programmable nuclease. The tracrRNA may be attached (e.g., covalently) by an artificial linker to a guide nucleic acid. A tracrRNA may include a nucleotide sequence that hybridizes with a portion of a guide nucleic acid. A tracrRNA may also form a secondary structure (e.g., one or more hairpin loops) that facilitates the binding of a programmable nuclease to a guide nucleic acid and/or modification activity of a programmable nuclease on a target nucleic acid. A tracrRNA may include a repeat hybridization region and a hairpin region. The repeat hybridization region may hybridize to all or part of the repeat sequence of a guide nucleic acid. The repeat hybridization region may be positioned 3′ of the hairpin region. The hairpin region may include a first sequence, a second sequence that is reverse complementary to the first sequence, and a stem-loop linking the first sequence and the second sequence.

In some embodiments, a target nucleic acid is a nucleic acid that is selected as the nucleic acid for modification, binding, hybridization or any other activity of or interaction with a nucleic acid, protein, polypeptide, or peptide described herein. A target nucleic acid may comprise RNA, DNA, or a combination thereof. A target nucleic acid may be single-stranded (e.g., single-stranded RNA or single-stranded DNA) or double-stranded (e.g., double-stranded DNA). The target nucleic acid may be from any organism, including, but not limited to, a bacterium, a virus, a parasite, a protozoon, a fungus, a mammal, a plant, and an insect. As another non-limiting example, the target nucleic acid may be responsible for a disease, contain a mutation (e.g., single strand polymorphism, point mutation, insertion, or deletion), be contained in an amplicon, or be uniquely identifiable from the surrounding nucleic acids (e.g., contain a unique sequence of nucleotides).

The guide nucleic acid can bind to a target nucleic acid such as a nucleic acid from a bacterium, a virus, a parasite, a protozoa, a fungus or other agents responsible for a disease, or an amplicon thereof, as described herein. The target nucleic acid can comprise a mutation, such as a single nucleotide polymorphism (SNP). A mutation can confer for example, resistance to a treatment, such as antibiotic treatment. In some embodiments, a treatment (or treating a recipient) is a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. In some embodiments, a subject is a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some instances, the subject is not necessarily diagnosed or suspected of being at high risk for the disease. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made. The guide nucleic acid can bind to a target nucleic acid such as DNA or RNA, from a cancer gene or gene associated with a genetic disorder, or an amplicon thereof, as described herein. The guide nucleic acid comprises a segment of nucleic acids that are reverse complementary to the target nucleic acid. Often the guide nucleic acid binds specifically to the target nucleic acid. The target nucleic acid may be RNA or other synthetic nucleic acids. The target nucleic acid can be RNA or DNA. An engineered guide nucleic acid may be a non-naturally occurring guide nucleic acid. A non-naturally occurring guide nucleic acid may comprise an engineered sequence having a repeat and a spacer that hybridizes to a target nucleic acid sequence of interest. A non-naturally occurring guide nucleic acid may be recombinantly expressed or chemically synthesized.

In some embodiments, recombinant proteins, polypeptides, peptides and nucleic acids may refer to proteins, polypeptides, peptides and nucleic acids that are products of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non translated sequences, or introns, which are typically present in eukaryotic genes. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions and may act to modulate production of a desired product by various mechanisms. Thus, for example, the term “recombinant polynucleotide” or “recombinant nucleic acid” refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. Similarly, the term “recombinant polypeptide” or “recombinant protein” refers to one which is not naturally occurring, e.g., is made by the artificial combination of two otherwise separated segments of amino sequences through human intervention. Thus, for example, a polypeptide that includes a heterologous amino acid sequence is a recombinant polypeptide.

An engineered guide nucleic acid (gRNA) sequence may hybridize to a target sequence of a target nucleic acid. The engineered guide nucleic acid can bind to a programmable nuclease.

In some embodiments, a gRNA comprises a crRNA. In some embodiments, a gRNA of a Type VI CRISPR/Cas polypeptide or variants thereof does not comprise a tracrRNA. In some embodiments, a programmable Cas13 nuclease disclosed herein does not require a tracrRNA to locate and/or cleave a target nucleic acid. A crRNA may comprise a repeat region. Specifically, the crRNA of the guide nucleic acid may comprise a repeat region and a spacer region. The repeat region refers to the sequence of the crRNA that binds to the programmable nuclease. The spacer region refers to the sequence of the crRNA that hybridizes to a sequence of the target nucleic acid. In some embodiments, the repeat region may comprise mutations or truncations with respect to the repeat sequences in pre-crRNA. The repeat sequence of the crRNA may interact with a programmable nuclease, allowing for the guide nucleic acid and the programmable nuclease to form a complex. This complex may be referred to as a ribonucleoprotein (RNP) complex. The crRNA may comprise a spacer sequence. The spacer sequence may hybridize to a target sequence of the target nucleic acid, where the target sequence is a segment of a target nucleic acid. The spacer sequences may be reverse complementary to the target sequence. In some cases, the spacer sequence may be sufficiently reverse complementary to a target sequence to allow for hybridization, however, may not necessarily be 100% reverse complementary.

In some embodiments, a programmable nuclease may cleave a precursor RNA (“pre-crRNA”) to produce a guide RNA, also referred to as a “mature guide RNA.” A programmable nuclease that cleaves pre-crRNA to produce a mature guide RNA is said to have pre-crRNA processing activity.

The guide nucleic acid can bind specifically to the target nucleic acid. A guide nucleic acid can comprise a sequence that is, at least in part, reverse complementary to the sequence of a target nucleic acid.

The guide nucleic acid may be a non-naturally occurring guide nucleic acid. A non-naturally occurring guide nucleic acid may comprise an engineered sequence having a repeat and a spacer that hybridizes to a target nucleic acid sequence of interest. A non-naturally occurring guide nucleic acid may be recombinantly expressed or chemically synthesized.

A guide nucleic acid can comprise RNA, DNA, or a combination thereof.

In some embodiments, the guide nucleic acid comprises a nucleotide sequence as described herein (e.g., TABLE 2). Such nucleotide sequences described herein (e.g., TABLE 2) may be described as a nucleotide sequence of either DNA or RNA, however, no matter the form the sequence is described, it is readily understood that such nucleotide sequences can be revised to be RNA or DNA, as needed, for describing a sequence within a guide nucleic acid itself or the sequence that encodes a guide nucleic acid, such as a nucleotide sequence described herein for a vector. Similarly, disclosure of the nucleotide sequences described herein (e.g., TABLE 2) also discloses the complementary nucleotide sequence, the reverse nucleotide sequence, and the reverse complement nucleotide sequence, any one of which can be a nucleotide sequence for use in a guide nucleic acid as described herein.

TABLE 2 provides illustrative crRNA sequences for use with the compositions and methods of the disclosure. In some embodiments, the crRNA sequence comprises at least 70%, at least 80%, at least 90%, at least 92%, at least 95%, at least 97%, or at least 99%, or 100% sequence identity to any one of SEQ ID NO: 28-SEQ ID NO: 32, or a reverse complement thereof. In some embodiments, the crRNA sequence comprises at least 70%, at least 80%, at least 90%, at least 92%, at least 95%, at least 97%, at least 99%, or 100% sequence identity to SEQ ID NO: 28 or a reverse complement thereof. In some embodiments, the crRNA sequence comprises at least 70%, at least 80%, at least 90%, at least 92%, at least 95%, at least 97%, at least 99%, or 100% sequence identity to SEQ ID NO: 29 or a reverse complement thereof. In some embodiments, the crRNA sequence comprises at least 70%, at least 80%, at least 90%, at least 92%, at least 95%, at least 97%, at least 99%, or 100% sequence identity to SEQ ID NO: 30 or a reverse complement thereof. In some embodiments, the crRNA sequence comprises at least 70%, at least 80%, at least 90%, at least 92%, at least 95%, at least 97%, at least 99%, or 100% sequence identity to SEQ ID NO: 31 or a reverse complement thereof. In some embodiments, the crRNA sequence comprises at least 70%, at least 80%, at least 90%, at least 92%, at least 95%, at least 97%, at least 99%, or 100% sequence identity to SEQ ID NO: 32 or a reverse complement thereof.

TABLE 2
Exemplary nucleotide sequences of crRNA repeats

| SEQ ID | |
|---|---|
| NO: | Sequence |
| 28 | AGUUGUGUCUACCUCCUUUUUGAGAGGUAAAAACAA |
| | C |
| 29 | GUUGCAUCUGCCUGCUGUUUGCAAGGUAAAAACAAC |
| 30 | GAUUUAGACUACCCCAAAAACGAAGGGGACUAAAAC |
| | C |
| 31 | GUUUUAGUCCCCUUCGUUUUUGGGGUAGUCUAAAUC |
| 32 | GUUGUAACUGCCCUUAUUUUGAAGGGUAAACACAAC |
| 60 | GUUAGAAUAUAACCCUGUUUGUAGGGGUAAUAAAAC |
| | CGACCUACUCUCCCAUACUCUUGUAUAUAG |
| 61 | UUGACUACACUCUCUAUCUCUUAGGGAGACUGAAAC |
| | CGACCUACUCUCCCAUACUCUUGUAUAUAG |
| 62 | GACUGAACCCUACCAUGUAGGGAAUUGAACCGACCU |
| | ACUCUCCCAUACUCUUGUAUAUAG |
| 63 | AUUUGACUAUACCCCUAUUUGAGAGGGGAAUAAAAC |
| | CGACCUACUCUCCCAUACUCUUGUAUAUAG |
| 64 | AUUGGAAUAUAACCCCGUUUGUAGGGGUAGUAAAAC |
| | CGACCUACUCUCCCAUACUCUUGUAUAUAG |
| 65 | GUUUGAAUAUAACCCUGUUUGUAGGGGUAAUAAAAC |
| | CGACCUACUCUCCCAUACUCUUGUAUAUAG |
| 66 | GAUAGAAUAUAACCCUGUUUGUAGGGGUAAUAAAAC |
| | CGACCUACUCUCCCAUACUCUUGUAUAUAG |
| 67 | AGUCAGACUAUACCCUCGUUUGUAGGGGGAAUAAAA |
| | C |
| 68 | GGUUAGAAUAUAACCCUGUUUGUAGGGGUAAUAAAA |
| | C |
| 70 | GUUUGAAUAUAACCCUGUUUGUAGGGGUAAUAAAAC |
| | UACUGACAGCCAGACAGCGACCAAA |
| 71 | GUUUGAAUAUAACCCUGUUUGUAGGGGUAAUAAAAC |
| | CUGACAGCCAGACAGCGACCAAAAG |
| 72 | GUUUGAAUAUAACCCUGUUUGUAGGGGUAAUAAAAC |
| | ACAGCCAGACAGCGACCAAAAGAAU |

In some embodiments, the programmable nuclease disclosed herein is used in conjunction with a crRNA sequence, such as a crRNA as disclosed in Table 2. In some embodiments, the crRNA sequence comprises at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to any one of SEQ ID NO: 29-SEQ ID NO: 32, or a reverse complement thereof. In some embodiments, the crRNA sequence comprises at least 50%, at least 55% at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 28 or a reverse complement thereof. In some embodiments, the crRNA sequence comprises at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 29 or a reverse complement thereof. In some embodiments, the crRNA sequence comprises at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 30 or a reverse complement thereof. In some embodiments, the crRNA sequence comprises at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 31 or a reverse complement thereof. In some embodiments, the crRNA sequence comprises at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 32 or a reverse complement thereof.

In some embodiments, the activity of a Type VI CRISPR/Cas protein can be supported by a crRNA comprising any of the crRNA repeat sequences recited in TABLE 2. In some embodiments, the activity of a Type VI CRISPR/Cas protein can be supported by a crRNA comprising a crRNA repeat sequence comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to any one of SEQ ID NO: 28-SEQ ID NO: 32.

The guide nucleic acid comprises a first region complementary to the target nucleic acid (FR1) and a second region that is not complementary to the target sequence (FR2). In some cases, the orientation can be FR1 followed by FR2 (FR1-FR2) or FR2 followed by FR1 (FR2-FR1). In some cases, the first region and second region are oriented: FR1-FR2. In some embodiments, the first region and second region are oriented FR2-FR1. In some embodiments, FR1 is a sequence comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 28-SEQ ID NO: 32. In some embodiments, FR2 is a sequence comprising at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity to SEQ ID NO: 41.

In some cases, the guide nucleic acid is not naturally occurring and made by artificial combination of otherwise separate segments of sequence. Often, the artificial combination is performed by chemical synthesis, by genetic engineering techniques, or by the artificial manipulation of isolated segments of nucleic acids. In some cases, the segment of a guide nucleic acid that comprises a sequence that is reverse complementary to the target nucleic acid is 20 nucleotides in length. A guide nucleic acid can have at least 10, 11, 12, 13, 14, 15, 16, 17, 18, 19, 20, 21, 22, 23, 24, 25, 26, 27, 28, 29, or 30 nucleotides reverse complementary to a target nucleic acid. In some cases, the guide nucleic acid can be 10, 11, 12, 13, 14, 15, 16, 17, 18, 19, 20, 21, 22, 23, 24, 25, 26, 27, 28, 29, or 30 nucleotides in length. For example, a guide nucleic acid may be at least 10 bases. In some embodiments, a guide nucleic acid may be from 10 to 50 bases. In some embodiments, a guide nucleic acid may be at least 25 bases. In some cases, the guide nucleic acid has from exactly or about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt reverse complementary to a target nucleic acid. In some cases, the guide nucleic acid has from about 10 nt to about 60 nt, from about 20 nt to about 50 nt, or from about 30 nt to about 40 nt reverse complementary to a target nucleic acid. It is understood that the sequence of a guide nucleic acid need not be 100% reverse complementary to that of its target nucleic acid to be specifically hybridizable, hybridizable, or bind specifically. The guide nucleic acid can have a sequence comprising at least one uracil in a region from nucleic acid residue 5 to 20 that is reverse complementary to a modification variable region in the target nucleic acid. The guide nucleic acid, in some cases, has a sequence comprising at least one uracil in a region from nucleic acid residue 5 to 9, 10 to 14, or 15 to 20 that is reverse complementary to a modification variable region in the target nucleic acid. The guide nucleic acid can have a sequence comprising at least one uracil in a region from nucleic acid residue 5 to 20 that is reverse complementary to a methylation variable region in the target nucleic acid. The guide nucleic acid, in some cases, has a sequence comprising at least one uracil in a region from nucleic acid residue 5 to 9, 10 to 14, or 15 to 20 that is reverse complementary to a methylation variable region in the target nucleic acid. The guide nucleic acid can hybridize with a target nucleic acid.

The guide nucleic acid (e.g., a non-naturally occurring guide nucleic acid) can be selected from a group of guide nucleic acids that have been tiled against the nucleic acid sequence of a strain of an infection or genomic locus of interest. The guide nucleic acid can be selected from a group of guide nucleic acids that have been tiled against the nucleic acid sequence of a target nucleic acid, for example, a strain of HPV16 or HPV18. Often, guide nucleic acids that are tiled against the nucleic acid of a strain of an infection or genomic locus of interest can be pooled for use in a method described herein. Often, these guide nucleic acids are pooled for detecting a target nucleic acid in a single assay. The pooling of guide nucleic acids that are tiled against a single target nucleic acid can enhance the detection of the target nucleic using the methods described herein. The pooling of guide nucleic acids that are tiled against a single target nucleic acid can ensure broad coverage of the target nucleic acid within a single reaction using the methods described herein. The tiling, for example, is sequential along the target nucleic acid. Sometimes, the tiling is overlapping along the target nucleic acid. In some instances, the tiling comprises gaps between the tiled guide nucleic acids along the target nucleic acid. In some instances, the tiling of the guide nucleic acids is non-sequential. Often, a method for detecting a target nucleic acid comprises contacting a target nucleic acid to a pool of guide nucleic acids and a programmable nuclease as disclosed herein, wherein a guide nucleic acid sequence of the pool of guide nucleic acids has a sequence selected from a group of tiled guide nucleic acid that correspond to nucleic acid sequence of a target nucleic acid; and assaying for a signal produce by cleavage of at least some nucleic acids of a reporter of a population of nucleic acids of a reporter. Pooling of guide nucleic acids can ensure broad spectrum identification, or broad coverage, of a target species within a single reaction. This can be particularly helpful in diseases or indications, like sepsis, that may be caused by multiple organisms.

A programmable nuclease of the present disclosure may be activated to exhibit cleavage activity (e.g., cis-cleavage of a target nucleic acid or trans-cleavage of a collateral nucleic acid) upon binding of a ribonucleoprotein (RNP) complex to a target nucleic acid, in which the spacer of the crRNA of the gRNA hybridizes to the target nucleic acid.

A wide array of samples are compatible with the compositions and methods disclosed herein. The samples, as described herein, may be used in the DETECTR assay methods disclosed herein. The samples, as described herein, are compatible with any of the programmable nucleases disclosed herein and use of said programmable nuclease in a method of detecting a target nucleic acid. The samples, as described herein, are compatible with any of the compositions comprising a programmable nuclease and a buffer. Described herein are samples that contain deoxyribonucleic acid (DNA), ribonucleic acid (RNA), or both, which can be modified or detected using a programmable nuclease of the present disclosure. As described herein, programmable nucleases are activated upon binding to a target nucleic acid of interest in a sample upon hybridization of a guide nucleic acid to the target nucleic acid. Subsequently, the activated programmable nucleases exhibit sequence-independent cleavage of a nucleic acid in a reporter. The reporter additionally includes a detectable moiety, which is released upon sequence-independent cleavage of the nucleic acid in the reporter. The detectable moiety emits or produces a detectable signal, which can be measured by various methods (e.g., spectrophotometry, fluorescence measurements, electrochemical measurements, visually, etc.).

Various sample types comprising a target nucleic acid of interest are consistent with the present disclosure. These samples can comprise a target nucleic acid sequence for detection. In some embodiments, the detection of the target nucleic indicates an ailment, such as a disease, cancer, or genetic disorder, or genetic information, such as for phenotyping, genotyping, or determining ancestry and are compatible with the reagents and support mediums as described herein. Generally, a sample from an individual or an animal or an environmental sample can be obtained to test for presence of a disease, cancer, genetic disorder, or any mutation of interest. A biological sample from the individual may be blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, or tissue. A tissue sample may be dissociated or liquified prior to application to detection system of the present disclosure. A sample from an environment may be from soil, air, or water. In some instances, the environmental sample is taken as a swab from a surface of interest or taken directly from the surface of interest. In some instances, the raw sample is applied to the detection system. In some instances, the sample is diluted with a buffer or a fluid or concentrated prior to application to the detection system or be applied neat to the detection system. Sometimes, the sample is contained in no more 20 μl. The sample, in some cases, is contained in no more than 1, 5, 10, 15, 20, 25, 30, 35 40, 45, 50, 55, 60, 65, 70, 75, 80, 90, 100, 200, 300, 400, 500 μl, or any of value from 1 μl to 500 μl, preferably from 10 μL to 200 μL, or more preferably from 50 μL to 100 μL. Sometimes, the sample is contained in more than 500 μl. In some embodiments, a cancer is a disease state characterized by the presence in a subject of cells demonstrating abnormal uncontrolled replication. Cancer may be used interchangeably with “carcino-,” “onco-,” and “tumor.” Non-limiting examples of cancers include: acute lymphoblastic leukemia; acute lymphoblastic lymphoma; acute lymphocytic leukemia; acute myelogenous leukemia; acute myeloid leukemia (adult/childhood); adrenocortical carcinoma; AIDS-related cancers; AIDS-related lymphoma; anal cancer; appendix cancer; astrocytoma; atypical teratoid/rhabdoid tumor; basal-cell carcinoma; bile duct cancer, extrahepatic (cholangiocarcinoma); bladder cancer; bone osteosarcoma/malignant fibrous histiocytoma; brain cancer (adult/childhood); brain tumor, cerebellar astrocytoma (adult/childhood); brain tumor, cerebral astrocytoma/malignant glioma brain tumor; brain tumor, ependymoma; brain tumor, medulloblastoma; brain tumor, supratentorial primitive neuroectodermal tumors; brain tumor, visual pathway and hypothalamic glioma; brainstem glioma; breast cancer; bronchial adenomas/carcinoids; bronchial tumor; Burkitt lymphoma; cancer of childhood; carcinoid gastrointestinal tumor; carcinoid tumor; carcinoma of adult, unknown primary site; carcinoma of unknown primary; central nervous system embryonal tumor; central nervous system lymphoma, primary; cervical cancer; childhood adrenocortical carcinoma; childhood cancers; childhood cerebral astrocytoma; chordoma, childhood; chronic lymphocytic leukemia; chronic myelogenous leukemia; chronic myeloid leukemia; chronic myeloproliferative disorders; colon cancer; colorectal cancer; craniopharyngioma; cutaneous T-cell lymphoma; desmoplastic small round cell tumor; emphysema; endometrial cancer; ependymoblastoma; ependymoma; esophageal cancer; Ewing sarcoma in the Ewing family of tumors; extracranial germ cell tumor; extragonadal germ cell tumor; extrahepatic bile duct cancer; gallbladder cancer; gastric (stomach) cancer; gastric carcinoid; gastrointestinal carcinoid tumor; gastrointestinal stromal tumor; germ cell tumor: extracranial, extragonadal, or ovarian gestational trophoblastic tumor; gestational trophoblastic tumor, unknown primary site; glioma; glioma of the brain stem; glioma, childhood visual pathway and hypothalamic; hairy cell leukemia; head and neck cancer; heart cancer; hepatocellular (liver) cancer; Hodgkin's lymphoma; hypopharyngeal cancer; hypothalamic and visual pathway glioma; intraocular melanoma; islet cell carcinoma (endocrine pancreas); Kaposi Sarcoma; kidney cancer (renal cell cancer); Langerhans cell histiocytosis; laryngeal cancer; lip and oral cavity cancer; liposarcoma; liver cancer (primary); lung cancer, non-small cell; lung cancer, small cell; lymphoma, primary central nervous system; macroglobulinemia, Waldenström; male breast cancer; malignant fibrous histiocytoma of bone/osteosarcoma; medulloblastoma; medulloepithelioma; melanoma; melanoma, intraocular (eye); Merkel cell cancer; Merkel cell skin carcinoma; mesothelioma; mesothelioma, adult malignant; metastatic squamous neck cancer with occult primary; mouth cancer; multiple endocrine neoplasia syndrome; multiple myeloma/plasma cell neoplasm; mycosis fungoides, myelodysplastic syndromes; myelodysplastic/myeloproliferative diseases; myelogenous leukemia, chronic; myeloid leukemia, adult acute; myeloid leukemia, childhood acute; myeloma, multiple (cancer of the bone-marrow); myeloproliferative disorders, chronic; nasal cavity and paranasal sinus cancer; nasopharyngeal carcinoma; neuroblastoma, non-small cell lung cancer; non-Hodgkin's lymphoma; oligodendroglioma; oral cancer; oral cavity cancer; oropharyngeal cancer; osteosarcoma/malignant fibrous histiocytoma of bone; ovarian cancer; ovarian epithelial cancer (surface epithelial-stromal tumor); ovarian germ cell tumor; ovarian low malignant potential tumor; pancreatic cancer; pancreatic cancer, islet cell; papillomatosis; paranasal sinus and nasal cavity cancer; parathyroid cancer; penile cancer; pharyngeal cancer; pheochromocytoma; pineal astrocytoma; pineal germinoma; pineal parenchymal tumors of intermediate differentiation; pineoblastoma and supratentorial primitive neuroectodermal tumors; pituitary tumor; pituitary adenoma; plasma cell neoplasia/multiple myeloma; pleuropulmonary blastoma; primary central nervous system lymphoma; prostate cancer; rectal cancer; renal cell carcinoma (kidney cancer); renal pelvis and ureter, transitional cell cancer; NUT midline carcinoma; retinoblastoma; rhabdomyosarcoma, childhood; salivary gland cancer; sarcoma, Ewing family of tumors; Sezary syndrome; skin cancer (melanoma); skin cancer (non-melanoma); small cell lung cancer; small intestine cancer soft tissue sarcoma; soft tissue sarcoma; spinal cord tumor; squamous cell carcinoma; squamous neck cancer with occult primary, metastatic; stomach (gastric) cancer; supratentorial primitive neuroectodermal tumor; T-cell lymphoma, cutaneous (Mycosis Fungoides and Sezary syndrome); testicular cancer; throat cancer; thymoma; thymoma and thymic carcinoma; thyroid cancer; thyroid cancer, childhood; transitional cell cancer of the renal pelvis and ureter; urethral cancer; uterine cancer, endometrial; uterine sarcoma; vaginal cancer; vulvar cancer; and Wilms Tumor. In some embodiments, a syndrome is a group of symptoms which, taken together, characterize a condition.

In some embodiments, the target nucleic acid is single-stranded RNA. The methods, reagents, enzymes, and kits disclosed herein may enable the direct detection of a RNA encoding a sequence of interest. A nucleic acid can encode a sequence from a genomic locus. In some cases, the target nucleic acid that binds to the guide nucleic acid is from 5 to 100, 5 to 90, 5 to 80, 5 to 70, 5 to 60, 5 to 50, 5 to 40, 5 to 30, 5 to 25, 5 to 20, 5 to 15, or 5 to 10 nucleotides in length. The nucleic acid can be from 10 to 90, from 20 to 80, from 30 to 70, or from 40 to 60 nucleotides in length. A nucleic acid can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 nucleotides in length. The target nucleic acid can encode a sequence reverse complementary to a guide nucleic acid sequence.

In some instances, the sample is taken from single-cell eukaryotic organisms; a plant or a plant cell; an algal cell; a fungal cell; an animal cell, tissue, or organ; a cell, tissue, or organ from an invertebrate animal; a cell, tissue, fluid, or organ from a vertebrate animal such as fish, amphibian, reptile, bird, and mammal; a cell, tissue, fluid, or organ from a mammal such as a human, a non-human primate, an ungulate, a feline, a bovine, an ovine, and a caprine. In some instances, the sample is taken from nematodes, protozoans, helminths, or malarial parasites. In some cases, the sample comprises nucleic acids from a cell lysate from a eukaryotic cell, a mammalian cell, a human cell, a prokaryotic cell, or a plant cell. In some cases, the sample comprises nucleic acids expressed from a cell.

The sample described herein may comprise at least one target nucleic acid. The target nucleic acid comprises a segment that is reverse complementary to a segment of a guide nucleic acid. Often, the sample comprises the segment of the target nucleic acid and at least one nucleic acid comprising at least 50% sequence identity to a segment of the target nucleic acid. Sometimes, the at least one nucleic acid comprises a segment comprising at least 60%, 70%, 75%, 80%, 85%, 90%, 91%, 92%, 93%, 94%, 95%, 96%, 97%, 98%, or 99% sequence identity to the segment of the target nucleic acid. Often, a sample comprises the segment of the target nucleic acid and at least one nucleic acid a segment comprising less than 100% sequence identity to the target nucleic acid but no less than 50% sequence identity to the segment of the target nucleic acid. Sometimes, a sample comprises the segment of the target nucleic acid and at least one nucleic acid a segment comprising less than 100% sequence identity to the target nucleic acid but no less than 60%, 70%, 75%, 80%, 85%, 90%, 91%, 92%, 93%, 94%, 95%, 96%, 97%, 98%, or 99% sequence identity to the segment of the target nucleic acid. For example, the segment of the target nucleic acid comprises a mutation as compared to at least one nucleic acid comprising a segment comprising less than 100% sequence identity to the segment of the target nucleic acid but no less than 50% sequence identity to the segment of the target nucleic acid.

In some embodiments, target nucleic acids comprise a mutation. In some embodiments, a composition, system or method described herein can be used to modify a target nucleic acid comprising a mutation such that the mutation is modified to be a wild-type nucleotide or nucleotide sequence. In some embodiments, a composition, system or method described herein can be used to detect a target nucleic acid comprising a mutation.

A mutation may be in an open reading frame of a target nucleic acid. A mutation may result in the insertion of at least one amino acid in a protein encoded by the target nucleic acid. A mutation may result in the deletion of at least one amino acid in a protein encoded by the target nucleic acid. A mutation may result in the substitution of at least one amino acid in a protein encoded by the target nucleic acid. A mutation that results in the deletion, insertion, or substitution of one or more amino acids of a protein encoded by the target nucleic acid may result in misfolding of a protein encoded by the target nucleic acid. A mutation may result in a premature stop codon, thereby resulting in a truncation of the encoded protein.

In some embodiments, mutations comprise a point mutation, a chromosomal mutation, a copy number mutation, or any combination thereof. A point mutation may be a substitution, insertion, or deletion of a single nucleotide. In some embodiments, mutations comprise a chromosomal mutation. A chromosomal mutation may comprise an inversion, a deletion, a duplication, or a translocation of one or more nucleotides. In some embodiments, mutations comprise a copy number variation. A copy number variation may comprise a gene amplification or an expanding trinucleotide repeat. In some embodiments, guide nucleic acids described herein hybridize to a target sequence of a target nucleic acid comprising the mutation. In some embodiments, mutations are located in a non-coding region of a gene.

Sometimes, the segment of the target nucleic acid comprises a mutation as compared to at least one nucleic acid comprising a segment comprising less than 100% sequence identity to the segment of the target nucleic acid but no less than 60%, 70%, 75%, 80%, 85%, 90%, 91%, 92%, 93%, 94%, 95%, 96%, 97%, 98%, or 99% sequence identity to the segment of the target nucleic acid. Often, the segment of the target nucleic acid comprises a mutation as compared to at least one nucleic acid comprising a segment comprising less than 100% sequence identity to the segment of the target nucleic acid but no less than 50% sequence identity to the segment of the target nucleic acid. The mutation can be a mutation of 1, 2, 3, 4, 5, 6, 7, 8, 9, 10, 11, 12, 13, 14, 15, 16, 17, 18, 19, 20 or more nucleotides. Often, the mutation is a single nucleotide mutation. The single nucleotide mutation can be a single nucleotide polymorphism (SNP), which is a single base pair variation in a DNA sequence present in less than 1% of a population and is present in an transcribed RNA. Sometimes, the target nucleic acid comprises a single nucleotide mutation, wherein the single nucleotide mutation comprises the wild type variant of the SNP. The single nucleotide mutation or SNP can be associated with a phenotype of the sample or a phenotype of the organism from which the sample was taken. The SNP, in some cases, is associated with altered phenotype from wild type phenotype. Often, the segment of the target nucleic acid sequence comprises a deletion as compared to at least one nucleic acid comprising a segment comprising less than 100% sequence identity to the segment of the target nucleic acid but no less than 50% sequence identity to the segment of the target nucleic acid. The mutation can be a deletion of 1, 2, 3, 4, 5, 6, 7, 8, 9, 10, 11, 12, 13, 14, 15, 16, 17, 18, 19, 20 or more nucleotides. The mutation can be a deletion of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. The mutation can be a deletion of from 1 to 5, from 5 to 10, from 10 to 15, from 15 to 20, from 20 to 25, from 25 to 30, from 30 to 35, from 35 to 40, from 40 to 45, from 45 to 50, from 50 to 55, from 55 to 60, from 60 to 65, from 65 to 70, from 70 to 75, from 75 to 80, from 80 to 85, from 85 to 90, from 90 to 95, from 95 to 100, from 100 to 200, from 200 to 300, from 300 to 400, from 400 to 500, from 500 to 600, from 600 to 700, from 700 to 800, from 800 to 900, from 900 to 1000, from 1 to 50, from 1 to 100, from 25 to 50, from 25 to 100, from 50 to 100, from 100 to 500, from 100 to 1000, or from 500 to 1000 nucleotides. The segment of the target nucleic acid that the guide nucleic acid of the methods describe herein binds to comprises the mutation, such as the SNP or the deletion. The mutation can be a single nucleotide mutation or a SNP. The SNP can be a synonymous substitution or a nonsynonymous substitution. The nonsynonymous substitution can be a missense substitution or a nonsense point mutation. The synonymous substitution can be a silent substitution. The mutation can be a deletion of one or more nucleotides. Often, the single nucleotide mutation, SNP, or deletion is associated with a disease such as cancer or a genetic disorder. The mutation, such as a single nucleotide mutation, a SNP, or a deletion, can be encoded in the sequence of a target nucleic acid from the germline of an organism or can be encoded in a target nucleic acid from a diseased cell, such as a cancer cell. In some examples, a mutation associated with a disease refers to a mutation whose presence in a subject indicates that the subject is susceptible to or suffers from, a disease, disorder, condition, or syndrome. In some examples, a mutation associated with a disease refers to a mutation which causes, contributes to the development of, or indicates the existence of the disease, disorder, condition, or syndrome. A mutation associated with a disease may also refer to any mutation which generates transcription or translation products at an abnormal level, or in an abnormal form, in cells affected by a disease relative to a control without the disease. In some embodiments, a mutation associated with a disease is the co-occurrence of a mutation and the phenotype of a disease. The mutation may occur in a gene, wherein transcription or translation products from the gene occur at a significantly abnormal level or in an abnormal form in a cell or subject harboring the mutation as compared to a non-disease control subject not having the mutation.

The sample used for disease testing may comprise at least one target nucleic acid that can bind to a guide nucleic acid of the reagents described herein. The sample used for disease testing may comprise at least nucleic acid of interest that is amplified to produce a target nucleic acid that can bind to a guide nucleic acid of the reagents described herein. The nucleic acid of interest can comprise DNA, RNA, or a combination thereof.

The target nucleic acid (e.g., a target RNA or DNA) may be a portion of a nucleic acid from a virus or a bacterium or other agents responsible for a disease in the sample. The target nucleic acid may be a portion of a nucleic acid from a gene expressed in a cancer or genetic disorder in the sample. In some cases, the sequence is a segment of a target nucleic acid sequence. A segment of a target nucleic acid sequence can be from a genomic locus, a transcribed mRNA, or a reverse transcribed cDNA. A segment of a target nucleic acid sequence can be from 5 to 100, 5 to 90, 5 to 80, 5 to 70, 5 to 60, 5 to 50, 5 to 40, 5 to 30, 5 to 25, 5 to 20, 5 to 15, or 5 to 10 nucleotides in length. A segment of a target nucleic acid sequence can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 nucleotides in length. The sequence of the target nucleic acid segment can be reverse complementary to a segment of a guide nucleic acid sequence. The target nucleic acid may comprise a genetic variation (e.g., a single nucleotide polymorphism), with respect to a standard sample, associated with a disease phenotype or disease predisposition.

In some embodiments, the target nucleic acid sequence comprises a nucleic acid sequence of a virus or a bacterium or other agents responsible for a disease in the sample. In some embodiments, the target nucleic acid comprises RNA or DNA. The target nucleic acid, in some cases, is a portion of a nucleic acid from a sexually transmitted infection or a contagious disease, in the sample. In some cases, the target nucleic acid is a portion of a nucleic acid from a genomic locus, or any DNA amplicon, such as a reverse transcribed mRNA or a cDNA from a gene locus, a transcribed mRNA, or a reverse transcribed cDNA from a gene locus in at least one of: human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitidis, Chlamydia trachomatis, and Candida albicans. Pathogenic viruses include but are not limited to coronavirus; immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, SARS CoV2/COVID, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae. In some cases, the target sequence is a portion of a nucleic acid from a genomic locus, a transcribed mRNA, or a reverse transcribed cDNA from a gene locus of bacterium or other agents responsible for a disease in the sample comprising a mutation that confers resistance to a treatment, such as a single nucleotide mutation that confers resistance to antibiotic treatment. In some cases, the mutation that confers resistance to a treatment is a deletion.

Compositions and methods of the disclosure can be used for cell line engineering (e.g., engineering a cell from a cell line for bioproduction). For example, compositions and methods of the disclosure can be used to express a desired protein from a cell line. In some embodiments, the target nucleic acid sequence comprises a nucleic acid sequence of a cell line. In some embodiments, the target nucleic acid sequence comprises a genomic nucleic acid sequence of a cell line. In some embodiments, the cell line is a Chinese hamster ovary cell line (CHO), human embryonic kidney cell line (HEK), cell lines derived from cancer cells, cell lines derived from lymphocytes, and the like. Non-limiting examples of cell lines includes: C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, CIR, Rat6, CV1, RPTE, A10, T24, J82, A375, ARH-77, Calu1, SW480, SW620, SKOV3, SK-UT, CaCo2, P388D1, SEM-K2, WEHI-231, HB56, T1B55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr−/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, and YAR. Non-limiting examples of other cells that can be used with the disclosure include immune cells, such as CART, T-cells, B-cells, NK cells, granulocytes, basophils, eosinophils, neutrophils, mast cells, monocytes, macrophages, dendritic cells, antigen-presenting cells (APC), or adaptive cells. In some embodiments, a T cell is a type of lymphocyte that matures in the thymus. T cells play an important role in cell-mediated immunity and are distinguished from other lymphocytes, such as B cells, by the presence of a T-cell receptor on the cell surface. A T cell includes all types of immune cells expressing CD3, including: naïve T cells (cells that have not encountered their cognate antigens), T-helper cells (CD4+ cells), cytotoxic T-cells (CD8+ cells), natural killer T-cells, T-regulatory cells (T-reg) and gamma-delta T cells. Non-limiting exemplary sources for commercially available T cell lines include the American Type Culture Collection, or ATCC, and the German Collection of Microorganisms and Cell Cultures. Non-limiting examples of cells that can be used with this disclosure also include plant cells, such as parenchyma, sclerenchyma, collenchyma, xylem, phloem, germline (e.g., pollen). Cells from lycophytes, ferns, gymnosperms, angiosperms, bryophytes, charophytes, chloropytes, rhodophytes, or glaucophytes. Non-limiting examples of cells that can be used with this disclosure also include stem cells, such as human stem cells, animal stem cells, stem cells that are not derived from human embryonic stem cells, embryonic stem cells, mesenchymal stem cells, pluripotent stem cells, induced pluripotent stem cells (iPS), somatic stem cells, adult stem cells, hematopoietic stem cells, tissue-specific stem cells.

Compositions and methods of the disclosure can be used for agricultural engineering. For example, compositions and methods of the disclosure can be used to confer desired traits on a plant. A plant can be engineered for the desired physiological and agronomic characteristic using the present disclosure. In some embodiments, the target nucleic acid sequence comprises a nucleic acid sequence of a plant. In some embodiments, the target nucleic acid sequence comprises a genomic nucleic acid sequence of a plant cell. In some embodiments, the target nucleic acid sequence comprises a nucleic acid sequence of an organelle of a plant cell. In some embodiments, the target nucleic acid sequence comprises a nucleic acid sequence of a chloroplast of a plant cell.

In some embodiments, the target nucleic acid sequence comprises a nucleic acid belonging to domestic animal such as common livestock and common pets. In some embodiments, domestic animals can include, but are not limited to, pigs, cattle, horses, dogs, cats, and other ruminant animals such as sheep, goats, oxen, musk ox, llamas, alpacas, guanicos, deer, bison, antelopes, camels, and giraffes.

The plant can be a monocotyledonous plant. The plant can be a dicotyledonous plant. Non-limiting examples of orders of dicotyledonous plants include Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales.

Non-limiting examples of orders of monocotyledonous plants include Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales. A plant can belong to the order, for example, Gymnospermae, Pinales, Ginkgoales, Cycadales, Araucariales, Cupressales and Gnetales.

Non-limiting examples of plants include plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses, wheat, maize, rice, millet, barley, tomato, apple, pear, strawberry, orange, acacia, carrot, potato, sugar beets, yam, lettuce, spinach, sunflower, rape seed, Arabidopsis, alfalfa, amaranth, apple, apricot, artichoke, ash tree, asparagus, avocado, banana, barley, beans, beet, birch, beech, blackberry, blueberry, broccoli, Brussel's sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, and zucchini. A plant can include algae.

In some embodiments, the target nucleic acid sequence comprises a nucleic acid sequence of a virus, a bacterium, or other pathogen responsible for a disease in a plant (e.g., a crop). Methods and compositions of the disclosure can be used to treat or detect a disease in a plant. For example, the methods of the disclosure can be used to target a viral nucleic acid sequence in a plant. A programmable nuclease of the disclosure (e.g., Cas13) can cleave the viral nucleic acid. In some embodiments, the target nucleic acid sequence comprises a nucleic acid sequence of a virus or a bacterium or other agents (e.g., any pathogen) responsible for a disease in the plant (e.g., a crop). In some embodiments, the target nucleic acid comprises RNA. The target nucleic acid, in some cases, is a portion of a nucleic acid from a virus or a bacterium or other agents responsible for a disease in the plant (e.g., a crop). In some cases, the target nucleic acid is a portion of a nucleic acid from a genomic locus, or any NA amplicon, such as a reverse transcribed mRNA or a cDNA from a gene locus, a transcribed mRNA, or a reverse transcribed cDNA from a gene locus in at a virus or a bacterium or other agents (e.g., any pathogen) responsible for a disease in the plant (e.g., a crop). A virus infecting the plant can be an RNA virus. A virus infecting the plant can be a DNA virus. Non-limiting examples of viruses that can be targeted with the disclosure include Tobacco mosaic virus (TMV), Tomato spotted wilt virus (TSWV), Cucumber mosaic virus (CMV), Potato virus Y (PVY), Cauliflower mosaic virus (CaMV) (RT virus), Plum pox virus (PPV), SARS-CoV-2/COVID, Brome mosaic virus (BMV) and Potato virus X (PVX).

The sample used for cancer testing may comprise at least one target nucleic acid that can bind to a guide nucleic acid of the reagents described herein. The target nucleic acid, in some cases, comprises a portion of a gene comprising a mutation associated with cancer, a gene whose overexpression is associated with cancer, a tumor suppressor gene, an oncogene, a checkpoint inhibitor gene, a gene associated with cellular growth, a gene associated with cellular metabolism, or a gene associated with cell cycle. Sometimes, the target nucleic acid encodes a cancer biomarker, such as a prostate cancer biomarker or non-small cell lung cancer. In some cases, the assay can be used to detect “hotspots” in target nucleic acids that can be predictive of lung cancer. In some cases, the target nucleic acid comprises a portion of a nucleic acid that is associated with a blood fever. In some cases, the target nucleic acid is a portion of a nucleic acid from a genomic locus, any DNA amplicon of, a reverse transcribed mRNA, or a cDNA from a locus of at least one of: ALK, APC, ATM, AXIN2, BAP1, BARD1, BLM, BMPR1A, BRCA1, BRCA2, BRIP1, CASR, CDC73, CDH1, CDK4, CDKN1B, CDKN1C, CDKN2A, CEBPA, CHEK2, CTNNA1, DICERI, DIS3L2, EGFR, EPCAM, FH, FLCN, GATA2, GPC3, GREM1, HOXB13, HRAS, KIT, MAX, MEN1, MET, MITF, MLH1, MSH2, MSH3, MSH6, MUTYH, NBN, NF1, NF2, NTHL1, PALB2, PDGFRA, PHOX2B, PMS2, POLD1, POLE, POT1, PRKAR1A, PTCH1, PTEN, RAD50, RAD51C, RAD51D, RB1, RECQL4, RET, RUNX1, SDHA, SDHAF2, SDHB, SDHC, SDHD, SMAD4, SMARCA4, SMARCB1, SMARCEl, STK11, SUFU, TERC, TERT, TMEM127, TP53, TSC1, TSC2, VHL, WRN, and WT1. Any region of the aforementioned gene loci can be probed for a mutation or deletion using the compositions and methods disclosed herein. For example, in the EGFR gene locus, the compositions and methods for detection disclosed herein can be used to detect a single nucleotide polymorphism or a deletion. The SNP or deletion can occur in a non-coding region or a coding region.

The sample used for genetic disorder testing may comprise at least one target nucleic acid that can bind to a guide nucleic acid of the reagents described herein. In some embodiments, the genetic disorder is hemophilia, sickle cell anemia, β-thalassemia, Duchene muscular dystrophy, severe combined immunodeficiency, Huntington's disease, or cystic fibrosis. The target nucleic acid, in some cases, is from a gene with a mutation associated with a genetic disorder, from a gene whose overexpression is associated with a genetic disorder, from a gene associated with abnormal cellular growth resulting in a genetic disorder, or from a gene associated with abnormal cellular metabolism resulting in a genetic disorder. In some cases, the target nucleic acid is a nucleic acid from a genomic locus, a transcribed mRNA, or a reverse transcribed mRNA, a DNA amplicon of or a cDNA from a locus of at least one of: CFTR, FMR1, SMN1, ABCB11, ABCC8, ABCD1, ACAD9, ACADM, ACADVL, ACAT1, ACOX1, ACSF3, ADA, ADAMTS2, ADGRG1, AGA, AGL, AGPS, AGXT, AIRE, ALDH3A2, ALDOB, ALG6, ALMS1, ALPL, AMT, AQP2, ARG1, ARSA, ARSB, ASL, ASNS, ASPA, ASS1, ATM, ATP6V1B1, ATP7A, ATP7B, ATRX, BBS1, BBS10, BBS12, BBS2, BCKDHA, BCKDHB, BCS1L, BLM, BSND, CAPN3, CBS, CDH23, CEP290, CERKL, CHM, CHRNE, CIITA, CLN3, CLN5, CLN6, CLN8, CLRN1, CNGB3, COL27A1, COL4A3, COL4A4, COL4A5, COL7A1, CPS1, CPT1A, CPT2, CRB1, CTNS, CTSK, CYBA, CYBB, CYP11B1, CYP11B2, CYP17A1, CYP19A1, CYP27A1, DBT, DCLRE1C, DHCR7, DHDDS, DLD, DMD, DNAH5, DNAI1, DNAI2, DYSF, EDA, EIF2B5, EMD, ERCC6, ERCC8, ESCO2, ETFA, ETFDH, ETHEl, EVC, EVC2, EYS, F9, FAH, FAM161A, FANCA, FANCC, FANCG, FH, FKRP, FKTN, G6PC, GAA, GALC, GALKI, GALT, GAMT, GBA, GBE1, GCDH, GFM1, GJB1, GJB2, GLA, GLB1, GLDC, GLE1, GNE, GNPTAB, GNPTG, GNS, GRHPR, HADHA, HAX1, HBA1, HBA2, HBB, HEXA, HEXB, HGSNAT, HLCS, HMGCL, HOGA1, HPS1, HPS3, HSD17B4, HSD3B2, HYAL1, HYLS1, IDS, IDUA, IKBKAP, IL2RG, IVD, KCNJ11, LAMA2, LAMA3, LAMB3, LAMC2, LCA5, LDLR, LDLRAP1, LHX3, LIFR, LIPA, LOXHD1, LPL, LRPPRC, MAN2B1, MCOLN1, MED17, MESP2, MFSD8, MKS1, MLC1, MMAA, MMAB, MMACHC, MMADHC, MPI, MPL, MPV17, MTHFR, MTM1, MTRR, MTTP, MUT, MYO7A, NAGLU, NAGS, NBN, NDRG1, NDUFAF5, NDUFS6, NEB, NPC1, NPC2, NPHS1, NPHS2, NR2E3, NTRK1, OAT, OPA3, OTC, PAH, PC, PCCA, PCCB, PCDH15, PDHA1, PDHB, PEX1, PEX10, PEX12, PEX2, PEX6, PEX7, PFKM, PHGDH, PKHD1, PMM2, POMGNT1, PPT1, PROP1, PRPS1, PSAP, PTS, PUS1, PYGM, RAB23, RAG2, RAPSN, RARS2, RDH12, RMRP, RPE65, RPGRIP1L, RS1, RTEL1, SACS, SAMHD1, SEPSECS, SGCA, SGCB, SGCG, SGSH, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMARCAL1, SMPD1, STAR, SUMF1, TAT, TCIRG1, TECPR2, TFR2, TGM1, TH, TMEM216, TPP1, TRMU, TSFM, TTPA, TYMP, USH1C, USH2A, VPS13A, VPS13B, VPS45, VRK1, VSX2, WNT10A, XPA, XPC, and ZFYVE26.

The sample used for phenotyping testing may comprise at least one target nucleic acid that can bind to a guide nucleic acid of the reagents described herein. The target nucleic acid, in some cases, is a nucleic acid encoding a sequence associated with a phenotypic trait.

The sample used for genotyping testing may comprise at least one target nucleic acid that can bind to a guide nucleic acid of the reagents described herein. The target nucleic acid, in some cases, is a nucleic acid encoding a sequence associated with a genotype of interest.

The sample used for ancestral testing may comprise at least one target nucleic acid that can bind to a guide nucleic acid of the reagents described herein. The target nucleic acid, in some cases, is a nucleic acid encoding a sequence associated with a geographic region of origin or ethnic group.

The sample can be used for identifying a disease status. For example, a sample is any sample described herein, and is obtained from a subject for use in identifying a disease status of a subject. The disease can be a cancer or genetic disorder. Sometimes, a method comprises obtaining a serum sample from a subject; and identifying a disease status of the subject. Often, the disease status is prostate disease status, but the status of any disease can be assessed.

In some instances, the target nucleic acid is a single stranded nucleic acid. Alternatively, or in combination, the target nucleic acid is a double stranded nucleic acid and is prepared into single stranded nucleic acids before or upon contacting the reagents. The target nucleic acid may be a RNA. The target nucleic acids include but are not limited to mRNA, rRNA, tRNA, non-coding RNA, long non-coding RNA, and microRNA (miRNA). In some cases, the target nucleic acid is single-stranded RNA (ssRNA) or mRNA. In some cases, the target nucleic acid is from a virus, a parasite, or a bacterium described herein.

In some embodiments, the target nucleic acid is a double stranded nucleic acid. In some embodiments, the double stranded nucleic acid is DNA.

A number of target nucleic acids are consistent with the methods and compositions disclosed herein. Some methods described herein can detect a target nucleic acid present in the sample in various concentrations or amounts as a target nucleic acid population. In some cases, the sample has at least 2 target nucleic acids. In some cases, the sample has at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 target nucleic acids. In some cases, the sample has from 1 to 10,000, from 100 to 8000, from 400 to 6000, from 500 to 5000, from 1000 to 4000, or from 2000 to 3000 target nucleic acids. In some cases, the method detects target nucleic acid present at least at one copy per 10 non-target nucleic acids, 102 non-target nucleic acids, 103 non-target nucleic acids, 104 non-target nucleic acids, 105 non-target nucleic acids, 106 non-target nucleic acids, 107 non-target nucleic acids, 108 non-target nucleic acids, 109 non-target nucleic acids, or 1010 non-target nucleic acids. Often, the target nucleic acid can be from 0.05% to 20% of total nucleic acids in the sample. Sometimes, the target nucleic acid is from 0.1% to 10% of the total nucleic acids in the sample. The target nucleic acid, in some cases, is from 0.1% to 5% of the total nucleic acids in the sample. The target nucleic acid can also be from 0.1% to 1% of the total nucleic acids in the sample. The target nucleic acid can be DNA or RNA. The target nucleic acid can be any amount less than 100% of the total nucleic acids in the sample. The target nucleic acid can be 100% of the total nucleic acids in the sample.

In some embodiments, the sample comprises a target nucleic acid at a concentration of less than 1 nM, less than 2 nM, less than 3 nM, less than 4 nM, less than 5 nM, less than 6 nM, less than 7 nM, less than 8 nM, less than 9 nM, less than 10 nM, less than 20 nM, less than 30 nM, less than 40 nM, less than 50 nM, less than 60 nM, less than 70 nM, less than 80 nM, less than 90 nM, less than 100 nM, less than 200 nM, less than 300 nM, less than 400 nM, less than 500 nM, less than 600 nM, less than 700 nM, less than 800 nM, less than 900 nM, less than 1 μM, less than 2 μM, less than 3 μM, less than 4 μM, less than 5 μM, less than 6 μM, less than 7 μM, less than 8 μM, less than 9 μM, less than 10 μM, less than 100 μM, or less than 1 mM. In some embodiments, the sample comprises a target nucleic acid sequence at a concentration of from 1 nM to 2 nM, from 2 nM to 3 nM, from 3 nM to 4 nM, from 4 nM to 5 nM, from 5 nM to 6 nM, from 6 nM to 7 nM, from 7 nM to 8 nM, from 8 nM to 9 nM, from 9 nM to 10 nM, from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1 μM, from 1 μM to 2 μM, from 2 μM to 3 μM, from 3 μM to 4 μM, from 4 μM to 5 μM, from 5 μM to 6 μM, from 6 μM to 7 μM, from 7 μM to 8 μM, from 8 μM to 9 μM, from 9 μM to 10 μM, from 10 μM to 100 μM, from 100 μM to 1 mM, from 1 nM to 10 nM, from 1 nM to 100 nM, from 1 nM to 1 μM, from 1 nM to 10 μM, from 1 nM to 100 μM, from 1 nM to 1 mM, from 10 nM to 100 nM, from 10 nM to 1 μM, from 10 nM to 10 μM, from 10 nM to 100 μM, from 10 nM to 1 mM, from 100 nM to 1 μM, from 100 nM to 10 μM, from 100 nM to 100 μM, from 100 nM to 1 mM, from 1 μM to 10 μM, from 1 μM to 100 μM, from 1 μM to 1 mM, from 10 μM to 100 μM, from 10 μM to 1 mM, or from 100 μM to 1 mM. In some embodiments, the sample comprises a target nucleic acid at a concentration of from 20 nM to 200 μM, from 50 nM to 100 μM, from 200 nM to 50 μM, from 500 nM to 20 μM, or from 2 μM to 10 μM. In some embodiments, the target nucleic acid is not present in the sample.

In some embodiments, the sample comprises fewer than 10 copies, fewer than 100 copies, fewer than 1000 copies, fewer than 10,000 copies, fewer than 100,000 copies, or fewer than 1,000,000 copies of a target nucleic acid sequence. In some embodiments, the sample comprises from 10 copies to 100 copies, from 100 copies to 1000 copies, from 1000 copies to 10,000 copies, from 10,000 copies to 100,000 copies, from 100,000 copies to 1,000,000 copies, from 10 copies to 1000 copies, from 10 copies to 10,000 copies, from 10 copies to 100,000 copies, from 10 copies to 1,000,000 copies, from 100 copies to 10,000 copies, from 100 copies to 100,000 copies, from 100 copies to 1,000,000 copies, from 1,000 copies to 100,000 copies, or from 1,000 copies to 1,000,000 copies of a target nucleic acid sequence. In some embodiments, the sample comprises from 10 copies to 500,000 copies, from 200 copies to 200,000 copies, from 500 copies to 100,000 copies, from 1000 copies to 50,000 copies, from 2000 copies to 20,000 copies, from 3000 copies to 10,000 copies, or from 4000 copies to 8000 copies. In some embodiments, the target nucleic acid is not present in the sample.

A number of target nucleic acid populations are consistent with the methods and compositions disclosed herein. Some methods described herein can detect two or more target nucleic acid populations present in the sample in various concentrations or amounts. In some cases, the sample has at least 2 target nucleic acid populations. In some cases, the sample has at least 3, 4, 5, 6, 7, 8, 9, 10, 20, 30, 40, or 50 target nucleic acid populations. In some cases, the sample has from 3 to 50, from 5 to 40, or from 10 to 25 target nucleic acid populations. In some cases, the method detects target nucleic acid populations that are present at least at one copy per 101 non-target nucleic acids, 102 non-target nucleic acids, 103 non-target nucleic acids, 104 non-target nucleic acids, 105 non-target nucleic acids, 106 non-target nucleic acids, 107 non-target nucleic acids, 108 non-target nucleic acids, 109 non-target nucleic acids, or 1010 non-target nucleic acids. The target nucleic acid populations can be present at different concentrations or amounts in the sample.

In some embodiments, the target nucleic acid as disclosed herein can activate the programmable nuclease to initiate sequence-independent cleavage of a nucleic acid-based reporter (e.g., a reporter comprising an RNA sequence, a reporter comprising a DNA sequence, or a reporter comprising DNA and RNA). For example, a programmable nuclease of the present disclosure is activated by a target nucleic acid to cleave reporters having an RNA (also referred to herein as an “RNA reporter”). Alternatively, a programmable nuclease of the present disclosure is activated by a target nucleic acid to cleave reporters having a DNA. Alternatively, a programmable nuclease of the present disclosure is activated by a target RNA to cleave reporters having an RNA (also referred to herein as a “RNA reporter”). Alternatively, a programmable nuclease of the present disclosure is activated by a target RNA to cleave reporters having a DNA (also referred to herein as a “DNA reporter”). The RNA reporter can comprise a single-stranded RNA or single-stranded DNA labelled with a detection moiety or can be any RNA or ssDNA reporter as disclosed herein.

In some embodiments, the target nucleic acid as described in the methods herein does not initially comprise a PAM sequence. However, any target nucleic acid of interest may be generated using the methods described herein to comprise a PAM sequence, and thus be a PAM target nucleic acid. A PAM target nucleic acid, as used herein, refers to a target nucleic acid that has been amplified to insert a PAM sequence that is recognized by a CRISPR/Cas system.

In some embodiments, the target nucleic acid is in a cell. In some embodiments, the cell is a single-cell eukaryotic organism; a plant cell an algal cell; a fungal cell; an animal cell; a cell from an invertebrate animal; a cell from a vertebrate animal such as fish, amphibian, reptile, bird, and mammal; or a cell from a mammal such as a human, a non-human primate, an ungulate, a feline, a bovine, an ovine, and a caprine. In preferred embodiments, the cell is a eukaryotic cell. In preferred embodiments, the cell is a mammalian cell, a human cell, or a plant cell.

Any of the above disclosed samples are consistent with the methods, compositions, reagents, enzymes, and kits disclosed herein and can be used as a companion diagnostic with any of the diseases disclosed herein, or can be used in reagent kits, point-of-care diagnostics, or over-the-counter diagnostics.

### Methods of Modifying or Editing a Target Nucleic Acid Sequence

Provided herein, is a method of altering the sequence of a nucleic acid, the method comprising contacting a target nucleic acid molecule with any one of the compositions or systems described herein. In some embodiments, the target nucleic acid is single stranded. In some embodiments, the target nucleic acid is double stranded. In some embodiments, the target nucleic acid comprises RNA. In some embodiments, the target nucleic acid comprises DNA. In some embodiments, the programmable nuclease further comprises an editing domain. In some embodiments, the editing domain comprises ADAR1/2 or a functional variant thereof. In some embodiments, the contacting occurs in vitro. In some embodiments, the contacting occurs ex vivo. In some embodiments, the contacting occurs in vivo. In some embodiments, the contacting occurs in a sample, wherein the sample is selected from an environmental sample and a biological sample. In some embodiments, the biological sample is selected from blood, plasma, saliva, a buccal swab, a nasal swab, and urine.

The disclosure provides compositions and methods for modifying or editing a target nucleic acid sequence. Compositions and methods of the disclosure can be used for introducing a site-specific cleavage in a target nucleic acid sequence. The site-specific cleavage can be a double-strand cleavage. The site-specific cleavage can be a single-strand cleavage. The modification can result in introducing a mutation (e.g., point mutations, deletions) in a target nucleic acid. The modification can result in removing a disease-causing mutation in a nucleic acid sequence. Methods of the disclosure can be targeted to any locus in a genome of a cell. They can generate point mutations, deletions, null mutations, or tissue-specific mutations in a target nucleic acid sequence. A complex comprising a programmable nuclease and guide nucleic acid of the disclosure can be used to generate gene knock-out, gene knock-in, gene editing, gene tagging, or a combination thereof.

The methods described herein may be used to edit or modify a target nucleic acid. Methods of modifying a target nucleic acid may use the compositions comprising a programmable Type VI CRISPR/Cas nuclease and an engineered guide nucleic acid as described herein. Modifying a target nucleic acid may comprise one or more of cleaving the target nucleic acid, deleting one or more nucleotides of the target nucleic acid, inserting one or more nucleotides into the target nucleic acid, mutating one or more nucleotides of the target nucleic acid, or modifying (e.g., methylating, demethylating, deaminating, or oxidizing) of one or more nucleotides of the target nucleic acid.

In some embodiments, modifying a target nucleic acid comprises genome editing. Genome editing may comprise modifying a genome, chromosome, plasmid, or other genetic material of a cell or organism. In some embodiments the genome, chromosome, plasmid, or other genetic material of the cell or organism is modified in vivo. In some embodiments the genome, chromosome, plasmid, or other genetic material of the cell or organism is modified in a cell. In some embodiments the genome, chromosome, plasmid, or other genetic material of the cell or organism is modified in vitro. In some embodiments, in vitro is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed. In vivo is used to describe an event that takes place in a subject's body. Ex vivo is used to describe an event that takes place outside of a subject's body. An ex vivo assay is not performed on a subject. Rather, it is performed upon a sample separate from a subject. An example of an ex vivo assay performed on a sample is an in vitro assay. For example, a plasmid may be modified in vitro using a composition described herein and introduced into a cell or organism. In some embodiments, modifying a target nucleic acid may comprise deleting a sequence from a target nucleic acid. For example, a mutated sequence or a sequence associated with a disease may be removed from a target nucleic acid. In some embodiments, modifying a target nucleic acid may comprise replacing a sequence in a target nucleic acid with a second sequence. For example, a mutated sequence or a sequence associated with a disease may be replaced with a second sequence lacking the mutation or that is not associated with the disease. In some embodiments, modifying a target nucleic acid may comprise introducing a sequence into a target nucleic acid. For example, a beneficial sequence or a sequence that may reduce or eliminate a disease may be inserted into the target nucleic acid.

In some embodiments, the present disclosure provides methods and compositions for editing a target nucleic acid sequence comprising a programmable Type VI CRISPR/Cas nuclease capable of introducing a break in a single stranded RNA (ssRNA) target sequence. The programmable Type VI CRISPR/Cas nuclease can be coupled to a guide nucleic acid that targets a particular region of interest in the ssRNA.

In some embodiments, the present disclosure provides methods and compositions for modifying or editing a target nucleic acid sequence comprising two or more programmable nucleases. For example, modifying a target nucleic acid may comprise introducing two or more single-stranded breaks in the target nucleic acid. In some embodiments, a break may be introduced by contacting a target nucleic acid with a programmable nuclease and a guide nucleic acid. The guide nucleic acid may bind to the programmable nuclease and hybridize to a region of the target nucleic acid, thereby recruiting the programmable nuclease to the region of the target nucleic acid. Binding of the programmable nuclease to the guide nucleic acid and the region of the target nucleic acid may activate the programmable nuclease, and the programmable nuclease may introduce a break (e.g., a single stranded break) in the region of the target nucleic acid. In some embodiments, modifying a target nucleic acid may comprise introducing a first break in a first region of the target nucleic acid and a second break in a second region of the target nucleic acid. For example, modifying a target nucleic acid may comprise contacting a target nucleic acid with a first guide nucleic acid that binds to a first programmable nuclease and hybridizes to a first region of the target nucleic acid and a second guide nucleic acid that binds to a second programmable nuclease and hybridizes to a second region of the target nucleic acid. The first programmable nuclease may introduce a first break in a first strand at the first region of the target nucleic acid, and the second programmable nuclease may introduce a second break in a second strand at the second region of the target nucleic acid. In some embodiments, a segment of the target nucleic acid between the first break and the second break may be removed, thereby modifying the target nucleic acid. In some embodiments, a segment of the target nucleic acid between the first break and the second break may be replaced (e.g., with an insert sequence), thereby modifying the target nucleic acid.

The donor polynucleotide can comprise a genomic nucleic acid. In some embodiments, a donor nucleic acid is a nucleic acid that is incorporated into a target nucleic acid or target sequence. In reference to a viral vector, a donor nucleic acid is a sequence of nucleotides that will be or has been introduced into a cell following transfection of the viral vector. In some embodiments, a viral vector is a nucleic acid to be delivered into a host cell via a recombinantly produced virus or viral particle. The nucleic acid may be single-stranded or double stranded, linear or circular, segmented or non-segmented. The nucleic acid may comprise DNA, RNA, or a combination thereof. Non-limiting examples of viruses or viral particles that can deliver a viral vector include retroviruses (e.g., lentiviruses and γ-retroviruses), adenoviruses, arenaviruses, alphaviruses, adeno-associated viruses (AAVs), baculoviruses, vaccinia viruses, herpes simplex viruses and poxviruses. A viral vector delivered by such viruses or viral particles may be referred to by the type of virus to deliver the viral vector (e.g., an AAV viral vector is a viral vector that is to be delivered by an adeno-associated virus). A viral vector referred to by the type of virus to be delivered by the viral vector can contain viral elements (e.g., nucleotide sequences) necessary for packaging of the viral vector into the virus or viral particle, replicating the virus, or other desired viral activities. A virus containing a viral vector may be replication competent, replication deficient or replication defective. The donor nucleic acid may be introduced into the cell by any mechanism of the transfecting viral vector, including, but not limited to, integration into the genome of the cell or introduction of an episomal plasmid or viral genome. As another example, when used in reference to the activity of a programmable nuclease, a donor nucleic acid is a sequence of nucleotides that will be or has been inserted at the site of cleavage by the programmable nuclease (cleaving (hydrolysis of a phosphodiester bond) of a nucleic acid resulting in a nick or double strand break-nuclease activity). As yet another example, when used in reference to homologous recombination, a donor nucleic acid is a sequence of DNA that serves as a template in the process of homologous recombination, which may carry the modification that is to be or has been introduced into the target nucleic acid. By using this donor nucleic acid as a template, the genetic information, including the modification, is copied into the target nucleic acid by way of homologous recombination.

The genomic nucleic acid can be derived from an animal, a mouse, a human, a non-human, a rodent, a non-human, a rat, a hamster, a rabbit, a pig, a bovine, a deer, a sheep, a goat, a chicken, a cat, a dog, a ferret, a primate (e.g., marmoset, rhesus monkey), domesticated mammal or an agricultural mammal, an avian, a bacterium, a archaeon, a virus, or any other organism of interest or a combination thereof.

Donor polynucleotides of any suitable size can be integrated into a genome. In some embodiments, the donor polynucleotide integrated into a genome is less than 3, about 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45, 50, 100, 150, 200, 250, 300, 350, 400, 450, 500 or more than 500 kilobases (kb) in length. In some embodiments, the donor polynucleotide integrated into a genome is at least about 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45, 50, 100, 150, 200, 250, 300, 350, 400, 450, 500 or more than 500 kb in length. In some embodiments, the donor polynucleotide integrated into a genome is up to about 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45, 50, 100, 150, 200, 250, 300, 350, 400, 450, 500 or more than 500 kb in length.

In some embodiments, gene modifying or gene editing is achieved by fusing a programmable nuclease such as a Type VI CRISPR/Cas protein to a heterologous sequence. The heterologous sequence can be a suitable fusion partner, e.g., a polypeptide that provides recombinase activity by acting on the target nucleic acid sequence. In some embodiments, the fusion protein comprises a programmable nuclease such as a Type VI CRISPR/Cas protein fused to a heterologous sequence by a linker. In some embodiments, a linker is a bond or molecule that links a first polypeptide to a second polypeptide. A peptide linker comprises at least two amino acids linked by an amide bond.

The heterologous sequence or fusion partner can be a base editing domain. The base editing domain can be an ADAR1/2 or any functional variant thereof.

The heterologous sequence or fusion partner can be fused to the C-terminus, N-terminus, or an internal portion (e.g., a portion other than the N- or C-terminus) of the programmable nuclease.

The heterologous sequence or fusion partner can be fused to the programmable nuclease by a linker. A linker can be a peptide linker or a non-peptide linker. In some embodiments, the linker is an XTEN linker. In some embodiments, the linker comprises one or more repeats a tri-peptide GGS. In some embodiments, the linker is from 1 to 100 amino acids in length. In some embodiments, the linker is more 100 amino acids in length. In some embodiments, the linker is from 10 to 27 amino acids in length. A non-peptide linker can be a polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, polycyanoacrylates, lipid polymers, chitins, hyaluronic acid, heparin, or an alkyl linker.

In some embodiments, the Type VI CRISPR/Cas protein can comprise an enzymatically inactive and/or “dead” (abbreviated by “d”) programmable nuclease in combination (e.g., fusion) with a polypeptide comprising recombinase activity. Although a programmable Type VI CRISPR/Cas nuclease normally has nuclease activity, in some embodiments, a programmable Type VI CRISPR/Cas nuclease does not have nuclease activity.

A programmable Type VI CRISPR/Cas nuclease can comprise a modified form of a wild type counterpart. The modified form of the wild type counterpart can comprise an amino acid change (e.g., deletion, insertion, or substitution) that reduces the nucleic acid-cleaving activity of the programmable nuclease. For example, a nuclease domain (e.g., HEPN domain) of a Type VI CRISPR/Cas polypeptide can be deleted or mutated so that it is no longer functional or comprises reduced nuclease activity. The modified form of the programmable nuclease can have less than 90%, less than 80%, less than 70%, less than 60%, less than 50%, less than 40%, less than 30%, less than 20%, less than 10%, less than 5%, or less than 1% of the nucleic acid-cleaving activity of the wild-type counterpart. The modified form of a programmable nuclease can have no substantial nucleic acid-cleaving activity. When a programmable nuclease is a modified form that has no substantial nucleic acid-cleaving activity, it can be referred to as enzymatically inactive and/or dead. A dead Type VI CRISPR/Cas polypeptide can bind to a target nucleic acid sequence but may not cleave the target nucleic acid sequence. A dead Type VI CRISPR/Cas polypeptide can associate with a guide nucleic acid to activate or repress transcription of a target nucleic acid sequence.

In some embodiments, a programmable nuclease is a dead Type VI CRISPR/Cas protein. A dead Type VI CRISPR/Cas polypeptide can comprise at least 50%, at least 55%, at least 60%, at least 65%, at least 70%, at least 75%, at least 80%, at least 85%, at least 90%, at least 92%, at least 95%, at least 97%, at least 98%, at least 99%, or 100% sequence identity with any one of SEQ ID NO: 1-SEQ ID NO: 27. In some embodiments, a programmable nuclease is a dead Type VI CRISPR/Cas polypeptide comprising at least 50% sequence identity to any one of SEQ ID NO: 1-SEQ ID NO: 27. In some embodiments, a programmable nuclease is a dead Type VI CRISPR/Cas polypeptide comprising at least 55% sequence identity to any one of SEQ ID NO: 1-SEQ ID NO: 27. In some embodiments, a programmable nuclease is a dead Type VI CRISPR/Cas polypeptide comprising at least 60% sequence identity to any one of SEQ ID NO: 1-SEQ ID NO: 27. In some embodiments, a programmable nuclease is a dead Type VI CRISPR/Cas polypeptide comprising at least 65% sequence identity to any one of SEQ ID NO: 1-SEQ ID NO: 27. In some embodiments, a programmable nuclease is a dead Type VI CRISPR/Cas polypeptide comprising at least 70% sequence identity to any one of SEQ ID NO: 1-SEQ ID NO: 27. In some embodiments, a programmable nuclease is a dead Type VI CRISPR/Cas polypeptide comprising at least 75% sequence identity to any one of SEQ ID NO: 1-SEQ ID NO: 27. In some embodiments, a programmable nuclease is a dead Type VI CRISPR/Cas polypeptide comprising at least 80% sequence identity to any one of SEQ ID NO: 1-SEQ ID NO: 27. In some embodiments, a programmable nuclease is a dead Type VI CRISPR/Cas polypeptide comprising at least 85% sequence identity to any one of SEQ ID NO: 1-SEQ ID NO: 27. In some embodiments, a programmable nuclease is a dead Type VI CRISPR/Cas polypeptide comprising at least 90% sequence identity to any one of SEQ ID NO: 1-SEQ ID NO: 27. In some embodiments, a programmable nuclease is a dead Type VI CRISPR/Cas polypeptide comprising at least 95% sequence identity to any one of SEQ ID NO: 1-SEQ ID NO: 27. In some embodiments, a programmable nuclease is a dead Type VI CRISPR/Cas polypeptide comprising at least 98% sequence identity to any one of SEQ ID NO: 1-SEQ ID NO: 27.

Enzymatically inactive can refer to a polypeptide that can bind to a nucleic acid sequence in a polynucleotide in a sequence-specific manner but may not cleave a target polynucleotide. An enzymatically inactive site-directed polypeptide can comprise an enzymatically inactive domain (e.g. a programmable nuclease domain). Enzymatically inactive can refer to no activity. Enzymatically inactive can refer to substantially no activity. Enzymatically inactive can refer to essentially no activity. Enzymatically inactive can refer to an activity less than 1%, less than 2%, less than 3%, less than 4%, less than 5%, less than 6%, less than 7%, less than 8%, less than 9%, or less than 10% activity compared to a wild-type exemplary activity (e.g., nucleic acid cleaving activity, wild-type Type VI CRISPR/Cas protein activity).

### Inducing Cell Death by Trans-Cleavage of RNA

Compositions and methods disclosed herein may induce cell death by trans-cleavage of RNA. In some embodiments, enzymes described herein, e.g., enzymes with identity to any one of SEQ ID NOs: 1-27 of the present application, may be used to perform trans-cleavage of RNA, causing cell cycle arrest, apoptosis, and/or cell death. In some embodiments, trans-cleavage activity causes non-specific cleavage of nearby single-stranded nucleic acids by an activated programmable nuclease. Cell cycle arrest, apoptosis, cell death, or a combination thereof may be induced by contacting a Cas protein and a guide nucleic acid molecule to a target nucleic acid within the cell, wherein the guide nucleic acid molecule is complementary to at least a portion of a target sequence in the target nucleic acid, and wherein hybridization of the guide nucleic acid molecule to the target sequence activates non-specific cleavage of RNA in the cell, thereby inducing cell cycle arrest, apoptosis, cell death, or a combination thereof, of the cell. In some instances, the target nucleic acid comprises a genetic mutation, and thus, cell death occurs primarily in cells comprising the genetic mutation. This method may be used to treat diseases such as cancer, autoimmune disease, and infectious disease. The guide nucleic acid molecule may be a nucleotide sequence that is identical or reverse complementary to a target sequence of a target nucleic acid, wherein the target sequence comprises a mutation of at least one nucleotide relative to a corresponding wildtype sequence. Exemplary target nucleic acids are described below and throughout.

### Methods of Detecting a Target Nucleic Acid

Provided herein, in some embodiments, is a method of detecting a target nucleic acid in a sample, comprising contacting a target nucleic acid with any one of the compositions or systems described herein. In some embodiments, the method comprises contacting the sample with a reporter nucleic acid. In some embodiments, the method comprises measuring a detectable signal produced by cleavage of the reporter nucleic acid. In some embodiments, a detectable signal is a signal that can be detected using optical, fluorescent, chemiluminescent, electrochemical and other detection methods known in the art.

In some embodiments, contacting occurs at a temperature of at least about 40° C., at least about 50° C., at least about 55° C., at least about 60° C., or at least about 65° C. In some embodiments, contacting occurs at a temperature of at least about 55° C. In some embodiments, contacting occurs at a temperature of at least about 60° C. In some embodiments, contacting occurs at a temperature of at least about 65° C. In some embodiments, contacting occurs at a temperature not greater than 45° C. In some embodiments, contacting occurs at a temperature of about 45° C. In some embodiments, contacting occurs at a temperature not greater than 70° C. In some embodiments, contacting occurs at a temperature of about 0° C., about 10° C., about 20° C., about 30° C., about 40° C., about 50° C., about 55° C., about 60° C., about 65° C., or about 70° C. In some embodiments, contacting occurs at a temperature of about 55° C. In some embodiments, contacting occurs at a temperature of about 60° C. In some embodiments, contacting occurs at a temperature of about 65° C. In some embodiments, contacting occurs at a temperature of about 70° C. In some embodiments, the method further comprises amplifying the target nucleic acid. In some embodiments, the amplifying is performed before contacting. In some embodiments, the amplifying is performed during contacting. In some embodiments, amplifying occurs at a temperature of at least about 55° C. In some embodiments, amplifying occurs at a temperature of at least about 60° C. In some embodiments, amplifying occurs at a temperature of at least about 65° C. In some embodiments, amplifying occurs at a temperature not greater than 70° C. In some embodiments, amplifying occurs at a temperature of about 55° C. In some embodiments, amplifying occurs at a temperature of about 60° C. In some embodiments, amplifying occurs at a temperature of about 65° C. In some embodiments, amplifying occurs at a temperature of about 70° C. In some embodiments, amplifying comprises isothermal amplification. In some embodiments, amplification and/or amplifying is a process by which a nucleic acid molecule is enzymatically copied to generate a plurality of nucleic acid molecules containing the same sequence as the original nucleic acid molecule or a distinguishable portion thereof. In some embodiments, amplification is isothermal amplification or polymerase chain reaction (PCR). In some embodiments, amplifying occurs at a temperature of around 20° C.-70° C. In some embodiments, amplifying occurs at a temperature of around 0° C.-10° C., 0° C.-20° C., 10° C.-20° C., 20° C.-40° C., 25° C.-40° C., 30° C.-40° C., 35° C.-40° C., 30° C.-50° C., 35° C.-50° C., 40° C.-50° C., 45° C.-50° C., 45° C.-60° C., 50° C.-60° C., 55° C.-60° C., 50° C.-70° C., 55° C.-70° C., or 60° C.-70° C. In some embodiments, the programmable nuclease is from a mesophilic organism. In some embodiments, the programmable nuclease is active between 20° C.-70° C. In some embodiments, the programmable nuclease is active between 0° C.-10° C., 0° C.-20° C., 10° C.-20° C., 20° C.-40° C., 25° C.-40° C., 30° C.-40° C., 35° C.-40° C., 30° C.-50° C., 35° C.-50° C., 40° C.-50° C., 45° C.-50° C., 45° C.-60° C., 50° C.-60° C., 55° C.-60° C., 50° C.-70° C., 55° C.-70° C., or 60° C.-70° C. In some embodiments, the programmable nuclease is active at room temperature. In some embodiments, the method further comprises transcribing DNA in the sample to produce the target nucleic acid. In some embodiments, the contacting and the transcribing are carried out at the same temperature. In some embodiments, the contacting, detecting, amplifying, transcribing, or any combination thereof, are carried out at the same temperature. In some embodiments, the contacting, detecting, amplifying, transcribing, or any combination thereof, are carried out in a single reaction chamber. In some embodiments, the sample, or portion thereof, is from a pathogen. In some embodiments, the pathogen is a virus or a bacterium. In some embodiments, the virus is a coronavirus. In some embodiments, the coronavirus is SARS-CoV-2 virus. In some embodiments, the virus is an influenza virus. In some embodiments, the influenza virus is influenza A virus or influenza B virus. In some embodiments, the virus is a human papillomavirus or a herpes simplex virus. In some embodiments, the virus is a respiratory syncytial virus, or a combination thereof. In some embodiments, the pathogen is a bacterium. In some embodiments, the bacterium is a Chlamydia trachomatis. In some embodiments, the programmable nuclease provides cis-cleavage activity on the target nucleic acid. In some embodiments, cis cleavage and/or cis-cleavage is cleavage (hydrolysis of a phosphodiester bond) of a target nucleic acid by a programmable nuclease complexed with a guide nucleic acid refers to cleavage of a target nucleic acid that is hybridized to a guide nucleic acid, wherein cleavage occurs within or directly adjacent to the region of the target nucleic acid that is hybridized to the guide nucleic acid. In some embodiments, the programmable nuclease provides transcollateral cleavage activity on the target nucleic acid. In some embodiments, trans cleavage (or transcollateral cleavage) is cleavage (hydrolysis of a phosphodiester bond) of one or more nucleic acids by a programmable nuclease that is complexed with a guide nucleic acid and a target nucleic acid. The one or more nucleic acids may include the target nucleic acid as well as non-target nucleic acids. Trans cleavage may occur near, but not within or directly adjacent to, the region of the target nucleic acid that is hybridized to the guide nucleic acid. Trans cleavage activity may be triggered by the hybridization of the guide nucleic acid to the target nucleic acid.

The present disclosure provides methods and compositions, which enable target nucleic acid detection by programmable nuclease platforms, such as the DNA/RNA Endonuclease Targeted CRISPR TransReporter (DETECTR) platform. In some embodiments, the target nucleic acid is an RNA.

A number of reagents are consistent with the compositions and methods disclosed herein. The reagents described herein may be used for target nucleic acids and for detection of target nucleic acids. The reagents disclosed herein can include programmable nucleases, guide nucleic acids, target nucleic acids, and buffers. As described herein, target nucleic acid comprising RNA may be modified or detected (e.g., the target nucleic acid hybridizes to the guide nucleic) using a programmable Type VI CRISPR/Cas nuclease and other reagents disclosed herein. As described herein, target nucleic acids comprising DNA may be an amplicon of a nucleic acid of interest and the amplicon can be detected using a programmable Type VI CRISPR/Cas nuclease and other reagents disclosed herein. Additionally, detection of multiple target nucleic acids is possible using two or more programmable nucleases or a programmable nuclease with a non-nuclease programmable nuclease complexed to guide nucleic acids that target the multiple target nucleic acids, wherein the programmable nucleases exhibit different sequence-independent cleavage of the nucleic acid of a reporter (e.g., cleavage of an RNA reporter by a first programmable nuclease and cleavage of a RNA reporter by a second programmable nuclease).

Certain programmable Type VI CRISPR/Cas nucleases of the disclosure can exhibit indiscriminate trans-cleavage of ssRNA or ssDNA, enabling their use for detection of RNA in samples. In some embodiments, target ssRNA are generated from many nucleic acid templates (RNA) in order to achieve cleavage of the reporter (e.g., FQ reporter) in the DETECTR platform. Certain programmable nucleases can be activated by ssRNA, upon which they can exhibit trans-cleavage of ssRNA and can, thereby, be used to cleave ssRNA FQ reporter molecules in the DETECTR system. These programmable nucleases can target ssRNA present in the sample, or generated and/or amplified from any number of nucleic acid templates (RNA).

The compositions, kits and methods disclosed herein may be implemented in methods of assaying for a target nucleic acid. In some embodiments, a method of assaying for a target nucleic acid in a sample, comprises: contacting the sample to a complex comprising a guide nucleic acid comprising a segment that is reverse complementary to a segment of the target nucleic acid and a programmable Type VI CRISPR/Cas nuclease of the disclosure that exhibits sequence independent cleavage upon forming a complex comprising the segment of the guide nucleic acid binding to the segment of the target nucleic acid, wherein the sample comprises at least one nucleic acid comprising at least 50% sequence identity to the segment of the target nucleic acid; and assaying for cleavage of at least one reporter nucleic acids of a population of reporter nucleic acids, wherein the cleavage indicates a presence of the target nucleic acid in the sample and wherein absence of the cleavage indicates an absence of the target nucleic acid in the sample.

The target nucleic acid can be from 0.05% to 20% of total nucleic acids in the sample. Sometimes, the target nucleic acid is from 0.1% to 10% of the total nucleic acids in the sample. The target nucleic acid, in some cases, is from 0.1% to 5% of the total nucleic acids in the sample. Often, a sample comprises the segment of the target nucleic acid and at least one nucleic acid comprising less than 100% sequence identity to the segment of the target nucleic acid but no less than 50% sequence identity to the segment of the target nucleic acid. For example, the segment of the target nucleic acid comprises a mutation as compared to at least one nucleic acid comprising less than 100% sequence identity to the segment of the target nucleic acid but no less than 50% sequence identity to the segment of the target nucleic acid. Often, the segment of the target nucleic acid comprises a single nucleotide mutation as compared to at least one nucleic acid comprising less than 100% sequence identity to the segment of the target nucleic acid but no less than 50% sequence identity to the segment of the target nucleic acid.

The concentrations of the various reagents in the programmable nuclease DETECTR reaction mix can vary depending on the particular scale of the reaction. For example, the final concentration of the programmable nuclease can vary from 1 pM to 1 nM, from 1 pM to 10 pM, from 10 pM to 100 pM, from 100 pM to 1 nM, from 1 nM to 10 nM, from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1000 nM. The final concentration of the sgRNA complementary to the target nucleic acid can be from 1 pM to 1 nM, from 1 pM to 10 pM, from 10 pM to 100 pM, from 100 pM to 1 nM, from 1 nM to 10 nM, from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1000 nM. The concentration of the ssDNA-FQ reporter can be from from 1 pM to 1 nM, from 1 pM to 10 pM, from 10 pM to 100 pM, from 100 pM to 1 nM, from 1 nM to 10 nM, from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1000 nM.

An example of a DETECTR reaction comprises, consists, or consists essentially of a final concentration of 100 nM Type VI CRISPR/Cas polypeptide or variant thereof, 125 nM sgRNA, and 50 nM ssRNA-FQ reporter in a total reaction volume of 20 μL. Reactions are incubated in a fluorescence plate reader (Tecan Infinite Pro 200 M Plex) for 2 hours at 37° C. with fluorescence measurements taken every 30 seconds (e.g., kex: 485 nm; kem: 535 nm). The fluorescence wavelength detected can vary depending on the reporter molecule.

Described herein are reagents comprising a single stranded reporter nucleic acid comprising a detection moiety, wherein the reporter nucleic acid (e.g., the ssDNA-FQ reporter described herein) is capable of being cleaved by the programmable nuclease, upon generation and amplification of ssRNA from a nucleic acid template using the methods disclosed herein, thereby generating a first detectable signal.

In some embodiments, methods described herein for detecting a target nucleic acid include wherein the target nucleic acid is from a sample, or portion thereof, of a diagnostic target of interest. For example, in some embodiments, the diagnostic target of interest selected from a coronavirus (229E, HKU1, NL63, OC43), MERS-CoV, SARS-CoV-2 (WT, alpha, beta, gamma, delta, epsilon, eta, iota, kappa, 1.617.3, mu, omicron, zeta, and other variants thereof), a human metapneumovirus, a rhinovirus, an enterovirus, influenza A (H1N1, H3N2, etc. including H1-H16 and N1-N9 proteins), influenza B (Victoria VIA, Yamagata Y1/Y2/Y3), parainfluenza 1, 2, 3, 4, 4a, a respiratory syncytial virus A (RSV-A), a respiratory syncytial virus B (RSV-B), a gammacoronavirus, a deltacoronavirus, a betacoronavirus, an alphacoronavirus, a sarbecovirus subgenus, a SARS-related virus, Bordetella pertussis, Bordetella parapertussis, Bordetella bronchoseptica, Bordetella holmesii, Chlamydophila pneumoniae, Legionella pneumophila, Mycoplasma pneumoniae, a human bocavirus, and a human adenovirus (Types A, B, C, D, E, F, or G). In some embodiments, the target nucleic acid is a combination of diagnostic targets of interest. Accordingly, in some embodiments, the methods described herein can detect a combination of target nucleic acids from a sample or samples from the diagnostic targets of interest, including, for example, detecting target nucleic acids from two, three, four, five, six, seven, eight, nine, ten or more different diagnostic targets of interest.

In some embodiments, methods described herein include use of a control. Accordingly, in some embodiments, the methods described herein include use of a positive control. In some embodiments, the methods described herein include the use of a negative control. In some embodiments, the methods described herein include use of a control for determining relative abundance of the target nucleic acid compared to the control. Examples of controls that can be used in the methods described herein include human 18S, 28S rRNA, GAPDH, RNaseP, human HRPT1, and human GUSB.

### Reporters

Described herein are reagents comprising a reporter. The reporter can comprise a single stranded nucleic acid and a detection moiety (e.g., a labeled single stranded RNA reporter), wherein the nucleic acid is capable of being cleaved by the activated programmable nuclease (e.g., a Type VI CRISPR/Cas protein as disclosed herein), releasing the detection moiety, and, generating a detectable signal. As used herein, “reporter” is used interchangeably with “reporter nucleic acid” or “reporter molecule”. The programmable nucleases disclosed herein, activated upon hybridization of a guide RNA to a target nucleic acid, can cleave the reporter. Cleaving the “reporter” may be referred to herein as cleaving the “reporter nucleic acid,” the “reporter molecule,” or the “nucleic acid of the reporter.”

A major advantage of the compositions and methods disclosed herein can be the design of excess reporters to total nucleic acids in an unamplified or an amplified sample, not including the nucleic acid of the reporter. Total nucleic acids can include the target nucleic acids and non-target nucleic acids, not including the nucleic acid of the reporter. The non-target nucleic acids can be from the original sample, either lysed or unlysed. The non-target nucleic acids can also be byproducts of amplification. Thus, the non-target nucleic acids can include both non-target nucleic acids from the original sample, lysed or unlysed, and from an amplified sample. The presence of a large amount of non-target nucleic acids, an activated programmable nuclease (e.g., a Type VI CRISPR/Cas protein as disclosed herein) may be inhibited in its ability to bind and cleave the reporter sequences. This is because the activated programmable nucleases collaterally cleaves any nucleic acids. If total nucleic acids are present in large amounts, they may outcompete reporters for the programmable nucleases. The compositions and methods disclosed herein are designed to have an excess of reporter to total nucleic acids, such that the detectable signals from DETECTR reactions are particularly superior. In some embodiments, the reporter can be present in at least 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold, from 1.5 fold to 100 fold, from 2 fold to 10 fold, from 10 fold to 20 fold, from 20 fold to 30 fold, from 30 fold to 40 fold, from 40 fold to 50 fold, from 50 fold to 60 fold, from 60 fold to 70 fold, from 70 fold to 80 fold, from 80 fold to 90 fold, from 90 fold to 100 fold, from 1.5 fold to 10 fold, from 1.5 fold to 20 fold, from 10 fold to 40 fold, from 20 fold to 60 fold, or from 10 fold to 80 fold excess of total nucleic acids.

Another significant advantage of the compositions and methods disclosed herein can be the design of an excess volume comprising the guide nucleic acid, the programmable nuclease (e.g., a Type VI CRISPR/Cas protein as disclosed herein), and the reporter, which contacts a smaller volume comprising the sample with the target nucleic acid of interest. The smaller volume comprising the sample can be unlysed sample, lysed sample, or lysed sample which has undergone any combination of reverse transcription, amplification, and in vitro transcription. The presence of various reagents in a crude, non-lysed sample, a lysed sample, or a lysed and amplified sample, such as buffer, magnesium sulfate, salts, the pH, a reducing agent, primers, dNTPs, NTPs, cellular lysates, non-target nucleic acids, primers, or other components, can inhibit the ability of the programmable nuclease to become activated or to find and cleave the nucleic acid of the reporter. This may be due to nucleic acids that are not the reporter outcompeting the nucleic acid of the reporter, for the programmable nuclease. Alternatively, various reagents in the sample may simply inhibit the activity of the programmable nuclease. Thus, the compositions and methods provided herein for contacting an excess volume comprising the engineered guide nucleic acid, the programmable nuclease, and the reporter to a smaller volume comprising the sample with the target nucleic acid of interest provides for superior detection of the target nucleic acid by ensuring that the programmable nuclease is able to find and cleaves the nucleic acid of the reporter. In some embodiments, the volume comprising the guide nucleic acid, the programmable nuclease, and the reporter (can be referred to as “a second volume”) is 4-fold greater than a volume comprising the sample (can be referred to as “a first volume”). In some embodiments, the volume comprising the guide nucleic acid, the programmable nuclease, and the reporter (can be referred to as “a second volume”) is at least 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold, from 1.5 fold to 100 fold, from 2 fold to 10 fold, from 10 fold to 20 fold, from 20 fold to 30 fold, from 30 fold to 40 fold, from 40 fold to 50 fold, from 50 fold to 60 fold, from 60 fold to 70 fold, from 70 fold to 80 fold, from 80 fold to 90 fold, from 90 fold to 100 fold, from 1.5 fold to 10 fold, from 1.5 fold to 20 fold, from 10 fold to 40 fold, from 20 fold to 60 fold, or from 10 fold to 80 fold greater than a volume comprising the sample (can be referred to as “a first volume”). In some embodiments, the volume comprising the sample is at least 0.5 μL, at least 1 μL, at least at least 1 μL, at least 2 μL, at least 3 μL, at least 4 μL, at least 5 μL, at least 6 μL, at least 7 μL, at least 8 μL, at least 9 μL, at least 10 μL, at least 11 μL, at least 12 μL, at least 13 μL, at least 14 μL, at least 15 μL, at least 16 μL, at least 17 μL, at least 18 μL, at least 19 μL, at least 20 μL, at least 25 μL, at least 30 μL, at least 35 μL, at least 40 μL, at least 45 μL, at least 50 μL, at least 55 μL, at least 60 μL, at least 65 μL, at least 70 μL, at least 75 μL, at least 80 μL, at least 85 μL, at least 90 μL, at least 95 μL, at least 100 μL, from 0.5 μL to 5 μL pL, from 5 μL to 10 μL, from 10 μL to 15 μL, from 15 μL to 20 μL, from 20 μL to 25 μL, from 25 μL to 30 μL, from 30 μL to 35 μL, from 35 μL to 40 μL, from 40 μL to 45 μL, from 45 μL to 50 μL, from 10 μL to 20 μL, from 5 μL to 20 μL, from 1 μL to 40 μL, from 2 μL to 10 μL, or from 1 μL to 10 μL. In some embodiments, the volume comprising the programmable nuclease, the guide nucleic acid, and the reporter is at least 10 μL, at least 11 μL, at least 12 μL, at least 13 μL, at least 14 μL, at least 15 μL, at least 16 μL, at least 17 μL, at least 18 μL, at least 19 μL, at least 20 μL, at least 21 μL, at least 22 μL, at least 23 μL, at least 24 μL, at least 25 μL, at least 26 μL, at least 27 μL, at least 28 μL, at least 29 μL, at least 30 μL, at least 40 μL, at least 50 μL, at least 60 μL, at least 70 μL, at least 80 μL, at least 90 μL, at least 100 μL, at least 150 μL, at least 200 μL, at least 250 μL, at least 300 μL, at least 350 μL, at least 400 μL, at least 450 μL, at least 500 μL, from 10 μL to 15 μL μL, from 15 μL to 20 μL, from 20 μL to 25 μL, from 25 μL to 30 μL, from 30 μL to 35 μL, from 35 μL to 40 μL, from 40 μL to 45 μL, from 45 μL to 50 μL, from 50 μL to 55 μL, from 55 μL to 60 μL, from 60 μL to 65 μL, from 65 μL to 70 μL, from 70 μL to 75 μL, from 75 μL to 80 μL, from 80 μL to 85 μL, from 85 μL to 90 μL, from 90 μL to 95 μL, from 95 μL to 100 μL, from 100 μL to 150 μL, from 150 μL to 200 μL, from 200 μL to 250 μL, from 250 μL to 300 μL, from 300 μL to 350 μL, from 350 μL to 400 μL, from 400 μL to 450 μL, from 450 μL to 500 μL, from 10 μL to 20 μL, from 10 μL to 30 μL, from 25 μL to 35 μL, from 10 μL to 40 μL, from 20 μL to 50 μL, from 18 μL to 28 μL, or from 17 μL to 22 μL.

In some cases, the reporter nucleic acid is a single-stranded nucleic acid sequence comprising ribonucleotides. The nucleic acid of a reporter can be a single-stranded nucleic acid sequence comprising at least one ribonucleotide. In some cases, the nucleic acid of a reporter is a single-stranded nucleic acid comprising at least one ribonucleotide residue at an internal position that functions as a cleavage site. In some cases, the nucleic acid of a reporter comprises at least 2, 3, 4, 5, 6, 7, 8, 9, or 10 ribonucleotide residues at an internal position. In some cases, the nucleic acid of a reporter comprises from 2 to 10, from 3 to 9, from 4 to 8, or from 5 to 7 ribonucleotide residues at an internal position. Sometimes the ribonucleotide residues are continuous. Alternatively, the ribonucleotide residues are interspersed in between non-ribonucleotide residues. In some cases, the nucleic acid of a reporter has only ribonucleotide residues. In some cases, the nucleic acid of a reporter has only deoxyribonucleotide residues. In some cases, the nucleic acid comprises nucleotides resistant to cleavage by the programmable nuclease described herein. In some cases, the nucleic acid of a reporter comprises synthetic nucleotides. In some cases, the nucleic acid of a reporter comprises at least one ribonucleotide residue and at least one non-ribonucleotide residue. In some cases, the nucleic acid of a reporter is 5-20, 5-15, 5-10, 7-20, 7-15, or 7-10 nucleotides in length. In some cases, the nucleic acid of a reporter is from 3 to 20, from 4 to 10, from 5 to 10, or from 5 to 8 nucleotides in length. In some cases, the nucleic acid of a reporter comprises at least one uracil ribonucleotide. In some cases, the nucleic acid of a reporter comprises at least two uracil ribonucleotides. Sometimes the nucleic acid of a reporter has only uracil ribonucleotides. In some cases, the nucleic acid of a reporter comprises at least one adenine ribonucleotide. In some cases, the nucleic acid of a reporter comprises at least two adenine ribonucleotide. In some cases, the nucleic acid of a reporter has only adenine ribonucleotides. In some cases, the nucleic acid of a reporter comprises at least one cytosine ribonucleotide. In some cases, the nucleic acid of a reporter comprises at least two cytosine ribonucleotide. In some cases, the nucleic acid of a reporter comprises at least one guanine ribonucleotide. In some cases, the nucleic acid of a reporter comprises at least two guanine ribonucleotide. A nucleic acid of a reporter can comprise only unmodified ribonucleotides, only unmodified deoxyribonucleotides, or a combination thereof. In some cases, the nucleic acid of a reporter is from 5 to 12 nucleotides in length. In some cases, the reporter nucleic acid is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides in length. In some cases, the reporter nucleic acid is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length.

In some cases, the reporter comprises a detection moiety. In some instances, the reporter comprises a cleavage site, wherein the detection moiety is located at a first site on the reporter, wherein the first site is separated from the remainder of reporter upon cleavage at the cleavage site. In some cases, the detection moiety is 3′ to the cleavage site. In some cases, the detection moiety is 5′ to the cleavage site. Sometimes the detection moiety is at the 3′ terminus of the nucleic acid of a reporter. In some cases, the detection moiety is at the 5′ terminus of the nucleic acid of a reporter.

In some embodiments, the detection moiety comprises an enzyme, a radioisotope, a member of a specific binding pair, a fluorophore, a fluorescent protein, a quantum dot, and the like.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Suitable enzymes include, but are not limited to, horseradish peroxidase (HRP), alkaline phosphatase (AP), beta-galactosidase (GAL), glucose-6-phosphate dehydrogenase, beta-N-acetylglucosaminidase, CE≤-glucuronidase, invertase, Xanthine Oxidase, firefly luciferase, glucose oxidase (GO), acetylcholinesterase, catalase, catacolase, tyronase, nitrocefelin, alkaline phosphatase, or invertase.

In some embodiments, the enzyme may bind with an enzyme substrate and produce a detectable signal. In some embodiments, the enzyme substrate may be 3,3′,5,5′-tetramethylbenzidine (TMB), 2,2′-Azinobis [3-ethylbenzothiazoline-6-sulfonic acid]-diammonium salt (ABTS), o-phenylenediamine dihydrochloride (OPD), p-Nitrophenyl Phosphate (PNPP), o-nitrophenyl-β-D-galactopyranoside (ONPG), 3,3′-diaminobenzidine (DAB), p-hydroxyphenylacetic acid, 3-(p-hydroxyphenyl)-propionic acid, homovanillic acid, or o-aminophenol. In some embodiments, the enzyme substrate may be a commercial enzyme substrate including SuperSignal ELISA Pico, SuperSignal Elisa Femto, CDP-Star Substrate, CSPD Substrate, DynaLight Substrate with RapidGlow Enhancer, QuantaBlu, QuantaRed, or Amplex.

In some instances, the detection moiety comprises an invertase. The substrate of the invertase may be sucrose. A DNS reagent may be included in the system to produce a colorimetric change when the invertase converts sucrose to glucose. In some cases, the reporter nucleic acid and invertase are conjugated using a heterobifunctional linker via sulfo-SMCC chemistry.

In some instances, the detection moiety comprises a horseradish peroxidase (HRP). The substrate of HRP may be TMB. In some embodiments, enzyme-modified reporters may be immobilized to a surface and configured to release the enzyme upon cleavage of a nucleic acid of the reporter by an activated programmable nuclease-guide complex bound to a target nucleic acid as described herein. Released HRP may then be contacted to its substrate, for example TMB, to generate a detectable signal indicative of cleavage of the reporter and presence of the target nucleic acid.

In some embodiments, the enzyme may generate a colorimetric signal, a fluorescent signal, an electrochemical signal, a chemiluminescent signal, or another type of signal. In some embodiments, the enzyme may induce color-change in substances.

The single stranded nucleic acid of a reporter comprises a detection moiety capable of generating a first detectable signal. Sometimes the detection moiety comprises a protein capable of generating a signal. A signal can be a calorimetric, potentiometric, amperometric, optical (e.g., fluorescent, colorimetric, etc.), or piezo-electric signal. In some cases, a detection moiety is on one side of the cleavage site. Optionally, a quenching moiety is on the other side of the cleavage site. Sometimes the quenching moiety is a fluorescence quenching moiety. In some cases, the quenching moiety is 5′ to the cleavage site and the detection moiety is 3′ to the cleavage site. In some cases, the detection moiety is 5′ to the cleavage site and the quenching moiety is 3′ to the cleavage site. Sometimes the quenching moiety is at the 5′ terminus of the nucleic acid of a reporter. Sometimes the detection moiety is at the 3′ terminus of the nucleic acid of a reporter. In some cases, the detection moiety is at the 5′ terminus of the nucleic acid of a reporter. In some cases, the quenching moiety is at the 3′ terminus of the nucleic acid of a reporter. In some cases, the single-stranded nucleic acid of a reporter is at least one population of the single-stranded nucleic acid capable of generating a first detectable signal. In some cases, the single-stranded nucleic acid of a reporter is a population of the single stranded nucleic acid capable of generating a first detectable signal. Optionally, there is more than one population of single-stranded nucleic acid of a reporter. In some cases, there are 2, 3, 4, 5, 6, 7, 8, 9, 10, 11, 12, 13, 14, 15, 20, 30, 40, 50, or greater than 50, or any number spanned by the range of this list of different populations of single-stranded nucleic acids of a reporter capable of generating a detectable signal. In some cases, there are from 2 to 50, from 3 to 40, from 4 to 30, from 5 to 20, or from 6 to 10 different populations of single-stranded nucleic acids of a reporter capable of generating a detectable signal.

TABLE 3
Examples of Single Stranded Nucleic Acids in a Reporter

| 5′ Detection | | 3′ |
|---|---|---|
| Moiety* | Sequence (SEQ ID NO:) | Quencher* |
| /56-FAM/ | rUrUrUrUrU (SEQ ID NO: 33) | /3IABKFQ/ |
| /5IRD700/ | rUrUrUrUrU (SEQ ID NO: 33) | /3IRQCIN/ |
| /5TYE665/ | rUrUrUrUrU (SEQ ID NO: 33) | /3IAbRQSp/ |
| /5Alex594N/ | rUrUrUrUrU (SEQ ID NO: 33) | /3IAbRQSp/ |
| /5ATTO633N/ | rUrUrUrUrU (SEQ ID NO: 33) | /3IAbRQSp/ |
| /56-FAM/ | rUrUrUrUrUrUrUrU (SEQ ID NO: 34) | /3IABKFQ/ |
| /5IRD700/ | rUrUrUrUrUrUrUrU (SEQ ID NO: 34) | /3IRQCIN/ |
| /5TYE665/ | rUrUrUrUrUrUrUrU (SEQ ID NO: 34) | /3IAbRQSp/ |
| /5Alex594N/ | rUrUrUrUrUrUrUrU (SEQ ID NO: 34) | /3IAbRQSp/ |
| /5ATTO633N/ | rUrUrUrUrUrUrUrU (SEQ ID NO: 34) | /3IAbRQSp/ |
| /56-FAM/ | rUrUrUrUrUrUrUrUrUrU (SEQ ID NO: | /3IABKFQ/ |
| | 35) | |
| /5IRD700/ | rUrUrUrUrUrUrUrUrUrU (SEQ ID NO: | /3IRQCIN/ |
| | 35) | |
| /5TYE665/ | rUrUrUrUrUrUrUrUrUrU (SEQ ID NO: | /3IAbRQSp/ |
| | 35) | |
| /5Alex594N/ | rUrUrUrUrUrUrUrUrUrU (SEQ ID NO: | /3IAbRQSp/ |
| | 35) | |
| /5ATTO633N/ | rUrUrUrUrUrUrUrUrUrU (SEQ ID NO: | /3IAbRQSp/ |
| | 35) | |
| /56-FAM/ | TTTTrUrUTTTT (SEQ ID NO: 36) | /3IABKFQ/ |
| /5IRD700/ | TTTTrUrUTTTT (SEQ ID NO: 36) | /3IRQCIN/ |
| /5TYE665/ | TTTTrUrUTTTT (SEQ ID NO: 36) | /3IAbRQSp/ |
| /5 Alex594N/ | TTTTrUrUTTTT (SEQ ID NO: 36) | /3IAbRQSp/ |
| /5ATTO633N/ | TTTTrUrUTTTT (SEQ ID NO: 36) | /3IAbRQSp/ |
| /56-FAM/ | TTrUrUTT (SEQ ID NO: 37) | /3IABKFQ/ |
| /5IRD700/ | TTrUrUTT (SEQ ID NO: 37) | /3IRQCIN/ |
| /5TYE665/ | TTrUrUTT (SEQ ID NO: 37) | /3IAbRQSp/ |
| /5Alex594N/ | TTrUrUTT (SEQ ID NO: 37) | /3IAbRQSp/ |
| /5ATTO633N/ | TTrUrUTT (SEQ ID NO: 37) | /3IAbRQSp/ |
| /56-FAM/ | TArArUGC (SEQ ID NO: 38) | /3IABKFQ/ |
| /5IRD700/ | TArArUGC (SEQ ID NO: 38) | /3IRQCIN/ |
| /5TYE665/ | TArArUGC (SEQ ID NO: 38) | /3IAbRQSp/ |
| /5Alex594N/ | TArArUGC (SEQ ID NO: 38) | /3IAbRQSp/ |
| /5ATTO633N/ | TArArUGC (SEQ ID NO: 38) | /3IAbRQSp/ |
| /56-FAM/ | TArUrGGC (SEQ ID NO: 39) | /3IABKFQ/ |
| /5IRD700/ | TArUrGGC (SEQ ID NO: 39) | /3IRQCIN/ |
| /5TYE665/ | TArUrGGC (SEQ ID NO: 39) | /3IAbRQSp/ |
| /5Alex594N/ | TArUrGGC (SEQ ID NO: 39) | /3IAbRQSp/ |
| /5ATTO633N/ | TArUrGGC (SEQ ID NO: 39) | /3IAbRQSp/ |
| /56-FAM/ | rUrUrUrUrU (SEQ ID NO: 40) | /3IABKFQ/ |
| /5IRD700/ | rUrUrUrUrU (SEQ ID NO: 40) | /3IRQCIN/ |
| /5TYE665/ | rUrUrUrUrU (SEQ ID NO: 40) | /3IAbRQSp/ |
| /5Alex594N/ | rUrUrUrUrU (SEQ ID NO: 40) | /3IAbRQSp/ |
| /5ATTO633N/ | rUrUrUrUrU (SEQ ID NO: 40) | /3IAbRQSp/ |

/56-FAM/: 5′ 6-Fluorescein (Integrated DNA Technologies)
/3IABKFQ/: 3′ Iowa Black FQ (Integrated DNA Technologies)
/5IRD700/: 5′ IRDye 700 (Integrated DNA Technologies)
/5TYE665/: 5′ TYE 665 (Integrated DNA Technologies)
/5Alex594N/: 5′ Alexa Fluor 594 (NHS Ester) (Integrated DNA Technologies)
/5ATTO633N/: 5′ ATTO TM 633 (NHS Ester) (Integrated DNA Technologies)
/3IRQCIN/: 3′ IRDye QC-1 Quencher (Li-Cor)
/3IAbRQSp/: 3′ Iowa Black RQ (Integrated DNA Technologies)
rU: uracil ribonucleotide
rG: guanine ribonucleotide
*This Table refers to the detection moiety and quencher moiety as their tradenames and their source is identified.
However, alternatives, generics, or non-tradename moieties with similar function from other sources can also be used.

A detection moiety can be an infrared fluorophore. A detection moiety can be a fluorophore that emits fluorescence in the range of from 500 nm and 720 nm. A detection moiety can be a fluorophore that emits fluorescence in the range of from 500 nm and 720 nm. In some cases, the detection moiety emits fluorescence at a wavelength of 700 nm or higher. In other cases, the detection moiety emits fluorescence at about 660 nm or about 670 nm. In some cases, the detection moiety emits fluorescence in the range of from 500 to 520, 500 to 540, 500 to 590, 590 to 600, 600 to 610, 610 to 620, 620 to 630, 630 to 640, 640 to 650, 650 to 660, 660 to 670, 670 to 680, 690 to 690, 690 to 700, 700 to 710, 710 to 720, or 720 to 730 nm. In some cases, the detection moiety emits fluorescence in the range from 450 nm to 750 nm, from 500 nm to 650 nm, or from 550 to 650 nm. A detection moiety can be a fluorophore that emits a detectable fluorescence signal in the same range as 6-Fluorescein, IRDye 700, TYE 665, Alex Fluor, or ATTO TM 633 (NHS Ester). A detection moiety can be fluorescein amidite, 6-Fluorescein, IRDye 700, TYE 665, Alex Fluor 594, or ATTO TM 633 (NHS Ester). A detection moiety can be a fluorophore that emits a fluorescence in the same range as 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A detection moiety can be fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). Any of the detection moieties described herein can be from any commercially available source, can be an alternative with a similar function, a generic, or a non-tradename of the detection moieties listed.

A quenching moiety can be chosen based on its ability to quench the detection moiety. A quenching moiety can be a non-fluorescent fluorescence quencher. A quenching moiety can quench a detection moiety that emits fluorescence in the range of from 500 nm and 720 nm. A quenching moiety can quench a detection moiety that emits fluorescence in the range of from 500 nm and 720 nm. In some cases, the quenching moiety quenches a detection moiety that emits fluorescence at a wavelength of 700 nm or higher. In other cases, the quenching moiety quenches a detection moiety that emits fluorescence at about 660 nm or about 670 nm. In some cases, the quenching moiety quenches a detection moiety that emits fluorescence in the range of from 500 to 520, 500 to 540, 500 to 590, 590 to 600, 600 to 610, 610 to 620, 620 to 630, 630 to 640, 640 to 650, 650 to 660, 660 to 670, 670 to 680, 690 to 690, 690 to 700, 700 to 710, 710 to 720, or 720 to 730 nm. In some cases, the quenching moiety quenches a detection moiety that emits fluorescence in the range from 450 nm to 750 nm, from 500 nm to 650 nm, or from 550 to 650 nm. A quenching moiety can quench fluorescein amidite, 6-Fluorescein, IRDye 700, TYE 665, Alex Fluor 594, or ATTO TM 633 (NHS Ester). A quenching moiety can be Iowa Black RQ, Iowa Black FQ or IRDye QC-1 Quencher. A quenching moiety can quench fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A quenching moiety can be Iowa Black RQ (Integrated DNA Technologies), Iowa Black FQ (Integrated DNA Technologies) or IRDye QC-1 Quencher (LiCor). Any of the quenching moieties described herein can be from any commercially available source, can be an alternative with a similar function, a generic, or a non-tradename of the quenching moieties listed.

The generation of the detectable signal from the release of the detection moiety indicates that cleavage by the programmable nucleases has occurred and that the sample contains the target nucleic acid. In some cases, the detection moiety comprises a fluorescent dye. Sometimes the detection moiety comprises a fluorescence resonance energy transfer (FRET) pair. In some cases, the detection moiety comprises an infrared (IR) dye. In some cases, the detection moiety comprises an ultraviolet (UV) dye. Alternatively or in combination, the detection moiety comprises a polypeptide. Alternatively, or in combination, the detection moiety comprises an enzyme. Sometimes the detection moiety comprises a biotin. Sometimes the detection moiety comprises at least one of avidin or streptavidin. In some instances, the detection moiety comprises a polysaccharide, a polymer, or a nanoparticle. In some instances, the detection moiety comprises a gold nanoparticle or a latex nanoparticle.

A detection moiety can be any moiety capable of generating a calorimetric, potentiometric, amperometric, optical (e.g., fluorescent, colorimetric, etc.), or piezo-electric signal. A nucleic acid of a reporter, sometimes, is protein-nucleic acid that is capable of generating a calorimetric, potentiometric, amperometric, optical (e.g., fluorescent, colorimetric, etc.), or piezo-electric signal upon cleavage of the nucleic acid. Often a calorimetric signal is heat produced after cleavage of the nucleic acids of a reporter. Sometimes, a calorimetric signal is heat absorbed after cleavage of the nucleic acids of a reporter. A potentiometric signal, for example, is electrical potential produced after cleavage of the nucleic acids of a reporter. An amperometric signal can be movement of electrons produced after the cleavage of nucleic acid of a reporter. Often, the signal is an optical signal, such as a colorimetric signal or a fluorescence signal. An optical signal is, for example, a light output produced after the cleavage of the nucleic acids of a reporter. Sometimes, an optical signal is a change in light absorbance between before and after the cleavage of nucleic acids of a reporter. Often, a piezo-electric signal is a change in mass between before and after the cleavage of the nucleic acid of a reporter. Other methods of detection can also be used, such as optical imaging, surface plasmon resonance (SPR), and/or interferometric sensing.

The detectable signal can be a colorimetric signal or a signal visible by eye. In some instances, the detectable signal can be fluorescent, electrical, chemical, electrochemical, or magnetic. In some cases, the first detection signal can be generated by binding of the detection moiety to the capture molecule in a detection region of a device (e.g., a capture pad of a lateral flow assay strip, a reaction volume of a microfluidic device, or the like), where the first detection signal indicates that the sample contained the target nucleic acid. Sometimes the system can be capable of detecting more than one type of target nucleic acid, wherein the system comprises more than one type of guide nucleic acid and more than one type of reporter nucleic acid. In some cases, the detectable signal can be generated directly by the cleavage event. Alternatively or in combination, the detectable signal can be generated indirectly by the signal event. Sometimes the detectable signal is not a fluorescent signal. In some instances, the detectable signal can be a colorimetric or color-based signal. In some cases, the detected target nucleic acid can be identified based on its spatial location on thea detection region of thea support medium. or surface of a device. In some cases, thea second detectable signal can be generated in a spatially distinct location than the first generated signal when two or more detectable signals are generated.

Often, the reporter is an enzyme-nucleic acid. The enzyme may be sterically hindered when present as in the enzyme-nucleic acid, but then functional upon cleavage from the nucleic acid. Often, the enzyme is an enzyme that produces a reaction with a substrate. An enzyme can be invertase. Often, the substrate of invertase is sucrose. A DNS reagent produces a colorimetric change when invertase converts sucrose to glucose. In some cases, it is preferred that the nucleic acid (e.g., RNA) and invertase are conjugated using a heterobifunctional linker via sulfo-SMCC chemistry. An enzyme can be HRP. Often, the substrate of HRP is TMB. Contact between HRP and TMB can produce a colorimetric change. Sometimes the reporter is a substrate-nucleic acid. Often the substrate is a substrate that produces a reaction with an enzyme. Release of the substrate upon cleavage by the programmable nuclease may free the substrate to react with the enzyme.

A reporter may be attached to a solid support. The solid support, for example, is a surface. A surface can be an electrode. Sometimes the solid support is a bead. Often the bead is a magnetic bead. Upon cleavage, the detection moiety (e.g., fluorophore, enzyme, etc.) is liberated from the solid support and interacts with other mixtures. For example, the detection moiety is an enzyme, and upon cleavage of the nucleic acid of the enzyme-nucleic acid reporter, the enzyme flows through a chamber of a device into a mixture comprising the substrate. When the enzyme meets the enzyme substrate, a reaction occurs, such as a colorimetric reaction, which is then detected. As another example, the detection moiety is an enzyme substrate, and upon cleavage of the nucleic acid of the enzyme substrate-nucleic acid reporter, the enzyme substrate flows through a chamber into a mixture comprising the enzyme. When the enzyme substrate meets the enzyme, a reaction occurs, such as a calorimetric reaction, which is then detected.

Often, the signal is a colorimetric signal or a signal visible by eye. In some instances, the signal is fluorescent, electrical, chemical, electrochemical, or magnetic. A signal can be a calorimetric, potentiometric, amperometric, optical (e.g., fluorescent, colorimetric, etc.), or piezo-electric signal. In some cases, the detectable signal is a colorimetric signal or a signal visible by eye. In some instances, the detectable signal is fluorescent, electrical, chemical, electrochemical, or magnetic. In some cases, the first detection signal is generated by binding of the detection moiety to the capture molecule in a detection region of a device, where the first detection signal indicates that the sample contained the target nucleic acid. Sometimes the system is capable of detecting more than one type of target nucleic acid, wherein the system comprises more than one type of guide nucleic acid and more than one type of nucleic acid of a reporter. In some cases, the detectable signal is generated directly by the cleavage event. Alternatively or in combination, the detectable signal is generated indirectly by the signal event. Sometimes the detectable signal is not a fluorescent signal. In some instances, the detectable signal is a colorimetric or color-based signal. In some cases, the detected target nucleic acid is identified based on its spatial location on the detection region of the support medium. In some cases, the second detectable signal is generated in a spatially distinct location than the first generated signal.

In some cases, the threshold of detection, for a subject method of detecting a single stranded target nucleic acid in a sample, is less than or equal to 10 nM. The term “threshold of detection” is used herein to describe the minimal amount of target nucleic acid that must be present in a sample in order for detection to occur. For example, when a threshold of detection is 10 nM, then a signal can be detected when a target nucleic acid is present in the sample at a concentration of 10 nM or more. In some cases, the threshold of detection is less than or equal to 5 nM, 1 nM, 0.5 nM, 0.1 nM, 0.05 nM, 0.01 nM, 0.005 nM, 0.001 nM, 0.0005 nM, 0.0001 nM, 0.00005 nM, 0.00001 nM, 10 pM, 1 pM, 500 fM, 250 fM, 100 fM, 50 fM, 10 fM, 5 fM, 1 fM, 500 attomole (aM), 100 aM, 50 aM, 10 aM, or 1 aM. In some cases, the threshold of detection is in a range of from 1 aM to 1 nM, 1 aM to 500 pM, 1 aM to 200 pM, 1 aM to 100 pM, 1 aM to 10 pM, 1 aM to 1 pM, 1 aM to 500 fM, 1 aM to 100 fM, 1 aM to 1 fM, 1 aM to 500 aM, 1 aM to 100 aM, 1 aM to 50 aM, 1 aM to 10 aM, 10 aM to 1 nM, 10 aM to 500 pM, 10 aM to 200 pM, 10 aM to 100 pM, 10 aM to 10 pM, 10 aM to 1 pM, 10 aM to 500 fM, 10 aM to 100 fM, 10 aM to 1 fM, 10 aM to 500 aM, 10 aM to 100 aM, 10 aM to 50 aM, 100 aM to 1 nM, 100 aM to 500 pM, 100 pM to 200 pM, 100 aM to 100 pM, 100 aM to 10 pM, 100 aM to 1 pM, 100 aM to 500 fM, 100 aM to 100 fM, 100 aM to 1 fM, 100 aM to 500 aM, 500 aM to 1 nM, 500 aM to 500 pM, 500 aM to 200 pM, 500 aM to 100 pM, 500 aM to 10 pM, 500 aM to 1 pM, 500 aM to 500 fM, 500 aM to 100 fM, 500 aM to 1 fM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, from 1 pM to 1 nM, 1 pM to 500 pM, 1 pM to 200 pM, 1 pM to 100 pM, or 1 pM to 10 pM. In some cases, the threshold of detection in a range of from 800 fM to 100 pM, 1 pM to 10 pM, 10 fM to 500 fM, 10 fM to 50 fM, 50 fM to 100 fM, 100 fM to 250 fM, or 250 fM to 500 fM. In some cases, the threshold of detection is in a range of from 2 aM to 100 pM, from 20 aM to 50 pM, from 50 aM to 20 pM, from 200 aM to 5 pM, or from 500 aM to 2 pM. In some cases, the minimum concentration at which a single stranded target nucleic acid is detected in a sample is in a range of from 1 aM to 1 nM, 10 aM to 1 nM, 100 aM to 1 nM, 500 aM to 1 nM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, from 1 pM to 200 pM, 1 pM to 100 pM, or 1 pM to 10 pM. In some cases, the minimum concentration at which a single stranded target nucleic acid is detected in a sample is in a range of from 2 aM to 100 pM, from 20 aM to 50 pM, from 50 aM to 20 pM, from 200 aM to 5 pM, or from 500 aM to 2 pM. In some cases, the minimum concentration at which a single stranded target nucleic acid can be detected in a sample is in a range of from 1 aM to 100 pM. In some cases, the minimum concentration at which a single stranded target nucleic acid can be detected in a sample is in a range of from 1 fM to 100 pM. In some cases, the minimum concentration at which a single stranded target nucleic acid can be detected in a sample is in a range of from 10 fM to 100 pM. In some cases, the minimum concentration at which a single stranded target nucleic acid can be detected in a sample is in a range of from 800 fM to 100 pM. In some cases, the minimum concentration at which a single stranded target nucleic acid can be detected in a sample is in a range of from 1 pM to 10 pM. In some cases, the devices, systems, fluidic devices, kits, and methods described herein detect a target single-stranded nucleic acid in a sample comprising a plurality of nucleic acids such as a plurality of non-target nucleic acids, where the target single-stranded nucleic acid is present at a concentration as low as 1 aM, 10 aM, 100 aM, 500 aM, 1 fM, 10 fM, 500 fM, 800 fM, 1 pM, 10 pM, 100 pM, or 1 pM.

In some embodiments, the target nucleic acid is present in the cleavage reaction at a concentration of about 10 nM, about 20 nM, about 30 nM, about 40 nM, about 50 nM, about 60 nM, about 70 nM, about 80 nM, about 90 nM, about 100 nM, about 200 nM, about 300 nM, about 400 nM, about 500 nM, about 600 nM, about 700 nM, about 800 nM, about 900 nM, about 1 μM, about 10 μM, or about 100 μM. In some embodiments, the target nucleic acid is present in the cleavage reaction at a concentration of from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1 μM, from 1 μM to 10 μM, from 10 μM to 100 μM, from 10 nM to 100 nM, from 10 nM to 1 μM, from 10 nM to 10 μM, from 10 nM to 100 μM, from 100 nM to 1 μM, from 100 nM to 10 μM, from 100 nM to 100 μM, or from 1 μM to 100 μM. In some embodiments, the target nucleic acid is present in the cleavage reaction at a concentration of from 20 nM to 50 μM, from 50 nM to 20 μM, or from 200 nM to 5 μM.

In some cases, the methods, compositions, reagents, enzymes, devices, systems, and kits described herein may be used to detect a target single-stranded nucleic acid in a sample where the sample is contacted with the reagents for a predetermined length of time sufficient for the trans-cleavage to occur or cleavage reaction to reach completion. In some cases, the devices, systems, fluidic devices, kits, and methods described herein detect a target single-stranded nucleic acid in a sample where the sample is contacted with the reagents for no greater than 60 minutes. Sometimes the sample is contacted with the reagents for no greater than 120 minutes, 110 minutes, 100 minutes, 90 minutes, 80 minutes, 70 minutes, 60 minutes, 55 minutes, 50 minutes, 45 minutes, 40 minutes, 35 minutes, 30 minutes, 25 minutes, 20 minutes, 15 minutes, 10 minutes, 5 minutes, 4 minutes, 3 minutes, 2 minutes, or 1 minute. Sometimes the sample is contacted with the reagents for at least 120 minutes, 110 minutes, 100 minutes, 90 minutes, 80 minutes, 70 minutes, 60 minutes, 55 minutes, 50 minutes, 45 minutes, 40 minutes, 35 minutes, 30 minutes, 25 minutes, 20 minutes, 15 minutes, 10 minutes, or 5 minutes. In some cases, the sample is contacted with the reagents for from 5 minutes to 120 minutes, from 5 minutes to 100 minutes, from 10 minutes to 90 minutes, from 15 minutes to 45 minutes, or from 20 minutes to 35 minutes. In some cases, the devices, systems, fluidic devices, kits, and methods described herein can detect a target nucleic acid in a sample in less than 10 hours, less than 9 hours, less than 8 hours, less than 7 hours, less than 6 hours, less than 5 hours, less than 4 hours, less than 3 hours, less than 2 hours, less than 1 hour, less than 50 minutes, less than 45 minutes, less than 40 minutes, less than 35 minutes, less than 30 minutes, less than 25 minutes, less than 20 minutes, less than 15 minutes, less than 10 minutes, less than 9 minutes, less than 8 minutes, less than 7 minutes, less than 6 minutes, or less than 5 minutes. In some cases, the devices, systems, fluidic devices, kits, and methods described herein can detect a target nucleic acid in a sample in from 5 minutes to 10 hours, from 10 minutes to 8 hours, from 15 minutes to 6 hours, from 20 minutes to 5 hours, from 30 minutes to 2 hours, or from 45 minutes to 1 hour.

When an engineered guide nucleic acid binds to a target nucleic acid, the programmable nuclease's trans-cleavage activity can be initiated, and nucleic acids of a reporter can be cleaved, resulting in the detection of a detectable signal (e.g., fluorescence). The guide nucleic acid may be a non-naturally occurring guide nucleic acid. A non-naturally occurring guide nucleic acid may comprise an engineered sequence having a repeat and a spacer that hybridizes to a target nucleic acid sequence of interest. A non-naturally occurring guide nucleic acid may be recombinantly expressed or chemically synthesized. Nucleic acid reporters can comprise a detection moiety, wherein the nucleic acid reporter can be cleaved by the activated programmable nuclease, thereby generating a signal as described herein. Some methods as described herein can a method of assaying for a target nucleic acid in a sample comprises contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some reporter nucleic acids of a population of reporter nucleic acids, wherein the signal indicates a presence of the target nucleic acid in the sample and wherein absence of the signal indicates an absence of the target nucleic acid in the sample. The cleaving of the nucleic acid of a reporter using the programmable nuclease may cleave with an efficiency of 50% as measured by a change in a signal that is calorimetric, potentiometric, amperometric, optical (e.g., fluorescent, colorimetric, etc.), or piezo-electric, as non-limiting examples. Some methods as described herein can be a method of detecting a target nucleic acid in a sample comprising contacting the sample comprising the target nucleic acid with a guide nucleic acid targeting a target nucleic acid segment, a programmable nuclease capable of being activated when complexed with the guide nucleic acid and the target nucleic acid segment, a single stranded nucleic acid of a reporter comprising a detection moiety, wherein the nucleic acid of a reporter is capable of being cleaved by the activated programmable nuclease, thereby generating a first detectable signal, cleaving the single stranded nucleic acid of a reporter using the programmable nuclease that cleaves as measured by a change in color, and measuring the first detectable signal on a support medium of a device. The cleaving of the single stranded nucleic acid of a reporter using the programmable nuclease may cleave with an efficiency of 50% as measured by a change in color. In some cases, the cleavage efficiency is at least 40%, 50%, 60%, 70%, 80%, 90%, or 95% as measured by a change in color. The change in color may be a detectable colorimetric signal or a signal visible by eye. The change in color may be measured as a first detectable signal. The first detectable signal can be detectable within 5 minutes of contacting the sample comprising the target nucleic acid with a guide nucleic acid targeting a target nucleic acid segment, a programmable nuclease capable of being activated when complexed with the guide nucleic acid and the target nucleic acid segment, and a single stranded nucleic acid of a reporter comprising a detection moiety, wherein the nucleic acid of a reporter is capable of being cleaved by the activated programmable nuclease. The first detectable signal can be detectable within 1, 2, 3, 4, 5, 6, 7, 8, 9, 10, 11, 12, 13, 14, 15, 20, 25, 30, 35, 40, 45, 50, 55, 60, 70, 80, 90, 100, 110, or 120 minutes of contacting the sample. In some embodiments, the first detectable signal can be detectable within from 1 to 120, from 5 to 100, from 10 to 90, from 15 to 80, from 20 to 60, or from 30 to 45 minutes of contacting the sample.

In some cases, the methods, reagents, enzymes, systems, devices, and kits described herein detect a target single-stranded nucleic acid with a programmable nuclease and a single-stranded nucleic acid of a reporter in a sample where the sample is contacted with the reagents for a predetermined length of time sufficient for trans-cleavage of the single stranded nucleic acid of a reporter.

Some methods as described herein can be a method of detecting a target nucleic acid in a sample comprising contacting the sample comprising the target nucleic acid with a guide nucleic acid targeting a target sequence, a programmable nuclease capable of being activated when complexed with the guide nucleic acid and the target sequence, a single stranded reporter nucleic acid comprising a detection moiety, wherein the reporter nucleic acid is capable of being cleaved by the activated nuclease, thereby generating a first detectable signal, cleaving the single stranded reporter nucleic acid using the programmable nuclease that cleaves as measured by a change in color, and measuring the first detectable signal on the support medium. The cleaving of the single stranded reporter nucleic acid using the programmable nuclease may cleave with an efficiency of 50% as measured by a change in color. In some cases, the cleavage efficiency is at least 40%, at least 50%, at least 60%, at least 70%, at least 80%, at least 90%, or at least 95% as measured by a change in color. The change in color may be a detectable colorimetric signal or a signal visible by eye. The change in color may be measured as a first detectable signal. The first detectable signal can be detectable within 5 minutes of contacting the sample comprising the target nucleic acid with a guide nucleic acid targeting a target sequence, a programmable nuclease capable of being activated when complexed with the guide nucleic acid and the target sequence, and a single stranded reporter nucleic acid comprising a detection moiety, wherein the reporter nucleic acid is capable of being cleaved by the activated nuclease. The first detectable signal can be detectable within 1, 2, 3, 4, 5, 6, 7, 8, 9, 10, 11, 12, 13, 14, 15, 20, 25, 30, 35, 40, 45, 50, 55, 60, 70, 80, 90, 100, 110, or 120 minutes of contacting the sample.

### Multiplexing Programmable Nucleases

Described herein are compositions comprising a programmable Type VI CRISPR/Cas nuclease capable of being activated when complexed with the guide nucleic acid and the target nucleic acid molecule. Furthermore, these reagents can be used with different types of programmable nuclease, e.g., for multiplexing programmable nucleases. In some embodiments, a programmable nuclease may be multiplexed with an additional programmable nuclease. For example, a programmable nuclease may be multiplexed with an additional programmable nuclease for modification or detection of a target nucleic acid. In some embodiments, a first programmable nuclease may be multiplexed with a second programmable nuclease. In some embodiments, the programmable nuclease may be a Type VI CRISPR/Cas programmable nuclease.

In some embodiments, an additional programmable nuclease used in multiplexing is any suitable programmable nuclease. Sometimes, the programmable nuclease is any Cas protein (also referred to as a Cas nuclease herein). In some cases, the programmable nuclease is Cas13. In some embodiments, the Cas13 is Cas13a, Cas13b, Cas13c, Cas13d, or Cas13e. In some cases, the programmable nuclease can be Mad7 or Mad2. In some cases, the programmable nuclease is a Cas12 protein. Sometimes the Cas12 is Cas12a, Cas12b, Cas12c, Cas12d, Cas12e, Cas12g, Cas12h, or Cas12i. In some cases, the programmable nuclease is another Cas13 protein. In some cases, the programmable nuclease is Cas3, Csm1, Cas9, C2c4, C2c8, C2c5, C2c10, C2c9, or CasZ. Sometimes, the Csm1 can be also called smCms1, miCms1, obCms1, or suCms1. Sometimes CasZ can be also called Cas14a, Cas14b, Cas14c, Cas14d, Cas14e, Cas14f, Cas14g, or Cas14h. Sometimes, the programmable nuclease can be a type V CRISPR-Cas system. In some cases, the programmable nuclease can be a type VI CRISPR-Cas system. In some embodiments, the Type V CRISPR/Cas enzyme is a CasΦ nuclease. A CasΦ polypeptide can function as an endonuclease that catalyzes cleavage at a specific sequence in a target nucleic acid. In non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof

In some cases, an additional programmable nuclease used in multiplexing can be from, for example, Leptotrichia shahii (Lsh), Listeria seeligeri (Lse), Leptotrichia buccalis (Lbu), Leptotrichia wadeu (Lwa), Rhodobacter capsulatus (Rca), Herbinix hemicellulosilytica (Hhe), Paludibacter propionicigenes (Ppr), Lachnospiraceae bacterium (Lba), Eubacterium rectale (Ere), Listeria newyorkensis (Lny), Clostridium aminophilum (Cam), Prevotella sp. (Psm), Capnocytophaga canimorsus (Cca, Lachnospiraceae bacterium (Lba), Bergeyella zoohelcum (Bzo), Prevotella intermedia (Pin), Prevotella buccae (Pbu), Alistipes sp. (Asp), Riemerella anatipestifer (Ran), Prevotella aurantiaca (Pau), Prevotella saccharolytica (Psa), Prevotella intermedia (Pin2), Capnocytophaga canimorsus (Cca), Porphyromonas gulae (Pgu), Prevotella sp. (Psp), Porphyromonas gingivalis (Pig), Prevotella intermedia (Pin3), Enterococcus italicus (Ei), Lactobacillus salivarius (Ls), or Thermus thermophilus (Tt). In some cases, an additional programmable nuclease used in multiplexing can be from, for example, a phage such as a bacteriophage also called a megaphage. The nucleases may come from a particular bacteriophage clade called Biggiephage. Any combination of programmable nucleases can be used in multiplexing. In some embodiments, multiplexing of programmable nucleases takes place in one reaction volume. In other embodiments, multiplexing of programmable nucleases takes place in separate reaction volumes in a single device.

### Direct Detection of a Target Nucleic Acid

Disclosed herein are methods of direct detection of a target nucleic acid using any of the methods, reagents, kits or devices described herein. Detection of the target nucleic acid can be performed directly without the need for amplification of the target nucleic acid. The target nucleic can be in sufficient quantity that the detection methods disclosed herein produce a quantifiable signal to determine the presence of the target nucleic acid in the sample.

In some embodiments, the target nucleic acids are not amplified prior to its use in a DETECTR assay method disclosed herein. The compositions for target nucleic acids and methods of use thereof, as described herein, are compatible with any of the programmable nucleases disclosed herein and use of said programmable nuclease in a method of detecting a target nucleic acid. The nucleic acid of interest may be any nucleic acid disclosed herein or from any sample as disclosed herein. The nucleic acid of interest may be an RNA that is reverse transcribed. The nucleic acid can be DNA that has been transcribed to produce RNA nucleic acids compatible with detection method disclosed herein.

### Amplification of a Target Nucleic Acid

Disclosed herein are methods of amplifying a target nucleic acid for detection using any of the methods, reagents, kits or devices described herein. The compositions for amplification of target nucleic acids and methods of use thereof, as described herein, are compatible with the DETECTR assay methods disclosed herein. The compositions for amplification of target nucleic acids and methods of use thereof, as described herein, are compatible with any of the programmable nucleases disclosed herein and use of said programmable nuclease in a method of detecting a target nucleic acid. A target nucleic acid can be an amplified nucleic acid of interest. The nucleic acid of interest may be any nucleic acid disclosed herein or from any sample as disclosed herein. The nucleic acid of interest may be an RNA that is reverse transcribed before amplification. The nucleic acid of interest may be amplified then the amplicons may be transcribed into RNA. This amplification can be thermal amplification (e.g., using PCR) or isothermal amplification. This nucleic acid amplification of the sample can improve at least one of sensitivity, specificity, or accuracy of the detection of the target nucleic acid. The reagents for nucleic acid amplification can comprise a recombinase, an oligonucleotide primer, a single-stranded DNA binding (SSB) protein, and a polymerase. The nucleic acid amplification can be transcription mediated amplification (TMA). Nucleic acid amplification can be helicase dependent amplification (HDA) or circular helicase dependent amplification (cHDA). In additional cases, nucleic acid amplification is strand displacement amplification (SDA). The nucleic acid amplification can be recombinase polymerase amplification (RPA). The nucleic acid amplification can be at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). The nucleic acid amplification can be performed for no greater than 1, 2, 3, 4, 5, 6, 7, 8, 9, 10, 11, 12, 13, 14, 15, 16, 17, 18, 19, 20, 25, 30, 40, 50, or 60 minutes. Sometimes, the nucleic acid amplification reaction is performed at a temperature of around 20-65° C. The nucleic acid amplification reaction can be performed at a temperature no greater than 20° C., 25° C., 30° C., 35° C., 37° C., 40° C., 45° C., 50° C., 55° C., 60° C., or 65° C. The nucleic acid amplification reaction can be performed at a temperature of at least 20° C., 25° C., 30° C., 35° C., 37° C., 40° C., 45° C., 50° C., 55° C., 60° C., or 65° C.

The compositions for amplification of target nucleic acids and methods of use thereof, as described herein, are compatible with any of the compositions comprising a programmable nuclease and a buffer, which has been developed to improve the function of the programmable nuclease and use of said compositions in a method of detecting a target nucleic acid. The compositions for amplification of target nucleic acids and methods of use thereof, as described herein, are compatible with any of the methods disclosed herein including methods of assaying for at least one base difference (e.g., assaying for a SNP or a base mutation) in a target nucleic acid sequence, methods of assaying for a target nucleic acid that lacks a PAM by amplifying the target nucleic acid sequence to introduce a PAM, and compositions used in introducing a PAM via amplification into the target nucleic acid sequence. In some cases, amplification of the target nucleic acid may increase the sensitivity of a detection reaction. In some cases, amplification of the target nucleic acid may increase the specificity of a detection reaction. Amplification of the target nucleic acid may increase the concentration of the target nucleic acid in the sample relative to the concentration of nucleic acids that do not correspond to the target nucleic acid. In some embodiments, amplification of the target nucleic acid may be used to modify the sequence of the target nucleic acid. For example, amplification may be used to insert a PAM sequence into a target nucleic acid that lacks a PAM sequence. In some cases, amplification may be used to increase the homogeneity of a target nucleic acid sequence. For example, amplification may be used to remove a nucleic acid variation that is not of interest in the target nucleic acid sequence.

An amplified target nucleic acid may be present in a DETECTR reaction in an amount relative to an amount of a programmable nuclease. In some embodiments, the amplified target nucleic acid is present in at least 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the programmable nuclease. In some embodiments, the amplified target nucleic acid is present in no more than 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the programmable nuclease. In some embodiments, the amplified target nucleic acid is present in from 1-fold to 2-fold, from 1-fold to 3-fold, from 1-fold to 4-fold, from 1-fold to 5-fold, from 1-fold to 10-fold, from 1-fold to 25-fold, from 1-fold to 50-fold, from 1-fold to 100-fold, from 1-fold to 500-fold, from 1-fold to 1000-fold, from 1-fold to 10,000-fold, from 1-fold to 100,000-fold, from 5-fold to 10-fold, from 5-fold to 25-fold, from 5-fold to 50-fold, from 5-fold to 100-fold, from 5-fold to 500-fold, from 5-fold to 1000-fold, from 5-fold to 10,000-fold, from 5-fold to 100,000-fold, from 10-fold to 25-fold, from 10-fold to 50-fold, from 10-fold to 100-fold, from 10-fold to 500-fold, from 10-fold to 1000-fold, from 10-fold to 10,000-fold, from 10-fold to 100,000-fold, from 100-fold to 500-fold, from 100-fold to 1000-fold, from 100-fold to 10,000-fold, from 100-fold to 100,000-fold, from 1000-fold to 10,000-fold, from 1000-fold to 100,000-fold, or from 10,000-fold to 100,000-fold molar excess relative to the amount of the programmable nuclease. In some embodiments, the programmable nuclease is present in at least 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the target nucleic acid. In some embodiments, the programmable nuclease is present in no more than 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the target nucleic acid. In some embodiments, the programmable nuclease is present in from 1-fold to 2-fold, from 1-fold to 3-fold, from 1-fold to 4-fold, from 1-fold to 5-fold, from 1-fold to 10-fold, from 1-fold to 25-fold, from 1-fold to 50-fold, from 1-fold to 100-fold, from 1-fold to 500-fold, from 1-fold to 1000-fold, from 1-fold to 10,000-fold, from 1-fold to 100,000-fold, from 5-fold to 10-fold, from 5-fold to 25-fold, from 5-fold to 50-fold, from 5-fold to 100-fold, from 5-fold to 500-fold, from 5-fold to 1000-fold, from 5-fold to 10,000-fold, from 5-fold to 100,000-fold, from 10-fold to 25-fold, from 10-fold to 50-fold, from 10-fold to 100-fold, from 10-fold to 500-fold, from 10-fold to 1000-fold, from 10-fold to 10,000-fold, from 10-fold to 100,000-fold, from 100-fold to 500-fold, from 100-fold to 1000-fold, from 100-fold to 10,000-fold, from 100-fold to 100,000-fold, from 1000-fold to 10,000-fold, from 1000-fold to 100,000-fold, or from 10,000-fold to 100,000-fold molar excess relative to the amount of the target nucleic acid. In some embodiments, the target nucleic acid is not present in the sample.

An amplified target nucleic acid may be present in a DETECTR reaction in an amount relative to an amount of a guide nucleic acid. In some embodiments, the amplified target nucleic acid is present in at least 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the guide nucleic acid. In some embodiments, the amplified target nucleic acid is present in no more than 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the guide nucleic acid. In some embodiments, the amplified target nucleic acid is present in from 1-fold to 2-fold, from 1-fold to 3-fold, from 1-fold to 4-fold, from 1-fold to 5-fold, from 1-fold to 10-fold, from 1-fold to 25-fold, from 1-fold to 50-fold, from 1-fold to 100-fold, from 1-fold to 500-fold, from 1-fold to 1000-fold, from 1-fold to 10,000-fold, from 1-fold to 100,000-fold, from 5-fold to 10-fold, from 5-fold to 25-fold, from 5-fold to 50-fold, from 5-fold to 100-fold, from 5-fold to 500-fold, from 5-fold to 1000-fold, from 5-fold to 10,000-fold, from 5-fold to 100,000-fold, from 10-fold to 25-fold, from 10-fold to 50-fold, from 10-fold to 100-fold, from 10-fold to 500-fold, from 10-fold to 1000-fold, from 10-fold to 10,000-fold, from 10-fold to 100,000-fold, from 100-fold to 500-fold, from 100-fold to 1000-fold, from 100-fold to 10,000-fold, from 100-fold to 100,000-fold, from 1000-fold to 10,000-fold, from 1000-fold to 100,000-fold, or from 10,000-fold to 100,000-fold molar excess relative to the amount of the guide nucleic acid. In some embodiments, the guide nucleic acid is present in at least 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the target nucleic acid. In some embodiments, the guide nucleic acid is present in no more than 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the target nucleic acid. In some embodiments, the guide nucleic acid is present in from 1-fold to 2-fold, from 1-fold to 3-fold, from 1-fold to 4-fold, from 1-fold to 5-fold, from 1-fold to 10-fold, from 1-fold to 25-fold, from 1-fold to 50-fold, from 1-fold to 100-fold, from 1-fold to 500-fold, from 1-fold to 1000-fold, from 1-fold to 10,000-fold, from 1-fold to 100,000-fold, from 5-fold to 10-fold, from 5-fold to 25-fold, from 5-fold to 50-fold, from 5-fold to 100-fold, from 5-fold to 500-fold, from 5-fold to 1000-fold, from 5-fold to 10,000-fold, from 5-fold to 100,000-fold, from 10-fold to 25-fold, from 10-fold to 50-fold, from 10-fold to 100-fold, from 10-fold to 500-fold, from 10-fold to 1000-fold, from 10-fold to 10,000-fold, from 10-fold to 100,000-fold, from 100-fold to 500-fold, from 100-fold to 1000-fold, from 100-fold to 10,000-fold, from 100-fold to 100,000-fold, from 1000-fold to 10,000-fold, from 1000-fold to 100,000-fold, or from 10,000-fold to 100,000-fold molar excess relative to the amount of the target nucleic acid. In some embodiments, the target nucleic acid is not present in the sample.

### Devices

Disclosed here are systems and devices for use to detect a target nucleic acid sequence as disclosed herein using the methods as discussed herein. In some embodiments, the device may be a handheld device. In some embodiments, the device may be a point-of-need or point-of-care device. In some embodiments, the device may function as a stand-alone device (e.g., without significant additional instrumentation). In some embodiments, the system may comprise a device configured to be coupled to an instrument to run the assay and/or detect the detectable signal after the assay is completed. In some embodiments, the device and/or instrument may be reusable. In some embodiments, the device may be disposable.

In some embodiments, systems and devices for target nucleic acid detection may include one or more reaction volumes such as tubes, wells, chambers, and/or channels in which to perform the detection methods described herein. In some embodiments, the system or device workflow may comprise: (1) sample collection and/or delivery to the device, (2) optional lysis, (3) optional amplification of the target nucleic acids, and (4) detection/readout. In some embodiments, amplification and detection are carried out in a single reaction volume. In some embodiments, sample amplification is carried in a first reaction volume and detection is carried out in a second reaction volume. In some embodiments, reporter cleavage and signal detection are carried out in a single reaction volume. In some embodiments, reporter cleavage is carried out in a first reaction volume and signal detection (e.g., detection of a colorimetric signal generated by an enzyme detection moiety contacting its enzyme substrate) is carried out in a second reaction volume. In some embodiments, multiple reactions can be carried out in multiple reaction volumes.

One or more components or reagents of a DETECTR reaction may be suspended in solution or immobilized on a surface of the system or device. Programmable nucleases, guide nucleic acids, and/or reporters may be suspended in solution or immobilized on a surface. For example, the reporter, programmable nuclease, and/or guide nucleic acid can be immobilized on the surface of a chamber in a device. In some cases, the reporter, programmable nuclease, and/or guide nucleic acid can be immobilized on beads, such as magnetic beads, in a chamber of a device where they are held in position by a magnet placed below the chamber. An immobilized programmable nuclease can be capable of being activated and cleaving a free-floating or immobilized reporter. An immobilized guide nucleic acid can be capable of binding a target nucleic acid and activating a programmable nuclease complexed thereto. An immobilized reporter can be capable of being cleaved by the activated programmable nuclease, thereby releasing a detection moiety and generating a detectable signal.

In some embodiments, a reporter is connected to a surface of the system or device by a linkage. In some embodiments, a reporter may comprise at least one of a nucleic acid, a chemical functionality, a detection moiety, a quenching moiety, or a combination thereof. In some embodiments, a reporter is configured for the detection moiety to remain immobilized to the surface and the quenching moiety to be released into solution upon cleavage of the reporter. In some embodiments, a reporter is configured for the quenching moiety to remain immobilized to the surface and for the detection moiety to be released into solution, upon cleavage of the reporter. Often the detection moiety is at least one of a label, a polypeptide, a dendrimer, an enzyme, or a nucleic acid, or a combination thereof. In some embodiments, the reporter contains a label. In some embodiments, the label may be FITC, DIG, TAMRA, Cy5, AF594, or Cy3. In some embodiments, the label may comprise a dye, a nanoparticle configured to produce a signal. In some embodiments, the dye may be a fluorescent dye. In some embodiments, the at least one chemical functionality may comprise biotin. In some embodiments, the at least one chemical functionality may be configured to be captured on a surface of the system or device by a capture probe (e.g., in a detection well of a multi-well plate, in a detection chamber of a microfluidic device, at a capture pad of a lateral flow assay strip, etc.). In some embodiments, the at least one chemical functionality may comprise biotin and the capture probe may comprise anti-biotin, streptavidin, avidin or other molecule configured to bind with biotin. In some embodiments, the dye is the chemical functionality. In some embodiments, a capture probe may comprise a molecule that is complementary to the chemical functionality. In some embodiments, the capture antibodies are anti-FITC, anti-DIG, anti-TAMRA, anti-Cy5, anti-AF594, or any other appropriate capture antibody capable of binding the detection moiety or conjugate. In some embodiments, the detection moiety can be the chemical functionality.

### Kits

Disclosed herein are kits for use to detect, modify, edit, or regulate a target nucleic acid sequence as disclosed herein using the methods as discussed herein. In some embodiments, the kit comprises the programmable Type VI CRISPR/Cas nuclease system, reagents, and the support medium. The reagents and programmable nuclease system can be provided in a reagent chamber or on the support medium. Alternatively, the reagent and programmable nuclease system can be placed into the reagent chamber or the support medium by the individual using the kit. Optionally, the kit further comprises a buffer and a dropper. The reagent chamber can be a test well or container. The opening of the reagent chamber can be large enough to accommodate the support medium. The buffer can be provided in a dropper bottle for ease of dispensing. The dropper can be disposable and transfer a fixed volume. The dropper can be used to place a sample into the reagent chamber or on the support medium.

The kit or system for detection of a target nucleic acid described herein further comprises reagents for nucleic acid amplification of target nucleic acids in the sample. Isothermal nucleic acid amplification allows the use of the kit or system in remote regions or low resource settings without specialized equipment for amplification. Often, the reagents for nucleic acid amplification comprise a recombinase, an oligonucleotide primer, a single-stranded DNA binding (SSB) protein, and a polymerase. Sometimes, nucleic acid amplification of the sample improves at least one of sensitivity, specificity, or accuracy of the assay in detecting the target nucleic acid. In some cases, the nucleic acid amplification is performed in a nucleic acid amplification region on the support medium. Alternatively, or in combination, the nucleic acid amplification is performed in a reagent chamber, and the resulting sample is applied to the support medium. Sometimes, the nucleic acid amplification is isothermal nucleic acid amplification. In some cases, the nucleic acid amplification is transcription mediated amplification (TMA). Nucleic acid amplification is helicase dependent amplification (HDA) or circular helicase dependent amplification (cHDA) in other cases. In additional cases, nucleic acid amplification is strand displacement amplification (SDA). In some cases, nucleic acid amplification is by recombinase polymerase amplification (RPA). In some cases, nucleic acid amplification is by at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). Often, the nucleic acid amplification is performed for no greater than 1, 2, 3, 4, 5, 6, 7, 8, 9, 10, 11, 12, 13, 14, 15, 16, 17, 18, 19, 20, 25, 30, 40, 50, or 60 minutes, or any value from 1 to 60 minutes. Sometimes, the nucleic acid amplification is performed for from 1 to 60, from 5 to 55, from 10 to 50, from 15 to 45, from 20 to 40, or from 25 to 35 minutes. Sometimes, the nucleic acid amplification reaction is performed at a temperature of around 20-45° C. In some cases, the nucleic acid amplification reaction is performed at a temperature no greater than 20° C., 25° C., 30° C., 35° C., 37° C., 40° C., 45° C., or any value from 20° C. to 45° C. In some cases, the nucleic acid amplification reaction is performed at a temperature of at least 20° C., 25° C., 30° C., 35° C., 37° C., 40° C., or 45° C., or any value from 20° C. to 45° C. In some cases, the nucleic acid amplification reaction is performed at a temperature of from 20° C. to 45° C., from 25° C. to 40° C., from 30° C. to 40° C., or from 35° C. to 40° C.

In some embodiments, a kit for detecting a target nucleic acid comprising a support medium; a guide nucleic acid targeting a target sequence; a programmable nuclease capable of being activated when complexed with the guide nucleic acid and the target sequence; and a reporter nucleic acid comprising a detection moiety, wherein the reporter nucleic acid is capable of being cleaved by the activated nuclease, thereby generating a first detectable signal. Often, the kit further comprises primers for amplifying a target nucleic acid of interest to produce a PAM target nucleic acid.

In some embodiments, a kit for detecting a target nucleic acid comprising a PCR plate; a guide nucleic acid targeting a target sequence; a programmable nuclease capable of being activated when complexed with the guide nucleic acid and the target sequence; and a single stranded reporter nucleic acid comprising a detection moiety, wherein the reporter nucleic acid is capable of being cleaved by the activated nuclease, thereby generating a first detectable signal. The wells of the PCR plate can be pre-aliquoted with the guide nucleic acid targeting a target sequence, a programmable nuclease capable of being activated when complexed with the guide nucleic acid and the target sequence, and at least one population of a single stranded reporter nucleic acid comprising a detection moiety. A user can thus add the biological sample of interest to a well of the pre-aliquoted PCR plate and measure for the detectable signal with a fluorescent light reader or a visible light reader.

In some embodiments, a kit for modifying a target nucleic acid comprising a support medium; a guide nucleic acid targeting a target sequence; and a programmable nuclease capable of being activated when complexed with the guide nucleic acid and the target sequence.

In some embodiments, a kit for modifying a target nucleic acid comprising a PCR plate; a guide nucleic acid targeting a target sequence; and a programmable nuclease capable of being activated when complexed with the guide nucleic acid and the target sequence. The wells of the PCR plate can be pre-aliquoted with the guide nucleic acid targeting a target sequence, and a programmable nuclease capable of being activated when complexed with the guide nucleic acid and the target sequence. A user can thus add the biological sample of interest to a well of the pre-aliquoted PCR plate.

In some instances, such kits may include a package, carrier, or container that is compartmentalized to receive one or more containers such as vials, tubes, and the like, each of the container(s) comprising one of the separate elements to be used in a method described herein.

Suitable containers include, for example, test wells, bottles, vials, and test tubes. In one embodiment, the containers are formed from a variety of materials such as glass, plastic, or polymers.

The kit or systems described herein contain packaging materials. Examples of packaging materials include, but are not limited to, pouches, blister packs, bottles, tubes, bags, containers, bottles, and any packaging material suitable for intended mode of use.

A kit typically includes labels listing contents and/or instructions for use, and package inserts with instructions for use. A set of instructions will also typically be included. In one embodiment, a label is on or associated with the container. In some instances, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

After packaging the formed product and wrapping or boxing to maintain a sterile barrier, the product may be terminally sterilized by heat sterilization, gas sterilization, gamma irradiation, or by electron beam sterilization. Alternatively, the product may be prepared and packaged by aseptic processing.

Methods of the disclosure can be performed in a subject. Compositions of the disclosure can be administered to a subject. A subject can be a human. A subject can be a mammal (e.g., rat, mouse, cow, dog, pig, sheep, horse). A subject can be a vertebrate or an invertebrate. A subject can be a laboratory animal. A subject can be a patient. A subject can be suffering from a disease. A subject can display symptoms of a disease. A subject may not display symptoms of a disease, but still have a disease. A subject can be under medical care of a caregiver (e.g., the subject is hospitalized and is treated by a physician). A subject can be a plant or a crop.

Methods of the disclosure can be performed in a cell. A cell can be in vitro. A cell can be in vivo. A cell can be ex vivo. A cell can be an isolated cell. A cell can be a cell inside of an organism. A cell can be an organism. A cell can be a cell in a cell culture. A cell can be one of a collection of cells. A cell can be a mammalian cell or derived from a mammalian cell. A cell can be a rodent cell or derived from a rodent cell. A cell can be a human cell or derived from a human cell. A cell can be a prokaryotic cell or derived from a prokaryotic cell. A cell can be a bacterial cell or can be derived from a bacterial cell. A cell can be an archaeal cell or derived from an archaeal cell. A cell can be a eukaryotic cell or derived from a eukaryotic cell. A cell can be a pluripotent stem cell. A cell can be a plant cell or derived from a plant cell. A cell can be an animal cell or derived from an animal cell. A cell can be an invertebrate cell or derived from an invertebrate cell. A cell can be a vertebrate cell or derived from a vertebrate cell. A cell can be a microbe cell or derived from a microbe cell. A cell can be a fungi cell or derived from a fungi cell. A cell can be from a specific organ or tissue.

Methods of the disclosure can be performed in a eukaryotic cell or cell line. In some embodiments, the eukaryotic cell is a Chinese hamster ovary (CHO) cell. In some embodiments, the eukaryotic cell is a Human embryonic kidney 293 cells (also referred to as HEK or HEK 293) cell.

### Specific Targets and Indications

Described herein are compositions and methods detecting a target nucleic acid, wherein the target nucleic acid is a gene, a portion thereof, a transcript thereof. In some embodiments, the target nucleic acid comprises a mutation, and the compositions and/or methods detect the mutation. In some embodiments, compositions and methods comprise inducing death of a cell that harbors a mutation in a target nucleic acid. In some embodiments, the target nucleic acid is a reverse transcript (e.g. a cDNA) of an mRNA transcribed from the gene, or an amplicon thereof. In some embodiments, the target nucleic acid is an amplicon of at least a portion of a gene. Non-limiting examples of genes are: AAVS1, ABCA4, ABCB11, ABCC8, ABCD1, ACAD9, ACADM, ACADVL, ACAT1, ACOX1, ACSF3, ADA, ADAMTS2, ADGRG1, AGA, AGL, AGPS, AGXT, AHI, AIRE, ALDH3A2, ALDOB, ALG6, ALK, ALKBH5, ALMS1, ALPL, AMRC9, AMT, ANAPC10, ANAPC11, ANGPTL3, APC, Apo(α), APOCIII, APOEε4, APOL1, APP, AQP2, AR, ARFRP1, ARG1, ARL13B, ARL6, ARSA, ARSB, ASL, ASNS, ASPA, ASS1, ATM, ATP6V1B1, ATP7A, ATP7B, ATRX, ATXN1, ATXN10, ATXN2, ATXN3, ATXN7, ATXN8OS, AXIN1, AXIN2, B2M, BACE-1, BAK1, BAP1, BARD1, BAX2, BBS1, BBS10, BBS12, BBS2, BCKDHA, BCKDHB, BCL2L2, BCS1L, BEST1, Betaglobin gene, BLM, BMPR1A, BRAF, BRAFV600E, BRCA1, BRCA2, BRIP1, BSND, C282Y, C9orf72, CA4, CACNA1A, CAPN3, CASR, CBS, CCNB1CC2D2A, CCR5, CDC73, CDH1, CDH23, CDK11, CDK4, CDKN1A, CDKN1B, CDKN1C, CDKN2A, CEBPA, CEL A3B, CEP290, CERKL, CFB, CFTR, CHCHD10, CHEK2, CHM, CHRNE, CIITA, CLN3, CLN5, CLN6, CLN8, CLRN1, CLTA, CNBP, CNGB1, CNGB3, COLLA1, COL1A2, COL27A1, COL4A3, COL4A4, COL4A5, COL7A1, CPS1, CPT1A, CPT2, CRB1, CREBBP, CRX, CRYAA, CTNNA1, CTNNB1, CTNND2, CTNS, CTSK, CYBA, CYBB, CYP11B1, CYP11B2, CYP17A1, CYP19A1, CYP27A1, DBT, DCC, DCLRE1C, DERL2, DFNA36, DFNB31, DGAT2, DHCR7, DHDDS, DICER1, DIS3L2, DLD, DMD, DMPK, DNAH5, DNAI1, DNAI2, DNM2, DNMT1, DPC4, DYSF, EDA, EDN3, EDNRB, EGFR, EIF2B5, EMC2, EMC3, EMD, EMX1, EN1, EPCAM, ERCC6, ERCC8, ESCO2, ETFA, ETFDH, ETHE1, EVC, EVC2, EYS, F5, F9, FXI, FAH, FAM161A, FANCA, FANCB, FANCC, FANCD1, FANCD2, FANCE, FANCF, FANCG, FANCI, FANCJ, FANCL, FANCM, FANCN, FANCP, FANCS, FBN1, FGF14, FGFR2, FGFR3, FH, FHL1, FKRP, FKTN, FLCN, FMR1, FOXP3, FSCN2, FUS, FUT8, FVIII, FXII, FXN, G6PC, GAA, GALC, GALK1, GALT, GAMT, GATA2, GBA, GBE1, GCDH, GCGR, GDNF, GFAP, GFM1, GHR, GJB1, GJB2, GLA, GLB1, GLDC, GLE1, GNE, GNPTAB, GNPTG, GNS, GPC3, GPR98, GREM1, GRHPR, GRIN2B, H2AFX H2AX HADHA, HAX1, HBA1, HBA2, HBB, HER2, HEXA, HEXB, HGSNAT, HLCS, HMGCL, HOGA1, HOXB13, HPRPF3, HPRT1, HPS1, HPS3, HRAS, HSD17B4, HSD3B2, HTT, HUS1, HYAL1, HYLS1, IDS, IDUA, IFITM5, IKBKAP, IL2RG, IMPDH1, INPP5E, IRF4, ITPR1, IVD, JAG1, JAK1, KCNC3, KCND3, KCNJ11, KLHL7, KRAS, LAMA2, LAMA3, LAMB3, LAMC2, LCA5, LDLR, LDLRAP1, LHX3, LIFR, LIPA, LMNA, LOR, LOXHD1, LPL, LRAT, LRP6, LRPPRC, LRRK2, MADR2, MAN2B1, MAPT, MAX, MCM6, MCOLN1, MECP2, MED17, MEFV, MEN1, MERTK, MESP2, MET, METex14, MFN2, MFSD8, MITF, MKS1, MLC1, MLH1, MLH3, MMAA, MMAB, MMACHC, MMADHC, MMD, MPI, MPL, MPV17, MSH2, MSH3, MSH6, MTHFR, MTM1, MTRR, MTTP, MUT, MUTYH, MYC, MYO7A, NAGLU, NAGS, NBN, NDRG1, NDUFAF5, NDUFS6, NEB, NF1, NF2, NOG, NOTCH2, NPC1, NPC2, NPHP1, NPHS1, NPHS2, NRAS, NR2E3, NTHL1, NTRK, NTRK1, OAT, OCT4, OFD1, OPA3, OTC, PAH, PALB2, PAQR8, PAX3, PC, PCCA, PCCB, PCDH15, PCSK9, PD1, PDCD1, PDE6B, PDGFRA, PDHA1, PDHB, PEX1, PEX10, PEX12, PEX13, PEX14, PEX16, PEX19, PEX2, PEX26, PEX3, PEX5, PEX6, PEX7, PFKM, PHGDH, PHOX2B, PKD1, PKD2, PKHD1, PKK, PLEKHG4, PMM2, PMP22, PMS1, PMS2, PNPLA3, POLD1, POLE, POMGNT1, POT1, POU5F1, PPM1A, PPP2R2B, PPT1, PRCD, PRKAR1A, PRKCG, PRNP, PROM1, PROP1, PRPF31, PRPF8, PRPH2, PRPS1, PSAP, PSD95, PSEN1, PSEN2, PTCH1, PTEN, PTS, PUS1, PYGM, RAB23, RAD50, RAD51C, RAD51D, RAG2, RAPSN, RARS2, RB1, RDH12, RECQL4, RET, RHO, RICTOR, RMRP, ROS1, RP1, RP2, RPE65, RPGR, RPGRIP1L, RPL32P3, RS1, RTCA, RTEL1, RUNX1, SACS, SAMHD1, SCN1A, SCN2A, SDHA, SDHAF2, SDHB, SDHC, SDHD, SEL1L, SEPSECS, SERPINA1, SERPING1, SGCA, SGCB, SGCG, SGSH, SIRT1, SLC12A3, SLC12A6, SLC17A5, SLC22A5, SLC25A13, SLC25A15, SLC26A2, SLC26A4, SLC35A3, SLC35B4 SLC37A4, SLC39A4, SLC4A11, SLC6A8, SLC7A7, SMAD4, SMARCA4, SMARCAL1, SMARCB1, SMARCE1, SMN1, SMPD1, SNAI2, SNCA, SNRNP200, SOD1, SOX10, SPARA7, SPTBN2, STAR, STAT3, STK11, SUFU, SUMF1, SYNE1, SYNE2, SYS1, TARDBP, TAT, TBK1, TBP, TCIRG1, TCTN3, TECPR2, TERC, TERT, TFR2, TGFBR2, TGM1, TH, TLE3, TMEM127, TMEM138, TMEM216, TMEM43, TMEM67, TMPRSS6, TOP1, TOPORS, TP53, TPP1, TRAC, TRMU, TSFM, TSPAN14, TTBK2, TTC8, TTPA, TTR, TULP1, TYMP, UBE2G2, UBE2J1, UBE3A, USH1C, USH1G, USH2A, VEGF, VHL, VPS13A, VPS13B, VPS35, VPS45, VRK1, VSX2, VWF, WDR19, WDR48, WNT10A, WRN, WS2B, WS2C, WT1, XPA, XPC, XPF, XRCC3, YAP1, ZAC1, ZFYVE26, and ZNF423.

The compositions and methods described herein may be used to treat, prevent, or inhibit a disease or syndrome in a subject. By way of non-limiting example, the disease may be a cancer, an ophthalmological disorder, a neurological disorder, a neurodegenerative disease, a blood disorder, or a metabolic disorder, or a combination thereof. The disease may be an inherited disorder, also referred to as a genetic disorder. The disease may be the result of an infection or associated with an infection. In some embodiments, the disease is a liver disease, a lung disease, an eye disease, or a muscle disease. A genetic disease may comprise a single mutation, multiple mutations, or a chromosomal aberration. In some embodiments, a genetic disease is a disease caused by one or more mutations in the DNA of an organism. In some instances, a disease is referred to as a disorder. Mutations may be due to several different cellular mechanisms, including, but not limited to, an error in DNA replication, recombination, or repair, or due to environmental factors. Mutations may be encoded in the sequence of a target nucleic acid from the germline of an organism. Exemplary diseases and syndromes include, but are not limited to: 11-hydroxylase deficiency; 17,20-desmolase deficiency; 17-hydroxylase deficiency; 3-hydroxyisobutyrate aciduria; 3-hydroxysteroid dehydrogenase deficiency; 46,XY gonadal dysgenesis; AAA syndrome; ABCA3 deficiency; ABCC8-associated hyperinsulinism; aceruloplasminemia; acromegaly; achondrogenesis type 2; acral peeling skin syndrome; acrodermatitis enteropathica; adrenocortical micronodular hyperplasia; adrenoleukodystrophies; adrenomyeloneuropathies; Aicardi-Goutieres syndrome; Alagille disease (also called Alagille Syndrome); Alexander Disease, Alpers syndrome; alpha-1 antitrypsin deficiency (AATD); alpha-mannosidosis; Alstrom syndrome; Alzheimer's disease; amebic dysentery; amelogenesis imperfecta; amish type microcephaly; amyotrophic lateral sclerosis (ALS); anauxetic dysplasia; androgen insensitivity syndrome; antiphospholipid syndrome; Antley-Bixler syndrome; APECED, Apert syndrome, aplasia of lacrimal and salivary glands, argininemia, arrhythmogenic right ventricular dysplasia, Arts syndrome, ARVD2, arylsulfatase deficiency type metachromatic leokodystrophy, ataxia telangiectasia, autoimmune lymphoproliferative syndrome; autoimmune polyglandular syndrome type 1; autosomal dominant anhidrotic ectodermal dysplasia; autosomal dominant polycystic kidney disease; autosomal recessive microtia; autosomal recessive renal glucosuria; autosomal visceral heterotaxy; babesiosis; balantidial dysentery; Bardet-Biedl syndrome; Bartter syndrome; basal cell nevus syndrome; Batten disease; benign recurrent intrahepatic cholestasis; beta-mannosidosis; Bethlem myopathy; Blackfan-Diamond anemia; blepharophimosis; Byler disease; C syndrome; CADASIL; carbamyl phosphate synthetase deficiency; cardiofaciocutaneous syndrome; Carney triad; camitine palmitoyltransferase deficiencies; cartilage-hair hypoplasia; cblC type of combined methylmalonic aciduria; CD18 deficiency; CD3Z-associated primary T-cell immunodeficiency; CD40L deficiency; CDAGS syndrome; CDG1A; CDG1B; CDG1M; CDG2C; CEDNIK syndrome; central core disease; centronuclear myopathy; cerebral capillary malformation; cerebrooculofacioskeletal syndrome type 4; cerebrooculogacioskeletal syndrome; cerebrotendinous xanthomatosis; Chaga's Disease; Charcot Marie Tooth Disesase; cherubism; CHILD syndrome; chronic granulomatous disease; chronic recurrent multifocal osteomyelitis; citrin deficiency; classic hemochromatosis; CNPPB syndrome; cobalamin C disease; Cockayne syndrome; coenzyme Q10 deficiency; Coffin-Lowry syndrome; Cohen syndrome; combined deficiency of coagulation factors V; common variable immune deficiency; complete androgen insentivity; cone rod dystrophies; conformational diseases; congenital bile adid synthesis defect type 1; congenital bile adid synthesis defect type 2; congenital defect in bile acid synthesis type; congenital erythropoietic porphyria; congenital generalized osteosclerosis; Comelia de Lange syndrome; Cousin syndrome; Cowden disease; COX deficiency; Cri du chat syndrome; Crigler-Najjar disease; Crigler-Najjar syndrome type 1; Crisponi syndrome; Crouzon syndrome; Currarino syndrome; Curth-Macklin type ichthyosis hystrix; cutis laxa; cystic fibrosis; cystinosis; d-2-hydroxyglutaric aciduria; DDP syndrome; Dejerine-Sottas disease; Denys-Drash syndrome; Dercum disease; desmin cardiomyopathy; desmin myopathy; DGUOK-associated mitochondrial DNA depletion; diabetes Type I; diabetes Type II; disorders of glutamate metabolism; distal spinal muscular atrophy type 5; DNA repair diseases; dominant optic atrophy; Doyne honeycomb retinal dystrophy; Dravet Syndrome; Duchenne muscular dystrophy; dyskeratosis congenita; Ehlers-Danlos syndrome type 4; Ehlers-Danlos syndromes; Elejalde disease; Ellis-van Creveld disease; Emery-Dreifuss muscular dystrophies; encephalomyopathic mtDNA depletion syndrome; encephalitis; enzymatic diseases; EPCAM-associated congenital tufting enteropathy; epidermolysis bullosa with pyloric atresia; epilepsy; facioscapulohumeral muscular dystrophy; Factor V Leiden Thrombophilia; Faisalabad histiocytosis; familial atypical mycobacteriosis; familial capillary malformation-arteriovenous; Familial Creutzfeld-Jakob Disease; familial esophageal achalasia; familial glomuvenous malformation; familial hemophagocytic lymphohistiocytosis; familial mediterranean fever; familial megacalyces; familial schwannomatosisl; familial spina bifida; familial splenic asplenia/hypoplasia; familial thrombotic thrombocytopenic purpura; Fanconi disease (Fanconi anemia); Feingold syndrome; FENIB; fibrodysplasia ossificans progressiva; FKTN; Fragile X syndrome; Francois-Neetens fleck comeal dystrophy; Frasier syndrome; Friedreich's ataxia; FTDP-17; fucosidosis; G6PD deficiency; galactosialidosis; Galloway syndrome; Gardner syndrome; Gaucher disease; Gitelman syndrome; GLUT1 deficiency; GM2-Gangliosidoses (e.g., Tay Sachs Disease, Sandhoff Disease) glycogen storage disease type 1b; glycogen storage disease type 2; glycogen storage disease type 3; glycogen storage disease type 4; glycogen storage disease type 9a; glycogen storage diseases; GM1-gangliosidosis; Greenberg syndrome; Greig cephalopolysyndactyly syndrome; hair genetic diseases; HANAC syndrome; harlequin type ichtyosis congenita; HDR syndrome; hearing loss; hemochromatosis type 3; hemochromatosis type 4; hemophilia A; hereditary angioedema type 3; hereditary angioedemas; hereditary hemorrhagic telangiectasia; hereditary hypofibrinogenemia; hereditary intraosseous vascular malformation; hereditary leiomyomatosis and renal cell cancer; hereditary neuralgic amyotrophy; hereditary sensory and autonomic neuropathy type; Hermansky-Pudlak disease; HHH syndrome; HHT2; hidrotic ectodermal dysplasia type 1; hidrotic ectodermal dysplasias; HNF4A-associated hyperinsulinism; HNPCC; homozygous familial hypercholesterolemia; human immunodeficiency with microcephaly; Huntington's disease; hyper-IgD syndrome; hyperinsulinism-hyperammonemia syndrome; hypercholesterolemia; hypertrophy of the retinal pigment epithelium; hypochondrogenesis; hypohidrotic ectodermal dysplasia; ICF syndrome; idiopathic congenital intestinal pseudo-obstruction; immunodeficiency with hyper-IgM type 1; immunodeficiency with hyper-IgM type 3; immunodeficiency with hyper-IgM type 4; immunodeficiency with hyper-IgM type 5; inbor errors of thyroid metabolism; infantile visceral myopathy; infantile X-linked spinal muscular atrophy; intrahepatic cholestasis of pregnancy; IPEX syndrome; IRAK4 deficiency; isolated congenital asplenia; Jeune syndrome; Johanson-Blizzard syndrome; Joubert syndrome; JP-HHT syndrome; juvenile hemochromatosis; juvenile hyalin fibromatosis; juvenile nephronophthisis; Kabuki mask syndrome; Kallmann syndromes; Kartagener syndrome; KCNJ11-associated hyperinsulinism; Keams-Sayre syndrome; Kostmann disease; Kozlowski type of spondylometaphyseal dysplasia; Krabbe disease; LADD syndrome; late infantile-onset neuronal ceroid lipofuscinosis; LCK deficiency; LDHCP syndrome; Leber Congenital Amaurosis Teyp 10; Legius syndrome; Leigh syndrome; lethal congenital contracture syndrome 2; lethal congenital contracture syndromes; lethal contractural syndrome type 3; lethal neonatal CPT deficiency type 2; lethal osteosclerotic bone dysplasia; Li Fraumeni syndrome; LIG4 syndrome; lipodystrophy; lissencephaly type 1 Imag; lissencephaly type 3; Loeys-Dietz syndrome; low phospholipid-associated cholelithiasis; Lynch Syndrome; lysinuric protein intolerance; a lysosomal storage disease (e.g., Hunter syndrome, Hurler syndrome); macular dystrophy; Maffucci syndrome; Majeed syndrome; mannose-binding protein deficiency; Marfan disease; Marshall syndrome; MASA syndrome; MCAD deficiency; McCune-Albright syndrome; MCKD2; Meckel syndrome; MECP2 Duplication Syndrome; Meesmann comeal dystrophy; megacystis-microcolon-intestinal hypoperistalsis; megaloblastic anemia type 1; MEHMO; MELAS; Melnick-Needles syndrome; MEN2s; meningitis; Menkes disease; metachromatic leukodystrophies; methylmalonic acidurias; methylvalonic aciduria; microcoria-congenital nephrosis syndrome; microvillous atrophy; migraine; mitochondrial neurogastrointestinal encephalomyopathy; monilethrix; monosomy X; mosaic trisomy 9 syndrome; Mowat-Wilson syndrome; mucolipidosis type 2; mucolipidosis type Ma; mucolipidosis type IV; mucopolysaccharidoses; mucopolysaccharidosis type 3A; mucopolysaccharidosis type 3C; mucopolysaccharidosis type 4B; multiminicore disease; multiple acyl-CoA dehydrogenation deficiency; multiple cutaneous and mucosal venous malformations; multiple endocrine neoplasia type 1; multiple sulfatase deficiency; myotonic dystrophy; NAIC; nail-patella syndrome; nemaline myopathies; neonatal diabetes mellitus; neonatal surfactant deficiency; nephronophtisis; Netherton disease; neurofibromatoses; neurofibromatosis type 1; Niemann-Pick disease type A; Niemann-Pick disease type B; Niemann-Pick disease type C; NKX2E; non-alcoholic fatty liver disease (NAFLD); non-alcoholic steatohepatitis (NASH); Noonan syndrome; North American Indian childhood cirrhosis; NROB1 duplication-associated DSD; ocular genetic diseases; oculo-auricular syndrome; OLEDAID; oligomeganephronia; oligomeganephronic renal hypolasia; Ollier disease; Opitz-Kaveggia syndrome; orofaciodigital syndrome type 1; orofaciodigital syndrome type 2; osseous Paget disease; osteogenesis imperfecta; otopalatodigital syndrome type 2; OXPHOS diseases; palmoplantar hyperkeratosis; panlobar nephroblastomatosis; Parkes-Weber syndrome; Parkinson's disease; partial deletion of 21q22.2-q22.3; Pearson syndrome; Pelizaeus-Merzbacher disease; Pendred syndrome; pentalogy of Cantrell; peroxisomal acyl-CoA-oxidase deficiency; Peutz-Jeghers syndrome; Pfeiffer syndrome; Pierson syndrome; pigmented nodular adrenocortical disease; pipecolic acidemia; Pitt-Hopkins syndrome; plasmalogens deficiency; pleuropulmonary blastoma and cystic nephroma; polycystic kidney disease; polycystic ovarian disease; polycystic lipomembranous osteodysplasia; Pompe disease; porphyrias; premature ovarian failure; primary erythermalgia; primary hemochromatoses; primary hyperoxaluria; progressive familial intrahepatic cholestasis; propionic acidemia; pyruvate decarboxylase deficiency; RAPADILINO syndrome; renal cystinosis; retinitis pigmentosa; Rett Syndrome; rhabdoid tumor predisposition syndrome; Rieger syndrome; ring chromosome 4; Roberts syndrome; Robinow-Sorauf syndrome; Rothmund-Thomson syndrome; severe combined immunodeficiency disorder (SCID); Saethre-Chotzen syndrome; Sandhoff disease; SC phocomelia syndrome; SCAS; Schinzel phocomelia syndrome; short rib-polydactyly syndrome type 1; short rib-polydactyly syndrome type 4; short-rib polydactyly syndrome type 2; short-rib polydactyly syndrome type 3; Shwachman disease; Shwachman-Diamond disease; sickle cell anemia; Silver-Russell syndrome; Simpson-Golabi-Behmel syndrome; Smith-Lemli-Opitz syndrome; SPG7-associated hereditary spastic paraplegia; spherocytosis; spinocerebellar ataxia; split-hand/foot malformation with long bone deficiencies; spondylocostal dysostosis; sporadic visceral myopathy with inclusion bodies; storage diseases; Stargardt macular dystrophy; STRA6-associated syndrome; stroke; Tay-Sachs disease; thanatophoric dysplasia; thyroid metabolism diseases; Tourette syndrome; transthyretin-associated amyloidosis; trisomy 13; trisomy 22; trisomy 2p syndrome; tuberous sclerosis; tufting enteropathy; urea cycle diseases; Usher Syndrome; Van Den Ende-Gupta syndrome; Van der Woude syndrome; variegated mosaic aneuploidy syndrome; VLCAD deficiency; von Hippel-Lindau disease; von Willebrand disease; Waardenburg syndrome; WAGR syndrome; Walker-Warburg syndrome; Werner syndrome; Wilson disease; Wolcott-Rallison syndrome; Wolfram syndrome; X-linked agammaglobulinemia; X-linked chronic idiopathic intestinal pseudo-obstruction; X-linked cleft palate with ankyloglossia; X-linked dominant chondrodysplasia punctata; X-linked ectodermal dysplasia; X-linked Emery-Dreifuss muscular dystrophy; X-linked lissencephaly; X-linked lymphoproliferative disease; X-linked visceral heterotaxy; xanthinuria type 1; xanthinuria type 2; xeroderma pigmentosum; XPV; and Zellweger disease.

In some embodiments, compositions and methods cause the death of a cell harboring a mutation in a gene associated with the disease or the expression thereof. In some embodiments, the disease is Alzheimer's disease and the gene is selected from APP, BACE-1, PSD95, MAPT, PSEN1, PSEN2, and APOEε4. In some embodiments, the disease is Parkinson's disease and the gene is selected from SNCA, GDNF, and LRRK2. In some embodiments, the disease comprises Centronuclear myopathy and the gene is DNM2. In some embodiments, the disease is Huntington's disease and the gene is HTT. In some embodiments, the disease is Alpha-1 antitrypsin deficiency (AATD) and the gene is SERPINA1. In some embodiments, the disease is amyotrophic lateral sclerosis (ALS) and the gene is selected from SOD1, FUS, C9ORF72, ATXN2, TARDBP, and CHCHD10. In some embodiments, the disease comprises Alexander Disease and the gene is GFAP. In some embodiments, the disease comprises Angelman Syndrome and the gene is UBE3A. In some embodiments, the disease comprises MECP2 Duplication syndrome and Rett syndrome and the gene is MECP2. In some embodiments, the disease comprises fragile X syndrome and the gene is FMR1. In some embodiments, the disease comprises CNS trauma and the gene is VEGF. In some embodiments, the disease comprises GM2-Gangliosidoses (e.g., Tay Sachs Disease, Sandhoff disease) and the gene is selected from HEXA and HEXB. In some embodiments, the disease comprises Hearing loss disorders and the gene is DFNA36. In some embodiments, the disease is Pompe disease and the gene is GAA. In some embodiments, the disease is Retinitis pigmentosa and the gene is selected from PDE6B, RHO, RP1, RP2, RPGR, PRPH2, IMPDH1, PRPF31, CRB1, PRPF8, TULP1, CA4, HPRPF3, ABCA4, EYS, CERKL, FSCN2, TOPORS, SNRNP200, PRCD, NR2E3, MERTK, USH2A, PROM1, KLHL7, CNGB1, TTC8, ARL6, DHDDS, BEST1, LRAT, SPARA7, CRX CLRN1, RPE65, and WDR19. In some embodiments, the disease comprises Leber Congenital Amaurosis Type 10 and the gene is CEP290. In some embodiments, the disease is cardiovascular disease and/or lipodystrophies and the gene is selected from APOA1, ANGPTL3, APOCIII, CFB, AGT, FXI, FXII, PKK, PCSK9, APOL1, and TTR. In some embodiments, the disease comprises acromegaly and the gene is GHR. In some embodiments, the disease is diabetes and the gene is GCGR. In some embodiments, the disease is NAFLD/NASH and the gene is selected from DGAT2 and PNPLA3. In some embodiments, the disease is cancer and the gene is selected from STAT3, YAP1, FOXP3, AR (Prostate cancer), and IRF4 (multiple myeloma). In some embodiments, the disease is cystic fibrosis and the gene is CFTR. In some embodiments, the disease is Duchenne Muscular Dystrophy and the gene is DMD. In some embodiments, the disease comprises angioedema and the gene is PKK. In some embodiments, the disease comprises thalassemia and the gene is TMPRSS6. In some embodiments, the disease comprises achondroplasia and the gene is FGFR3. In some embodiments, the disease comprises Cri du chat syndrome and the gene is selected from CTNND2. In some embodiments, the disease comprises cystic fibrosis and the gene is CFTR. In some embodiments, the disease comprises sickle cell anemia and the gene is Beta globin gene. In some embodiments, the disease comprises Alagille Syndrome and the gene is selected from JAG1 and NOTCH2. In some embodiments, the disease comprises Charcot Marie Tooth Disease and the gene is selected from PMP22 and MFN2. In some embodiments, the disease comprises Crouzon syndrome and the gene is selected from FGFR2, FGFR3, and FGFR3. In some embodiments, the disease comprises Dravet Syndrome and the gene is selected from SCN1A and SCN2A. In some embodiments, the disease comprises Emery-Dreifuss syndrome and the gene is selected from EMD, LMNA, SYNE1, SYNE2, FHL1, and TMEM43. In some embodiments, the disease comprises Factor V Leiden Thrombophilia and the gene is F5. In some embodiments, the disease comprises Fanconi anemia and the gene is selected from FANCA, FANCB, FANCC, FANCD1, FANCD2, FANCE, FANCF, FANCG, FANCI, FANCJ, FANCL, FANCM, FANCN, FANCP, FANCS, RAD51C, and XPF. In some embodiments, the disease comprises Familial Creutzfeld-Jakob Disease and the gene is PRNP. In some embodiments, the disease comprises Familial Mediterranean Fever and the gene is MEFV. In some embodiments, the disease comprises Friedreich's ataxia and the gene is FXN. In some embodiments, the disease comprises Gaucher disease and the gene is GBA. In some embodiments, the disease comprises Hemochromatosis and the gene is C282Y. In some embodiments, the disease comprises Hemophilia and the gene is FVIII. In some embodiments, the disease comprises Joubert syndrome and the gene is selected from INPP5E, TMEM216, AHI1, NPHP1, CEP290, TMEM67, RPGRIP1L, ARL13B, CC2D2A, OFD1, TMEM138, TCTN3, ZNF423, and AMRC9. In some embodiments, the disease comprises Li-Fraumeni syndrome and the gene is TP53. In some embodiments, the disease comprises Lynch syndrome and the gene is selected from MSH2, MLH1, MSH6, PMS2, PMS1, TGFBR2, and MLH3. In some embodiments, the disease comprises Marfan syndrome and the gene is FBN1. In some embodiments, the disease comprises methylmalonic acidemia and the gene is selected from MMAA, MMAB, and MUT. In some embodiments, the disease is myotonic dystrophy and the gene is selected from CNBP and DMPK. In some embodiments, the disease comprises neurofibromatosis and the gene is selected from NF1, and NF2. In some embodiments, the disease comprises osteogenesis imperfecta and the gene is selected from COL1A1, COL1A2, and IFITM5. In some embodiments, the disease is non-small cell lung cancer and the gene is selected from KRAS, EGFR, ALK, METex14, BRAF V600E, ROS1, RET, and NTRK. In some embodiments, the disease comprises Peutz-Jeghers syndrome and the gene is STK11. In some embodiments, the disease comprises polycystic kidney disease and the gene is selected from PKD1 and PKD2. In some embodiments, the disease comprises Spinocerebellar ataxia and the gene is selected from ATXN1, ATXN2, ATXN3, PLEKHG4, SPTBN2, CACNA1A, ATXN7, ATXN8OS, ATXN10, TTBK2, PPP2R2B, KCNC3, PRKCG, ITPR1, TBP, KCND3, and FGF14. In some embodiments, the disease comprises Usher Syndrome and the gene is selected from MYO7A, USH1C, CDH23, PCDH15, USH1G, USH2A, GPR98, DFNB31, and CLRN1. In some embodiments, the disease comprises von Willebrand disease and the gene is VWF. In some embodiments, the disease comprises Waardenburg syndrome and the gene is selected from PAX3, MITF, WS2B, WS2C, SNAI2, EDNRB, EDN3, and SOX10. In some embodiments, the disease comprises von Hippel-Lindau disease and the gene is VHL. In some embodiments, the disease comprises Zellweger syndrome and the gene is selected from PEX1, PEX2, PEX3, PEX5, PEX6, PEX10, PEX12, PEX13, PEX14, PEX16, PEX19, and PEX26.

### Cancer

In some embodiments, compositions and methods cause the death of a cell harboring a mutation in a gene associated with a cancer. In some embodiments, the cancer is a solid cancer (i.e., a tumor). In some embodiments, the cancer is selected from a blood cell cancer, a leukemia, and a lymphoma. The cancer can be a leukemia, such as, by way of non-limiting example, acute myeloid (or myelogenous) leukemia (AML), chronic myeloid (or myelogenous) leukemia (CML), acute lymphocytic (or lymphoblastic) leukemia (ALL), and chronic lymphocytic leukemia (CLL). In some embodiments, the cancer is any one of colon cancer, rectal cancer, renal-cell carcinoma, liver cancer, bladder cancer, cancer of the kidney or ureter, lung cancer, non small cell lung cancer, cancer of the small intestine, esophageal cancer, melanoma, bone cancer, pancreatic cancer, skin cancer, brain cancer (e.g., glioblastoma), cancer of the head or neck, melanoma, uterine cancer, ovarian cancer, breast cancer, testicular cancer, cervical cancer, stomach cancer, Hodgkin's Disease, non-Hodgkin's lymphoma, and thyroid cancer.

In some embodiments, mutations are associated with cancer or are causative of cancer. The target nucleic acid, in some embodiments, comprises a portion of a gene comprising a mutation associated with cancer, a gene whose overexpression is associated with cancer, a tumor suppressor gene, an oncogene, a checkpoint inhibitor gene, a gene associated with cellular growth, a gene associated with cellular metabolism, a gene associated with cell cycle, or a combination thereof. Non-limiting examples of genes comprising a mutation associated with cancer are ABL, AF4/HRX AKT-2, ALK, ALK/NPM, AML1, AML1/MTG8, APC, ATM, AXIN2, AXL, BAP1, BARD1, BCL-2, BCL-3, BCL-6, BCR/ABL, BLM, BMPR1A, BRCA1, BRCA2, BRIP1, c-MYC, CASR, CDC73, CDH1, CDK4, CDKN1B, CDKN1C, CDKN2A, CEBPA, CHEK2, CREBBP, CTNNA1, DBL, DEK/CAN, DICER1, DIS3L2, E2A/PBX1, EGFR, ENL/HRX EPCAM, ERG/TLS, ERBB, ERBB-2, ETS-1, EWS/FLI-1, FH, FLCN, FMS, FOS, FPS, GATA2, GLI, GPGSP, GREM1, HER2/neu, HOX11, HOXB13, HST, IL-3, INT-2, JAK1, JUN, KIT, KS3, K-SAM, LBC, LCK, LMO1, LMO2, L-MYC, LYL-1, LYT-10, LYT-10/Cα1, MAS, MAX, MDM-2, MEN1, MET, MITF, MLH1, MLL, MOS, MSH1, MSH2, MSH3, MSH6, MTG8/AML1, MUTYH, MYB, MYH11/CBFB, NBN, NEU, NF1, NF2, N-MYC, NTHL1, OST, PALB2, PAX-5, PBX1/E2A, PDGFRA, PHOX2B, PIM-1, PMS2, POLD1, POLE, POT1, PRAD-1, PRKAR1A, PTCH1, PTEN, RAD50, RAD51C, RAD51D, RAF, RAR/PML, RAS-H, RAS-K, RAS-N, RB1, RECQL4, REL/NRG, RET, RHOM1, RHOM2, ROS, RUNX1, SDHA, SDHAF, SDHB, SDHC, SDHD, SET/CAN, SIS, SKI, SMAD4, SMARCA4, SMARCB1, SMARCE1, SRC, STK11, SUFU, TAL1, TAL2, TAN-1, TIAM1, TERC, TERT, TMEM127, TP53, TSC1, TSC2, TRK, VHL, WRN, and WT1. Non-limiting examples of oncogenes are KRAS, NRAS, BRAF, MYC, CTNNB1, and EGFR. In some instances, the oncogene is a gene that encodes a cyclin dependent kinase (CDK). Non-limiting examples of CDKs are Cdk1, Cdk4, Cdk5, Cdk7, Cdk8, Cdk9, Cdk11 and Cdk20. Non-limiting examples of tumor suppressor genes are TP53, RB1, and PTEN.

### Infections

In some embodiments, compositions and methods cause the death of a cell harboring a pathogen. Infections may be caused by a pathogen, e.g., bacteria, viruses, fungi, and parasites. Compositions and methods may modify a target nucleic acid associated with the pathogen or parasite causing the infection. In some embodiments, the target nucleic acid may be in the pathogen or parasite itself or in a cell, tissue or organ of the subject that the pathogen or parasite infects. In some embodiments, the methods described herein include treating an infection caused by one or more bacterial pathogens. Non-limiting examples of bacterial pathogens include Acholeplasma laidlawii, Brucella abortus, Chlamydia psittaci, Chlamydia trachomatis, Cryptococcus neoformans, Escherichia coli, Legionella pneumophila, Lyme disease spirochetes, methicillin-resistant Staphylococcus aureus, Mycobacterium leprae, Mycobacterium tuberculosis, Mycoplasma arginini, Mycoplasma arthritidis, Mycoplasma genitalium, Mycoplasma hyorhinis, Mycoplasma orale, Mycoplasma pneumoniae, Mycoplasma salivarium, Neisseria gonorrhoeae, Neisseria meningitidis, Pneumococcus, Pseudomonas aeruginosa, sexually transmitted infection, Streptococcus agalactiae, Streptococcus pyogenes, and Treponema pallidum.

In some embodiments, compositions and methods cause the death of a cell harboring a viral pathogen. Non-limiting examples of viral pathogens include adenovirus, blue tongue virus, chikungunya, coronavirus (e.g., SARS-CoV-2), cytomegalovirus, Dengue virus, Ebola, Epstein-Barr virus, feline leukemia virus, Hemophilus influenzae B, Hepatitis Virus A, Hepatitis Virus B, Hepatitis Virus C, herpes simplex virus I, herpes simplex virus II, human papillomavirus (HPV), human serum parvo-like virus, human T-cell leukemia viruses, immunodeficiency virus (e.g., HIV), influenza virus, lymphocytic choriomeningitis virus, measles virus, mouse mammary tumor virus, mumps virus, murine leukemia virus, polio virus, rabies virus, Reovirus, respiratory syncytial virus (RSV), rubella virus, Sendai virus, simian virus 40, Sindbis virus, varicella-zoster virus, vesicular stomatitis virus, wart virus, West Nile virus, yellow fever virus, or any combination thereof.

In some embodiments, compositions and methods cause the death of a cell harboring a parasite. Non-limiting examples of parasites include helminths, annelids, platyhelminthes, nematodes, and thorny-headed worms. In some embodiments, parasitic pathogens comprise, without limitation, Babesia bovis, Echinococcus granulosus, Eimeria tenella, Leishmania tropica, Mesocestoides corti, Onchocerca volvulus, Plasmodium falciparum, Plasmodium vivax, Schistosoma japonicum, Schistosoma mansoni, Schistosoma spp., Taenia hydatigena, Taenia ovis, Taenia saginata, Theileria parva, Toxoplasma gondii, Toxoplasma spp., Trichinella spiralis, Trichomonas vaginalis, Trypanosoma brucei, Trypanosoma cruzi, Trypanosoma rangeli, Trypanosoma rhodesiense, Balantidium coli, Entamoeba histolytica, Giardia spp., Isospora spp., Trichomonas spp., or any combination thereof.

### Compositions, Methods, and Systems for Modifying Target Nucleic Acids

Disclosed herein are compositions, methods, and systems for modifying a target nucleic acid. Such compositions, methods, and systems, in some embodiments, can include a programmable nuclease as described herein (e.g., a programmable nuclease comprising at least one HEPN or HEPN-like domain; or the programmable nuclease comprising at least 65%, at least 70%, at least 75%, at least 80%, at least 85%, at least 90%, at least 95%, at least 97%, or at least 98%, at least 99%, or 100% identical to any one of SEQ ID NOs: 1-27) and an engineered guide nucleic acid, wherein the engineered guide nucleic acid comprises a nucleotide sequence that can bind to the target nucleic acid. The target nucleic acid may be a gene or a portion thereof. Compositions, methods, or systems may modify a coding portion of a gene, a non-coding portion of a gene, or a combination thereof. Modifying at least one gene using the compositions, methods, and systems described herein may reduce or increase expression of one or more genes. In some embodiments, compositions, methods, and systems reduce expression of one or more genes by at least 10%, at least 20%, at least 30%, at least 40%, at least 50%, at least 60%, at least 70%, at least 80%, at least 90%, or at least 95%. In some embodiments, compositions, methods, and systems remove all expression of a gene, also referred to as genetic knock out. In some embodiments, compositions, methods, and systems increase expression of one or more genes by at least 10%, at least 20%, at least 30%, at least 40%, at least 50%, at least 60%, at least 70%, at least 80%, at least 90%, or at least 100%.

In some instances, compositions, methods, and systems use Cas proteins that are fused to a heterologous protein. Heterologous proteins include, but are not limited to, transcriptional activators, transcriptional repressors, deaminases, methyltransferases, acetyltransferases, and other nucleic acid modifying proteins. In some cases, Cas proteins need not be fused to a partner protein to accomplish the required protein (expression) modification. In some embodiments, a transcriptional activator is a polypeptide or a fragment thereof that can activate or increase transcription of a target nucleic acid molecule. In some embodiments, a transcriptional repressor is a polypeptide or a fragment thereof that is capable of arresting, preventing, or reducing transcription of a target nucleic acid.

In some embodiments, compositions, methods, and systems comprise a nucleic acid expression vector, or use thereof, to introduce a Cas protein, guide nucleic acid, donor template or any combination thereof to a cell. In some embodiments, a nucleic acid expression vector is a plasmid that can be used to express a nucleic acid of interest. In some embodiments, the nucleic acid expression vector is a viral vector. Viral vectors include, but are not limited to, retroviruses, adenoviruses, adeno-associated viruses, and herpes simplex viruses. In some embodiments, the viral vector is a replication-defective viral vector, comprising an insertion of a therapeutic gene inserted in genes essential to the lytic cycle, preventing the virus from replicating and exerting cytotoxic effects. In some embodiments, the viral vector is an adeno associated viral (AAV) vector. In some embodiments, the nucleic acid expression vector is a non-viral vector. In some embodiments, compositions, methods, and systems comprise a lipid, polymer, nanoparticle, or a combination thereof, or use thereof, to introduce a Cas protein, guide nucleic acid, donor template or any combination thereof to a cell. Non-limiting examples of lipids and polymers are cationic polymers, cationic lipids, or bio-responsive polymers. In some embodiments, the bio-responsive polymer exploits chemical-physical properties of the endosomal environment (e.g., pH) to preferentially release the genetic material in the intracellular space.

### Fusion Partners

Provided herein are fusion programmable nucleases that comprise at least one fusion partner. In some embodiments, fusion partners provide enzymatic activity that modifies a target nucleic acid. In some embodiments, fusion partners provide enzymatic activity that modifies expression of a target nucleic acid. The target nucleic acid may be a gene. The target nucleic acid may be DNA. The target nucleic acid may be RNA. Such enzymatic activities include, but are not limited to, nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, and glycosylase activity. Examples of enzymatic activity that modifies the target nucleic acid include, but are not limited to: nuclease activity such as that provided by a restriction enzyme (e.g., FokI nuclease); methyltransferase activity such as that provided by a methyltransferase (e.g., HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants)); demethylase activity such as that provided by a demethylase (e.g., Ten-Eleven Translocation (TET) dioxygenase 1 (TET1CD), TET1, DME, DML1, DML2, ROS1); DNA repair activity; DNA damage (e.g., oxygenation) activity; deamination activity such as that provided by a deaminase (e.g., a cytosine deaminase enzyme such as rat APOBEC1); dismutase activity; alkylation activity; depurination activity; oxidation activity; pyrimidine dimer forming activity; integrase activity such as that provided by an integrase and/or resolvase (e.g., Gin invertase such as the hyperactive mutant of the Gin invertase, GinH106Y; human immunodeficiency virus type 1 integrase (IN); Tn3 resolvase); transposase activity, recombinase activity such as that provided by a recombinase (e.g., catalytic domain of Gin recombinase); as well as polymerase activity, ligase activity, helicase activity, photolyase activity, and glycosylase activity.

In some embodiments, fusion partners have enzymatic activity that modifies a protein associated with a target nucleic acid. The protein may be a histone, an RNA binding protein, or a DNA binding protein. Such enzymatic activities include, but are not limited to, methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, de-ribosylation activity, myristoylation activity, and demyristoylation activity. Examples of such enzymatic activities include methyltransferase activity such as that provided by a histone methyltransferase (HMT) (e.g., suppressor of variegation 3-9 homolog 1 (SUV39H1, also known as KMT1A), euchromatic histone lysine methyltransferase 2 (G9A, also known as KMT1C and EHMT2), SUV39H2, ESET/SETDB1, SET1A, SET1B, MLL1 to 5, ASH1, SYMD2, NSD1, DOT1L, Pr-SET7/8, SUV4-20H1, EZH2, RIZ1); demethylase activity such as that provided by a histone demethylase (e.g., Lysine Demethylase 1A (KDM1A also known as LSD1), JHDM2a/b, JMJD2A/JHDM3A, JMJD2B, JMJD2C/GASC1, JMJD2D, JARID1A/RBP2, JARID1B/PLU-1, JARID1C/SMCX, JARID1D/SMCY, UTX, JMJD3); acetyltransferase activity such as that provided by a histone acetylase transferase (e.g., catalytic core/fragment of the human acetyltransferase p300, GCN5, PCAF, CBP, TAF1, TIP60/PLIP, MOZ/MYST3, MORF/MYST4, HBO1/MYST2, HMOF/MYST1, SRC1, ACTR, P160, CLOCK); deacetylase activity such as that provided by ahistone deacetylase (e.g., HDAC1, HDAC2, HDAC3, HDAC8, HDAC4, HDAC5, HDAC7, HDAC9, SIRT1, SIRT2, HDAC11); kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, and demyristoylation activity.

In some instances, the programmable nuclease does not modify the target nucleic acid, but it is fused to a fusion partner protein that modifies the target nucleic acid when the complex contacts the target nucleic acid. In some embodiments, fusion programmable nucleases, fusion proteins, and fusion polypeptides are proteins comprising at least two heterologous polypeptides. Often a fusion programmable nuclease comprises a programmable nuclease and a fusion partner protein. In general, the fusion partner protein is not a programmable nuclease. Examples of fusion partner proteins are provided herein. In some embodiments, fusion partner proteins or fusion partners, are proteins, polypeptides or peptides that are fused to a programmable nuclease. The fusion partner generally imparts some function to the fusion protein that is not provided by the programmable nuclease. The fusion partner may provide a detectable signal. The fusion partner may modify a target nucleic acid, including changing a nucleobase of the target nucleic acid and making a chemical modification to one or more nucleotides of the target nucleic acid. The fusion partner may be capable of modulating the expression of a target nucleic acid. The fusion partner may inhibit, reduce, activate or increase expression of a target nucleic acid via additional proteins or nucleic acid modifications to the target sequence.

It is understood that a fusion partner may comprise an entire protein or a functional fragment of the protein (e.g., a functional domain). In some embodiments, a functional fragment is a fragment of a protein that retains some function relative to the entire protein. In some embodiments, a functional domain is a region of one or more amino acids in a protein that is required for an activity of the protein, or the full extent of that activity, as measured in an in vitro assay. Activities include, but are not limited to nucleic acid binding, nucleic acid modification, nucleic acid cleavage, protein binding. The absence of the functional domain, including mutations of the functional domain, would abolish or reduce activity. Non-limiting examples of functions are nucleic acid binding, protein binding, nuclease activity, nickase activity, deaminase activity, demethylase activity, or acetylation activity. In some embodiments, the functional domain interacts with or binds a target nucleic acid, including intramolecular and/or intermolecular secondary structures thereof, e.g., hairpins, stem-loops, etc. The functional domain may interact transiently or irreversibly, directly or indirectly with a target nucleic acid. In some embodiments, the functional domain has nuclease activity. A functional domain may be a domain of a protein selected from the group comprising endonucleases; proteins and protein domains capable of stimulating RNA cleavage; exonucleases; deadenylases; proteins and protein domains having nonsense mediated RNA decay activity; proteins and protein domains capable of stabilizing RNA; proteins and protein domains capable of repressing translation; proteins and protein domains capable of stimulating translation; proteins and protein domains capable of modulating translation (e.g., translation factors such as initiation factors, elongation factors, release factors, etc., e.g., eIF4G); proteins and protein domains capable of polyadenylation of RNA; proteins and protein domains capable of polyuridinylation of RNA; proteins and protein domains having RNA localization activity; proteins and protein domains capable of nuclear retention of RNA; proteins and protein domains having RNA nuclear export activity; proteins and protein domains capable of repression of RNA splicing; proteins and protein domains capable of stimulation of RNA splicing; proteins and protein domains capable of reducing the efficiency of transcription; and proteins and protein domains capable of stimulating transcription.

### Recombinant Nucleic Acids, Host Cells and Methods of Producing Programmable Nucleases

In some aspects, also provided herein is a recombinant nucleic acid encoding a programmable nuclease described herein (e.g., TABLE 1). Accordingly, in some embodiments, provided herein is a recombinant nucleic acid comprising an amino acid sequence that at least 65%, at least 70%, at least 75%, at least 80%, at least 85%, at least 90%, at least 95%, at least 97%, or at least 98%, at least 99%, or 100% identical to any one of SEQ ID NOs: 1-27. In some embodiments, the nucleic acid comprises a nucleotide sequence encoding the programmable nuclease operatively linked to a promoter. In some embodiments, a vector comprises a recombinant nucleic acid as described herein.

In some aspects, also provided herein is a non-naturally occurring host cell that comprises a recombinant nucleic acid as described herein. In some embodiments, the non-naturally occurring host cell is a microbial organism. In some embodiments, the host cell is a bacterial cell, a yeast cell, a plant cell, or a mammalian cell. In some embodiments, the host cell is a human cell. In some embodiments, the host cell is a non-human mammalian cell. In some embodiments, the host cell is an insect cell. In some embodiments, the host cell is an arthropod cell. In some embodiments, the host cell is a fungal cell. In some embodiments, the host cell is an algal cell. Methods for generating such host cells are well known to those skilled in the art and include those described in Rosano and Ceccarelli, Front Microbiol. 5:172 (2014), Kaur et al., Int. J. Biol. Macromol., 106:803-822 (2018), and Rosano et al., Protein Sci., 28(8):1412-1422 (2019). In some embodiments, the introduction of the recombinant nucleic acid into the host cell comprises electroporation, nucleofection, chemical methods, transfection, transduction, transformation, or microinjection. In some embodiments, the host cell is a prokaryotic cell or a eukaryotic cell. In some embodiments, the host cell is in vivo. In some embodiments, the host cell is ex vivo. In some embodiments, the host cell is in vitro.

In another aspect, also provided herein are methods for producing a programmable nuclease. Such a method can comprise culturing a non-naturally occurring host cell as described herein under a condition suitable for production of the programmable nuclease. Alternatively, such a method can comprise introducing into the host cell a recombinant nucleic acid as described herein or a vector as described herein and culturing the host cell under a condition suitable for production of the programmable nuclease. Conditions suitable for production of the programmable nuclease can be readily determined by a person skilled in the art, using well known culturing conditions for the host cell, which can vary depending upon the host cell. For example, production of the programmable nuclease can include fed-batch fermentation as described in Wyre et al., J. Ind. Microbiol. Biotechnol., 41(9):1391-404 (2014), multi-stage continuous high cell density culture systems as described in Chang et al., Biotechnol. Adv., 32(2):514-25 (2014), or integrated continuous production as described in Warikoo et al., Biotechnol. Bioeng., 109(12):3018-29 (2012).

In some embodiments, the method can include isolating the programmable nuclease. Isolation of the programmable nuclease can be done by methods well known in the art. For example, the produced programmable nuclease can be isolated from other components in the cell culture medium using extraction procedures, including extraction using organic solvents such as methanol, butanol, ethyl acetate, and the like, as well as methods that include continuous liquid-liquid extraction, solid-liquid extraction, solid phase extraction, pervaporation, membrane filtration, membrane separation, reverse osmosis, electrodialysis, dialysis, distillation, crystallization, centrifugation, extractive filtration, ion exchange chromatography, size exclusion chromatography, adsorption chromatography, ultrafiltration, medium pressure liquid chromatograpy (MPLC), and high pressure liquid chromatography (HPLC). All of the above methods are well known in the art and can be implemented in either analytical or preparative modes.

### EXAMPLES

### Example 1: Quantifying Trans-Collateral Activity via DETECTR

Type VI CRISPR/Cas proteins represented by SEQ ID NOs: 1-5 were assessed in their ability to detect a target nucleic acid in a sample using a DETECTR assay, using the spacer sequence, “CGACCUACUCUCCCAUACUCUUGUAUAUAG” (SEQ ID NO: 41), a single stranded RNA (ssRNA) target nucleic acid (“on-target 5S87”) comprising the sequence, “CUAUAUACAAGAGUAUGGGAGAGUAGGUCG (SEQ ID NO: 42),” and a random 12-mer ribonucleotide reporter. The assay was also run with positive control Cas protein, LbuCas13a (SEQ ID NO: 69). A reaction with nucleic acid, “target C,” having the sequence, “CAUGGCAUUCCACUUAUCAC (SEQ ID NO: 46),” was included as an off-target control. A reaction without any target sequence (“no target”) was included as a negative control.

Briefly, Type VI CRISPR/Cas proteins were mixed with crRNA at 160 nM and complexed for 30 minutes at room temperature in 1× M Buffer 1 (Imidazole pH 7.5, KCl, MgCl2, BSA, Igepal Ca-630, glycerol) to create 4× ribonucleoprotein particles (“RNP”). For trans cleavage reactions, 1× RNP was incubated with 500 μM ssRNA target and 250 nM ssRNA reporter for 60 minutes at 37° C. in 1× M Buffer 1. Trans cleavage activity was detected by fluorescence signal upon cleavage of a fluorophore-quencher reporter in a DETECTR reaction.

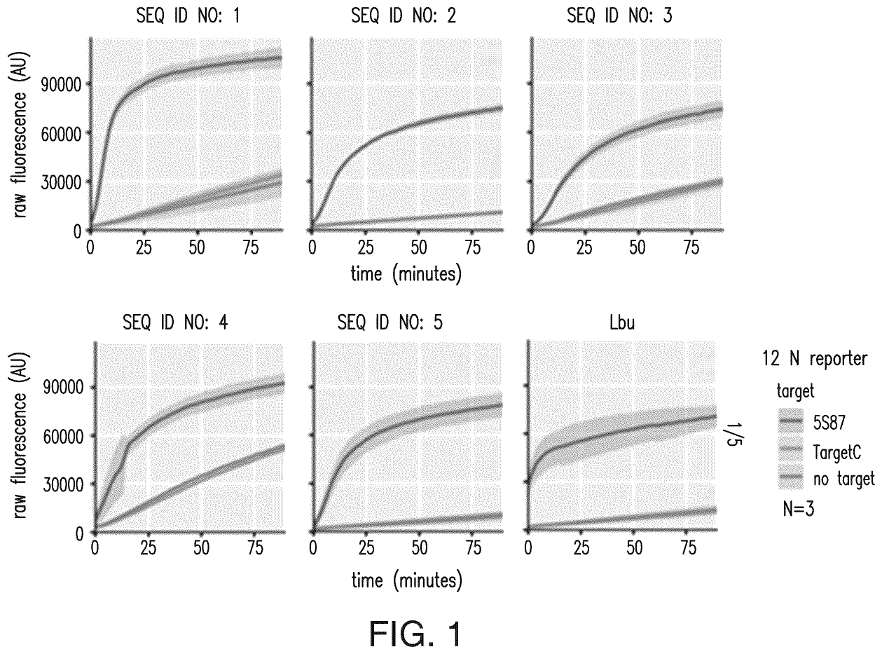

FIG. 1 shows fluorescence was detected in the presence of on-target 5S87. However, the assay with target C (off-target) did not generate any fluorescence above that of the assay with no target.

### Example 2: Screen of Type VI CRISPR/Cas Proteins for Trans Cleavage Activity with an ssRNA Target and an ssRNA Reporter

A high throughput assay was conducted to identify Cas programmable nucleases capable of producing trans cleavage of a single-stranded RNA reporter. Briefly, Type VI CRISPR/Cas proteins were mixed with crRNA at 160 nM and complexed for 15 minutes at 37° C. in 0.5× M Buffer 1 (Imidazole pH 7.5, KCl, MgCl2, BSA, Igepal Ca-630, glycerol) to create 4× ribonucleoprotein particles (“RNP”). For trans cleavage reactions, 1× RNP was incubated with 5 nM ssRNA target and 200 nM ssRNA reporter for 60 minutes at 37° C. in 1× M Buffer 1.

Trans cleavage activity was detected by fluorescence signal upon cleavage of a fluorophore-quencher reporter in a DETECTR reaction.

Table 4 shows these proteins achieved above 1.5 fold change in RNA-directed trans-cleavage activity (with ssRNA target and ssRNA reporter).

TABLE 4
Programmable nuclease Trans Cleavage Activity Score

| Cas | | | Activity Score |
|---|---|---|---|
| Effector | | | (max rate fold |
| SEQ ID | | crRNA SEQ ID | activity over |
| NO: | Name | NO: | negative control) |
| 15 | CasM.1584 | 60 | 17.9 |
| 16 | CasM.1730 | 61 | 28.7 |
| 17 | CasM.1770 | 62 | 5.9 |
| 18 | CasM.1816 | 63 | 5.0 |
| 19 | CasM.1862939 | 60 | 15.6 |
| 20 | CasM.1862895 | 64 | 26.0 |
| 21 | CasM.1862903 | 61 | 65.0 |
| 22 | CasM.1862909 | 65 | 82.0 |
| 23 | CasM.1862917 | 60 | 25.5 |
| 24 | CasM.1862921 | 65 | 18.8 |
| 25 | CasM.1862947 | 66 | 12.3 |

### Example 3: Type VI CRISPR/Cas Proteins Spacer Length Titration

This example describes experiments performed to test preferred spacer lengths for Type VI CRISPR/Cas proteins, CasM.1422-SEQ ID NO: 26, and CasM.1740-SEQ ID NO: 27. The assay was designed such that spacer length was shortened from both the 5′ end and the 3′ end of the spacer region, allowing the profiles of the two sets to be compared.

Type VI CRISPR/Cas proteins were incubated with crRNA and tracrRNA or sgRNAs in M Buffer 1 (Imidazole pH 7.5, KCl, MgCl2, BSA, Igepal Ca-630, glycerol) at 37°, followed by addition of target nucleic acid (5S87; SEQ ID NO: 42) at a final concentration of 0 pM, 1 pM, 10 pM, 100 pM, or 1000 pM. Cleavage activity was detected by fluorescence signal produced upon cleavage of a fluorophore-quencher reporter (included in the assay at 200 nM) in a DETECTR reaction.

The results of the samples with 10 pM, 100 pM and 1000 pM 5S87 reporter are presented in Table 6 and Table 7 show that CasM.1422-SEQ ID NO: 26 and CasM.1740-SEQ ID NO: 27 respectively can provide trans cleavage activity and exhibit a preference for an approximately 25 nucleotide spacer. Values provided are the mean of replicates. Standard deviations are available.

TABLE 6
CasM.1422 - SEQ ID NO: 26 trans cleavage
activity with varying spacer lengths

| Spacer length | 10 pM 5S87 | 100 pM 5S87 | 1000 pM 5S87 |
|---|---|---|---|
| and ID | target | target | target |
| 30 nt- 4754 | 20571.79 | 110493.30 | 456117.55 |
| 28 nt- 5017 | 39766.73 | 179018.06 | 455078.22 |
| 25 nt- 5018 | 47234.59 | 250641.38 | 480762.27 |
| 21 nt- 5019 | 5661.82 | 25077.17 | 138442.21 |
| 20 nt-5020 | 2860.29 | 3582.18 | 8334.36 |
| 19 nt- 5021 | 2822.38 | 4292.18 | 12594.99 |
| 16 nt- 5022 | 3654.32 | 5282.36 | 13063.04 |
| 28 nt-5023 | 18128.24 | 109481.22 | 429523.78 |
| 25 nt-5024 | 29433.90 | 189155.39 | 496316.23 |
| 21 nt- 5025 | 9734.68 | 69197.91 | 359949.71 |
| 20 nt- 5026 | 3595.65 | 10619.26 | 39632.86 |
| 19 nt- 5027 | 3780.12 | 3456.60 | 4532.53 |
| 16 nt-5028 | 4221.94 | 6718.91 | 3690.60 |

TABLE 7
CasM.1740 - SEQ ID NO: 27 trans cleavage
activity with varying spacer lengths

| Spacer length | 10 pM 5S87 | 100 pM 5S87 | 1000 pM 5S87 |
|---|---|---|---|
| and ID | target | target | target |
| 30 nt- 4754 | 20950.30 | 153682.03 | 376316.2 |
| 28 nt- 5017 | 23712.09 | 160906.05 | 385241.21 |
| 25 nt- 5018 | 23548.66 | 201797.96 | 448298.15 |
| 21 nt- 5019 | 7743.01 | 40464.78 | 223694.52 |
| 20 nt-5020 | 3384.41 | 5154.80 | 17851.64 |
| 19 nt- 5021 | 7124.04 | 21799.12 | 121540.37 |
| 16 nt- 5022 | 3835.29 | 7107.51 | 17667.67 |
| 28 nt-5023 | 17682.45 | 130378.91 | 404869.88 |
| 25 nt-5024 | 31106.44 | 214999.77 | 348405.33 |
| 21 nt- 5025 | 9997.37 | 68755.74 | 396100.08 |
| 20 nt- 5026 | 5933.76 | 24678.82 | 169621.67 |
| 19 nt- 5027 | 6942.39 | 14047.08 | 89959.12 |
| 16 nt-5028 | 4200.37 | 5602.73 | 7939.74 |

### Example 4: Thermostability Screen

This example describes experiments to test the ability of Type VI CRISPR/Cas proteins of the disclosure to exhibit trans cleavage activity above room temperature. The proteins tested were CasM.1862909-SEQ ID NO: 22, CasM.1862947-SEQ ID NO: 25 and CasM.1862921-SEQ ID NO: 24. All three proteins have a length between 780 and 850 amino acids.

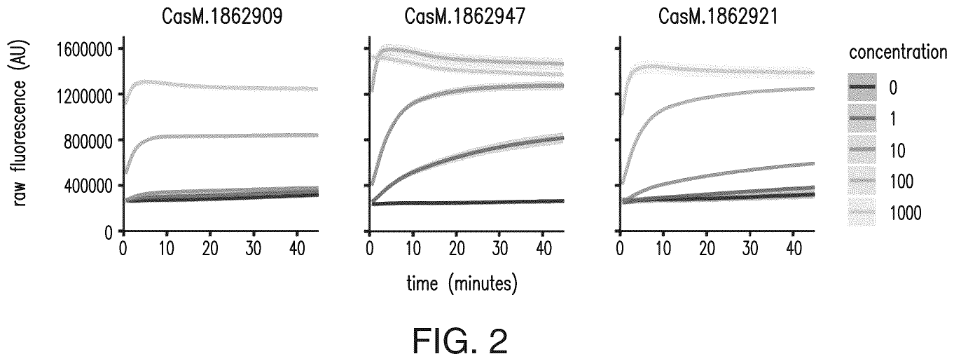

Type VI CRISPR/Cas proteins were incubated with crRNA and tracrRNA or sgRNAs in M Buffer 1 (Imidazole pH 7.5, KCl, MgCl2, BSA, Igepal Ca-630, glycerol), followed by addition of target nucleic acid (5S87; SEQ ID NO: 20). Systems were first screened at 40° C., 50° C., and 60° C. with saturating target concentration (5 nM). The most active systems at 60° C. were rescreened with a target titration (0 pM, 1 pM, 10 pM, 100 pM, 1000 pM) to avoid signal saturation before time course data could be taken. Trans cleavage activity was detected by fluorescence signal produced upon cleavage of a fluorophore-quencher reporter (included in the assay at 200 nM) in a DETECTR reaction. Results are presented in FIG. 2. This example demonstrates that Cas13 programmable nucleases having a length of 780 to 850 amino acids can provide trans cleavage activity at 60° C.

### Example 5: Phylogenetic Analysis

A phylogenetic analysis was conducted on the amino acid sequences of Cas proteins of the disclosure that demonstrated trans-cleavage activity in Examples 2 and 3. Bootstrap is a measure to indicate the percent of the times a branch is located at the same position in a tree. The bootstrap range in this experiment was 0.7-1.

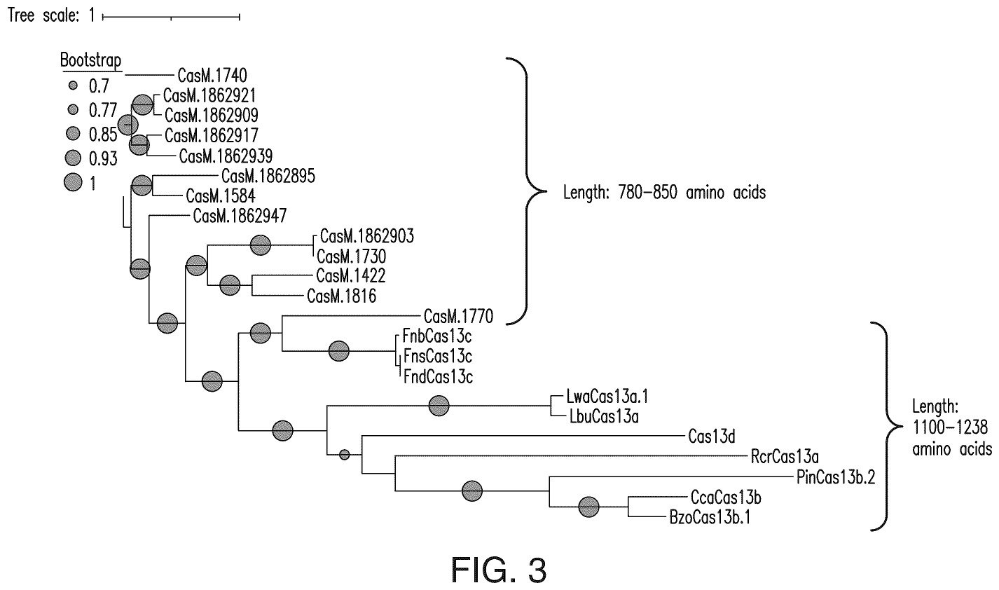

As shown in FIG. 3, two distinct clusters of active Cas sequences were observed. One cluster contains named Cas13 proteins with lengths between 1100 and 1238 amino acids (left). The other cluster forms a group that contains Type VI CRISPR/Cas proteins of the disclosure that are 780-850 amino acids in length.

### Example 6: Quantifying Trans-Collateral Activity Via DETECTR

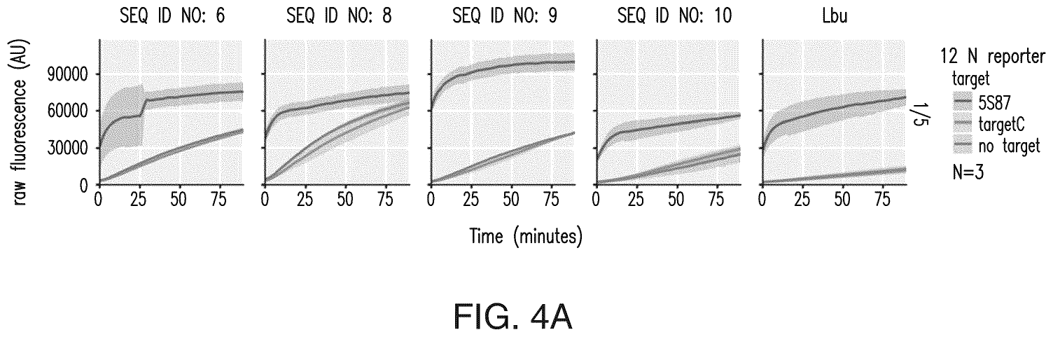

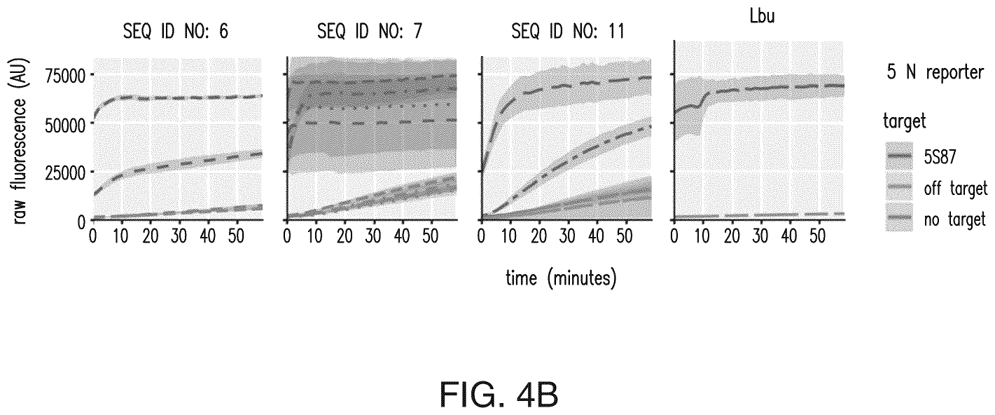

Identified Type VI CRISPR/Cas proteins were assessed in their ability to detect a target nucleic acid in a sequence using the spacer sequence, “CGACCUACUCUCCCAUACUCUUGUAUAUAG” (SEQ ID NO: 41) to detect a target 5S87 sequence “CUAUAUACAAGAGUAUGGGAGAGUAGGUCG (SEQ ID NO: 42)” in a sample. FIG. 4A shows fluorescence measured using an on-target 5S87, and target C “CAUGGCAUUCCACUUAUCAC (SEQ ID NO: 46)” (off-target), and no target control using the DETECTR assay to generate fluorescence in a presence of a target RNA nucleic acid sequence. A random 12-mer ribonucleotide (A, U, G, C) reporter was used in this assay. A positive control Cas protein, LbuCas13a (SEQ ID NO: 69), was also used in the assay.

In FIG. 4B, a shorter reporter was used to assess the trans-collateral activity compared to the 12 nucleotide reporter used in FIG. 4A.

### Example 7: Quantifying Trans-Collateral Activity Via DETECTR

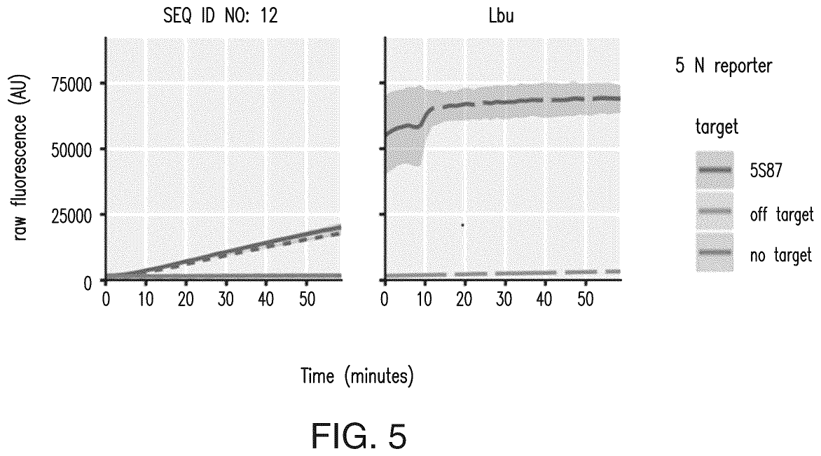

Identified Type VI CRISPR/Cas proteins were assessed in their ability to detect a target nucleic acid in a sequence using the spacer sequence, “CGACCUACUCUCCCAUACUCUUGUAUAUAG” (SEQ ID NO: 41) to detect a target 5S87 sequence “CUAUAUACAAGAGUAUGGGAGAGUAGGUCG (SEQ ID NO: 42)” in a sample. FIG. 5 shows fluorescence measured using an on-target 5S87, and target C “CAUGGCAUUCCACUUAUCAC (SEQ ID NO: 46)” (off-target), and no target control using the DETECTR assay in the presence of a target RNA nucleic acid sequence. A random 5-mer ribonucleotide (A, U, G, C) reporter was used in this assay. A positive control Cas protein, LbuCas13a (SEQ ID NO: 69), was also used in the assay.

### Example 8: Quantifying Trans-Collateral Activity Via DETECTR

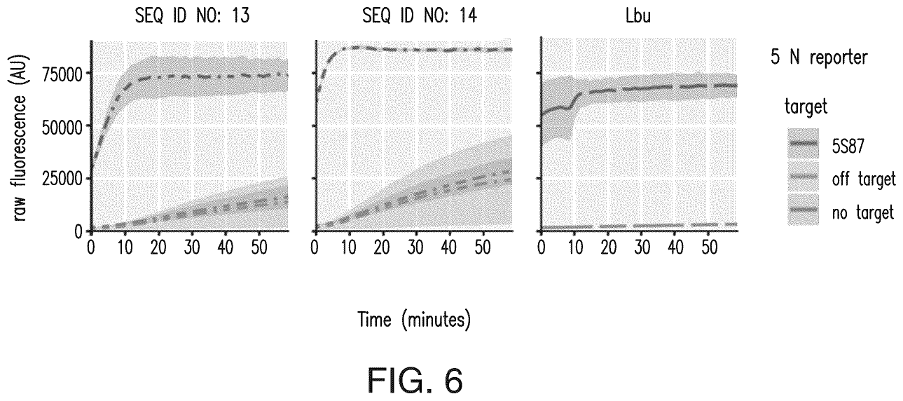

Identified Type VI CRISPR/Cas proteins were assessed in their ability to detect a target nucleic acid in a sequence using the spacer sequence, “CGACCUACUCUCCCAUACUCUUGUAUAUAG” (SEQ ID NO: 41) to detect a target 5S87 sequence “CUAUAUACAAGAGUAUGGGAGAGUAGGUCG (SEQ ID NO: 42)” in a sample. FIG. 6 shows fluorescence measured using an on-target 5S87, and target C “CAUGGCAUUCCACUUAUCAC (SEQ ID NO: 46)” (off-target), and no target fluorescence control using the DETECTR assay in the presence of a target RNA nucleic acid sequence. A random 5-mer ribonucleotide (A, U, G, C) reporter was used in this assay. A positive control Cas protein, LbuCas13a (SEQ ID NO: 69), was also used in the assay.

### Example 9: Trans-Cleavage Reporter Screen

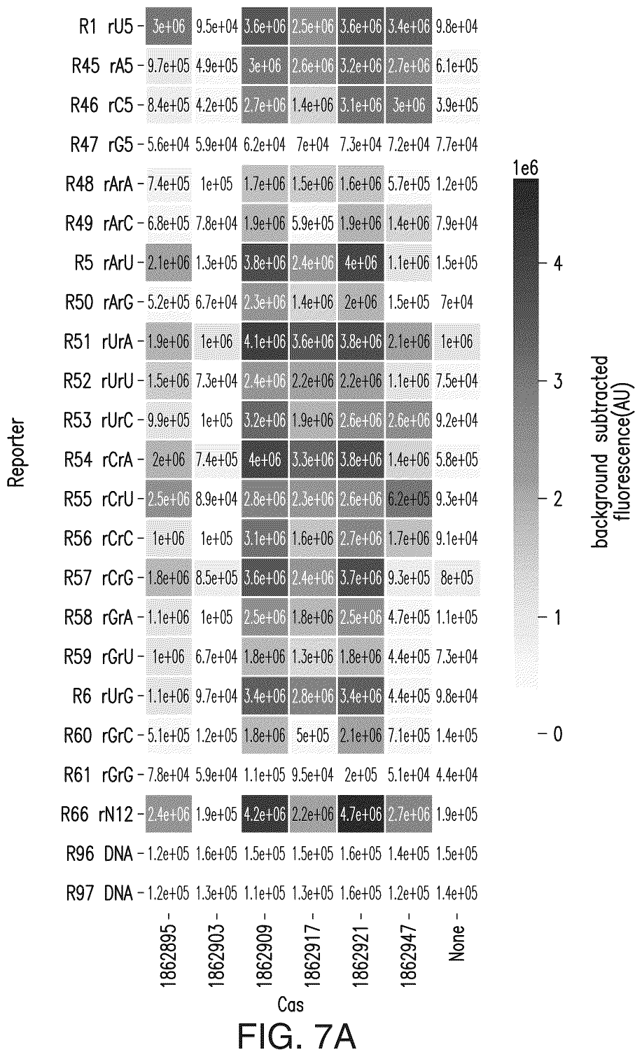

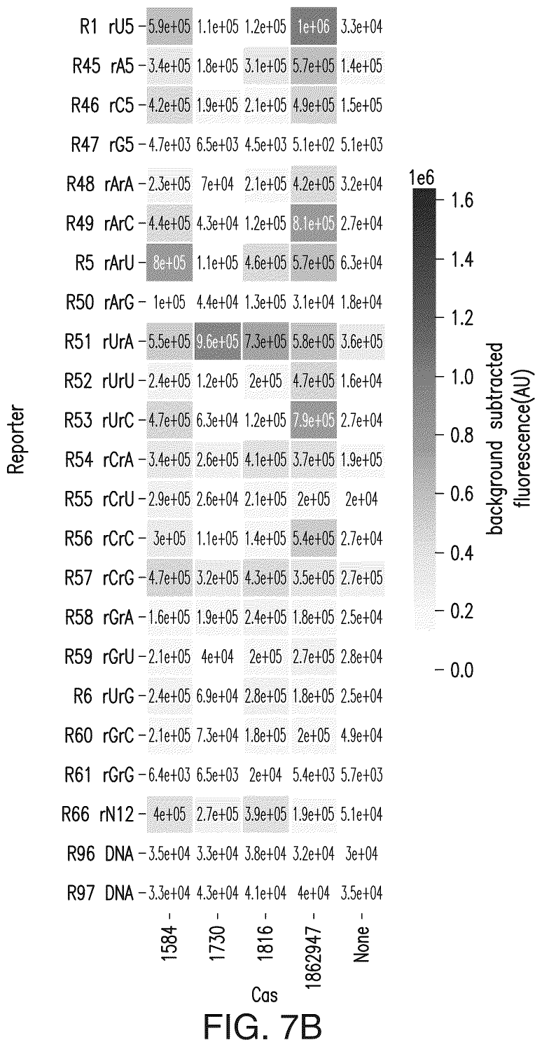

This example describes experiments to determine the trans-cleavage reporter preferences of various enzymes described herein. Briefly, effector protein was incubated at 37° C. for 15 minutes with crRNA to form a complex having a final concentrations of 40 nM protein and 40 nM crRNA. 5 μL of the complex was combined with a 15 μL mix of the following components for a total volume of 20 uL (listed in final concentration): trans cleavage buffer, target nucleic acid (125 pM), and a fluorophore-quencher (FQ) reporter (200 nM). Reporter preference was determined by varying the nucleic acid sequence of the nucleic acid between the fluorophore and quencher as shown in FIGS. 7A-7B, with the reporters following the 5′ to 3′ pattern of F-TA-X-GC-Q, where F is the fluorophore (56-FAM), Q is the quencher (3IABkFQ), T is thymine, A is adenine, G is guanine, C is cytosine, and X is the RNA component varied as shown in FIG. 7A-7B (e.g. “rU5”=rUrUrUrUrU=five ribouridines (SEQ ID NO: 33), “rArA”=two adenosines, “N12”=12 random RNA nucleotides, “DNA”=no RNA components, etc.). Systems were screened for 60 minutes at 37° C. Trans cleavage activity was detected by fluorescence signal produced upon cleavage of the fluorophore-quencher reporter. Results showing varied RNA dinucleotide and other reporter preferences for CasM.1862895-SEQ ID NO: 20, CasM.1862903-SEQ ID NO: 21, CasM.1862909-SEQ ID NO: 22, CasM.1862917-SEQ ID NO: 23, CasM.1862921-SEQ ID NO: 24, CasM.1862947-SEQ ID NO: 25, CasM.1584-SEQ ID NO: 15, CasM.1730-SEQ ID NO: 16, CasM.1816-SEQ ID NO: 18, and CasM.1862947-SEQ ID NO: 25 after 10 minutes of trans-cleavage are presented in FIG. 7A-7B.

### Example 10: Temperature Profiling for Cas13c Enzymes (CasM.26-SEQ ID NO: 69 and CasM.1740-SEQ ID NO: 27)

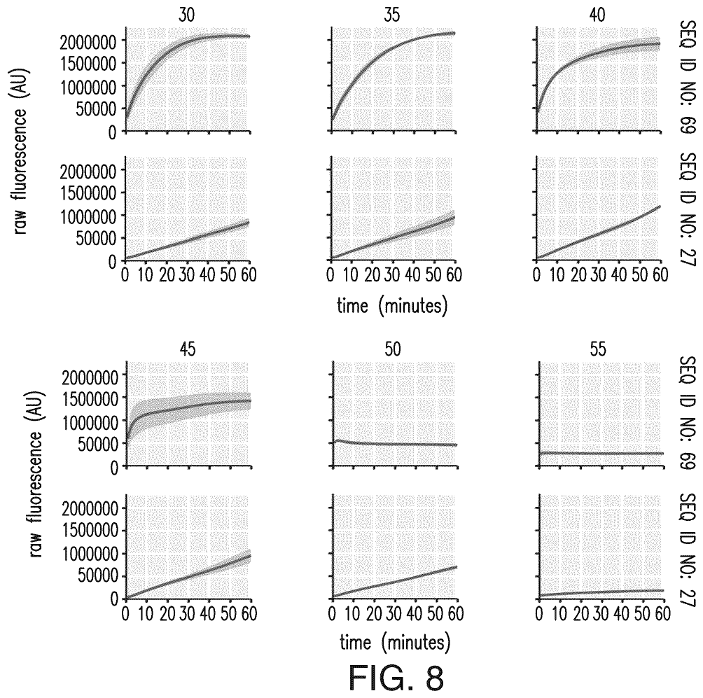

This example describes experiments to test the ability of CasM.26-SEQ ID NO: 69 and CasM.1740-SEQ ID NO: 27 to exhibit trans cleavage activity above room temperature. Briefly, effector protein was incubated at 37° C. for 15 minutes with crRNA to form a complex having a final concentrations of 40 nM protein and 40 nM crRNA. 5 μL of the complex was combined with a 15 μL mix of the following components for a total volume of 20 uL (listed in final concentration): trans cleavage buffer, target nucleic acid (50 pM), and a fluorophore-quencher reporter (200 nM). Systems were screened for 60 minutes at 30° C., 35° C., 40° C., 45° C., 50° C., and 55° C. Trans cleavage activity was detected by fluorescence signal produced upon cleavage of the fluorophore-quencher reporter at temperatures up to 50° C. for CasM.1740-SEQ ID NO: 27. Results are presented in FIG. 8.

### Example 11: Temperature Profiling for Cas13c Enzymes (CasM.1422-SEQ ID NO: 26)

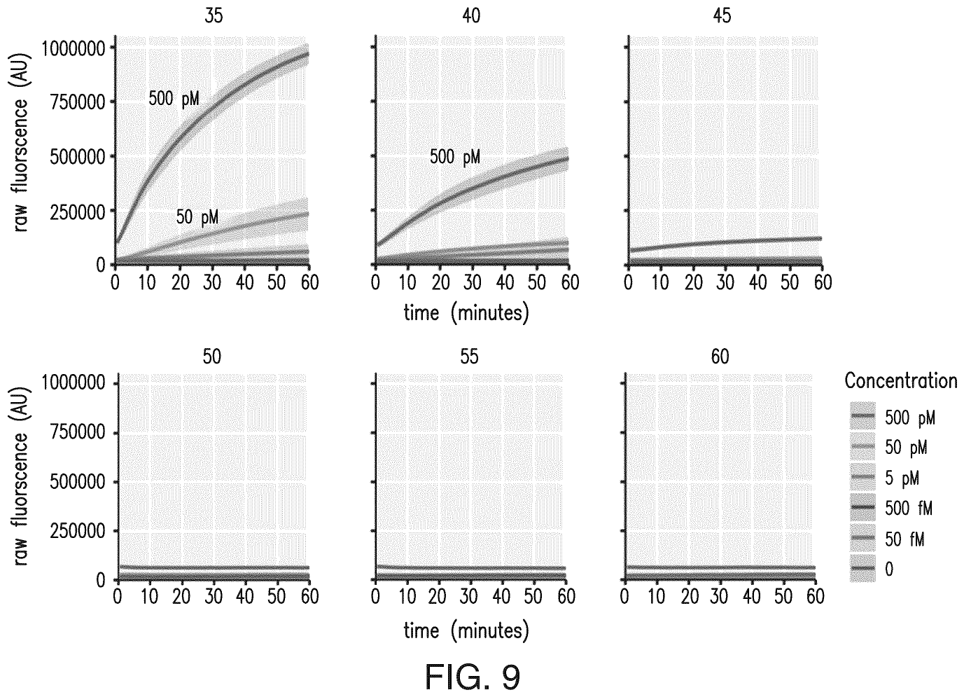

This example describes experiments to test the ability of CasM.1422-SEQ ID NO: 26 to exhibit trans cleavage activity above room temperature. Briefly, 40 nM effector protein was incubated at 37° C. for 15 minutes with 40 nM crRNA to form a complex, followed by addition varying concentrations of target. 5 μL of the complex was combined with a 15 μL mix of the following components for a total volume of 20 μL (listed in final concentration): trans cleavage buffer, target nucleic acid (list concentrations in legend of figure) or nuclease-free water (NFW), and a fluorophore-quencher (FQ) reporter (200 nM). Systems were screened at 35° C., 40° C., 45° C., 50° C., 55° C., and 60° C. Trans cleavage activity was detected by fluorescence signal produced upon cleavage of the fluorophore-quencher reporter at temperatures up to 45° C. Results are presented in FIG. 9.

### Example 12: Temperature Profiling for Cas13c Enzymes (CasM.1862921-SEQ ID NO: 24, CasM.1862895-SEQ ID NO: 20, CasM.1862909-SEQ ID NO: 22, CasM.1862903-SEQ ID NO: 21, and CasM.1862917-SEQ ID NO: 23)

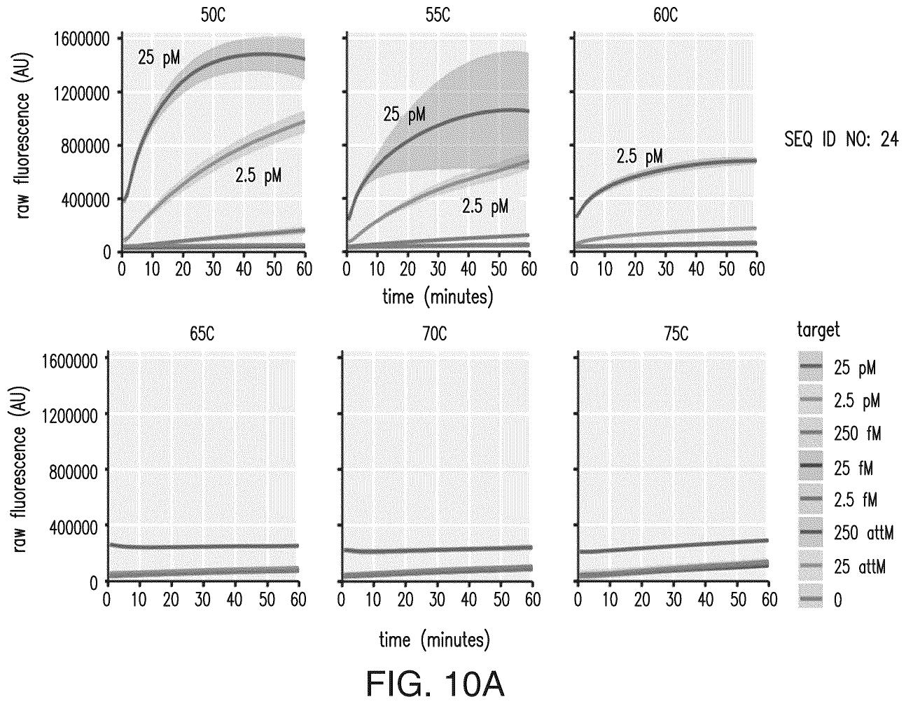

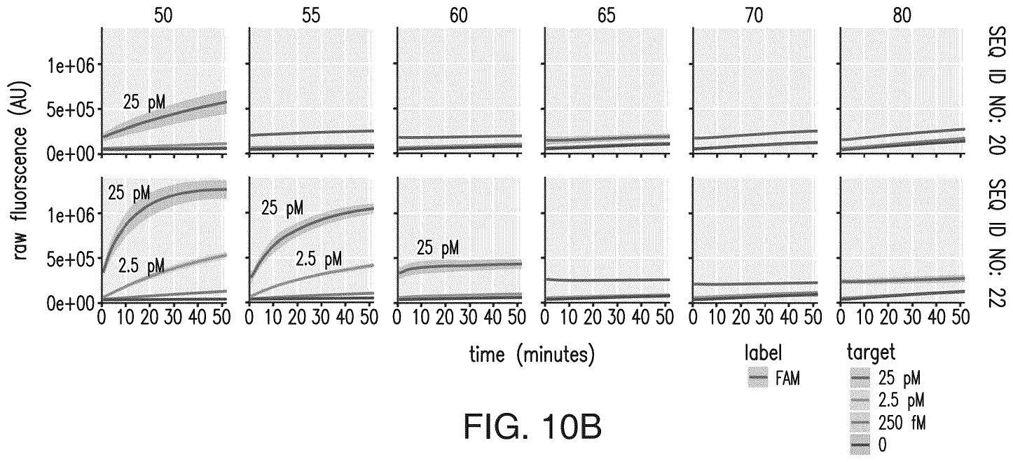

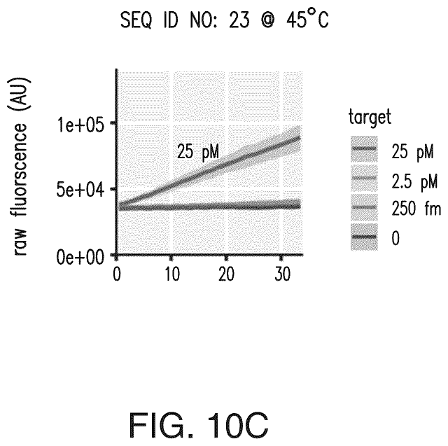

This example describes experiments to test the ability of CasM.1862921-SEQ ID NO: 24, CasM.1862895-SEQ ID NO: 20, CasM.1862909-SEQ ID NO: 22, CasM.1862903-SEQ ID NO: 21, and CasM.1862917-SEQ ID NO: 23 to exhibit trans cleavage activity above room temperature. Briefly, 40 nM effector protein was incubated at 37° C. for 15 minutes with 40 nM crRNA to form a complex, followed by addition varying concentrations of target (list concentrations in figure legend). 5 μL of the complex was combined with a 15 μL mix of the following components for a total volume of 20 μL (listed in final concentration): trans cleavage buffer, target nucleic acid (list concentrations) or nuclease-free water (NFW), and a fluorophore-quencher (FQ) reporter (200 nM). Systems were screened at temperatures selected from 45° C., 50° C., 55° C., 60° C., 65° C., 70° C., 75° C., and 80° C. Trans cleavage activity was detected by fluorescence signal produced upon cleavage of the fluorophore-quencher reporter at temperatures up to: 60° C. for CasM.1862921-SEQ ID NO: 24 with 25 pM reporter; 50° C. for CasM.1862895-SEQ ID NO: 20; 55° C. for CasM.1862909-SEQ ID NO: 22; and 45° C. for CasM.1862917-SEQ ID NO: 23. Results are presented in FIGS. 10A, 10B, and 10C.

### Example 13: Comparing CasM.26 to Cas13c's

This example demonstrates that CasM.1862909 (SEQ ID NO: 22) and 1862921 (SEQ ID NO: 24) exhibit trans cleavage activity on an HRP-based reporter immobilized to a solid support. In this instance, the HRP-based reporter was bound to a streptavidin-coated multi-well plate. The reporter, rep194, comprised a biotin functionality on its 3′ end and an HRP enzyme conjugated to its 5′ end, with a nucleic acid linker therebetween comprising a cleavable RNA-based linker section flanked by two uncleavable DNA-based linker sections: 5′ HRP-TTT TTT TTT TTT rUrUrUrUrU-TTT TTT TTT TTT-3′ Biotin-TEG, where T=thymine and rU=ribouridine (SEQ ID NO: 75).

Briefly, effector protein was incubated at 37° C. for 30 minutes with crRNA to form a complex having a final concentrations of 40 nM protein and 40 nM crRNA.

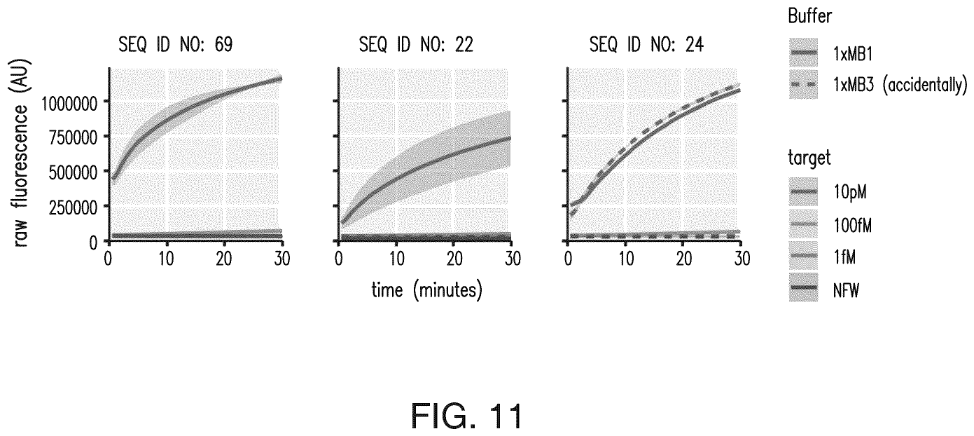

5 μL of the complex was combined with a 20 μL mix of the following components for a total volume of 25 μL (listed in final concentration): trans cleavage buffer, target nucleic acid (10 pM, 100fM, 1 fM) or nuclease-free water (NFW), and a fluorophore-quencher (FQ) reporter (200 nM). Systems were screened for 60 minutes at 37° C. Trans cleavage activity was detected by fluorescence signal produced upon cleavage of the fluorophore-quencher reporter. Results showing CasM.1862909-SEQ ID NO: 22 and 1862921-SEQ ID NO: 24 exhibit trans cleavage activity are presented in FIG. 11.

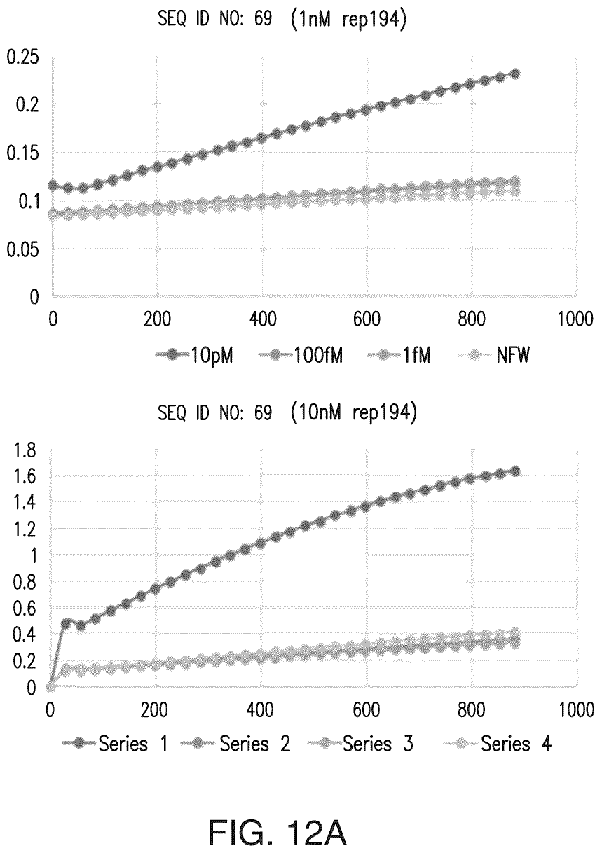

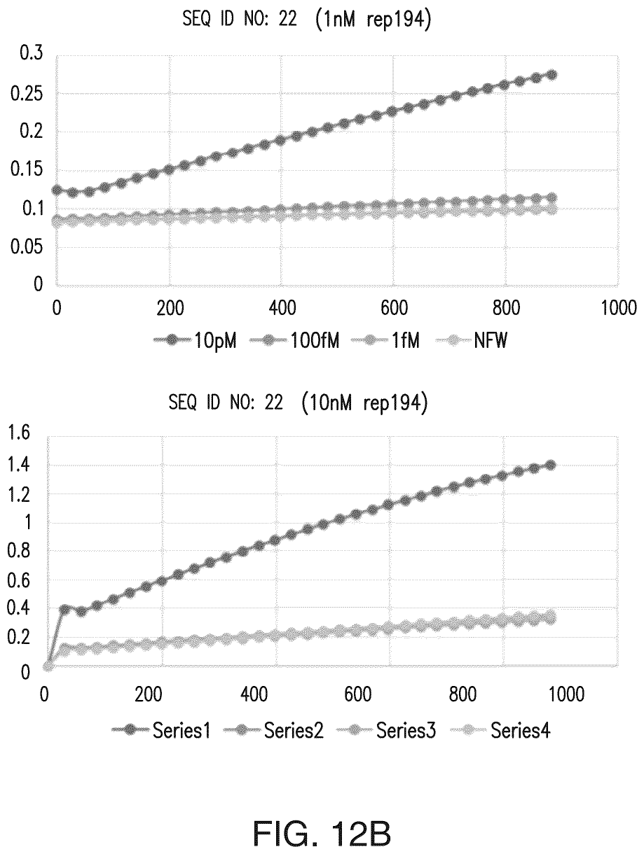

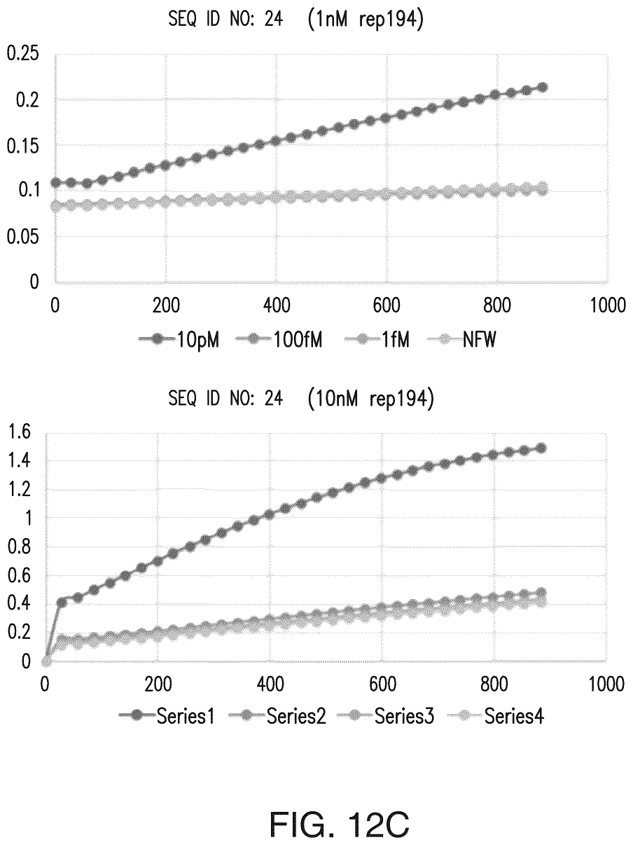

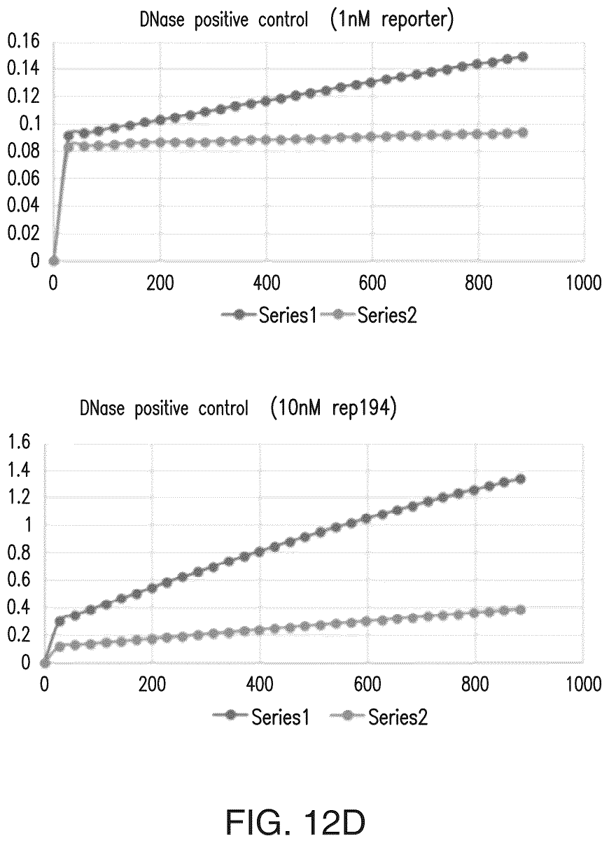

25 μL of 1 nM or 10 nM reporter, rep194, was incubated on a streptavidin-coated 96-well plate at 25° C. for 45 minutes with intermittent shaking in order to immobilize the reporter to the surface of the well. Excess reporter was washed off before 5 μL of the complex was combined with a 20 μL mix of the following components for a total volume of 25 μL (listed in final concentration) was added to each HRP-reporter-immobilized well: trans cleavage buffer with target nucleic acid (10 pM, 100 fM, 1 fM) or nuclease-free water (NFW). For the DNAse positive control, a 25 μL mix of DNAse and buffer was added to the HRP-reporter-immobilized wells in place of the complex mixture. Reactions were allowed to proceed for 60 mins at 37° C. on thermomixer with intermittent shaking at 500 RPM (15 seconds on, 2 minutes off). 25 μL of supernatant was then transferred to a clear greiner 96-well plate before 50 μL of TMB stabilized chromagen was to each well. Absorbance was measured at 650 nm for 15 min at R.T. Trans cleavage activity was detected by 650 nM absorbance signal produced upon presence of the HRP detection moiety in the supernatant following cleavage of the immobilized HRP-based reporter. Results showing CasM.1862909-SEQ ID NO: 22 and CasM.1862921-SEQ ID NO: 24 exhibit trans cleavage activity with HRP-reporters immobilized onto a surface are presented in FIGS. 12A-12D.

### Example 14: CasM.1862921-SEQ ID NO: 24 FluA gRNA Screen

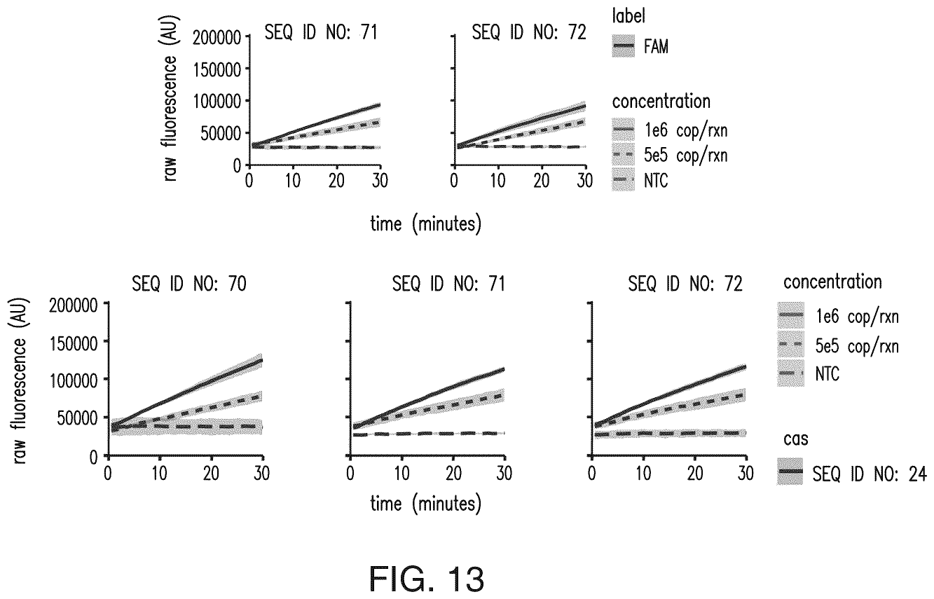

In this example, CasM.1862921-SEQ ID NO: 24 was tested for its ability to directly detect two strains of Influenza A RNA. 5 μM effector protein was incubated at 37° C. for 30 minutes with 20 μM crRNA to form a complex, followed by addition 100 μM fluorophore-quencher reporter for final concentrations of 40 nM protein, 40 nM crRNA, and 200 nM fluorophore-quencher reporter. The reporter used in this experiment was rep001, FAM-U5-IowaFQ, also written/5-6FAM/rUrUrUrUrU/3IABkFQ/(SEQ ID NO: 33). The target concentrations used were 1*10\hat{ }6 copies/reaction, 5.5*10\hat{ }6 copies/reaction, and 0 copies/reaction. FIG. 13 depicts the ability of CasM1862921-SEQ ID NO: 24 to detect two strains of Influenza A RNA with the various guide RNA (SEQ ID NOs: 70-72).

### Example 15: Cas13 Enzymes Function at Room Temperature and Below

Cas13 DETECTR was run using 40 nM Cas13, 40 nM crRNA, 1 U/uL Rnase Inhibitor, 200 nM FQ reporter, in a buffer consisting of 20 mM Imidizole (pH 7.5), 50 mM KCl, 5 mM MgCl2, 10 ug/mL BSA, 0.01% IGEPAL CA-630, and 5% glycerol. Reactions were incubated with 10 pM of target RNA for 60 minutes on a plate reader with varied temperature settings.

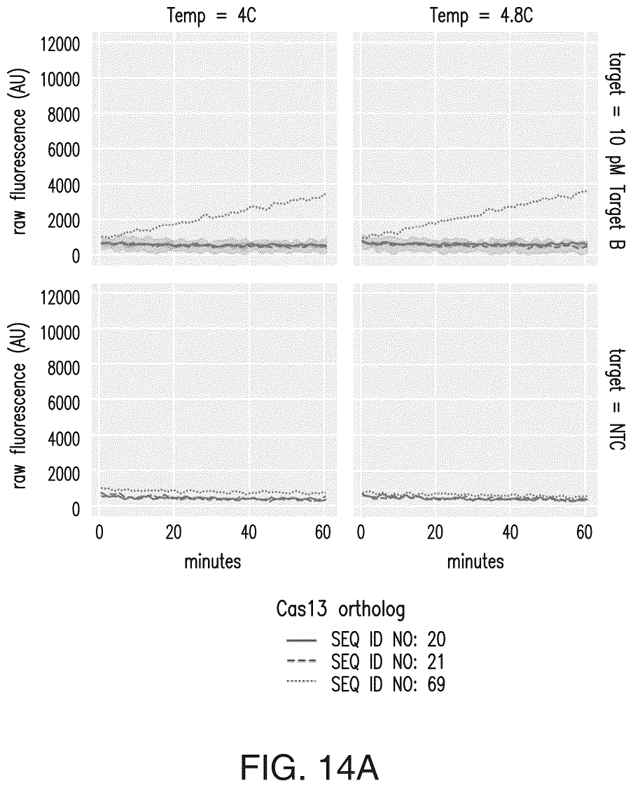

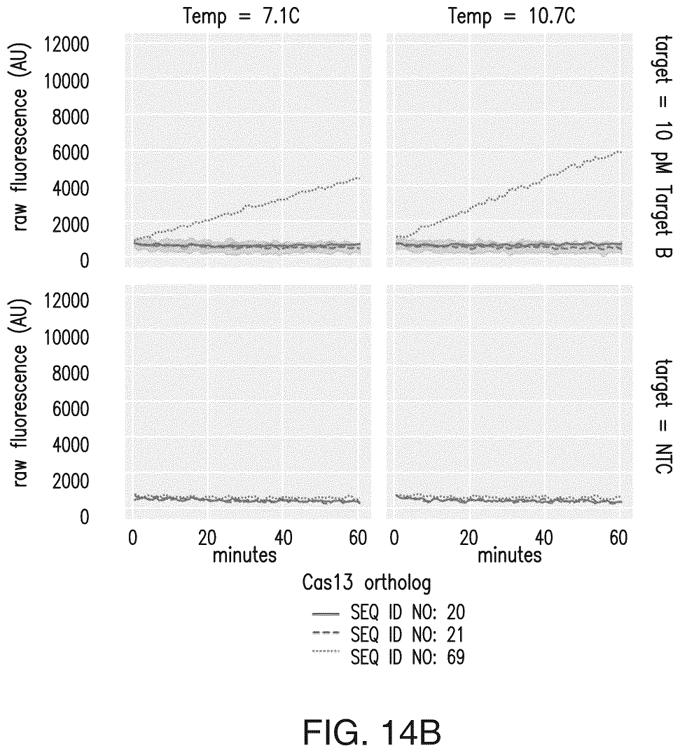

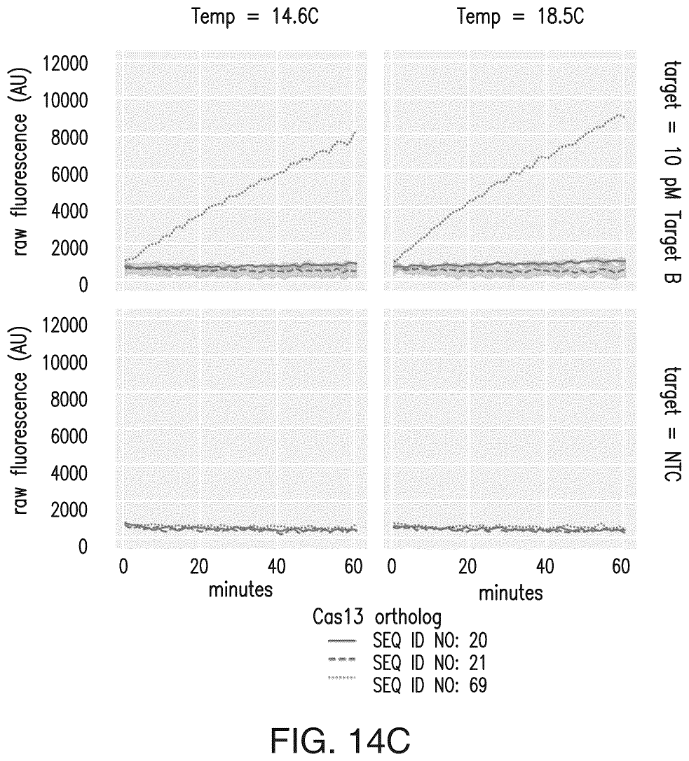

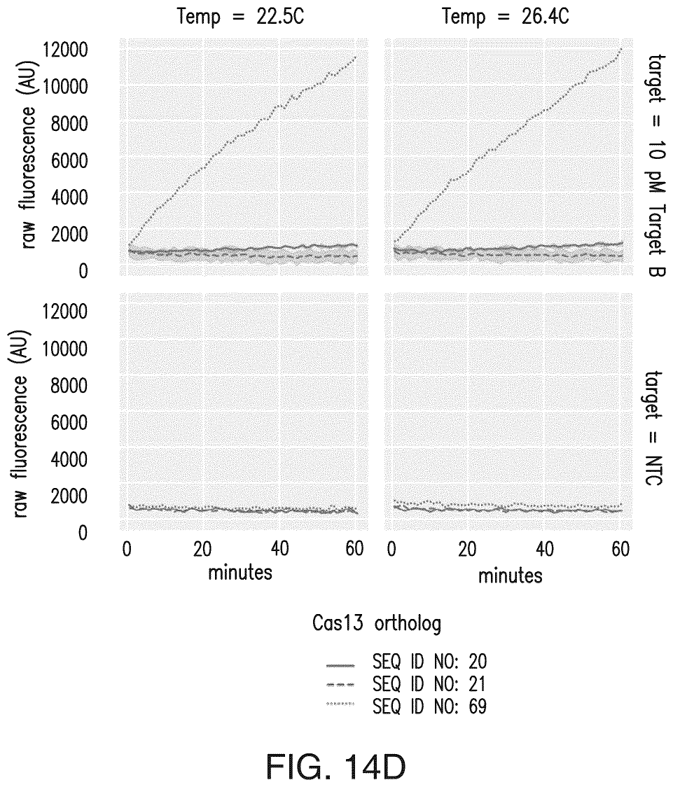

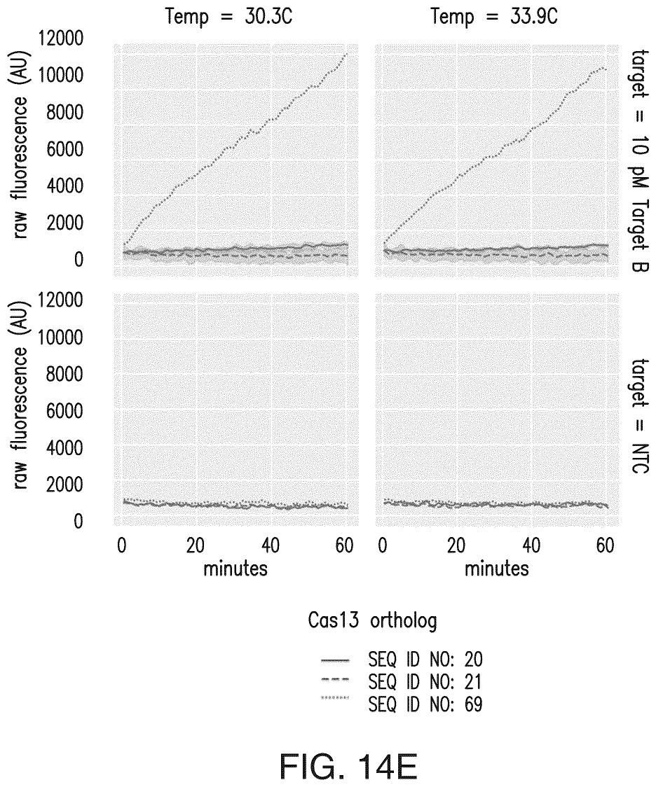

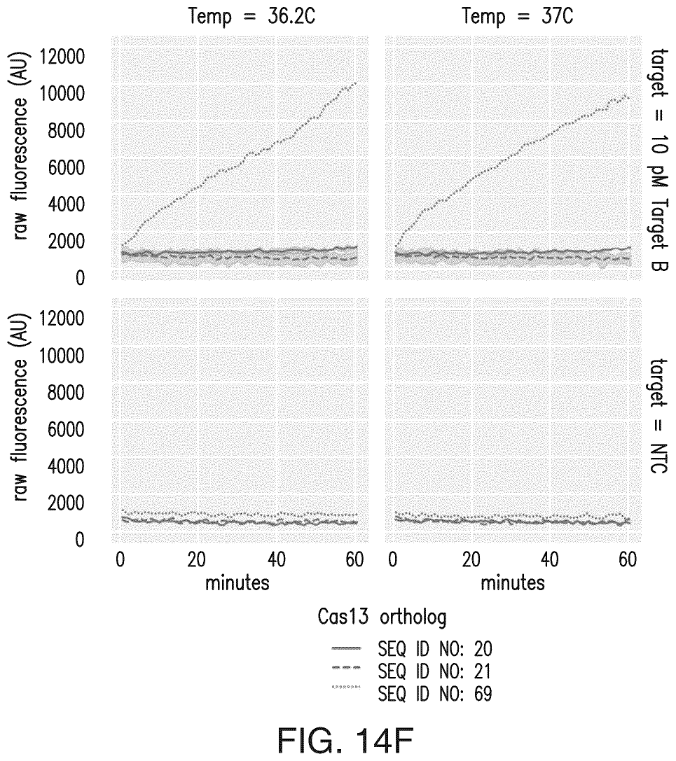

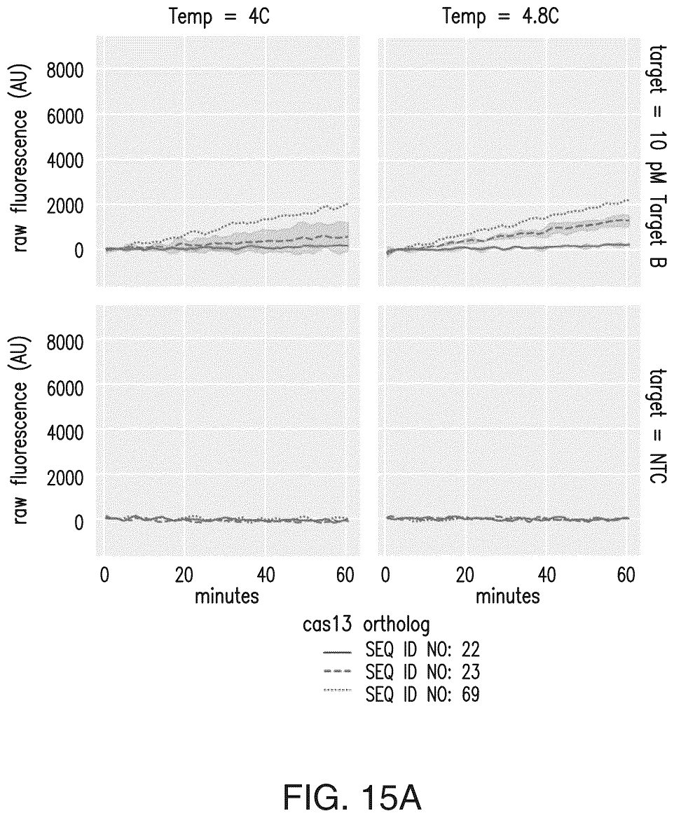

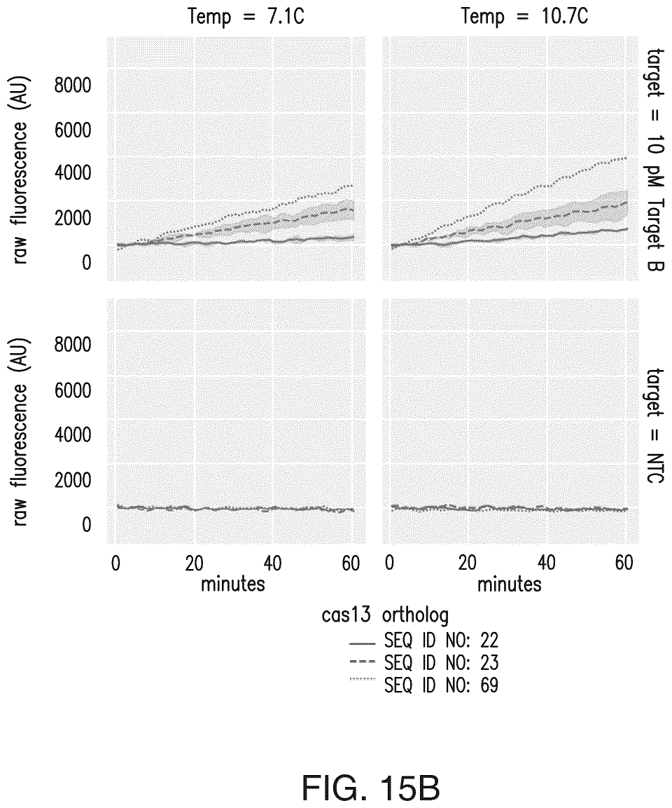

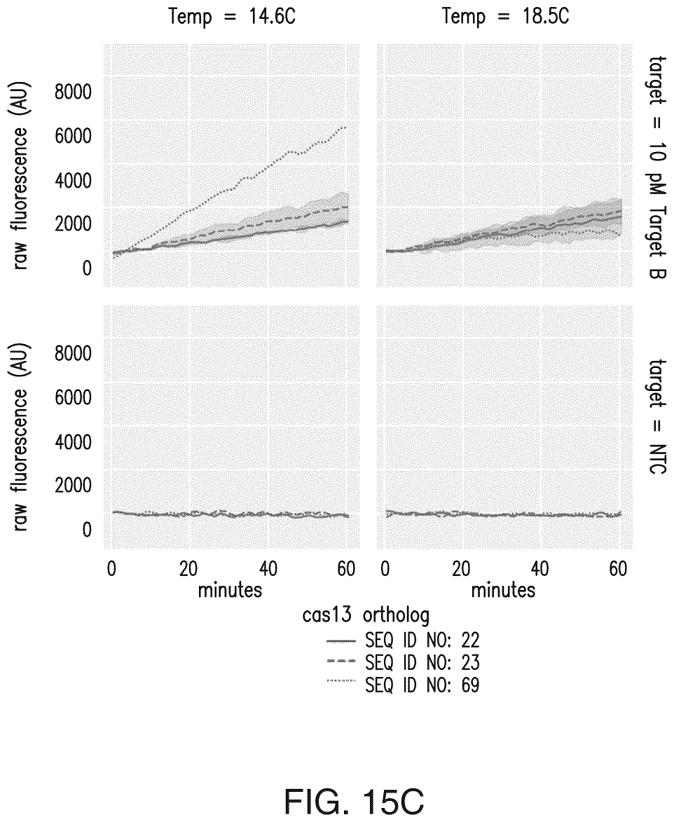

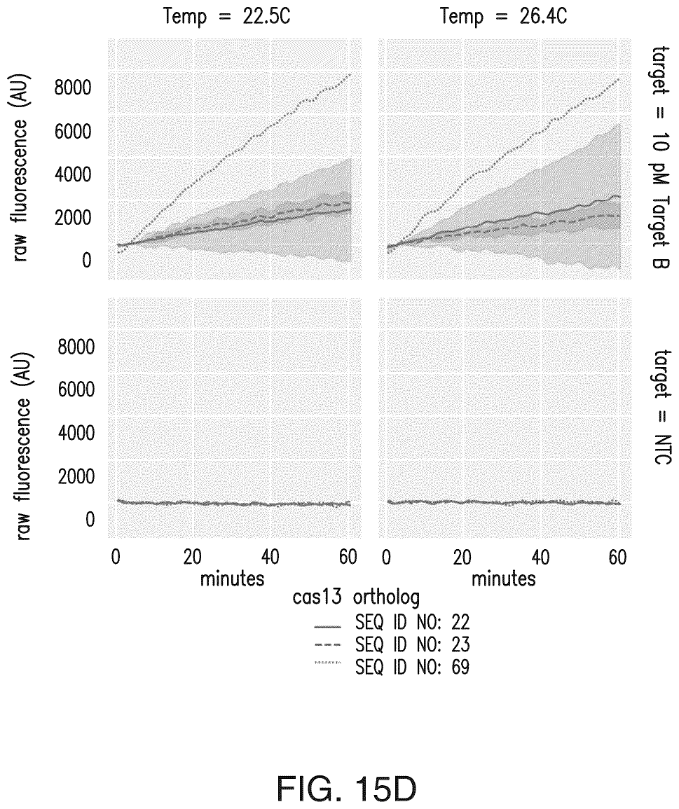

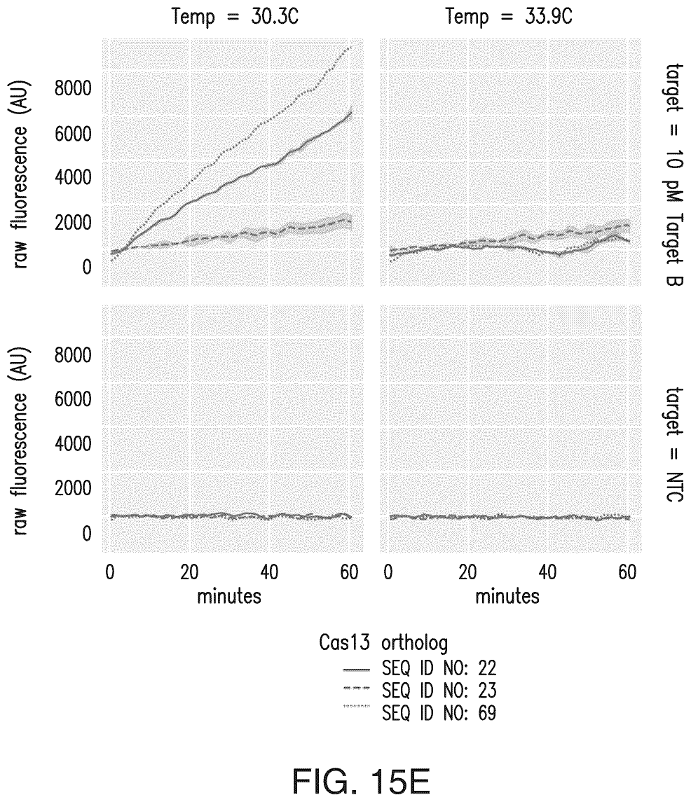

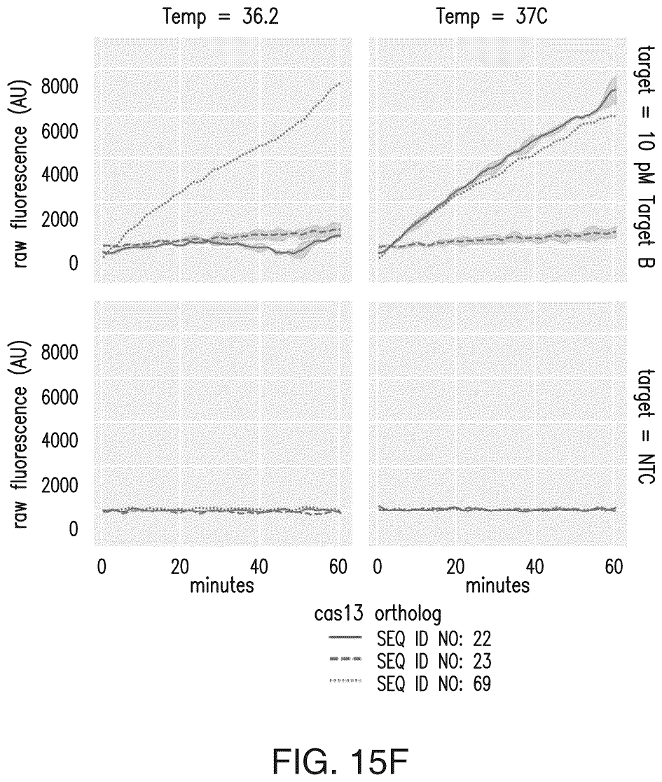

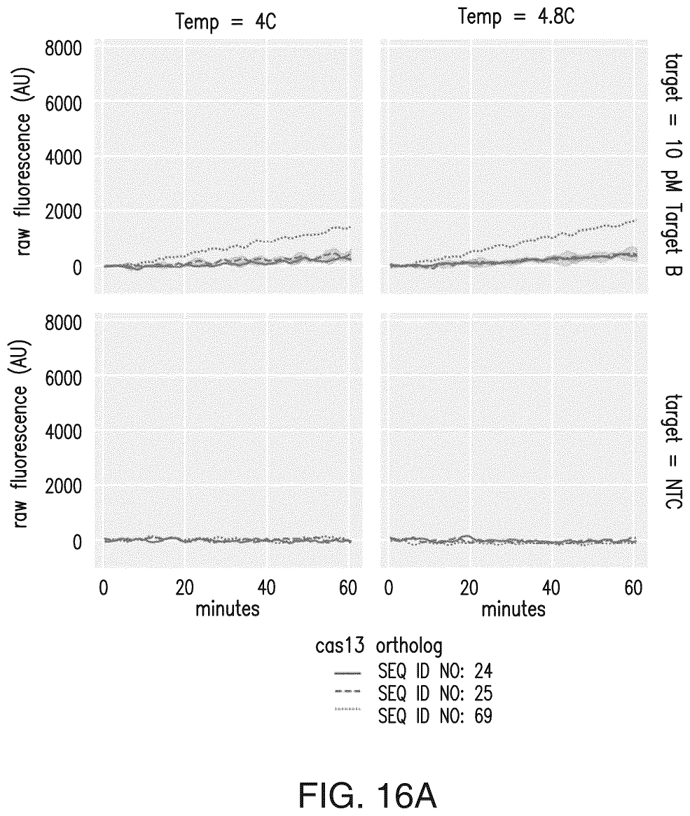

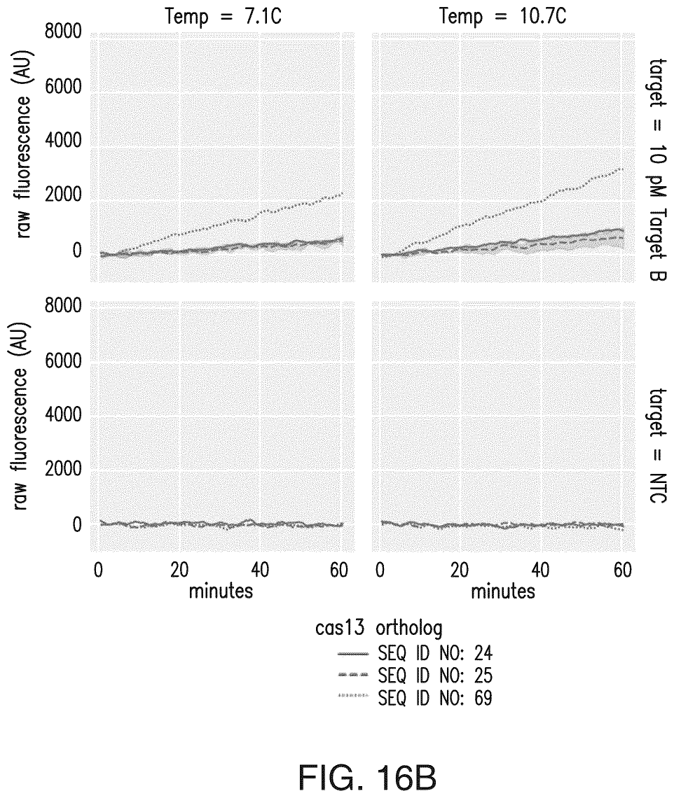

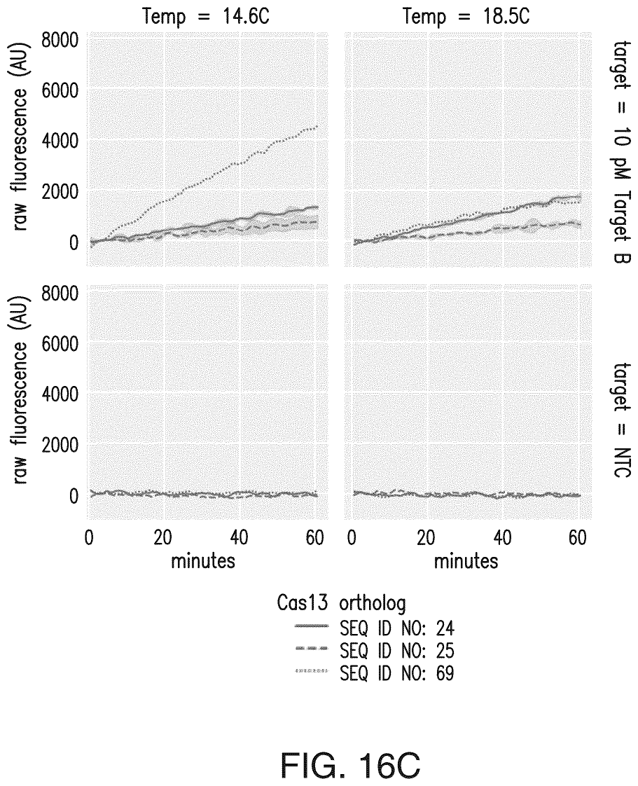

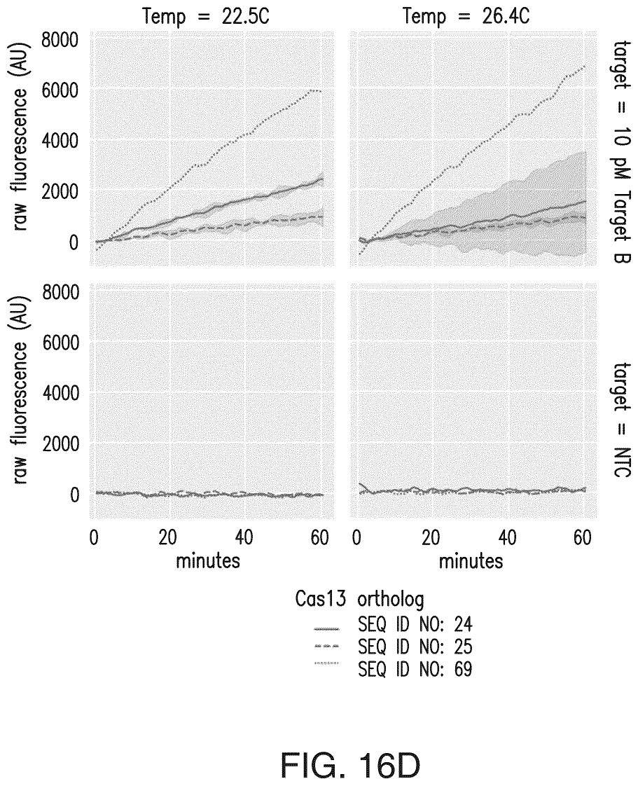

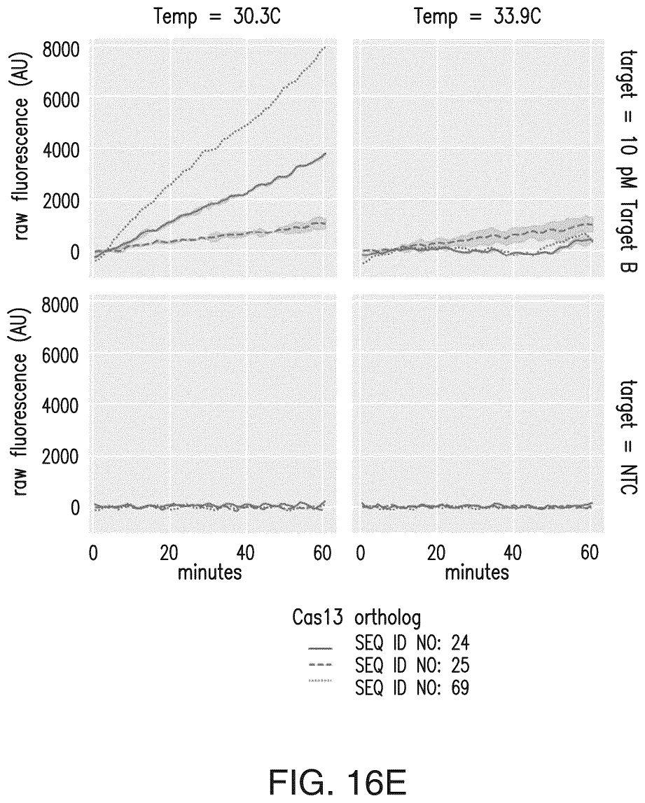

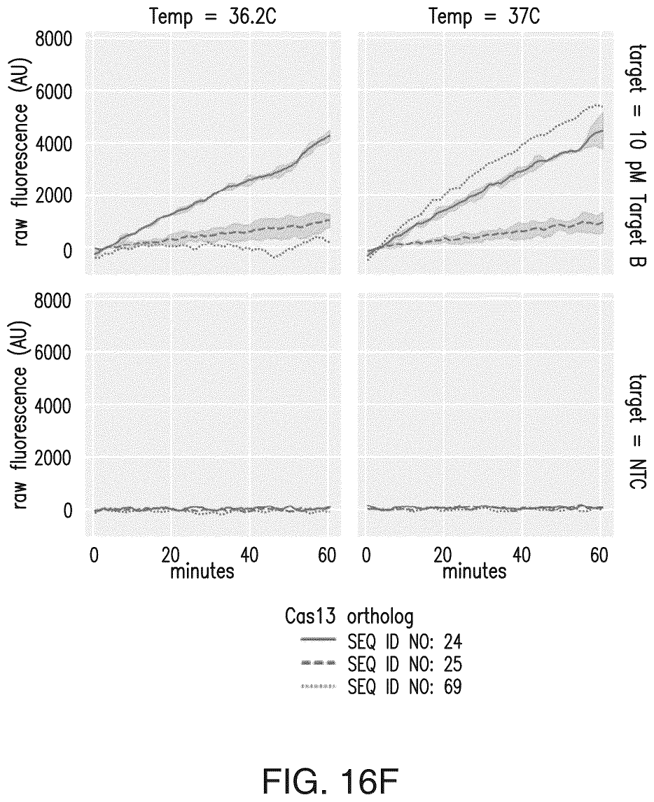

Orthologs tested were: SEQ ID NO: 20, SEQ ID NO: 21, and control SEQ ID NO: 69 (FIG. 14A-14F); SEQ ID NO: 22, SEQ ID NO: 23, and control SEQ ID 69 (FIG. 15A-15F); and SEQ ID NO: 24, SEQ ID NO: 25, and control SEQ ID NO: 69 (FIG. 16A-16F).

SEQ ID NO: 69 and orthologs SEQ ID NOs: 22, 23, 24, and 25 all show trans cleavage activity down to 4° C.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A non-naturally occurring composition comprising a programmable nuclease and an engineered guide nucleic acid, wherein the programmable nuclease comprises an amino acid sequence that is at least 75% identical to any one of SEQ ID NOs: 1-27.

2.-16. (canceled)

17. The non-naturally occurring composition of claim 1, wherein:
a) the programmable nuclease comprises an amino acid sequence that is at least 75% identical to SEQ ID NO: 1, and the engineered guide nucleic acid comprises a sequence that is at least 75% identical to SEQ ID NO: 28;
b) the programmable nuclease comprises an amino acid sequence that is at least 75% identical to SEQ ID NO: 2, and the engineered guide nucleic acid comprises a sequence that is at least 75% identical to SEQ ID NO: 29;
c) the programmable nuclease comprises an amino acid sequence that is at least 75% identical to SEQ ID NO: 3, and the engineered guide nucleic acid comprises a sequence that is at least 75% identical to SEQ ID NO: 30;
d) the programmable nuclease comprises an amino acid sequence that is at least 75% identical to SEQ ID NO: 4, and the engineered guide nucleic acid comprises a sequence that is at least 75% identical to SEQ ID NO: 31;
e) the programmable nuclease comprises an amino acid sequence that is at least 75% identical to SEQ ID NO: 5, and the engineered guide nucleic acid comprises a sequence that is at least 75% identical to SEQ ID NO: 32;
f) the programmable nuclease comprises an amino acid sequence that is at least 75% identical to SEQ ID NO: 6, and the engineered guide nucleic acid comprises a sequence that is at least 75% identical to any one of SEQ ID NOs: 28-32;
g) the programmable nuclease comprises an amino acid sequence that is at least 75% identical to SEQ ID NO: 7, and the engineered guide nucleic acid comprises a sequence that is at least 75% identical to any one of SEQ ID NOs: 28-32;
h) the programmable nuclease comprises an amino acid sequence that is at least 75% identical to SEQ ID NO: 8, and the engineered guide nucleic acid comprises a sequence that is at least 75% identical to any one of SEQ ID NOs: 28-32;
i) the programmable nuclease comprises an amino acid sequence that is at least 75% identical to SEQ ID NO: 9, and the engineered guide nucleic acid comprises a sequence that is at least 75% identical to any one of SEQ ID NOs: 28-32;
j) the programmable nuclease comprises an amino acid sequence that is at least 75% identical to SEQ ID NO: 10, and the engineered guide nucleic acid comprises a sequence that is at least 75% identical to any one of SEQ ID NOs: 28-32;
k) the programmable nuclease comprises an amino acid sequence that is at least 75% identical to SEQ ID NO: 11, and the engineered guide nucleic acid comprises a sequence that is at least 75% identical to any one of SEQ ID NOs: 28-32;
l) the programmable nuclease comprises an amino acid sequence that is at least 75% identical to SEQ ID NO: 12, and the engineered guide nucleic acid comprises a sequence that is at least 75% identical to any one of SEQ ID NOs: 28-32;
m) the programmable nuclease comprises an amino acid sequence that is at least 75% identical to SEQ ID NO: 13, and the engineered guide nucleic acid comprises a sequence that is at least 75% identical to any one of SEQ ID NOs: 28-32;
n) the programmable nuclease comprises an amino acid sequence that is at least 75% identical to SEQ ID NO: 14, and the engineered guide nucleic acid comprises a sequence that is at least 75% identical to any one of SEQ ID NOs: 28-32;
o) the programmable nuclease comprises an amino acid sequence that is at least 75% identical to SEQ ID NO: 15, and the engineered guide nucleic acid comprises a sequence that is at least 75% identical to SEQ ID NO: 60;
p) the programmable nuclease comprises an amino acid sequence that is at least 75% identical to SEQ ID NO: 16, and the engineered guide nucleic acid comprises a sequence that is at least 75% identical to SEQ ID NO: 61;
q) the programmable nuclease comprises an amino acid sequence that is at least 75% identical to SEQ ID NO: 17, and the engineered guide nucleic acid comprises a sequence that is at least 75% identical to SEQ ID NO: 62;
r) the programmable nuclease comprises an amino acid sequence that is at least 75% identical to SEQ ID NO: 18, and the engineered guide nucleic acid comprises a sequence that is at least 75% identical to SEQ ID NO: 63;
s) the programmable nuclease comprises an amino acid sequence that is at least 75% identical to SEQ ID NO: 19, and the engineered guide nucleic acid comprises a sequence that is at least 75% identical to SEQ ID NO: 60;
t) the programmable nuclease comprises an amino acid sequence that is at least 75% identical to SEQ ID NO: 20, and the engineered guide nucleic acid comprises a sequence that is at least 75% identical to SEQ ID NO: 64;
u) the programmable nuclease comprises an amino acid sequence that is at least 75% identical to SEQ ID NO: 21, and the engineered guide nucleic acid comprises a sequence that is at least 75% identical to SEQ ID NO: 61;
v) the programmable nuclease comprises an amino acid sequence that is at least 75% identical to SEQ ID NO: 22, and the engineered guide nucleic acid comprises a sequence that is at least 75% identical to SEQ ID NO: 65;
w) the programmable nuclease comprises an amino acid sequence that is at least 75% identical to SEQ ID NO: 23, and the engineered guide nucleic acid comprises a sequence that is at least 75% identical to SEQ ID NO: 60;
x) the programmable nuclease comprises an amino acid sequence that is at least 75% identical to SEQ ID NO: 24, and the engineered guide nucleic acid comprises a sequence that is at least 75% identical to SEQ ID NO: 65;
y) the programmable nuclease comprises an amino acid sequence that is at least 75% identical to SEQ ID NO: 25, and the engineered guide nucleic acid comprises a sequence that is at least 75% identical to SEQ ID NO: 66;
z) the programmable nuclease comprises an amino acid sequence that is at least 75% identical to SEQ ID NO: 26, and the engineered guide nucleic acid comprises a sequence that is at least 75% identical to SEQ ID NO: 67; or
aa) the programmable nuclease comprises an amino acid sequence that is at least 75% identical to SEQ ID NO: 27, and the engineered guide nucleic acid comprises a sequence that is at least 75% identical to SEQ ID NO: 68.

18.-24. (canceled)

25. The non-naturally occurring composition of claim 1, wherein the engineered guide nucleic comprises at least 75% sequence identity to a sequence selected from a group consisting of: SEQ ID NO: 28-SEQ ID NO: 32.

26. (canceled)

27. The non-naturally occurring composition of claim 1, wherein the engineered guide nucleic acid comprises a first region (FR1) complementary to a target sequence and the second region (FR2) that is not complementary to the target sequence.

28. The non-naturally occurring composition of claim 27, wherein the first region and second region are oriented: FR1−FR2.

29. The non-naturally occurring composition of claim 27, wherein the first region and second region are oriented FR2−FR1.

30. The non-naturally occurring composition of claim 27, wherein FR1 comprises at least 75% sequence identity to a sequence selected from a group consisting of: SEQ ID NO: 28-SEQ ID NO: 32.

31. The non-naturally occurring composition of claim 27, wherein FR2 is a sequence comprising at least 75% sequence identity to SEQ ID NO: 41.

32. A non-naturally occurring composition comprising a programmable nuclease and an engineered guide nucleic acid capable of catalyzing cRNA-directed, RNA-targeted trans-cleavage activity at a temperature of at least about 55° C. to at least about 85° C., wherein the programmable nuclease comprises at least one HEPN or HEPN-like domain.

33. (canceled)

34. A non-naturally occurring composition comprising a programmable nuclease comprising at least one HEPN or HEPN-like domain and an engineered guide nucleic acid capable of catalyzing at least a 1.5 fold change in cRNA-directed, RNA-targeted trans-cleavage activity.

35. The non-naturally occurring composition of claim 34, wherein fold change is determined, by quantifying cleavage of a labeled detector RNA present in an in vitro sample in a reaction, performed at a temperature of about 37° C. and comprising:
at least 160 nM of the RNA-guided endonuclease,
at least 160 nM of the guide RNA,
at least 5 nM of a target RNA, and
200 nM of the labeled detector RNA.

36.-39. (canceled)

40. The non-naturally occurring composition of claim 1, wherein the amino acid sequence of the programmable nuclease is about 700 to about 900 amino acids in length.

41. The non-naturally occurring composition of claim 1, wherein the programmable nuclease exhibits increased trans-cleavage activity when the guide RNA comprises a spacer region of about 25 nucleotides in length, as compared to the cleavage produced by a composition comprising the same programmable nuclease and a guide nucleic acid comprising a spacer region less than 20 nucleotides in length, or greater than 30 nucleotides in length.

42.-49. (canceled)

50. The non-naturally occurring composition of claim 32, wherein the programmable nuclease comprises an amino acid sequence that is at least 75% identical to any one of SEQ ID NOS: 15-27.

51.-54. (canceled)

55. The non-naturally occurring composition of claim 1, wherein the programmable nuclease and engineered guide nucleic acid are capable of catalyzing cRNA-directed, RNA-targeted trans-cleavage activity at a temperature of about 50° C. to about 70° C.

56.-66. (canceled)

67. A system for detecting a target nucleic acid comprising the composition of claim 1 and at least one of a buffering agent, a salt, a crowding agent, a detergent, a reducing agent, a competitor, and a reporter nucleic acid.

68.-173. (canceled)

174. A method of detecting a nucleic acid in a sample, comprising the steps of:
i) contacting a sample with:
a) a programmable nuclease, wherein the programmable nuclease comprises at least 75% sequence identity to a sequence selected from a group consisting of SEQ ID NOs: 1-27;
b) a reporter; and
c) an engineered guide nucleic acid;
ii) measuring a detectable signal produced by cleavage of the reporter, wherein the measuring provides detection of the target nucleic acid in the sample.

175.-200. (canceled)

201. The method of claim 174, wherein:
a) the programmable nuclease comprises SEQ ID NO: 22, and contacting occurs at a temperature of less than 30° C.;
b) the programmable nuclease comprises SEQ ID NO: 23, and contacting occurs at a temperature of less than 30° C.;
c) the programmable nuclease comprises SEQ ID NO: 24, and contacting occurs at a temperature of less than 30° C.; or
d) the programmable nuclease comprises SEQ ID NO: 25, and contacting occurs at a temperature of less than 30° C.

202.-256. (canceled)


SEQUENCE LISTING (75 sequences)

SEQ ID NO: 1 - protein, 1119 aa, synthetic construct
  REGION 1..1119: note = Description of Artificial Sequence: Synthetic polypeptide
MEDDKKTKESTNMLDNKHFWAAFLNLARHNVYITVNHINKILEEGEINRDGYETTLKNSWDEIKDINKKDRLSKLIIKHFPFLEAATYRQNPTDTTKQKEEKQAEAQSLESLKKSFFVFIYKLRDLRNHYSHYKHSKSLERPKFEEDLQEKMYNIFDVSICLVKKDYQYNKDINPDEDFKHLDRKRKGKFKYSFADNEGNITESGLLFFVSLFLEKKDAIWVQKKLEGFKCSNESYQKMTNEVFCRSRMLLPKLRLESTQTQDWILLDMLNELIRCPKSLYERLRGDDREKFRVPLDPIDEDYDAEQEPFKNTLVRHQDRFPYFALRYFDYNEIFTNLRFQIDLGTYHFSIYKKLIGGQKEDRHLTHKLYGFERIQEFDKQNRPDEWKAIVKDSDTFKKKEEKEEEKPYISETTPHYHLENKKIGIAFKNHNIWPSTQTELTNNKRKKYNLGTSIKAEAFLSVHELLPMMFYYLLLKTENTKNDNKVEGKKETKKQEKHKIESIIESKIKDMYALYDAFANGEINSEKELEEYLKGKDIEIVHLPKQMIAILKNEHKDMAEKAEAKQEKMKLATENRLKTLDKQLKGKIQNGKRYNSAPKSGEIASWLVNDMMRFQPVQRDENGEPLNNSKANSTEYQLLQRTLAFFGSEHERLAPYFKQTKLIESSNPHPFLNDTEWEKCSNILSFYRSYLKAKKNFLESLKPEDWEKNQYFLMLKEPKTNRKTLVQGWKNGFNLPRGIFTEPIRKWFMEHWKNIKVDDSKRVGLVAKVIPLFFSEKYKDSVQPFYNYPFNVGDVNKPKEEDFLHREERIELWDKKKDKFKGYKAKKKFKEMTDKEKEEHRSYLEFQSWNKFERELRLVRNQDIVTWLLCTELINKLKIDELNIKELKKLRLKDINTDTAKEEKNNILNRVMPMELPVTVYEVNKGGYIIKNKPLHTIYIKEAKTKLLKQGNFKALVKDRRLNGLFSFVKTPSEAESESNPISKSRVESELGKYQNARLNIIEDMLALEKTLIDKYNSLDTDNFHNMLTGWLKLKGKANKARFQNEVDLLVAVRNAFSHNQYPMYNENLFGNIERFSLSSSNIIESKGLDIAVKLKEEVSKAAKKIQNEEDNKKEKET

SEQ ID NO: 2 - protein, 1098 aa, synthetic construct
  REGION 1..1098: note = Description of Artificial Sequence: Synthetic polypeptide
MENDKKLEESACYTLNDKHFWAAFLNLARHNVYITVNHINKTLGEGEINRDGYETTLENTWNEIKDINKKARLRELIIKHFPFLEAATYQQRSTDSTKQKEEKQAEAQSLESLKHCLFPFLKKLQKSRDHYSHYKHSKSLERPKFEEDLQKKMYNIFDVSIRLVKEDYKHNTDINLKEDFKHLNRTGKFKYSFADNKGNITESGLLFFISLFLEKKDAIWMQKKLKGFKDSREKYQKMTNEVFCRSRILLPKLRLESTQTQDWILLDMLNELIRCPKSLYERLREKERKEFKVPIEIADEDYDAEQEPFRNTLVRHQDRFPYFALRYFDYNEIFTNLRFQIDLGTYHFSIYKKLIGGNKEDRHLTHKLYGFERIQEFAKQNRPDEWKALVKDLDTFNKEEEKLYISETTPHYHLENEKIGIVFKNHNIWPSTQTELTNNNRKKYNLGVSIKAEAFLSVHELLPMMFYYLLLKTKNTHNGNEVEAKKKGTKNKKQEKHKIEAIIESKIKDIYNLYDAFANGEINSIEELEEHCKGKDIEIGHLPKQMIAILKDEHKDMAKKAETKQEKMILATENRLKTLDKKLKGKIRNGKRCNSALKSGEIASWLVNDMMRFQPVQRDEEGKPLNNSKANSTEYQLLQRTLAFFGSEHERLDPYFKQMNLIGDNNPHPFLNDTEWEKCNNILSFYRSYLKARKNFLESLKPEDWEKNQYFLMLKEPKTNRKTLVQGWKNGFNLPRGIFTEPIRKWFMEHWKSIKVDDLKRVGLVAKVIPLFFSEKYKDSVQPFYNYPFNIGNINKPEEKDFLDYEKRKKLWDKKQAKFKGKTDKEKREDPSYPDFKSWKKFERELRLVRNQDIVTWMLCMELFEEQKVDGLNTGQIHLKDIVTNVDEEGNNNILNQIKPMQLPITVYDKENSPKRKPLHTIYIEETGTKLLKQGNFKALVKDRRLNGLFSFVKTSSEAESKSKPISKLRVEYELGAYQKARIDIIKDMLALEKTLIDNDENLPTNKFSDMLKSWLKGKGEANKARLQNDVDLLVAVRNAFSHNQYPMYNSEVFKGMKLLSLSSDIPEKEGLGIAKQLKDKIKETIERIIEIEKEIRN

SEQ ID NO: 3 - protein, 1091 aa, synthetic construct
  REGION 1..1091: note = Description of Artificial Sequence: Synthetic polypeptide
MKEEEKGKTPVVSTYNKDDKHFWAAFLNLARHNVYITVNHINKILGEAEINIDGYDISLKNIWSGIRDINKKARLEELMIKHFPFLEAMTHQQRLTGNTKQKEEKQAKIQSFEILKDNLFLLLKMLQDLRNHYSHYKHSKSLERPKFDEDIRNKLYNIFDTSIRLVKEDYKHNPDIKPKEHFKHLDRKRNEKFRYSFADNEGNITISGLLFFVSLFLEKKDAIWVQKKLEGFRCSNESYQKMTNEVFCRNRILMPKLRLESTQTQDWILLDMLNELIRCPKSLYERLKGEDRKKFEVPFDSTDEDYDAEQDPFKNTLIRYQDRFPYFVLRYFDYNEIFENLRFQIDLGTYHFSIYKKLIGGQKEDRHLTHKLYGFERLQEFAKQNRPDEWKAIIKDLDTFKKQEDEKVNEKPYISETTPHYHLENEKIGIAFKNHNIWPTTNTEFTNNKRKKYNLGTSIKAEAFLSVHELLPMMFYYMLLKIKNEGPRKVEAIIESKIKDIFTLYDAFANDEIDNTDELEKYCEDKDIKIGHLPKQMIAILYNKHKDMVEKAKVKQEKMKLATENRLKTLDKLIEGKMKYAKRGISPLKSGEIASWLVNDMMRFQPVQRDENGEPLNNSKANSTEYQLLQRTLALFGSEHKRLTPYFKQMNLIGDNNSHPFLKQTEWEECNNILSFYRSYLEARKNFLKNLEPKDWKKNQYFLKIKEPKSNRQTLVQGWKNGFNLPRGIFTEPIKKWFIEHRKKMKVDELKSVGLVAKVIPLYFNEKYKDSVQPFYNYPFNVGDINKPKEKDFLDNGKRRKLWDKKQLEFKGKTEEEKRKNPSYLDFKSWKKFERELRLIRNQDIVTWLMCIELYNNQKVENLNTGHIYLKDIITNVKEERNNNILNQIKSMQLPVTVYENESSPKKKYLRTIYIEENGTKLLKQGNFKALVKDRRLNGLFSFVKTSSEAESKRNPISKLRVEHELREYQKARIDIIKDMLALEKTLIDNYDSLDTDNFHNMLTGWLELKGEAIKTKFNNDVKLLTAVRNAFSHNQYPMYDENLFINIERFSLSSSRILESKGLGIAVKLKEEVSNAIEKIKNEEDAKKET

SEQ ID NO: 4 - protein, 1158 aa, synthetic construct
  REGION 1..1158: note = Description of Artificial Sequence: Synthetic polypeptide
MKNQNSLPSNPTDILEDKPFWAAFFNLARHNVYLTVNHINKLIDLEKLYDEDKHEEIFEREDILNISDDVMNDVNSNGKKRKLDIKKIWDDLDTDLTRKYLLRELILKHFPFIQPAIIGAQTKERTTIDKDKRNTSTSNDSLKQTGEGDINDPLSLSNVKSMFFSLLQMLEQLRNYYSHVKHSKSATMPNFDEDLLNWMRYIFIDSVNKVKEDYSSNSVIDPNTCFSHLIYKDKQGKIKPCRYPFTSKDGSINAFGLLFFVSLFLEKQDSIWMQKKIPGFKKASENYMKMTNEVFCRNHILLPKIRLETVYDKDWMLLDMLNEVVRCPLSLYKRLTPADQNKFKVPEKSSDNANRQEDDNPFSRILVRHQNRFPYFVLRFFDLNEVFTTLRFQINLGCYHFAICKKQIGDKKEVHHLTRTLYGFSRLQNFTQNTRPEEWNTLVKTTEPSSGNDGKTVQGVPLPYISYTIPHYQIENEKIGIKIFDGDTAVDTDIWPSVSTEKQLNKPDKYTLTPGFKADVFLSVHELLPMMFYYQLLLCEGMLKTDAGNAVEKVLIDTRNAIFNLYDAFVQEKINTITDLENYLQDKPILIGHLPKQMIDLLKGHQRDMLKAAEQKKAMLIKDTERRLERLNKQPEQKPNVAAKNIGALLRNGQIADWLVKDMMRFQPVKRDKEGNPINCSKANSTEYQMLQRAFAFYATDSCRLPRYFEQLHLINCDNSHLFLSRFEYDKQPNLIAFYAAYLKAKLDFLNELQPQNWVSDNYFLLLRAPKNNRQKLAEGWKNGFNLPRGLFTEKIKTWFNEHKTIVDISDCDIFKNRVGQVARLIPVFFDKKFKDHSQPFYRYNFNVGNVSKPTEAKYLSKEKREELFKSYQNKFKNNIPAEKTKEYREYKNFSLWKKFERELRLIKNQDILTWLMCKNLFDEKIEQGIDIPYIKLDSLQTNTSTKGSLNALAQVLPMVLAIYIGNSESNNGTGANEEENKGPMVYIKEEGTKLLKWGNFKTLLADRRIKGLFSYIEHDDIDLKQHPLTKRRVDLELDLYQTCRIDIFQQTLGLEAQLLNKYSDLNTDNFYQMLIGWRKKEGIPRDIKEDTDFLKDVRNAFSHNQYPDSKKIAFSRIRKFNPKKTILNEKKGLGIAKQMYEEVEKVVNRIKGIELFD

SEQ ID NO: 5 - protein, 1165 aa, synthetic construct
  REGION 1..1165: note = Description of Artificial Sequence: Synthetic polypeptide
MKNQNTLPSNPTDILKDKPFWAAFFNLARHNVYLTVNHINKLLDLEKLYNKDKHKEIFEHEDIFNISDDVMNDVNSNGKKRKLDIKKIWANLDTDLTRKYQLRELILKHFPFIQPAIIGAQTKERTTIDKDKRSTSTSNDSLKPTGEGDINDPLSLSNVKSIFFRLLQMLEQLRNYYSHVKHSKSATMPNFDEGLLKSMYNIFIDSVNKVKEDYSSNSVIDPNTSFSHLISKDEQGEIKPCRYSFTSKDGSINASGLLFFVSLFLEKQDSIWMQKKIPGFKKTSENYMKMTNEVFCRNHILLPKMRLETVYDKDWMLLDMLNEVVRCPLSLYKRLAPADQNKFKVPEKSSDNANRQEDDNPFSRILVRHQNRFPYFALRFFDLNEVFTTLRFQINLGCYHFAICKKQIGDKKEVHHLTRTLYGFSRLQNFTQNTRPEEWNTLVKTTEPSSGNDGKTVQGVPLPYISYTIPHYQIENEKIGIKIFDGDTAVDTDIWPSVSTEKQLNKPDKYTLTPGFKADVFLSVHELLPMMFYYQLLLCEGMLKTDAGNAVEKVLIDTRNAIFNLYDAFVQEKINTITDLENYLQDKPILIGHLPKQMIDLLKGHQRDMLKAVEQKKAMLIKDTERRLERLNKQPEQKPNVAAKNTGTLLRNGQIADWLVKDMMRFQPVKRDKEGNPINCSKANSTEYQMLQRAFAFYTTDSYRLPRYFEQLHLINCDNSHLFLSRFEYDKQPNLIAFYAAYLEAKLEFLNELQPQNWASDNYFLLLRAPKNDRQKLAEGWKNGFNLPRGLFTEKIKTWFNEHKTIVDISDCDIFKNRVGQVARLIPVFFDKKFKDHSQPFYTYNFNVGNVSKITEANYLSKEKRENLFKSYQNKFKNNIPAEKTKEYREYKNFSSWKKFERELRLIKNQDILTWLMCKNLFDEKIKPKKDILEPRIAVSYIKLDSLQTNTSTAGSLNALAKVVPMTLAIHIDSPKPKGKAGNNEKENKEFTVYIKEEGTKLLKWGNFKTLLADRRIKGLFSYIEHDDINLEKYPLTKYQVDSELDLYQKYRIDIFKQTLDLEAQLLDKYSDLNTDNFNQMLSGWSEKEGIPRNIKQDVAFLIGVRNGFSHNQYPDSKRIAFSRIKKFNPKTSSLQESEGLNIAKQMYEEAQQVVNKIKNIESFD

SEQ ID NO: 6 - protein, 1168 aa, synthetic construct
  REGION 1..1168: note = Description of Artificial Sequence: Synthetic polypeptide
MKITKIDGISHKKYIKEGKLVKSTSEENKTDERLSELLTIRLDTYIKNPDNASEEENRIRRENLKEFFSNKVLYLKDGILYLKDRREKNQLQNKNYSEEDISEYDLKNKNSFLVLKKILLNEDINSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIRECYHKIIGRKNDKENFAKIIYEEIQNVNNIKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIAGNRQNEAFLRNIIGVSSVAYFSLRNILETENKDDITGKMRGKTRIDSKTGEEKYIPGEVDQIYYENKQNEVKNKLKMFYGYDFDMDNKKEIEDFFANIDEAISSIRHGIVHFNLDLDGKDIFAFKNIVPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISREIIELNKNDKRNLKTGFYKLQKFEDIQEKTPKKYLANIQSLYMINAGNQDEEEKDTYIDFIQKIFLKGFMTYLANNGRLSLMYIGNDEQINTSLAGKKQEFDKFLKKYEQNNNIEIPHEINEFLREIKLGKILKYTESLNMFYLILKLLNHKELTNLKGSLEKYQSANKEETFSDELELINLLNLDNNRVTEDFELEANEIGKFLDFNGNKIKDRKELKKFDTKKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENQYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSVVDILKEYGFVATFKIGADKKIGIQTLESEKIVHLKNLKKKKLMTDRNSEELCKLVKVMFEYKMEEKNLKTKKCKVI

SEQ ID NO: 7 - protein, 1168 aa, synthetic construct
  REGION 1..1168: note = Description of Artificial Sequence: Synthetic polypeptide
MKITKIDGILHKKYIKEGKLVKSTSEENKTDERLSELLTIRLDTYIKNPDNASEEENRIRRETLKEFFSNKVLYLKDSILYLKDRREKNQLQNKNYSEEDISEYDLKNKNSFLVLKKILLNEDINSEELEIFRNDFEKKLDKINSLKYSLEENKANYQKINENNIKKVEGKSKRNIFYNYYKDSAKRNDYINNIQEAFDKLYKKEDIENLFFLIENSKKHEKYKIRECYHKIIGRKNDKENFATIIYEEIQNVNNMKELIEKVPNVSELKKSQVFYKYYLNKEKLNDENIKYVFCHFVEIEMSKLLKNYVYKKPSNISNDKVKRIFEYQSLKKLIENKLLNKLDTYVRNCGKYSFYLQDGEIATSDFIVGNRQNEAFLRNIIGVSSTAYFSLRNILETENENDITGRIKGKTVKNKKGEEKYISGEIDKLYDNNKQNEVKKNLKMFYSYDFNMNRKKEIEDFFSNIDEAISSIRHGIVHFNLELEGKDIFTFKNIVPSQISKKMFQNEINEKKLKLKIFRQLNSANVFRYLEKYKILNYLNRTRFEFVNKNIPFVPSFTKLYSRIDDLKNSLCIYWKIPKANDNNKTKEITDAQIYLLKNIYYGEFLNYFMSNNGNFFEIIKEIIELNKNDKRNLKTGFYKLQKFENLQEKTPKEYLANIQSFYMIDAGNKDEEEKDAYIDFIQKIFLKGFMTYLANNGRLSLMYIGNDEQINTSLAGKKQEFDKFLKKYEQNNNIEIPHEINEFVREIKLGKILKYTESLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGILNLLEKISDEAKYKISIEELKNYSNKKIEIEKNHTTQENLHRKYARPRKDEKFNDEDYKKYEKTIRNIQQYTHLKNKVEFNELNLLQSLLLRILHRLVGYTSIWERDLRFRLKGEFPENQYIEEIFNFDNSKNVKYKNGQIVEKYISFYKELYKDDMEKISIYSDKKVKELKKEKKDLYIRNYIAHFNYIPNAEVSLLEVLENLRKLLSYDRKLKNAIMKSIVDILKEYGFVVTFKIEKDKKIRIESLKSEEVVHLKKLKLKDNDKKKEPIKTYRNSKELCKLVKVMFEYKMKEKKSEN

SEQ ID NO: 8 - protein, 1194 aa, synthetic construct
  REGION 1..1194: note = Description of Artificial Sequence: Synthetic polypeptide
MKVTKIDGISHKKFEDEGKLVKFTGHFNIKNEMKERLEKLKELKLSNYIKNPENVKNKDKNKEKETKSRRENLKKYFSEIILRKKEEKYLLKKTRKFKNITEEINYDDIKKRENQQKIFDVLKELLEQRINENDKEEILNFDSVKLKEAFEEDFIKKELKIKAIEESLEKNRADYRKDYVELENEKYEDVKGQNKRSLVFEYYKNPENREKFKENIKYAFENLYTEENIKNLYSEIKEIFEKVHLKSKVRYFYQNEIIGESEFSEKDEEGISILYKQIINSVEKKEKFIEFLQKVKIKDLTRSQIFYKYFLENEELNDENIKYVFSYFVEIEVNKLLKENVYKTKKFNEGNKYRVKNIFNYDKLKNLVVYKLENKLNNYVRNCGKYNYHMENGDIATSDINMKNRQTEAFLRSILGVSSFGYFSLRNILGVNDDDFYKIEKDERKNENFILKKAKEDFTSKNIFEKVVDKSFEKKGIYQIKENLKMFYGNSFDKVDKDELKKFFVNMLEAITSVRHRIVHYNINTNSENIFDFSNIEVSKLLKNIFEKEIDTRELKLKIFRQLNSAGVFDYWESWVIKKYLENVKFEFVNKNVPFVPSFKKLYDRIDNLKGWNALKLGNNINIPKRKEAKDSQIYLLKNIYYGEFVEKFVNDNKNFEKIVKEIIEINRGAGTNKKTGFYKLEKFETLKANTPTKYLEKLQSLHKISYDKEKIEEDKDVYVDFVQKIFLKGFVNYLKKLDSLKSLNLLNLRKDETITDKKSVHDEKLKLWENSGSNLSKMPEEIYEYVKKIKISNINYNDRMSIFYLLLKLIDYRELTNLRGNLEKYESMNKNKIYSEELTIINLVNLDNNKVRTNFSLEAEDIGKFLKSSITIKNIAQLNNFSKIFADGENVIKHRSFYNIKKYGILDLLEKIVAKADLKITKEEIKKYENLQNELKRNDFYKIQEQIHRNYNQKPFSIKKIENKKDFEKYKKVIEKIQDYTQLKNKIEFNDLNLLQSLIFRILHRLAGYTSLWERDLQFKLKGEFPEDKYIDEIFNSDGNNNQKYKHGGIADKYANFLIEKKEEKSGEILNKKQRKKKIKEDLEIRNYIAHFNYLPNAEKSILEILEELRELLKHDRKLKNAVMKSIKDIFREYGFIVEFTISHTKNGKKIKVCSVKSEKIKHLKNNELITTRNSEDLCELVKIMLEHKELQK

SEQ ID NO: 9 - protein, 1145 aa, synthetic construct
  REGION 1..1145: note = Description of Artificial Sequence: Synthetic polypeptide
MCMKITKIDGISHKKYKEKGKLIKNNDTAKDIIEERFNDIEKKTKELFQKTLDFYVKNYEKCKEQNKERREKAKNYFSKVKILVDNKKITICNENTEKMEIEDFNEYDVRSGKYFNVLNKILNGENYTEEDLEVFENDLQKRTGRIKSIKNSLEENKAHFKKESINNNIIYDRVKGNNKKSLFYEYYRISSKHQEYVNNIFEAFDKLYSNSHEAMNNLFSEITKDSKDRNIRKIREAYHEILNKNKTEFGEELYKKIQDNRNNFDKLLEIEPEIKELTKSQIFYKYYIDKVNLDETSIKHCFCHLVEIEVNQLLKNYVYSKRNINKEKLENIFEYCKLKNLIKNKLVNKLNNYIRNCGKYNAYISNNDVVVNSEKISEIRTKEAFLRSIIGVSSSAYFSLRNILNTDNTQDITNKVDKEVDKLYQENKKIELEERLKLFFGNYFDINNQQEIKVFLMNIDKIISSIRHEIIHFKMETNAQNIFDFNNVNLGNTAKNIFSNEINEEKIKFKIFKQLNSANVFDYLSNKDITEYMDKVVFSFTNRNVSFVPSFTKIYNRVQDLANSLEIKKWKIPDKSEGKDAQIYLLKNIYYGKFLDEFLNEENGIFISIKDKIIELNRNQNKRTGFYKLEKFEKIEETNPKKYLEIIQSLYMINIEEIDSEGKNIFLDFIQKIFLKGFFEFIKNNYNYLLELKKIQDKKNIFDSEMSEYIAGEKTLEDIGEINEIIQDIKITEIDKILNQTDKINCFYLLLKLLNYKEITELKGNLEKYQILSKTNVYEKELMLLNIVNLDNNKVKIENFKILAEEIGKFIEKINIEEINKNKKIKTFEELRNFEKGENTGEYYNIYSDDKNIKNIRNLYNIKKYGMLDLLEKISEKTNYCIKKKDLEEYSELRKQLEDEKTNFYKIQEYLHSKYQQKPKKILLKNNKNDYEKYKKSIENIEKYVHLKNKIEFNELNLLQSLLLKILHRLVGFTSIWERDLRFRLIGEFPDELDVEDIFDHRKRYKGTGKGICKKYDRFINTHTEYKNNNKMENVKFADNNPVRNYIAHFNYLPNPKYSILKMMEKLRKLLDYDRKLKNAVMKSIKDILEEYGFKAEFIINSDKEIILNLVKSVEIIHLGKEDLKSRRNSEDLCKLVKAMLEYSK

SEQ ID NO: 10 - protein, 1193 aa, synthetic construct
  REGION 1..1193: note = Description of Artificial Sequence: Synthetic polypeptide
MKVTKIDGLSHKKFEDEGKLVKFKNNKNINEIKERLKKLKELKLDNYIKNPENVKNKDKDAEKETKIRRTNLKKYFSEIILRKEDEKYILKKTKKFKNINQEIDYYDVKSKKNQQEIFDVLKEIKIKETEKEEIITFDSEKLKKVFGEDFVKKEAKIKAIEKSLKINKANYKKDSIKIGDDKYSNVKGENKRSCIYEYYKKSENLKKFEENIREAFEKLYTEENIKELYSKIEEVLKKTHLKSIVREFYQNEIIGESEFSKKNGDGISILYNQIKDSIKKEENFIEFIENIGNLELKDLTKSQIFYKYFLENEELNDENIKFAFCYFVEIEVNNLLKENVYKIKRFNEGNKKRIKNIFEYDKLKKLIVYKLENKLNNYVRNCGKYNYHMENGDIATSDINMRNRQTEAFLRSIIGVSSFGYFSLRNILGVNDDDFYEIEEGLTEEERKNESNVLKKAKEDITSKSIFEKVVDKSFEKKGIRSIKENLKMFYGDSFDKANGDELKQFFVNMLNAITSIRHSVVHYDMNTNSENIFNFSDIEVSRLLKSIFEKETDKRELKLKIFRQLNSAGVFDYWENWKIKKYLENIKFEFVNKNIPFVPSFTKLYNRIDNLKGSNALNLGYINIPKRKEARDSQIYLLKNIYYGKFVENFVNNKENFEKVFKKIIEINKNAGRNKQTNFYKLEKFETLKANTPTEYLEKLQSLHKINYDKEKVEEDKDTYVDFVQKIFLKGFINYLQKSNSLKPLNLLNLKKDEVINSEKSSYDEKLKQWENNGSKLSEMPKEIYEYIKKIQINKINYSDRMSIFYLLLKLIDHKELTNLRGNLEKYESMNKNKIYSEELNIVNLVSLDNNKVRTNFSVEAEDIGKFLKTETSIKNNKQLNNFSEIFADGQNVIKHRSFYNIKKYGILDLLEKIVAKADLKITKEEIKKYKNLQKELEKNDFYKIQEKIHRKYNQKPNLISRTENKKDFNDYKKAIENIQNYTQLKNKIEFNDLNLLQDLLFRILHRLAGYTSLWERDLQFKLKGEFPEDKYIDEIFNSDRNNNQKYKSGGIAYKYVDFLIEKEEGKRAGKNKVKKRSEKEGSFIIRNYIAHFNYIPDAEKSILEILEELRELLKYDRKLKNAVMKSIKDIFKEYGFIIEFGISHESNSKKIKVVNVESEKIKHLKNNGLVTTRNSEDLCNLVKVMLEYKKS

SEQ ID NO: 11 - protein, 1210 aa, synthetic construct
  REGION 1..1210: note = Description of Artificial Sequence: Synthetic polypeptide
MKITKRKWEDVPPLFFHRDKDSGRLIRQDDREHDYTDELFRKTTVDSIFIRSLQNRLLRSQDKEGNKIGRNEITELVENLCKGAEIQKSIDEIKDSKSNVKALCDKTLTDYGKSFAAEEGRTTLYEAILNLAKGNAIQKAMAEAELKQIHEALKKEKEDRINRVQNSIKNNKIPLKVNEDGTISANNDRASWLLELLKPVASEHDKGDRYPVLTEMEGIVSFDKLCEEVRNKIHGRKGRTRSIAMSVDEAVKEYLKELWKKHADRHRDMRYYFQAVKEYFKENFPIRTKQKGAGIRQSLLKEDTVVARLLDPKHMAKAVRRKLINQSIQMHILYGKLYEYCCREDRNLPANSETLQLIQVLEAFKKQLMTAVLWSVSRLCYFYEFYSGDILLAEEYRRDFLNQKNDHKDIQACKEKLQDFFPLAELQEKIRECADKDINNIKDKITNDVLACNNLLEECVFCISKLRNNIFHYKNLSFTQALKHIANENKEEDIPIILRLYKQDCENVNKAFANRISSMNLPLYYSTEFLLRIFKKQGAELSLYSTKNQMTPSFKRVYERGKNLRLEYEREPMRSNIANEYDKDPQSDYWMRWFSQFATGTVDTNAGIKGEMEDYPHASVNLGIEIEAGTNIDAQRAMRNLLQLIYKHHFLPEVQKDETVVTDVIPKVLERNRKLSEVRNRSDKTYGYKEIEELCKKNKGKKDSLSELMKDLQRKVSETEKEKRELAREKTDYAQRFILDIFAEAFCNFMDERYEDEYQEIMSPIKNEEEAKKWTEMFTDKADKDNKDKLLVLKTSFDKDNVEKYLLVLYPLMRLLDDKELSELQQQMLRFRASVSGWQGGTDFSEDIAMAEKIEELAELVKLTEPESQYAEEAWKNRAIGVFEQFVEGKMAEYGTFYIQNDSLTQVPRGNMRKLLRSGVIGVYNKVLASCKQATKADYGAYSGDHWSIEDPDGNPIKSAEQAQALLQRLHKKYTESPSQFSDDDHKLYDKTLRQLDAYNRAMKNLSFGSLYEICRIHMETLSRWVGFVQDWERDMYFLLLAWTKQGKLLNVTETDVIEIFEKGSVIYKMNKKLKNGDRYAFTSIYRNNNDEKLEFLQIRNFIAHLHLLRKSEWKGDKKAGYSVIEDYINKLRFLLSYDQKRMNAVTKAMQQIFEKHKVIVKFMVEKGGMLKVKDVKPGIIEHLKQTKRIEIPCHGERFTDSLLVLMKYQ

SEQ ID NO: 12 - protein, 1160 aa, synthetic construct
  REGION 1..1160: note = Description of Artificial Sequence: Synthetic polypeptide
MEQKQLESRFNQIFNNKGHTGPIAEKAVKNFETIRQHKVSPRERLHYFAVGHALRNIDKDLKESIFEYNLDEEQKKQKPTQFTTLQSDFFRFENALLTLLKDIRNCNGHYVHTFDKLQLDEILKLQEKNKEHGILNKDAGCQIIEFLKEAFEFSILIQFLKEKPKEYEKFKKRKNENKNQSLRNLIGGYEKKLVKYLCDKFFPNEEKQKEIRDKFIEHNLEEAIEDLLFIPVDEDIEWKLGEEHVVFVIKKGKYLSFYAQLFLLSMFLYKQEANQLISKIRGFKRSEDEFQYKRNIFTFFSKKVSSQDIHSEEKHLIYFRDIIQYLNRFPTAWNEYLSPERKNLPMTKLLEKYILEEEIFRTFSTYKNDCNRELFLKYTIKRLFCKKAELFDAEKISIDDNLRKKFNYEIDTSPELKNIHEKLKGKLKPKDYYKNIKRKEELEKEENPEKLKLTKKVTEEKLFTAYGRNRDRFMDFAVRYLAEQNYFGKDAEFKMYMFETTNEQENYLKEQKNTADKKVIDQNKYHQGRLTCFKTYQKHKDDYQNWDDPFVFQNNAFQIILTFSNGERKKFSIQRKLLIYLLEDALFNHSDSIEDKGKQLLEDYFFNTLMPDFEDAKESYKTSDDVNWKHRKLLPKRLIYTVHPPRRTDSEEQIHPFEKILRETQEQERRYRLLLGKAKNMKLKEEFIKRNKGKHFKLRFIRKAWHLMYFREIYERRAKEHSHHKSFHITRDEINDFSRWMYAFDEVPPYKVYLRNMLQRKKFMENEEFAELFEKGKSLDDFYRITKKEFSKRIKNNLFQLKVDSERQYAEILSKKLVYINLSHFIKYLNAKGKLTVENGIIQYKASTNKKYLIDEYYYTEVLPREEYKVHKHLFNKLRATKLEDALLYELAMKYLREDNDIVEKAKSKVSDIQSSEISFDIKDYYGNHLYTLIVPFNKLETLSILIRYKTKQEKNSKLQRTSFLGNIYHLLAFLDKNYKQLSKYKDDGGFKKIVKNFKNKKRLSFVELNTINGYIISGAVKFSKVHMELERYFINKHKIKANHIYIDLEDIKDDSNKQVFGNYYDSKLRIRNKAFHFGVPTNFFFNIEIEKIEKKFILEEVKLQNVSSFDKLNQNAKSVCRVFMEVLHSDLYRRDKNKSKEELRREFEEKYFNEIITTV

SEQ ID NO: 13 - protein, 847 aa, synthetic construct
  REGION 1..847: note = Description of Artificial Sequence: Synthetic polypeptide
MMTKKPANRHALPKVIISEVDNTNILEFKIKYEKLARLDRVEVKAMHYEDGRIIFDEVVVNGGLIEVEYQDDHKTLFVQVGEKSYSISGQKVGGKQRLLEDRVSKTKVQLELSDGSSERVSRTERELIVADNIKLYSQIVGHEVKTTKEIYLAKRFLGYRSDLLFYYGFVDNFFRESKNLKYGKQPVELWEDKFQVNDKLTAYTKFMFNDDLQNSESYLKEYVKDNHKIKNDLESARDIFATFRHNLMHFNYSFFTRLFNGEDVKIKNLQTKKFESLSDVLRNVEFLNKVIQSIDKLNIDTRKEFIDKEKITLFNEELDLQQLYGFFAYTAINRVAFNKLINSFIIKDGIENEQLKEYFNQRVDGTAYEIDIHQNREYKELYKKHKNLVSKVSTLSDGKEIARGNTEISVLKEQMNKITKANSLKRLEHKLRLAFGFIYTEYGSYKAFVSRFNEDTKRKKIKNVEFEKIGVEKQKEYYESTFTSNNKDKLGELIQEYEKLSLNDLIENDTFLKVILLLFIFMPKEVKGDFLGFIKKYYHDTKHIEEDTKEKDEGFTNTLPIGLKLKIVERNIAKLSVLKHSLSLKVKYNRGQYEEDNTYRKVFKKLNISHNQEEFHKSMFSPLLRYYASLYKLINDFEIYTLSHYITDKYSTLNKVIASEQFHYRYGWNREEKKGELVKTDNYTFSTLLSKKYGHKNSQEISEMRNKISHFDEKILFKFPLEEVSSVPKGKGKYKKDEPIKSLKEKREEIVSLMEKQTDMQKVLGYDAINDFRMKTVQFQTKLKVYSNKEETIKKMIVEAKTPNDYYNIYKVKGVEGINEHLLNVIGETEAEKSIQEQIAEGNKVNV

SEQ ID NO: 14 - protein, 835 aa, synthetic construct
  REGION 1..835: note = Description of Artificial Sequence: Synthetic polypeptide
MEKIKKPSNRNSIPSIIISDYDANKIKEIKVKYLKLARLDKITIQDMEIVDNIVEFKKILLNGVEHTIIDNQKIEFDNYEITGCIKPSNKRRDGRISQAKYVVTITDKYLRENEKEKRFKSTERELPNNTLLSRYKQISGFDTLTSKDIYKIKRYIDFKNEMLFYFQFIEEFFNPLLPKGKNFYDLNIEQNKDKVAKFIVYRLNDDFKNKSLNSYITDTCMIINDFKKIQKILSDFRHALAHFDFDFIQKFFDDQLDKNKFDINTISLIETLLDQKEEKNYQEKNNYIDDNDILTIFDEKGSKFSKLHNFYTKISQKKPAFNKLINSFLSQDGVPNEEFKSYLVTKKLDFFEDIHSNKEYKKIYIQHKNLVIKKQKEESQEKPDGQKLKNYNDELQKLKDEMNTITKQNSLNRLEVKLRLAFGFIANEYNYNFKNFNDEFTNDVKNEQKIKAFKNSSNEKLKEYFESTFIEKRFFHFSVNFFNKKTKKEETKQKNIFNSIENETLEELVKESPLLQIITLLYLFIPRELQGEFVGFILKIYHHTKNITSDTKEDEISIEDAQNSFSLKFKILAKNLRGLQLFHYSLSHNTLYNNKQCFFYEKGNRWQSVYKSFQISHNQDEFDIHLVIPVIKYYINLNKLMGDFEIYALLKYADKNSITVKLSDITSRDDLKYNGHYNFATLLFKTFGIDTNYKQNKVSIQNIKKTRNNLAHQNIENMLKAFENSEIFAQREEIVNYLQTEHRMQEVLHYNPINDFTMKTVQYLKSLSVHSQKEGKIADIHKKESLVPNDYYLIYKLKAIELLKQKVIEVIGESEDEKKIKNAIAKEEQIKKGNN

SEQ ID NO: 15 - protein, 809 aa, synthetic construct
  REGION 1..809: note = Description of Artificial Sequence: Synthetic polypeptide
MIKNPSNRHSLPKVIISEVDHEKILEFKIKYEKLARLDRFEVKAMHYEGKEIVFDEVLVNGGLIEVEYQDDNKTLFVKVGEKSYSIRGKKVGGKQRLLEDRVSKTKVQLELSDGVVDNKGNLRKSRTERELIVADNIKLYSQIVGREVTTTKEIYLVKRFLAYRSDLLFYYSFVDNFFKVAGNEKELWKINFDDATSAQFMGYIPFMVNDNLKNDNAYLKDYVRNDVQIKDDLKKVQTIFSALRHTLLHFNYEFFEKLFNGEDVGFDFDIGFLNLLIENIDKLNIDAKKEFIDNEKIRLFGENLSLAKVYRLYSDICVNRVGFNKFINSMLIKDGVENQVLKAEFNRKFGGNAYTIDIHSNQEYKRIYNEHKKLVIKVSTLKDGQAIRRGNKKISELKEQMKSMTKKNSLARLECKMRLAFGFLYGEYNNYKAFKNNFDTNIKNSQFDVNDVEKSKAYFLSTYERRKPRTREKLEKVAKDIESLELKTVIANDTLLKFILLMFVFMPQELKGDFLGFVKKYYHDVHSIDDDTKEQEEDVVEAMSTSLKLKILGRNIRSLTLFKYALSSQVNYNSTDNIFYVEGNRYGKIYKKLGISHNQEEFDKTLVVPLLRYYSSLFKLMNDFEIYSLAKANPTAVSLQELVDDETSPYKQGNYFNFNKMLRDIYGLTSDEIKSGQVVFMRNKIAHFDTEVLLSKPLLGQTKMNLQRKDIVSFIEARGDIKELLGYDAINDFRMKVIHLRTKMRVYSDKLQTMMDLLRNAKTPNDFYNVYKVKGVESINKHLLEVLAQTAEERTVEKQIRDGNEKYDL

SEQ ID NO: 16 - protein, 840 aa, synthetic construct
  REGION 1..840: note = Description of Artificial Sequence: Synthetic polypeptide
MLKHKRKNKNSLARVVLSNYDSNNIYEIKIKYEKLAKLDKINIIEMDYDADNNVMFKKVLFNNKEIDLSHKDKTKINIELDNKKYNISAKKQIGKTHLVVRDKQTSKISRIKKIQDTYYRGKDVFILDNNIEILDKKQTKDKFIVTLNDITNDKTTSTEAELIDDTKDIFKKISAKKDLKSSDIYKIKRFISIRSNFSFYYTFVDNYFKIFHAKKDKNKEELYKIKFKDEINIKPYLENILDNMKNKNGILYDYADDREKVLNDLKNIQYVFTEFRHKLAHFDYNFLDNFFSNSVTDQYKQKVNEIKLLDILLDNIDSLNVVPKQNYIEDETISVFDAKDIKLKRLYTYYIKLTINYPGFKKLINSFFIQDGIENQELKEYINNKEKDTQVLKELDNKAYYMDISQYRKYKNIYNKHKELVSEKELSSDGQKINSLNQKINKLKIEMKNITKPNALNRLIYRLRVAFGFIYKEYATINNFNKSFLQDTKIKRFENISQQDIKNYLDISYQDKGKFFVKSKKTFKNKTTIKYTFEDLDLTLNEIITQDDIFVKVIFLFSIFMPKELNGDFFGFINMYYHKMKNISYDTKDIDMLDTISQNMKLKILEQNIKKTYVFKYYLDLDSSIYSKLVQNIKITEDIDSKKYLYAKIFKYYQHLYKLISDVEIYLLYKYNSKENLSITIDKDELKHRGYYNFQSLLIKNNINKDDAYWSIVNMRNNLSHQNIDELVGHFCKGCLRKSTTDIAELWLRKDILTITNEIINKIESFKDIKITLGYDCVNDFTQKVKQYKQKLKASNERLAKKIEEKQNQVVDEKNKEELEKKILNMKNIQKINRYILDIL

SEQ ID NO: 17 - protein, 1238 aa, synthetic construct
  REGION 1..1238: note = Description of Artificial Sequence: Synthetic polypeptide
MNQYIHANKKENKKRPNKSSIIRIMVSDFDDEYIQEIKVLYIKQGGVDTFKINKMSYDSASKKIIFEEVATQNMLSVEDSNLNFKRPMVECKNGDIYVVKPSEKVNEKGQAIEPLRSYKIHGKYLDLTEEIGKDNAEQSGKKQIYLHVEDLLGMHTTADSIDRRRLESETQRTLLSKEVMENYALIMGHEIKLDESDETYKLASSKEIYKANRFLDYRSRLLYYYSFINHFLVGLSKGATYEYAGKSLVAKIPDGEVWQLCELEVTAYHGIYINKKRNELVNENSLKIDAIYAQMAKNMKETINEFVDNYNAMIEKQNELKDNKSYKINKKAQNTTKFENFTEEKINQDLHNIVYILSDLRHKLMHFEYHYFELLMTGKKMGEKSEVIVCVPDRNSELPASKNEPSKDKFRKEKKLSELLDLNILKELDTFVKVKESYQTTYLETNDKIEILGKLKTAKSIYQIYHQICQRKNGFNKFINSFFTVDGEENTEVKDCINEVFRKEIHYFEMVIAKSNEDTLNDKNKNKSKKTRDRMKNQIQECKRYQDDESNIDTWVAYHKDIHYSKRYKKLYCEHITLVDNLNSAVSNGLNGQVIKEINDDIAIKKREMNEITKANSKSRLRYKMQMAYGFLFVEYGLKIPKFLNDFDLSHVRTASKIKGYKSPVKVTQYLTNDEGNKDNFNLETLMEDIDKKSKINFEFLKSNEDNNLIKLYILIYQLLPRELKGDFLGFVKNNYYDLKHVDFQTRETEAKDQFFHNMRLFEKNVKAFDLIQYSIGDEMSQLGNETFNFSQALSKIVSDDTILNSATEIPNINRLVYGSLLKYYENAFRLSSEIEIRALIKIARGKQIDNHSIDEAYKDALIFQKSGQTVKFSSILKYFNIDDLNKKNDSKIYNKASNLRNKIAHCDYTVLFITNIICEAENINVKAKYLIDVSNKIGLNTVDLGNDMVNDYLMSYDKQMTYLAKTSEELLKESSLDKSKEKKERRDNLKNETRSHSEIYMEYEWISDYLVKLKDNHEILSKKNKTEDLKLNRAYELIKNYNVALNKKSKIKIRALTTDHINNNWVNIWGALTHELQEIKGLYLYKVTPKIKKGIYFVLTDKKNYCLNINLYTLKKVPSKDDSEMTEERYLRDISEKYYFTVGEDSIKALKERLQTINDKCIITYVNEIDCKNENIKEKPEFKIAEDYNSWDKKYLSISNEELHHWSVETRRDKKYKENLILNLMEKSSFKLNLHQL

SEQ ID NO: 18 - protein, 849 aa, synthetic construct
  REGION 1..849: note = Description of Artificial Sequence: Synthetic polypeptide
MSQLKNPSNKNSLPRIIISDFNEIKINEIKIKYHKLDRLDKIIVKEMEIINNKIFFKKILFNNQIKDINSENIELENYILAGEVKPSNTKIILNRDGKEKSFIVYDGFTFKYKPNDKRISETKTNAKYILTIKDKTRHRESSTQRDILKSSIIETYKQISGFENITSKDIYTIKRYIDFKNEMMFYYTFIDDFFFPITGKNKQDKKNNFYNYKIKENAKKFISLINYRINDDFKNKNGILYDYLSNKEEIIINDFIHIQTILKDVRHAIAHFNFDFIQKLFDNEQAFNSKFDGIEILNILFNQKQEKYFEAQTNYIEEETIKILDEKELSFKKLHSFYSQICQKKPAFNKLINSFIIQDGIENKELKDYISQKYNSKFDYYLDIHTCKIYKDIYNQHKKFVADKQFLENQKTDGQKIKKLNDQINQLKTKMNNLTKKNSLKRLEIKFRLAFGFIFTEYQTFKNFNERFIEDIKANKYSTKIELLDYGKIKEYISITHEEKRFFNYKTFNKKTNKNINKTIFQSLEKETFENLVKNDNLIKMMFLFQLLLPRELKGEFLGFILKIYHDLKNIDNDTKPDEKSLSELNISTALKLKILVKNIRQINLFNYTISNNTKYEEKEKRFYEEGNQWKDIYKKLYISHDFDIFDIHLIIPIIKYNINLYKLIGDFEVYLLLKYLERNTNYKTLDKLIEAEELKYKGYYNFTTLLSKAINIALNDKEYHNITHLRNNTSHQDIQNIISSFKNNKLLEQRENIIELISKESLKKKLHFDPINDFTMKTLQLLKSLEVHSDKSEKIENLLKKEPLLPNDVYLLYKLKGIEFIKKELISNIGITKYEEKIQEKIAKGVEK

SEQ ID NO: 19 - protein, 779 aa, synthetic construct
  REGION 1..779: note = Description of Artificial Sequence: Synthetic polypeptide
ELCKIDFTDARSSSLIEYFKFAINDNLKNDRGYLKAYVNDVEQIRADLKKVGGKQRNLEDRVSRTKVQLTLTNHIEDREGKQRVSRTERELIVPQNIKLYSQIVGREVKTTKEIYLIKRFLEYRSDLLFYYGFVDNFFKVEGNKKELCKIDFTDARSSSLIEYFKFAINDNLKNDRGYLKAYVNDVEQIRTDLQKVKTIFSKLRHALMHFDYDFFEKLFNGEEVGFDFDIKFLNIMIDKVEKLNIETKKEFIEDEVITLFGERLSLKKLYGLFSHIAINRVAFNKFINSFLIKDGIENRALKDFFNDEKGSQAYEIDIHSNAEYKALYVQHKKLVMATSAMSDGNEIAKKNQEISELKEKMNAITKANSLARLEYKLRLAFGFIYTEYGDYTAFKNSFDRDVKSAKYKELSVERLKAYYLATFKASKPQSHEKLEEVAKKIDRLSLKQLIENETLLKFVLLLFTFMPQELKGEFLGFIKKYYHDKKHIEQDTKEKEEEREGLSTGLKLKVLEKNIRSLSILKHALSFQVKYNKKDKNFYEEGNLHGKFYKKLAISHNQEEFNKSVYAPLFRYYVALYKLINDFEIYSLAQHIVNNETLADQVGKAQFRQRGYFNFRKLVNCTYATAQNSSYNVLIFMRNDISHLSYEPLFNCPLEEKASYKQKIRGREKIISVKPLSESRAEIVRFIASQTDMKKLLGYDAVNDFNMKMVQLRRRLSVYANKQETIEKMINKAKTPNDFYNLYKLKGIECINQHLLKVIGVTEAEKRIEKQIEEGNEKY

SEQ ID NO: 20 - protein, 802 aa, synthetic construct
  REGION 1..802: note = Description of Artificial Sequence: Synthetic polypeptide
MLKKPSNRYALPKVILSTVDHEKILEFKVKYEKLARLDRLVVERMHFDGESVVFDEVIANSGDLEIAYQDDHRKLLIQAAGKSYTITGKKVGGKKRKLEERISRAKIQLTLTDGQEDQHRRIRATVTEKALLEPKEDRDIYSKISDRKIKTSKEIYLVKRFLSYRSDLLFYYFFVDNFFKVGNNKQELWKIKFQNQPELIEYFRFIINDRFKNAKNDKFDNYLKNDKAIQEDLEKIQKVFEKLRHALMHYDYGFFEKLFGGEDQGFDLDIAFLDNFVKKIDKLNIDTKKEFVDDEKIKIFGEDLNLADLYKLYASISINRVGFNRVVNEMIIKDGIEKSELKRAFEKKLDKTYALDIHSDPSYKKLYNEHKRLVTEVSTYTDGNKIKEGNQKIAKLKYEMKEITKKNALVRLECKMRLAFGLIYGRYDTHEAFKNGFDTDLKRGEFAQIGSEEAIGYFNTTFEKSKPKSKEEIKKIARQIDNLSLSTLIEDDPLMKFIVLMFLFVPRELKGEFLGFWRKYYHDIHSIDSDAKSDEMPDEVSLSLKLKILTRNIRRLNLFEYSLSEKIKYSPKNTQFYTDKSPYQKVYKRLKISHNKEEFDKTLLVPLFRYYSILFKLINDFEIYSLAKANPDASSLSELTKTKHGFRGHYNFTTLMMDAHKVSQGDSKKHFGIRGEIAHINTKDLIYDPLFRKSKMAQQRNDVIDFVLKYEKEIKAVLGYDAINDFRMKVVQLRTKLKVYSDKTQTIEKLLNEVEAPDDFYVLYKVKGVEAINKYLLEIVSVTQAEEEIERKIITGNKRYNT

SEQ ID NO: 21 - protein, 840 aa, synthetic construct
  REGION 1..840: note = Description of Artificial Sequence: Synthetic polypeptide
MLKHKRKNKNSLARVVLSNYDSNNIYEIKIKYEKLAKLDKINIIEMDYDADNNVMFKKVLFNNKEIDLSHKDKTKINIELDNKKYNISAKKQIGKTHLVVRNKQTSKISRIKKIQDTYYRGKDVFILDNNIEILDKKQTKDKFIVTLNDITNNKTTSTEAELIDDTKDIFKKISAKKDLKSSDIYKIKRFISIRSNFSFYYTFVDNYFKIFHAKKDKNKEELYKIKFKDEINIKPYLENILDNMKNKNGILYNYANDRKKVLNDLRNIQYVFKEFRHKLAHFDYNFLDNFFSNSVEEKYKQKVNEIKLLDILLDNIDSLNVVPKQNYIEDETISVFDAKDIKLKRLYTYYIKLTINYPGFKKLINSFFIQDGIENQELKEYINNKEKDTQVLKELDNKAYYMDISQYRKYKNIYNKHKELVSEKELSSDGKKINSLNQKINKLKIDMKNITKPNALNRLIYRLRVAFGFIYKEYATINNFNKSFLQDTKTKRFENISQQDIKSYLDISYQDKGKFFVKSKKTFKNKTTVKYTFEDLDLTLNEIITQDDIFVKVIFLFSIFMPKELNGDFFGFINMYYHKMKNISYDTKDIDMLDTISQNMKLKILEQNIKKTYVFKYYLDLDSSIYSKLVQNIKITEDIDSKKYLYAKIFKYYQHLYKLISDVEIYLLYKYNSKENLSITIDKDELKHRGYYNFQSLLIKNNINKDDAYWSIVNMRNNLSHQNIDELVGHFCKGCLRKSTTDIAELWLRKDILTITNEIINKIESFKDIKITLGYDCVNDFTQKVKQYKQKLKASNERLAKKIEEKQNQVVDEKNKEELEKNILNMKNIQKINRYILDIL

SEQ ID NO: 22 - protein, 845 aa, synthetic construct
  REGION 1..845: note = Description of Artificial Sequence: Synthetic polypeptide
MIKKPSNRHALPKVIISKVDNQNILEFKIKYKKLSRLDRVEIKTMHYDDRAIVFDEVIINGGLIDVEYRDNHKTIFVKVGDKSYSISGQKVGGKERLLENRISQTKVQLELKDEATNRVSKTERELIVDDNIKLYSQIVGRDVKTTKDIYLIKRFLGYRSDLLFYYGFVNNFFHVANNRPEFWKIDFNDNRNSKLIEYFIFTINDHLKNDENYLKDYISDRGQIVDDLENIKHIFSALRHGLMHFDYDFFEALFNGEDIDIKMDNQGNTQPLSSLNIKFLDIMIDKLDKLNIDTKKEFIDAEKITIFGEELSLAKLYRFYAHTAINRVAFNKLINSFIIENGVENQSLKEYFNQQAGGIAYEIDIHQNREYKNLYNEHKKLVSRVLSISDGQEIATLNQKIVELKEQMKQITKINSIKRLEYKLRLAFGFIYTEYKNYEEFKNSFDTDIKNGRFTPKDEDGNKRAFDSRELEHLKGYYKATLQTQKPQTDEKMEEVSKRVDRLSLKSLIGDDTLLKFILLMFTFMPQELKGEFLGFIKKYYHDTKHIDQDTISDSDDTIEEGLSIGLKLKILDKNIRSLSILKHSLSFQTKYNKKDRSYYEDGNIHGKFFKKLGISHNQEEFNKSVYAPLFRYYSALYKLINDFEIYTLSLHIVGNETLSDQVNKPQFLSGRYFNFRKLLTQSYNISNNSTHSVIFNAVINMRNDISHLSYEPLLDCPLNGKKSYKRKIRNQFRTINIKPLVESRKMIIDFITLQTDMQKVLGCDAVNDFTMKIVQLRTRLKAYANKEQTIEKMITEAKTPNDFYNIYKVKGVEAINKYLLEVIGETQVEKEIREEIERGNIANS

SEQ ID NO: 23 - protein, 822 aa, synthetic construct
  REGION 1..822: note = Description of Artificial Sequence: Synthetic polypeptide
MVKNPANRHALPKVIISEVDNNNILEFKIKYEKLARLDKVEVKSMHFDNNKQVVFDEVVINGGLIEPTYEDKHKKLVVTAGEKSYSIVGQKVGGKPRLLEDRVSKTKVQLELTNYVEDKEGKKRVSKTERELIVADNIELYSQIVGREVKTTKEIYLIKRFLEYRSDLLFYYGFVDNFFKVAGNGKELWKIDFTNSDSLHLIEYFKFSINDNLKNDENYLKNYVSDNTKIENDLVKCQNNFNSLRHALMHFDYDFFEKLFNGEDVGFDFDIEFLNIMIDKVDKLNIDTKKEFIDDEEVTLFGEALSLKKLYGLFSHIAINRVAFNKLINSFIIEDGIENKELKDFFNNKKESQAYEIDIHSNAEYKALYVQHKKLVMATSAMTDGDEIAKKNQEISDLKEKMKVITKENSLARLEHKLRLAFGFIYTEYKDYKTFKKHFDQDIKGAKYKGLNVEKLKEYYETTLKNSKPKTDEKLEDVAKKIDKLSLKELIDDDTLLKFVLLLFIFMPQELKGDFLGFIKKYYHDKKHIDQDTKDKDTEIEELSTGLKLKVLDKNIRSLSILKHSFSFQVKYNRKDKNFYEDGNLHGKFYKKLSISHNQEEFNKSVYAPLFRYYSALYKLINDFEIYALAQHVENHETLADQVNKSQFIQKSYFNFRKLLDNTDSISQSSSYNTLIVMRNDISHLSYEPLFNYPLDERKSYKKKTQKGVKTFHVELLYISRAKIIELISLQTDMKKLLGYDAVNDFNMKVVHLRKRLSVYANKEESIRKMQADAKTPNDFYNIYKVKGVESINQHLLKVIGVTEAEKSIEKQINEGNKKHNT

SEQ ID NO: 24 - protein, 832 aa, synthetic construct
  REGION 1..832: note = Description of Artificial Sequence: Synthetic polypeptide
MIKNPSNRYALPKVIISKIDNQNILEFKIKYKKLSKLDIVKVKSMHYDDRAIIFDEVIVNDGLIDVEYRDNHKTIFVKVGNKSYSISGQKVGGKERLLENRVSKTKVQLELKDKATNRVSKTERELIVDDNIKIYSQIVGRDVKTTKDIYLIKRFLAYRSDLLFYYGFVNNFFHVANNRSEFWKIDFNDSNNSKLIEYFKFTINDHLKNDENYLKDYISDNEKLKNDLIKVKNSFEKIRHALMHFDYDFFVKLFNGEDVGLELDIEFLDIMIDKLDKLNIDTKKEFIDDEKITIFGEELSLAKLYRFYAHTAINRVAFNKLINSFIIENGVENQSLKEYFNQQAGGIAYEIDIHQNREYKNLYNEHKKLVSRVLSISDGQEIAILNQKIAKLKDQMKQITKANSIKRLEYKLRLALGFIYTEYENYEEFKNNFDTDIKNGRFTPKDNDGNKRAFDSRELEQLKGYYEATIQTQKPKTDEKIEEVSKKIDRLSLKSLIADDILLKFILLMFTFMPQELKGEFLGFIKKYYHDTKHIDQDTISDSDDTIETLSIGLKLKILDKNIRSLSILKHSLSFQTKYNKKDRNYYEDGNIHGKFFKKLGISHNQEEFNKSVYAPLFRYYSALYKLINDFEIYTLSLHIVGSETLTDQVNKSQFLSGRYFNFRKLLTQSYHINNNSTHSTIFNAVINMRNDISHLSYEPLFDCPLNGKKSYKRKIRNQFKTINIKPLVESRKIIIDFITLQTDMQKVLGYDAVNDFTMKIVQLRTRLKAYANKEQTIQKMITEAKTPNDFYNIYKVQGVEEINKYLLEVIGETQAEKEIREKIERGNIANF

SEQ ID NO: 25 - protein, 806 aa, synthetic construct
  REGION 1..806: note = Description of Artificial Sequence: Synthetic polypeptide
MTKKPSNRNSLPKVIINKVDESSILEFKIKYEKLARLDRFEVRSMRYDGDGRIIFDEVVANAGLLDVDYEDDNRTIVVKIENKAYNIYGKKVGGEKRLNGKISKAKVQLILTDSIRKNANDTHRHSLTERELINKNEVDLYSKIAEREISTTKDIYLVKRFLAYRSDLLLYYAFINHYVRVNGNKKEFWKTEIDDKIIDYFIYTINDTLKNKEGYLEKYIVDRDQIKKDLEKIKQIFSHLRHKLMHYDFRFFTDLFDGKDVDIKVDNSIQKISELLDIEFLNIVIDKLEKLNIDAKKEFIDDEKITLFGQEIELKKLYSLYAHTSINRVAFNKLINSFLIKDGVENKELKEYFNAHNQGKESYYIDIHQNQEYKKLYIEHKNLVAKLSATTDGKEIAKINRELADKKEQMKQITKANSLKRLEYKLRLAFGFIYTEYKDYERFKNSFDTDTKKKKFDAIDNAKIIEYFEATNKAKKIEKLEEILKGIDKLSLKTLIQDDILLKFLLLFFTFLPQEIKGEFLGFIKKYYHDITSLDEDTKDKDDEITELPRSLKLKIFSKNIRKLSILKHSLSYQIKYNKKESSYYEAGNVFNKMFKKQAISHNLEEFGKSIYLPMLKYYSALYKLINDFEIYALYKDMDTSETLSQQVDKQEYKRNEYFNFETLLRKKFGNDIEKVLVTYRNKIAHLDFNFLYDKPINKFISLYKSREKIVNYIKNHDIQAVLKYDAVNDFVMKVIQLRTKLKVYADKEQTIESMIQNTQNPNGFYNIYKVKAVENINRHLLKVIGYTESEKAVEEKIRAGNTSKS

SEQ ID NO: 26 - protein, 835 aa, synthetic construct; identical to SEQ ID NO: 14
  REGION 1..835: note = Description of Artificial Sequence: Synthetic polypeptide

SEQ ID NO: 27 - protein, 847 aa, synthetic construct; identical to SEQ ID NO: 13
  REGION 1..847: note = Description of Artificial Sequence: Synthetic polypeptide

SEQ ID NO: 28 - RNA, 37 nt, synthetic construct
  misc_feature 1..37: note = Description of Artificial Sequence: Synthetic oligonucleotide
  source 1..37: mol_type = other RNA
agttgtgtctacctcctttttgagaggtaaaaacaac

SEQ ID NO: 29 - RNA, 36 nt, synthetic construct
  misc_feature 1..36: note = Description of Artificial Sequence: Synthetic oligonucleotide
  source 1..36: mol_type = other RNA
gttgcatctgcctgctgtttgcaaggtaaaaacaac

SEQ ID NO: 30 - RNA, 37 nt, synthetic construct
  misc_feature 1..37: note = Description of Artificial Sequence: Synthetic oligonucleotide
  source 1..37: mol_type = other RNA
gatttagactaccccaaaaacgaaggggactaaaacc

SEQ ID NO: 31 - RNA, 36 nt, synthetic construct
  misc_feature 1..36: note = Description of Artificial Sequence: Synthetic oligonucleotide
  source 1..36: mol_type = other RNA
gttttagtccccttcgtttttggggtagtctaaatc

SEQ ID NO: 32 - RNA, 36 nt, synthetic construct
  misc_feature 1..36: note = Description of Artificial Sequence: Synthetic oligonucleotide
  source 1..36: mol_type = other RNA
gttgtaactgcccttattttgaagggtaaacacaac

SEQ ID NO: 33-34 - skipped (2 entries)

SEQ ID NO: 35 - RNA, 10 nt, synthetic construct
  misc_feature 1..10: note = Description of Artificial Sequence: Synthetic oligonucleotide
  source 1..10: mol_type = other RNA
tttttttttt

SEQ ID NO: 36 - DNA, 10 bp, synthetic construct
  misc_feature 1..10: note = Description of Artificial Sequence: Synthetic oligonucleotide
  misc_feature 1..10: note = Description of Combined DNA/RNA Molecule: Synthetic oligonucleotide
  source 1..10: mol_type = other DNA
  misc_feature 1..4: note = DNA
  misc_feature 5..6: note = RNA
  misc_feature 7..10: note = DNA
tttttttttt

SEQ ID NO: 37-40 - skipped (4 entries)

SEQ ID NO: 41 - RNA, 30 nt, synthetic construct
  misc_feature 1..30: note = Description of Artificial Sequence: Synthetic oligonucleotide
  source 1..30: mol_type = other RNA
cgacctactctcccatactcttgtatatag

SEQ ID NO: 42 - RNA, 30 nt, unidentified
  misc_feature 1..30: note = Description of Unknown: Target 5S87 sequence
  source 1..30: mol_type = other RNA
ctatatacaagagtatgggagagtaggtcg

SEQ ID NO: 43 - protein, 18 aa, unidentified
  REGION 1..18: note = Description of Unknown: NLS sequence
KRPAATKKAGQAKKKKEF

SEQ ID NO: 44 - protein, 7 aa, Simian virus 40
PKKKRKV

SEQ ID NO: 45 - protein, 9 aa, unidentified
  REGION 1..9: note = Description of Unknown: C-myc NLS sequence
PAAKRVKLD

SEQ ID NO: 46 - RNA, 20 nt, unidentified
  misc_feature 1..20: note = Description of Unknown: Target C sequence
  source 1..20: mol_type = other RNA
catggcattccacttatcac

SEQ ID NO: 47 - protein, 11 aa, unidentified
  REGION 1..11: note = Description of Unknown: C-myc NLS sequence
RQRRNELKRSP

SEQ ID NO: 48 - protein, 38 aa, Homo sapiens
NQSSNFGPMKGGNFGGRSSGPYGGGGQYFAKPRNQGGY

SEQ ID NO: 49 - protein, 42 aa, unidentified
  REGION 1..42: note = Description of Unknown: IBB domain from importin-alpha sequence
RMRIZFKNKGKDTAELRRRRVEVSVELRKAKKDEQILKRRNV

SEQ ID NO: 50 - protein, 8 aa, unidentified
  REGION 1..8: note = Description of Unknown: Myoma T protein sequence
VSRKRPRP

SEQ ID NO: 51 - protein, 8 aa, unidentified
  REGION 1..8: note = Description of Unknown: Myoma T protein sequence
PPKKARED

SEQ ID NO: 52 - protein, 8 aa, Homo sapiens
PQPKKKPL

SEQ ID NO: 53 - protein, 12 aa, Mus musculus
SALIKKKKKMAP

SEQ ID NO: 54 - protein, 5 aa, Influenza virus
DRLRR

SEQ ID NO: 55 - protein, 7 aa, Influenza virus
PKQKKRK

SEQ ID NO: 56 - protein, 10 aa, Hepatitis delta virus
RKLKKKIKKL

SEQ ID NO: 57 - protein, 10 aa, Mus musculus
REKKKFLKRR

SEQ ID NO: 58 - protein, 20 aa, Homo sapiens
KRKGDEVDGVDEVAKKKSKK

SEQ ID NO: 59 - protein, 17 aa, Homo sapiens
RKCLQAGMNLEARKTKK

SEQ ID NO: 60 - RNA, 66 nt, synthetic construct
  misc_feature 1..66: note = Description of Artificial Sequence: Synthetic oligonucleotide
  source 1..66: mol_type = other RNA
gttagaatataaccctgtttgtaggggtaataaaaccgacctactctcccatactcttgtatatag

SEQ ID NO: 61 - RNA, 66 nt, synthetic construct
  misc_feature 1..66: note = Description of Artificial Sequence: Synthetic oligonucleotide
  source 1..66: mol_type = other RNA
ttgactacactctctatctcttagggagactgaaaccgacctactctcccatactcttgtatatag

SEQ ID NO: 62 - RNA, 60 nt, synthetic construct
  misc_feature 1..60: note = Description of Artificial Sequence: Synthetic oligonucleotide
  source 1..60: mol_type = other RNA
gactgaaccctaccatgtagggaattgaaccgacctactctcccatactcttgtatatag

SEQ ID NO: 63 - RNA, 66 nt, synthetic construct
  misc_feature 1..66: note = Description of Artificial Sequence: Synthetic oligonucleotide
  source 1..66: mol_type = other RNA
atttgactatacccctatttgagaggggaataaaaccgacctactctcccatactcttgtatatag

SEQ ID NO: 64 - RNA, 66 nt, synthetic construct
  misc_feature 1..66: note = Description of Artificial Sequence: Synthetic oligonucleotide
  source 1..66: mol_type = other RNA
attggaatataaccccgtttgtaggggtagtaaaaccgacctactctcccatactcttgtatatag

SEQ ID NO: 65 - RNA, 66 nt, synthetic construct
  misc_feature 1..66: note = Description of Artificial Sequence: Synthetic oligonucleotide
  source 1..66: mol_type = other RNA
gtttgaatataaccctgtttgtaggggtaataaaaccgacctactctcccatactcttgtatatag

SEQ ID NO: 66 - RNA, 66 nt, synthetic construct
  misc_feature 1..66: note = Description of Artificial Sequence: Synthetic oligonucleotide
  source 1..66: mol_type = other RNA
gatagaatataaccctgtttgtaggggtaataaaaccgacctactctcccatactcttgtatatag

SEQ ID NO: 67 - RNA, 37 nt, synthetic construct
  misc_feature 1..37: note = Description of Artificial Sequence: Synthetic oligonucleotide
  source 1..37: mol_type = other RNA
agtcagactataccctcgtttgtagggggaataaaac

SEQ ID NO: 68 - RNA, 37 nt, synthetic construct
  misc_feature 1..37: note = Description of Artificial Sequence: Synthetic oligonucleotide
  source 1..37: mol_type = other RNA
ggttagaatataaccctgtttgtaggggtaataaaac

SEQ ID NO: 69 - protein, 1159 aa, Leptotrichia buccalis
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 70 - RNA, 61 nt, synthetic construct
  misc_feature 1..61: note = Description of Artificial Sequence: Synthetic oligonucleotide
  source 1..61: mol_type = other RNA
gtttgaatataaccctgtttgtaggggtaataaaactactgacagccagacagcgaccaaa

SEQ ID NO: 71 - RNA, 61 nt, synthetic construct
  misc_feature 1..61: note = Description of Artificial Sequence: Synthetic oligonucleotide
  source 1..61: mol_type = other RNA
gtttgaatataaccctgtttgtaggggtaataaaacctgacagccagacagcgaccaaaag

SEQ ID NO: 72 - RNA, 61 nt, synthetic construct
  misc_feature 1..61: note = Description of Artificial Sequence: Synthetic oligonucleotide
  source 1..61: mol_type = other RNA
gtttgaatataaccctgtttgtaggggtaataaaacacagccagacagcgaccaaaagaat

SEQ ID NO: 73 - protein, 8 aa, synthetic construct
  REGION 1..8: note = Description of Artificial Sequence: Synthetic peptide
  MOD_RES 3: note = Any amino acid
  MOD_RES 4: note = W, F, or Y
  MOD_RES 5: note = Any amino acid
  MOD_RES 6: note = Any amino acid
KRXXXXAF

SEQ ID NO: 74 - protein, 6 aa, synthetic construct
  REGION 1..6: note = Description of Artificial Sequence: Synthetic peptide
  MOD_RES 5: note = K or R
  MOD_RES 6: note = W, F, or Y
LGKRXX

SEQ ID NO: 75 - DNA, 29 bp, synthetic construct
  misc_feature 1..29: note = Description of Artificial Sequence: Synthetic oligonucleotide
  misc_feature 1..29: note = Description of Combined DNA/RNA Molecule: Synthetic oligonucleotide
  source 1..29: mol_type = other DNA
  misc_feature 1..12: note = DNA
  misc_feature 13..17: note = RNA
  misc_feature 18..29: note = DNA
ttttttttttttttttttttttttttttt